SEQUENCE LISTING (16 sequences)

SEQ ID NO: 1 - protein, 585 aa, Homo sapiens
MAAAAAAVGPGAGGAGSAVPGGAGPCATVSVFPGARLLTIGDANGEIQRHAEQQALRLEVRAGPDSAGIALYSHEDVCVFKCSVSRETECSRVGKQSFIITLGCNSVLIQFATPNDFCSFYNILKTCRGHTLERSVFSERTEESSAVQYFQFYGYLSQQQNMMQDYVRTGTYQRAILQNHTDFKDKIVLDVGCGSGILSFFAAQAGARKIYAVEASTMAQHAEVLVKSNNLTDRIVVIPGKVEEVSLPEQVDIIISEPMGYMLFNERMLESYLHAKKYLKPSGNMFPTIGDVHLAPFTDEQLYMEQFTKANFWYQPSFHGVDLSALRGAAVDEYFRQPVVDTFDIRILMAKSVKYTVNFLEAKEGDLHRIEIPFKFHMLHSGLVHGLAFWFDVAFIGSIMTVWLSTAPTEPLTHWYQVRCLFQSPLFAKAGDTLSGTCLLIANKRQSYDISIVAQVDQTGSKSSNLLDLKNPFFRYTGTTPSPPPGSHYTSPSENMWNTGSTYNLSSGMAVAGMPTAYDLSSVIASGSSVGHNNLIPLGSSGAQGSGGGSTSAHYAVNSQFTMGGPAISMASPMSIPTNTMHYGS

SEQ ID NO: 2 - protein, 384 aa, Homo sapiens
MAAAAAAVGPGAGGAGSAVPGGAGPCATVSVFPGARLLTIGDANGEIQRHAEQQALRLEVRAGPDSAGIALYSHEDVCVFKCSVSRETECSRVGKQSFIITLGCNSVLIQFATPNDFCSFYNILKTCRGHTLERSVFSERTEESSAVQYFQFYGYLSQQQNMMQDYVRTGTYQRAILQNHTDFKDKIVLDVGCGSGILSFFAAQAGARKIYAVEASTMAQHAEVLVKSNNLTDRIVVIPGKVEEVSLPEQVDIIISEPMGYMLFNERMLESYLHAKKYLKPSGNMFPTIGDVHLAPFTDEQLYMEQFTKANFWYQPSFHGVDLSALRGAAVDEYFRQPVVDTFDIRILMAKSVKYTVNFLEAKEGDLHSACLASPAATALCLPG

SEQ ID NO: 3 - protein, 608 aa, Homo sapiens
MAAAAAAVGPGAGGAGSAVPGGAGPCATVSVFPGARLLTIGDANGEIQRHAEQQALRLEVRAGPDSAGIALYSHEDVCVFKCSVSRETECSRVGKQSFIITLGCNSVLIQFATPNDFCSFYNILKTCRGHTLERSVFSERTEESSAVQYFQFYGYLSQQQNMMQDYVRTGTYQRAILQNHTDFKDKIVLDVGCGSGILSFFAAQAGARKIYAVEASTMAQHAEVLVKSNNLTDRIVVIPGKVEEVSLPEQVDIIISEPMGYMLFNERMLESYLHAKKYLKPSGNMFPTIGDVHLAPFTDEQLYMEQFTKANFWYQPSFHGVDLSALRGAAVDEYFRQPVVDTFDIRILMAKSVKYTVNFLEAKEGDLHRIEIPFKFHMLHSGLVHGLAFWFDVAFIGSIMTVWLSTAPTEPLTHWYQVRCLFQSPLFAKAGDTLSGTCLLIANKRQSYDISIVAQVDQTGSKSSNLLDLKNPFFRYTGTTPSPPPGSHYTSPSENMWNTGSTYNLSSGMAVAGMPTAYDLSSVIASGSSVGHNNLIPLANTGIVNHTHSRMGSIMSTGIVQGSSGAQGSGGGSTSAHYAVNSQFTMGGPAISMASPMSIPTNTMHYGS

SEQ ID NO: 4 - protein, 651 aa, Homo sapiens
MLRLQMTDGHISCTAVEFSYMSKISLNTPPGTKVKLSGIVDIKNGFLLLNDSNTTVLGGEVEHLIEKWELQRSLSKHNRSNIGTEGGPPPFVPFGQKCVSHVQVDSRELDRRKTLQVTMPVKPTNDNDEFEKQRTAAIAEVAKSKETKTFGGGGGGARSNLNMNAAGNRNREVLQKEKSTKSEGKHEGVYRELVDEKALKHITEMGFSKEASRQALMDNGNNLEAALNVLLTSNKQKPVMGPPLRGRGKGRGRIRSEDEEDLGNARPSAPSTLFDFLESKMGTLNVEEPKSQPQQLHQGQYRSSNTEQNGVKDNNHLRHPPRNDTRQPRNEKPPRFQRDSQNSKSVLEGSGLPRNRGSERPSTSSVSEVWAEDRIKCDRPYSRYDRTKDTSYPLGSQHSDGAFKKRDNSMQSRSGKGPSFAEAKENPLPQGSVDYNNQKRGKRESQTSIPDYFYDRKSQTINNEAFSGIKIEKHFNVNTDYQNPVRSNSFIGVPNGEVEMPLKGRRIGPIKPAGPVTAVPCDDKIFYNSGPKRRSGPIKPEKILESSIPMEYAKMWKPGDECFALYWEDNKFYRAEVEALHSSGMTAVVKFIDYGNYEEVLLSNIKPIQTEAWEEEGTYDQTLEFRRGGDGQPRRSTRPTQQFYQPPRARN

SEQ ID NO: 5 - protein, 650 aa, Homo sapiens
MLRLQMTDGHISCTAVEFSYMSKISLNTPPGTKVKLSGIVDIKNGFLLLNDSNTTVLGGEVEHLIEKWELQRSLSKHNRSNIGTEGGPPPFVPFGQCVSHVQVDSRELDRRKTLQVTMPVKPTNDNDEFEKQRTAAIAEVAKSKETKTFGGGGGGARSNLNMNAAGNRNREVLQKEKSTKSEGKHEGVYRELVDEKALKHITEMGFSKEASRQALMDNGNNLEAALNVLLTSNKQKPVMGPPLRGRGKGRGRIRSEDEEDLGNARPSAPSTLFDFLESKMGTLNVEEPKSQPQQLHQGQYRSSNTEQNGVKDNNHLRHPPRNDTRQPRNEKPPRFQRDSQNSKSVLEGSGLPRNRGSERPSTSSVSEVWAEDRIKCDRPYSRYDRTKDTSYPLGSQHSDGAFKKRDNSMQSRSGKGPSFAEAKENPLPQGSVDYNNQKRGKRESQTSIPDYFYDRKSQTINNEAFSGIKIEKHFNVNTDYQNPVRSNSFIGVPNGEVEMPLKGRRIGPIKPAGPVTAVPCDDKIFYNSGPKRRSGPIKPEKILESSIPMEYAKMWKPGDECFALYWEDNKFYRAEVEALHSSGMTAVVKFIDYGNYEEVLLSNIKPIQTEAWEEEGTYDQTLEFRRGGDGQPRRSTRPTQQFYQPPRARN

SEQ ID NO: 6 - protein, 744 aa, Homo sapiens
MAQVAGAALSQAGWYLSDEGIEACTSSPDKVNVNDIILIALNTDLRTIGKKFLPSDINSGKVEKLEGPCVLQIQKIRNVAAPKDNEESQAAPRMLRLQMTDGHISCTAVEFSYMSKISLNTPPGTKVKLSGIVDIKNGFLLLNDSNTTVLGGEVEHLIEKWELQRSLSKHNRSNIGTEGGPPPFVPFGQKCVSHVQVDSRELDRRKTLQVTMPVKPTNDNDEFEKQRTAAIAEVAKSKETKTFGGGGGGARSNLNMNAAGNRNREVLQKEKSTKSEGKHEGVYRELVDEKALKHITEMGFSKEASRQALMDNGNNLEAALNVLLTSNKQKPVMGPPLRGRGKGRGRIRSEDEEDLGNARPSAPSTLFDFLESKMGTLNVEEPKSQPQQLHQGQYRSSNTEQNGVKDNNHLRHPPRNDTRQPRNEKPPRFQRDSQNSKSVLEGSGLPRNRGSERPSTSSVSEVWAEDRIKCDRPYSRYDRTKDTSYPLGSQHSDGAFKKRDNSMQSRSGKGPSFAEAKENPLPQGSVDYNNQKRGKRESQTSIPDYFYDRKSQTINNEAFSGIKIEKHFNVNTDYQNPVRSNSFIGVPNGEVEMPLKGRRIGPIKPAGPVTAVPCDDKIFYNSGPKRRSGPIKPEKILESSIPMEYAKMWKPGDECFALYWEDNKFYRAEVEALHSSGMTAVVKFIDYGNYEEVLLSNIKPIQTEAWEEEGTYDQTLEFRRGGDGQPRRSTRPTQQFYQPPRARN

SEQ ID NO: 7 - protein, 2177 aa, Homo sapiens
MAAFGILSYEHRPLKRPRLGPPDVYPQDPKQKEDELTALNVKQGFNNQPAVSGDEHGSAKNVSFNPAKISSNFSSIIAEKLRCNTLPDTGRRKPQVNQKDNFWLVTARSQSAINTWFTDLAGTKPLTQLAKKVPIFSKKEEVFGYLAKYTVPVMRAAWLIKMTCAYYAAISETKVKKRHVDPFMEWTQIITKYLWEQLQKMAEYYRPGPAGSGGCGSTIGPLPHDVEVAIRQWDYTEKLAMFMFQDGMLDRHEFLTWVLECFEKIRPGEDELLKLLLPLLLRYSGEFVQSAYLSRRLAYFCTRRLALQLDGVSSHSSHVISAQSTSTLPTTPAPQPPTSSTPSTPFSDLLMCPQHRPLVFGLSCILQTILLCCPSALVWHYSLTDSRIKTGSPLDHLPIAPSNLPMPEGNSAFTQQVRAKLREIEQQIKERGQAVEVRWSFDKCQEATAGFTIGRVLHTLEVLDSHSFERSDFSNSLDSLCNRIFGLGPSKDGHEISSDDDAVVSLLCEWAVSCKRSGRHRAMVVAKLLEKRQAEIEAERCGESEAADEKGSIASGSLSAPSAPIFQDVLLQFLDTQAPMLTDPRSESERVEFFNLVLLFCELIRHDVFSHNMYTCTLISRGDLAFGAPGPRPPSPFDDPADDPEHKEAEGSSSSKLEDPGLSESMDIDPSSSVLFEDMEKPDFSLFSPTMPCEGKGSPSPEKPDVEKEVKPPPKEKIEGTLGVLYDQPRHVQYATHFPIPQEESCSHECNQRLVVLFGVGKQRDDARHAIKKITKDILKVLNRKGTAETDQLAPIVPLNPGDLTFLGGEDGQKRRRNRPEAFPTAEDIFAKFQHLSHYDQHQVTAQVSRNVLEQITSFALGMSYHLPLVQHVQFIFDLMEYSLSISGLIDFAIQLLNELSVVEAELLLKSSDLVGSYTTSLCLCIVAVLRHYHACLILNQDQMAQVFEGLCGVVKHGMNRSDGSSAERCILAYLYDLYTSCSHLKNKFGELFSDFCSKVKNTIYCNVEPSESNMRWAPEFMIDTLENPAAHTFTYTGLGKSLSENPANRYSFVCNALMHVCVGHHDPDRVNDIAILCAELTGYCKSLSAEWLGVLKALCCSSNNGTCGFNDLLCNVDVSDLSFHDSLATFVAILIARQCLLLEDLIRCAAIPSLLNAACSEQDSEPGARLTCRILLHLFKTPQLNPCQSDGNKPTVGIRSSCDRHLLAASQNRIVDGAVFAVLKAVFVLGDAELKGSGFTVTGGTEELPEEEGGGGSGGRRQGGRNISVETASLDVYAKYVLRSICQQEWVGERCLKSLCEDSNDLQDPVLSSAQAQRLMQLICYPHRLLDNEDGENPQRQRIKRILQNLDQWTMRQSSLELQLMIKQTPNNEMNSLLENIAKATIEVFQQSAETGSSSGSTASNMPSSSKTKPVLSSLERSGVWLVAPLIAKLPTSVQGHVLKAAGEELEKGQHLGSSSRKERDRQKQKSMSLLSQQPFLSLVLTCLKGQDEQREGLLTSLYSQVHQIVNNWRDDQYLDDCKPKQLMHEALKLRLNLVGGMFDTVQRSTQQTTEWAMLLLEIIISGTVDMQSNNELFTTVLDMLSVLINGTLAADMSSISQGSMEENKRAYMNLAKKLQKELGERQSDSLEKVRQLLPLPKQTRDVITCEPQGSLIDTKGNKIAGFDSIFKKEGLQVSTKQKISPWDLFEGLKPSAPLSWGWFGTVRVDRRVARGEEQQRLLLYHTHLRPRPRAYYLEPLPLPPEDEEPPAPTLLEPEKKAPEPPKTDKPGAAPPSTEERKKKSTKGKKRSQPATKTEDYGMGPGRSGPYGVTVPPDLLHHPNPGSITHLNYRQGSIGLYTQNQPLPAGGPRVDPYRPVRLPMQKLPTRPTYPGVLPTTMTGVMGLEPSSYKTSVYRQQQPAVPQGQRLRQQLQQSQGMLGQSSVHQMTPSSSYGLQTSQGYTPYVSHVGLQQHTGPAGTMVPPSYSSQPYQSTHPSTNPTLVDPTRHLQQRPSGYVHQQAPTYGHGLTSTQRFSHQTLQQTPMISTMTPMSAQGVQAGVRSTAILPEQQQQQQQQQQQQQQQQQQQQQQQQQQYHIRQQQQQQILRQQQQQQQQQQQQQQQQQQQQQQQQQQHQQQQQQQAAPPQPQPQSQPQFQRQGLQQTQQQQQTAALVRQLQQQLSNTQPQPSTNIFGRY

SEQ ID NO: 8 - protein, 2180 aa, Homo sapiens
MAAFGILSYEHRPLKRPRLGPPDVYPQDPKQKEDELTALNVKQGFNNQPAVSGDEHGSAKNVSFNPAKISSNFSSIIAEKLRCNTLPDTGRRKPQVNQKDNFWLVTARSQSAINTWFTDLAGTKPLTQLAKKVPIFSKKEEVFGYLAKYTVPVMRAAWLIKMTCAYYAAISETKVKKRHVDPFMEWTQIITKYLWEQLQKMAEYYRPGPAGSGGCGSTIGPLPHDVEVAIRQWDYTEKLAMFMFQDGMLDRHEFLTWVLECFEKIRPGEDELLKLLLPLLLRYSGEFVQSAYLSRRLAYFCTRRLALQLDGVSSHSSHVISAQSTSTLPTTPAPQPPTSSTPSTPFSDLLMCPQHRPLVFGLSCILQTILLCCPSALVWHYSLTDSRIKTGSPLDHLPIAPSNLPMPEGNSAFTQQVRAKLREIEQQIKERGQAVEVRWSFDKCQEATAGFTIGRVLHTLEVLDSHSFERSDFSNSLDSLCNRIFGLGPSKDGHEISSDDDAVVSLLCEWAVSCKRSGRHRAMVVAKLLEKRQAEIEAERCGESEAADEKGSIASGSLSAPSAPIFQDVLLQFLDTQAPMLTDPRSESERVEFFNLVLLFCELIRHDVFSHNMYTCTLISRGDLAFGAPGPRPPSPFDDPADDPEHKEAEGSSSSKLEDPGLSESMDIDPSSSVLFEDMEKPDFSLFSPTMPCEGKGSPSPEKPDVEKEVKPPPKEKIEGTLGVLYDQPRHVQYATHFPIPQEESCSHECNQRLVVLFGVGKQRDDARHAIKKITKDILKVLNRKGTAETDQLAPIVPLNPGDLTFLGGEDGQKRRRNRPEAFPTAEDIFAKFQHLSHYDQHQVTAQVSRNVLEQITSFALGMSYHLPLVQHVQFIFDLMEYSLSISGLIDFAIQLLNELSVVEAELLLKSSDLVGSYTTSLCLCIVAVLRHYHACLILNQDQMAQVFEGLCGVVKHGMNRSDGSSAERCILAYLYDLYTSCSHLKNKFGELFSDFCSKVKNTIYCNVEPSESNMRWAPEFMIDTLENPAAHTFTYTGLGKSLSENPANRYSFVCNALMHVCVGHHDPDRVNDIAILCAELTGYCKSLSAEWLGVLKALCCSSNNGTCGFNDLLCNVDVSDLSFHDSLATFVAILIARQCLLLEDLIRCAAIPSLLNAACSEQDSEPGARLTCRILLHLFKTPQLNPCQSDGNKPTVGIRSSCDRHLLAASQNRIVDGAVFAVLKAVFVLGDAELKGSGFTVTGGTEELPEEEGGGGSGGRRQGGRNISVETASLDVYAKYVLRSICQQEWVGERCLKSLCEDSNDLQDPVLSSAQAQRLMQLICYPHRLLDNEDGENPQRQRIKRILQNLDQWTMRQSSLELQLMIKQTPNNEMNSLLENIAKATIEVFQQSAETGSSSGSTASNMPSSSKTKPVLSSLERSGVWLVAPLIAKLPTSVQGHVLKAAGEELEKGQHLGSSSRKERDRQKQKSMSLLSQQPFLSLVLTCLKGQDEQREGLLTSLYSQVHQIVNNWRDDQYLDDCKPKQLMHEALKLRLNLVGGMFDTVQRSTQQTTEWAMLLLEIIISGTVDMQSNNELFTTVLDMLSVLINGTLAADMSSISQGSMEENKRAYMNLAKKLQKELGERQSDSLEKVRQLLPLPKQTRDVITCEPQGSLIDTKGNKIAGFDSIFKKEGLQVSTKQKISPWDLFEGLKPSAPLSWGWFGTVRVDRRVARGEEQQRLLLYHTHLRPRPRAYYLEPLPLPPEDEEPPAPTLLEPEKKAPEPPKTDKPGAAPPSTEERKKKSTKGKKRSQPATKTEDYGMGPGRSGPYGVTVPPDLLHHPNPGSITHLNYRQGSIGLYTQNQPLPAGGPRVDPYRPVRLPMQKLPTRPTYPGVLPTTMTGVMGLEPSSYKTSVYRQQQPAVPQGQRLRQQLQAKIQSQGMLGQSSVHQMTPSSSYGLQTSQGYTPYVSHVGLQQHTGPAGTMVPPSYSSQPYQSTHPSTNPTLVDPTRHLQQRPSGYVHQQAPTYGHGLTSTQRFSHQTLQQTPMISTMTPMSAQGVQAGVRSTAILPEQQQQQQQQQQQQQQQQQQQQQQQQQQYHIRQQQQQQILRQQQQQQQQQQQQQQQQQQQQQQQQQQHQQQQQQQAAPPQPQPQSQPQFQRQGLQQTQQQQQTAALVRQLQQQLSNTQPQPSTNIFGRY

SEQ ID NO: 9 - protein, 2176 aa, Homo sapiens
MAAFGILSYEHRPLKRPRLGPPDVYPQDPKQKEDELTALNVKQGFNNQPAVSGDEHGSAKNVSFNPAKISSNFSSIIAEKLRCNTLPDTGRRKPQVNQKDNFWLVTARSQSAINTWFTDLAGTKPLTQLAKKVPIFSKKEEVFGYLAKYTVPVMRAAWLIKMTCAYYAAISETKVKKRHVDPFMEWTQIITKYLWEQLQKMAEYYRPGPAGSGGCGSTIGPLPHDVEVAIRQWDYTEKLAMFMFQDGMLDRHEFLTWVLECFEKIRPGEDELLKLLLPLLLRYSGEFVQSAYLSRRLAYFCTRRLALQLDGVSSHSSHVISAQSTSTLPTTPAPQPPTSSTPSTPFSDLLMCPQHRPLVFGLSCILQTILLCCPSALVWHYSLTDSRIKTGSPLDHLPIAPSNLPMPEGNSAFTQQVRAKLREIEQQIKERGQAVEVRWSFDKCQEATAGFTIGRVLHTLEVLDSHSFERSDFSNSLDSLCNRIFGLGPSKDGHEISSDDDAVVSLLCEWAVSCKRSGRHRAMVVAKLLEKRQAEIEAERCGESEAADEKGSIASGSLSAPSAPIFQDVLLQFLDTQAPMLTDPRSESERVEFFNLVLLFCELIRHDVFSHNMYTCTLISRGDLAFGAPGPRPPSPFDDPADDPEHKEAEGSSSSKLEDPGLSESMDIDPSSSVLFEDMEKPDFSLFSPTMPCEGKGSPSPEKPDVEKEVKPPPKEKIEGTLGVLYDQPRHVQYATHFPIPQEESCSHECNQRLVVLFGVGKQRDDARHAIKKITKDILKVLNRKGTAETDQLAPIVPLNPGDLTFLGGEDGQKRRRNRPEAFPTAEDIFAKFQHLSHYDQHQVTAQVSRNVLEQITSFALGMSYHLPLVQHVQFIFDLMEYSLSISGLIDFAIQLLNELSVVEAELLLKSSDLVGSYTTSLCLCIVAVLRHYHACLILNQDQMAQVFEGLCGVVKHGMNRSDGSSAERCILAYLYDLYTSCSHLKNKFGELFSDFCSKVKNTIYCNVEPSESNMRWAPEFMIDTLENPAAHTFTYTGLGKSLSENPANRYSFVCNALMHVCVGHHDPDRVNDIAILCAELTGYCKSLSAEWLGVLKALCCSSNNGTCGFNDLLCNVDVSDLSFHDSLATFVAILIARQCLLLEDLIRCAAIPSLLNAACSEQDSEPGARLTCRILLHLFKTPQLNPCQSDGNKPTVGIRSSCDRHLLAASQNRIVDGAVFAVLKAVFVLGDAELKGSGFTVTGGTEELPEEEGGGGSGGRRQGGRNISVETASLDVYAKYVLRSICQQEWVGERCLKSLCEDSNDLQDPVLSSAQAQRLMQLICYPHRLLDNEDGENPQRQRIKRILQNLDQWTMRQSSLELQLMIKQTPNNEMNSLLENIAKATIEVFQQSAETGSSSGSTASNMPSSSKTKPVLSSLERSGVWLVAPLIAKLPTSVQGHVLKAAGEELEKGQHLGSSSRKERDRQKQKSMSLLSQQPFLSLVLTCLKGQDEQREGLLTSLYSQVHQIVNNWRDDQYLDDCKPKQLMHEALKLRLNLVGGMFDTVQRSTQQTTEWAMLLLEIIISGTVDMQSNNELFTTVLDMLSVLINGTLAADMSSISQGSMEENKRAYMNLAKKLQKELGERQSDSLEKVRQLLPLPKQTRDVITCEPQGSLIDTKGNKIAGFDSIFKKEGLQVSTKQKISPWDLFEGLKPSAPLSWGWFGTVRVDRRVARGEEQQRLLLYHTHLRPRPRAYYLEPLPLPPEDEEPPAPTLLEPEKKAPEPPKTDKPGAAPPSTEERKKKSTKGKKRSQPATKTEDYGMGPGRSGPYGVTVPPDLLHHPNPGSITHLNYRQGSIGLYTQNQPLPAGGPRVDPYRPVRLPMQKLPTRPTYPGVLPTTMTGVMGLEPSSYKTSVYRQQQPAVPQGQRLRQQLQSQGMLGQSSVHQMTPSSSYGLQTSQGYTPYVSHVGLQQHTGPAGTMVPPSYSSQPYQSTHPSTNPTLVDPTRHLQQRPSGYVHQQAPTYGHGLTSTQRFSHQTLQQTPMISTMTPMSAQGVQAGVRSTAILPEQQQQQQQQQQQQQQQQQQQQQQQQQQYHIRQQQQQQILRQQQQQQQQQQQQQQQQQQQQQQQQQQHQQQQQQQAAPPQPQPQSQPQFQRQGLQQTQQQQQTAALVRQLQQQLSNTQPQPSTNIFGRY

SEQ ID NO: 10 - DNA, 3295 bp, Homo sapiens
ctcggcctgcacggcggctgcggcggcggtagcggcagcggcggcggcggcggcggcggcggcggcggcggcggcggcggcggcggcagcggcggcggcctgggcccgggcgcagcggcggcggcggcggggcctggagccggatctaagatggcagcggcggcggcggcggtggggccgggcgcgggcggcgcggggtcggcggtcccgggcggcgcggggccctgcgctaccgtgtcggtgttccccggcgcccgcctcctcaccatcggcgacgcgaacggcgagatccagcggcacgcggagcagcaggcgctgcgcctcgaggtgcgcgccggcccggactcggcgggcatcgccctctacagccatgaagatgtgtgtgtctttaagtgctcagtgtcccgagagacagagtgcagccgtgtgggcaagcagtccttcatcatcaccctgggctgcaacagcgtcctcatccagttcgccacacccaacgatttctgttccttctacaacatcctgaaaacctgccggggccacaccctggagcggtctgtgttcagcgagcggacggaggagtcttctgccgtgcagtacttccagttttatggctacctgtcccagcagcagaacatgatgcaggactacgtgcggacaggcacctaccagcgcgccatcctgcaaaaccacaccgacttcaaggacaagatcgttcttgatgttggctgtggctctgggatcctgtcgttttttgccgcccaagctggagcacggaaaatctacgcggtggaggccagcaccatggcccagcacgctgaggtcttggtgaagagtaacaacctgacggaccgcatcgtggtcatcccgggcaaggtggaggaggtgtcactccccgagcaggtggacatcatcatctcggagcccatgggctacatgctcttcaacgagcgcatgctggagagctacctccacgccaagaagtacctgaagcccagcggaaacatgtttcctaccattggtgacgtccaccttgcacccttcacggatgaacagctctacatggagcagttcaccaaggccaacttctggtaccagccatctttccatggagtggacctgtcggccctccgaggtgccgcggtggatgagtatttccggcagcctgtggtggacacatttgacatccggatcctgatggccaagtctgtcaagtacacggtgaacttcttagaagccaaagaaggagatttgcacaggatagaaatcccattcaaattccacatgctgcattcagggctggtccacggcctggctttctggtttgacgttgctttcatcggctccataatgaccgtgtggctgtccacagccccgacagagcccctgacccactggtaccaggtgcggtgcctgttccagtcaccactgttcgccaaggcaggggacacgctctcagggacatgtctgcttattgccaacaaaagacagagctacgacatcagtattgtggcccaggtggaccagaccggctccaagtccagtaacctcctggatctgaaaaaccccttctttagatacacgggcacaacgccctcacccccacccggctcccactacacatctccctcggaaaacatgtggaacacgggcagcacctacaacctcagcagcgggatggccgtggcagggatgccgaccgcctatgacttgagcagtgttattgccagtggctccagcgtgggccacaacaacctgattcctttagccaacacggggattgtcaatcacacccactcccggatgggctccataatgagcacggggattgtccaagggtcctccggcgcccagggcagtggtggtggcagcacgagtgcccactatgcagtcaacagccagttcaccatgggcggccccgccatctccatggcgtcgcccatgtccatcccgaccaacaccatgcactacgggagctaggggcccgccccgcggactgacagcaccaggaaaccaaatgatgtccctgcccgccgcccccgccgggcggctttcccccttgtactggagaagctcgaacacccggtcacagctctctttgctatgggaactgggacacttttttacacgatgttgccgccgtccccaccctaacccccacctcccggccctgagcgtgtgtcgctgccatattttacacaaaatcatgttgtgggagccctcgtcccccctcctgcccgctctaccctgacctgggcttgtcatctgctggaacaggcgccatggggcctgccagccctgcctgccaggtcccttagcacctgtccccctgcctgtctccagtgggaaggtagcctggccaggcggggcctccccttcgacgaccaggcctcggtcacaacggacgtgacatgctgctttttttaattttatttttttatgaaaagaaccagtgtcaatccgcagaccctctgtgaagccaggccggccgggccgagccagcagcccctctccctagactcagaggcgccgcggggaggggtggccccgccgaggcttcaggggccccctccccaccaaagggttcacctcacacttgaatgtacaacccaccccactgtcgggaaggcctccgtcctcggcccctgcctcttgctgctgtcctgtccccgagcccctgcaggtccccccccgcccccccactcaagagttagagcaggtggctgcaggccttgggcccggagggaaggccactgccggccacttggggcagacacagacacctcaaggatctgtcacggaaggcgtcctttttccttgtagctaacgttaggcctgagtagctcccctccatccttgtagacgctccagtccctactactgtgacggcatttccatccctcccctgcccgggaagggaccttgcagggacctctccctccaaaaaaagaaaaaaagaaaaagaaagaaaaaataaatgaggaaacgtgttgcagcacaggcagttttcttctccttctgctcccctgtttctcatacccccaaactcagatgctggagctcaggcccgccgtgtgtgcacccaggcaggagcgggcgctgtccaggctgggccgcccccttggctctccctcctgttccaggggagccataggagggaaagcaggtggcccgggggggatatgggggccccagccctgtcccaaagctccctgctcggctgcccctcgcccgcctttatataaattctctgaatcacctttgcatagaaaataaaagtgtttgctttgtaagaaaa

SEQ ID NO: 11 - DNA, 3226 bp, Homo sapiens
ctcggcctgcacggcggctgcggcggcggtagcggcagcggcggcggcggcggcggcggcggcggcggcggcggcggcggcggcggcagcggcggcggcctgggcccgggcgcagcggcggcggcggcggggcctggagccggatctaagatggcagcggcggcggcggcggtggggccgggcgcgggcggcgcggggtcggcggtcccgggcggcgcggggccctgcgctaccgtgtcggtgttccccggcgcccgcctcctcaccatcggcgacgcgaacggcgagatccagcggcacgcggagcagcaggcgctgcgcctcgaggtgcgcgccggcccggactcggcgggcatcgccctctacagccatgaagatgtgtgtgtctttaagtgctcagtgtcccgagagacagagtgcagccgtgtgggcaagcagtccttcatcatcaccctgggctgcaacagcgtcctcatccagttcgccacacccaacgatttctgttccttctacaacatcctgaaaacctgccggggccacaccctggagcggtctgtgttcagcgagcggacggaggagtcttctgccgtgcagtacttccagttttatggctacctgtcccagcagcagaacatgatgcaggactacgtgcggacaggcacctaccagcgcgccatcctgcaaaaccacaccgacttcaaggacaagatcgttcttgatgttggctgtggctctgggatcctgtcgttttttgccgcccaagctggagcacggaaaatctacgcggtggaggccagcaccatggcccagcacgctgaggtcttggtgaagagtaacaacctgacggaccgcatcgtggtcatcccgggcaaggtggaggaggtgtcactccccgagcaggtggacatcatcatctcggagcccatgggctacatgctcttcaacgagcgcatgctggagagctacctccacgccaagaagtacctgaagcccagcggaaacatgtttcctaccattggtgacgtccaccttgcacccttcacggatgaacagctctacatggagcagttcaccaaggccaacttctggtaccagccatctttccatggagtggacctgtcggccctccgaggtgccgcggtggatgagtatttccggcagcctgtggtggacacatttgacatccggatcctgatggccaagtctgtcaagtacacggtgaacttcttagaagccaaagaaggagatttgcacaggatagaaatcccattcaaattccacatgctgcattcagggctggtccacggcctggctttctggtttgacgttgctttcatcggctccataatgaccgtgtggctgtccacagccccgacagagcccctgacccactggtaccaggtgcggtgcctgttccagtcaccactgttcgccaaggcaggggacacgctctcagggacatgtctgcttattgccaacaaaagacagagctacgacatcagtattgtggcccaggtggaccagaccggctccaagtccagtaacctcctggatctgaaaaaccccttctttagatacacgggcacaacgccctcacccccacccggctcccactacacatctccctcggaaaacatgtggaacacgggcagcacctacaacctcagcagcgggatggccgtggcagggatgccgaccgcctatgacttgagcagtgttattgccagtggctccagcgtgggccacaacaacctgattcctttagggtcctccggcgcccagggcagtggtggtggcagcacgagtgcccactatgcagtcaacagccagttcaccatgggcggccccgccatctccatggcgtcgcccatgtccatcccgaccaacaccatgcactacgggagctaggggcccgccccgcggactgacagcaccaggaaaccaaatgatgtccctgcccgccgcccccgccgggcggctttcccccttgtactggagaagctcgaacacccggtcacagctctctttgctatgggaactgggacacttttttacacgatgttgccgccgtccccaccctaacccccacctcccggccctgagcgtgtgtcgctgccatattttacacaaaatcatgttgtgggagccctcgtcccccctcctgcccgctctaccctgacctgggcttgtcatctgctggaacaggcgccatggggcctgccagccctgcctgccaggtcccttagcacctgtccccctgcctgtctccagtgggaaggtagcctggccaggcggggcctccccttcgacgaccaggcctcggtcacaacggacgtgacatgctgctttttttaattttatttttttatgaaaagaaccagtgtcaatccgcagaccctctgtgaagccaggccggccgggccgagccagcagcccctctccctagactcagaggcgccgcggggaggggtggccccgccgaggcttcaggggccccctccccaccaaagggttcacctcacacttgaatgtacaacccaccccactgtcgggaaggcctccgtcctcggcccctgcctcttgctgctgtcctgtccccgagcccctgcaggtccccccccgcccccccactcaagagttagagcaggtggctgcaggccttgggcccggagggaaggccactgccggccacttggggcagacacagacacctcaaggatctgtcacggaaggcgtcctttttccttgtagctaacgttaggcctgagtagctcccctccatccttgtagacgctccagtccctactactgtgacggcatttccatccctcccctgcccgggaagggaccttgcagggacctctccctccaaaaaaagaaaaaaagaaaaagaaagaaaaaataaatgaggaaacgtgttgcagcacaggcagttttcttctccttctgctcccctgtttctcatacccccaaactcagatgctggagctcaggcccgccgtgtgtgcacccaggcaggagcgggcgctgtccaggctgggccgcccccttggctctccctcctgttccaggggagccataggagggaaagcaggtggcccgggggggatatgggggccccagccctgtcccaaagctccctgctcggctgcccctcgcccgcctttatataaattctctgaatcacctttgcatagaaaataaaagtgtttgctttgtaagaaaa

SEQ ID NO: 12 - DNA, 3020 bp, Homo sapiens
gagtaccgagttccaccgctgtgcgaggtgtggcgttcaggacccccgaatgggaaacttcaggatctggggagagggcccatgcggggacggtgggagccgcagggtgagctcctcgtggaaacgtggacgggacgggccccttcctgatgccgggcttcgaaatgaagatgtgtgtgtctttaagtgctcagtgtcccgagagacagagtgcagccgtgtgggcaagcagtccttcatcatcaccctgggctgcaacagcgtcctcatccagttcgccacacccaacgatttctgttccttctacaacatcctgaaaacctgccggggccacaccctggagcggtctgtgttcagcgagcggacggaggagtcttctgccgtgcagtacttccagttttatggctacctgtcccagcagcagaacatgatgcaggactacgtgcggacaggcacctaccagcgcgccatcctgcaaaaccacaccgacttcaaggacaagatcgttcttgatgttggctgtggctctgggatcctgtcgttttttgccgcccaagctggagcacggaaaatctacgcggtggaggccagcaccatggcccagcacgctgaggtcttggtgaagagtaacaacctgacggaccgcatcgtggtcatcccgggcaaggtggaggaggtgtcactccccgagcaggtggacatcatcatctcggagcccatgggctacatgctcttcaacgagcgcatgctggagagctacctccacgccaagaagtacctgaagcccagcggaaacatgtttcctaccattggtgacgtccaccttgcacccttcacggatgaacagctctacatggagcagttcaccaaggccaacttctggtaccagccatctttccatggagtggacctgtcggccctccgaggtgccgcggtggatgagtatttccggcagcctgtggtggacacatttgacatccggatcctgatggccaagtctgtcaagtacacggtgaacttcttagaagccaaagaaggagatttgcacaggatagaaatcccattcaaattccacatgctgcattcagggctggtccacggcctggctttctggtttgacgttgctttcatcggctccataatgaccgtgtggctgtccacagccccgacagagcccctgacccactggtaccaggtgcggtgcctgttccagtcaccactgttcgccaaggcaggggacacgctctcagggacatgtctgcttattgccaacaaaagacagagctacgacatcagtattgtggcccaggtggaccagaccggctccaagtccagtaacctcctggatctgaaaaaccccttctttagatacacgggcacaacgccctcacccccacccggctcccactacacatctccctcggaaaacatgtggaacacgggcagcacctacaacctcagcagcgggatggccgtggcagggatgccgaccgcctatgacttgagcagtgttattgccagtggctccagcgtgggccacaacaacctgattcctttagggtcctccggcgcccagggcagtggtggtggcagcacgagtgcccactatgcagtcaacagccagttcaccatgggcggccccgccatctccatggcgtcgcccatgtccatcccgaccaacaccatgcactacgggagctaggggcccgccccgcggactgacagcaccaggaaaccaaatgatgtccctgcccgccgcccccgccgggcggctttcccccttgtactggagaagctcgaacacccggtcacagctctctttgctatgggaactgggacacttttttacacgatgttgccgccgtccccaccctaacccccacctcccggccctgagcgtgtgtcgctgccatattttacacaaaatcatgttgtgggagccctcgtcccccctcctgcccgctctaccctgacctgggcttgtcatctgctggaacaggcgccatggggcctgccagccctgcctgccaggtcccttagcacctgtccccctgcctgtctccagtgggaaggtagcctggccaggcggggcctccccttcgacgaccaggcctcggtcacaacggacgtgacatgctgctttttttaattttatttttttatgaaaagaaccagtgtcaatccgcagaccctctgtgaagccaggccggccgggccgagccagcagcccctctccctagactcagaggcgccgcggggaggggtggccccgccgaggcttcaggggccccctccccaccaaagggttcacctcacacttgaatgtacaacccaccccactgtcgggaaggcctccgtcctcggcccctgcctcttgctgctgtcctgtccccgagcccctgcaggtccccccccgcccccccactcaagagttagagcaggtggctgcaggccttgggcccggagggaaggccactgccggccacttggggcagacacagacacctcaaggatctgtcacggaaggcgtcctttttccttgtagctaacgttaggcctgagtagctcccctccatccttgtagacgctccagtccctactactgtgacggcatttccatccctcccctgcccgggaagggaccttgcagggacctctccctccaaaaaaagaaaaaaagaaaaagaaagaaaaaataaatgaggaaacgtgttgcagcacaggcagttttcttctccttctgctcccctgtttctcatacccccaaactcagatgctggagctcaggcccgccgtgtgtgcacccaggcaggagcgggcgctgtccaggctgggccgcccccttggctctccctcctgttccaggggagccataggagggaaagcaggtggcccgggggggatatgggggccccagccctgtcccaaagctccctgctcggctgcccctcgcccgcctttatataaattctctgaatcacctttgcatagaaaataaaagtgtttgctttgtaagaaaa

SEQ ID NO: 13 - DNA, 2645 bp, Homo sapiens
actgagcatgcccagttgcagagccgaccagaggagttttttcttttcttttctttttttttttttaagggggggggtctcaagtaggaggcctccccatcacccccaccccagccccccaccacccccggcctaagcagctaccatggcccaggtggccggcgcggcgttgtcccaggcgggttggtatctttcagatgaaggcattgaagcttgcacaagctctccagacaaagtcaatgtaaatgacatcatcctgattgctctcaatacagatctgagaacaattggcaagaaattcctccccagtgacatcaatagtggaaaggtagaaaagctcgaaggtccatgtgttttgcaaattcaaaaaattcgcaatgttgctgcaccaaaggataatgaagaatctcaggctgcaccaaggatgctgcgattacagatgactgatggtcatataagttgcacagcagtagaatttagttatatgtcaaaaataagcctgaacacaccacctggaactaaagttaagctctcaggcattgttgacataaaaaatggattcctgctcttgaatgactctaacaccacagttcttggtggtgaagtggaacaccttattgagaaatgggagttacagagaagcttatcaaaacacaatagaagcaatattggaactgaaggtggaccaccgccttttgtgccttttggacagaagtgtgtatctcatgtccaagtggatagcagagaacttgatcgaagaaaaacattgcaagttacaatgcctgtcaaacctacaaatgataatgatgaatttgaaaagcaaaggacggctgctattgctgaagttgcaaagagcaaggaaaccaagacatttggaggaggtggtggtggtgctagaagtaatctcaatatgaatgctgctggtaaccgaaatagggaagttttacagaaagaaaagtcaaccaaatcagagggaaaacatgaaggtgtctatagagaactggttgatgagaaagctctgaagcacataacggaaatgggcttcagtaaggaagcatcgaggcaagctcttatggataatggcaacaacttagaagcagcactgaacgtacttcttacaagcaataaacagaaacctgttatgggtcctcctctgagaggtagaggaaaaggcagggggcgaataagatctgaagatgaagaggacctgggaaatgcaaggccatcagcaccaagcacattatttgatttcttggaatctaaaatgggaactttgaatgtggaagaacctaaatcacagccacagcagcttcatcagggacaatacagatcatcaaatactgagcaaaatggagtaaaagataataatcatctgagacatcctcctcgaaatgataccaggcagccaagaaatgaaaaaccgcctcgttttcaaagagactcccaaaattcaaagtcagttttagaaggcagtggattacctagaaatagaggttctgaaagaccaagtacttcttcagtatctgaagtatgggctgaagacagaatcaaatgtgatagaccgtattctagatatgacagaactaaagatacttcatatcctttaggttctcagcatagtgatggtgcttttaaaaaaagagataactctatgcaaagcagatcaggaaaaggtccctcctttgcagaggcaaaagaaaatccacttcctcaaggatctgtagattataataatcaaaaacgtggaaaaagagaaagccaaacatctattcctgattatttttatgacaggaaatcacaaacaataaataatgaagctttcagtggtataaaaattgaaaaacattttaatgtaaatactgattatcagaatccagttcgaagtaatagtttcattggtgttccaaatggagaagtagaaatgccactgaaaggaagacgaataggacctattaagccagcaggacctgtcacagctgtaccctgtgatgataaaatattttacaatagtgggcccaaacgaagatctgggccaattaagccagaaaaaatactagaatcatctattcctatggagtatgcaaaaatgtggaaacctggagatgaatgttttgcactttattgggaagacaacaagttttaccgggcagaagttgaagccctccattcttcgggtatgacagcagttgttaaattcattgactacggaaactatgaagaggtgctactgagcaatatcaagcccattcaaacagaggcatgggaggaagaaggcacctacgatcaaactctggagttccgtaggggaggtgatggccagccaagacgatccactcggccaacccaacagttttaccaaccaccccgggctcggaactaataggaaaagactctttgtgaagaaacgagccagtgactgaaacaccctggtggaaacctgttgacagaccttccactttctcttcagaataagtagctgtggtggatattattatttgaagaaagaaaaaacagattttagggtggaaaaaacagtcaactcacacaaagaatggaaaaaaatactgagttaaattaagcaaataccttttacaagtgaaaggaagaatttttcttctgccgtcaataaaaccattgtgctatta

SEQ ID NO: 14 - DNA, 3032 bp, Homo sapiens
acccaccctgcgccccagccctctacagcgcgggcgaagggcatcggcggggaagcggctgtggttgccagatctggcagtacagagccaaggtggcagggccgcgctaggacacggatggtcccagggggagggcaacaggaaaaacaagcactgacacaactgccccatttccccatgtcccctaaaagcaggttttcttgggctgaccagggcattttcactgtggtgtccgactggggcaactcgatctgcccgcggggactcaaaaaagcttgagctgcattttttctttgcgtccgggcggaccaaaaggcgaggtgaagggaacgagtgtttataagaaagtaaacttttgtgcagaatgggctggggacacctctgagttttgctgcccgcaacgtgacctttttagcgcggtcctggacgcgttccctcgcagccaacccgtcagtctcctcaggccttatcctgccctctctgaggtggccgcctgtgggcgctgcaaacgccacacttttgctctacgaagaacctcctcgcgactttccaaggtatctttcagatgaaggcattgaagcttgcacaagctctccagacaaagtcaatgtaaatgacatcatcctgattgctctcaatatctgagaacaattggcaagaaattcctccccagtgacatcaatagtggaaaggtagaaaagctcgaaggtccatgtgttttgcaaattcaaaaaattcgcaatgttgctgcaccaaaggataatgaagaatctcaggctgcaccaaggatgctgcgattacagatgactgatggtcatataagttgcacagcagtagaatttagttatatgtcaaaaataagcctgaacacaccacctggaactaaagttaagctctcaggcattgttgacataaaaaatggattcctgctcttgaatgactctaacaccacagttcttggtggtgaagtggaacaccttattgagaaatgggagttacagagaagcttatcaaaacacaatagaagcaatattggaactgaaggtggaccaccgccttttgtgccttttggacagaagtgtgtatctcatgtccaagtggatagcagagaacttgatcgaagaaaaacattgcaagttacaatgcctgtcaaacctacaaatgataatgatgaatttgaaaagcaaaggacggctgctattgctgaagttgcaaagagcaaggaaaccaagacatttggaggaggtggtggtggtgctagaagtaatctcaatatgaatgctgctggtaaccgaaatagggaagttttacagaaagaaaagtcaaccaaatcagagggaaaacatgaaggtgtctatagagaactggttgatgagaaagctctgaagcacataacggaaatgggcttcagtaaggaagcatcgaggcaagctcttatggataatggcaacaacttagaagcagcactgaacgtacttcttacaagcaataaacagaaacctgttatgggtcctcctctgagaggtagaggaaaaggcagggggcgaataagatctgaagatgaagaggacctgggaaatgcaaggccatcagcaccaagcacattatttgatttcttggaatctaaaatgggaactttgaatgtggaagaacctaaatcacagccacagcagcttcatcagggacaatacagatcatcaaatactgagcaaaatggagtaaaagataataatcatctgagacatcctcctcgaaatgataccaggcagccaagaaatgaaaaaccgcctcgttttcaaagagactcccaaaattcaaagtcagttttagaaggcagtggattacctagaaatagaggttctgaaagaccaagtacttcttcagtatctgaagtatgggctgaagacagaatcaaatgtgatagaccgtattctagatatgacagaactaaagatacttcatatcctttaggttctcagcatagtgatggtgcttttaaaaaaagagataactctatgcaaagcagatcaggaaaaggtccctcctttgcagaggcaaaagaaaatccacttcctcaaggatctgtagattataataatcaaaaacgtggaaaaagagaaagccaaacatctattcctgattatttttatgacaggaaatcacaaacaataaataatgaagctttcagtggtataaaaattgaaaaacattttaatgtaaatactgattatcagaatccagttcgaagtaatagtttcattggtgttccaaatggagaagtagaaatgccactgaaaggaagacgaataggacctattaagccagcaggacctgtcacagctgtaccctgtgatgataaaatattttacaatagtgggcccaaacgaagatctgggccaattaagccagaaaaaatactagaatcatctattcctatggagtatgcaaaaatgtggaaacctggagatgaatgttttgcactttattgggaagacaacaagttttaccgggcagaagttgaagccctccattcttcgggtatgacagcagttgttaaattcattgactacggaaactatgaagaggtgctactgagcaatatcaagcccattcaaacagaggcatgggaggaagaaggcacctacgatcaaactctggagttccgtaggggaggtgatggccagccaagacgatccactcggccaacccaacagttttaccaaccaccccgggctcggaactaataggaaaagtagactctttgtgaagaaacgagccagtgactgaaacaccctggtggaaacctgttgacagaccttccactttctcttcagaataagtagctgtggtggatattattatttgaagaaagaaaaaacagattttagggtggaaaaaacagtcaactcacacaaagaatggaaaaaaatactgagttaaattaagcaaataccttttacaagtgaaaggaagaatttttcttctgccgtcaataaaaccattgtgctattattgttttaaaaaaaaaaaaa

SEQ ID NO: 15 - DNA, 3032 bp, Homo sapiens; identical to SEQ ID NO: 14

SEQ ID NO: 16 - DNA, 6925 bp, Homo sapiens
agtttccggcaatggtcgagagtttctaacgtgcccccttgttgtctctcggccgccgtcctctcaaccaccgccccccttttcggctccctctcccccttcccgttcccccagtcagcctggccctgctggtgcctccggcgctacgggctgggcaagatggcggccttcgggatcttgagctacgaacaccggcccctgaagcggccgcggctggggcctcccgatgtttaccctcaggaccccaaacagaaggaggatgaactgacggccttgaatgtaaaacaaggtttcaataaccagcctgctgtctctggggatgagcatggcagtgccaagaacgtcagcttcaatcctgccaagatcagttccaacttcagcagcattattgcagagaaattacgttgtaatacccttcctgacactggtcgcaggaagccccaagtgaaccagaaggataacttctggctggtgactgcacgatcccagagtgccattaacacttggttcactgacttggctggcaccaagccactcacgcaactagccaaaaaggtccccattttcagtaagaaggaagaggtgtttgggtacttagccaaatacacagtgcctgtgatgcgggctgcctggctcattaagatgacctgtgcctactatgcagcaatctctgagaccaaggttaagaagagacatgttgaccctttcatggaatggactcagatcatcaccaagtacttatgggagcagttacagaagatggctgaatactaccggccagggcctgcaggaagtgggggctgtggttccacgatagggcccttgccccatgatgtagaggtggcaatccggcagtgggattacaccgagaagctggccatgttcatgtttcaggatggaatgctggacagacatgagttcctgacctgggtgcttgagtgttttgagaagatccgccctggagaggatgaattgcttaaactgctgctgcctctgcttctccgatactctggggaatttgttcagtctgcatacctgtcccgccggcttgcctacttctgtacacggagactggccctgcagctggatggtgtgagcagtcactcatctcatgttatatctgctcagtcaacaagcacgctacccaccacccctgctcctcagcccccaactagcagcacaccctcgactccctttagtgacctgcttatgtgccctcagcaccggcccctggtttttggcctcagctgtatcctacagaccatcctcctgtgctgtcctagtgccttggtttggcactactcactgactgatagcagaattaagaccggctcaccacttgaccacttgcctattgccccgtccaacctgcccatgccagagggtaacagtgccttcactcagcaggtccgtgcaaagttgcgggagatcgagcagcagatcaaggagcggggacaggcagttgaagttcgctggtctttcgataaatgccaggaagctactgcaggcttcaccattggacgggtacttcatactttggaagtgctggacagccatagttttgaacgctctgacttcagcaactctcttgactccctttgtaaccgaatctttggattgggacctagcaaggatgggcatgagatctcctcagatgatgatgctgtggtgtcattgctatgtgaatgggctgtcagctgcaagcgttctggtcggcatcgtgctatggtggtagccaagctcctggagaagagacaggcggagattgaggctgagcgttgtggagaatcagaagccgcagatgagaagggttccatcgcctctggctccctttctgctcccagtgctcccattttccaggatgtcctcctgcagtttctggatacacaggctcccatgctgacggaccctcgaagtgagagtgagcgggtggaattctttaacttagtactgctgttctgtgaactgattcgacatgatgttttctcccacaacatgtatacttgcactctcatctcccgaggggaccttgcctttggagcccctggtccccggcctccctctccctttgatgatcctgccgatgacccagagcacaaggaggctgaaggcagcagcagcagcaagctggaagatccagggctctcagaatctatggacattgaccctagttccagtgttctctttgaggacatggagaagcctgatttctcattgttctcccctactatgccctgtgaggggaagggcagtccatcccctgagaagccagatgtcgagaaggaggtgaagcccccacccaaggagaagattgaagggacccttggggttctttacgaccagccacgacacgtgcagtacgccacccattttcccatcccccaggaggagtcatgcagccatgagtgcaaccagcggttggtcgtactgtttggggtgggaaagcagcgagatgatgcccgccatgccatcaagaaaatcaccaaggatatcttgaaggttctgaaccgcaaagggacagcagaaactgaccagcttgctcctattgtgcctctgaatcctggagacctgacattcttaggtggggaggatgggcagaagcggcgacgcaaccggcctgaagccttccccactgctgaagatatctttgctaagttccagcacctttcacattatgaccaacaccaggtcacggctcaggtctcccggaatgttctggagcagatcacgagctttgcccttggcatgtcataccacttgcctctggtgcagcatgtgcagttcatcttcgacctcatggaatattcactcagcatcagtggcctcatcgactttgccattcagctgctgaatgaactgagtgtagttgaggctgagctgcttctcaaatcctcggatctggtgggcagctacactactagcctgtgcctgtgcatcgtggctgtcctgcggcactatcatgcctgcctcatcctcaaccaggaccagatggcacaggtctttgaggggctgtgtggcgtcgtgaagcatgggatgaaccggtccgatggctcctctgcagagcgctgtatccttgcttatctctatgatctgtacacctcctgtagccatttaaagaacaaatttggggagctcttcagcgacttttgctcaaaggtgaagaacaccatctactgcaacgtggagccatcggaatcaaatatgcgctgggcacctgagttcatgatcgacactctagagaaccctgcagctcacaccttcacctacacggggctaggcaagagtcttagtgagaaccctgctaaccgctacagctttgtctgcaatgcccttatgcacgtctgtgtggggcaccatgatcccgatagggtgaatgacatcgcaatcctgtgtgcagagctgaccggctattgcaagtcactgagtgcagaatggctaggagtgcttaaggccttgtgctgctcctctaacaatggcacttgtggtttcaacgatctcctctgcaatgttgatgtcagtgacctatcttttcatgactcgctggctacttttgttgccatcctcatcgctcggcagtgtttgctcctggaagatctgattcgctgtgctgccatcccttcactccttaatgctgcttgtagtgaacaggactctgagccaggggcccggcttacctgccgcatcctccttcaccttttcaagacaccgcagctcaatccttgccagtctgatggaaacaagcctacagtaggaatccgctcctcctgcgaccgccacctgctggctgcctcccagaaccgcatcgtggatggagccgtgtttgctgttctcaaggctgtgtttgtacttggggatgcggaactgaaaggttcaggcttcactgtgacaggaggaacagaagaacttccagaggaggagggaggaggtggcagtggtggtcggaggcagggtggccgcaacatctctgtggagacagccagtctggatgtctatgccaagtacgtgctgcgcagcatctgccaacaggaatgggtaggagaacgttgccttaagtctctgtgtgaggacagcaatgacctgcaagacccagtgttgagtagtgcccaggcgcagcgcctcatgcagctcatttgctatccacatcgactgctggacaatgaggatggggaaaacccccagcggcagcgcataaagcgcattctccagaacttggaccagtggaccatgcgccagtcttccttggagctgcagctcatgatcaagcagacccctaacaatgagatgaactccctcttggagaacatcgccaaggccacaatcgaggttttccaacagtcagcagagacagggtcatcttctggaagtactgcaagcaacatgcccagcagcagcaagaccaagcctgtgctcagctctctagagcgctctggtgtatggctggtggcccccctcattgctaaactgcccacctcagtccagggacatgtgttaaaggctgctggggaagaattggagaagggtcagcacctgggttcctcttcacgcaaagaacgtgatcgacaaaagcagaagagcatgtccctattgagccagcagcccttcttatcgctggtgctaacatgtctgaaagggcaggatgaacaacgcgagggactccttacctccctctacagccaggtgcaccagattgtgaataattggcgagatgaccagtacttagatgattgcaaaccaaagcagcttatgcatgaggcactcaaactgcggctcaacctggtggggggcatgtttgacacggtgcagcgcagcacccagcagaccacggagtgggccatgctcctcctggagatcatcatcagcggcactgtcgacatgcagtccaacaatgagctcttcactactgtgttggacatgctgagcgtgctcatcaatgggacattggctgcagacatgtctagcatctcgcaaggtagcatggaggaaaacaagcgtgcatacatgaacctggcgaagaagttgcagaaggagttgggggagcgccagtcagacagtctggaaaaggttcgccagctgctgccactgcccaagcagacccgagatgtcatcacgtgtgagccacagggctcccttatcgataccaagggcaacaagattgctggcttcgattccatcttcaagaaggagggtctacaggtttccaccaaacagaagatctcgccctgggatctttttgaggggttgaagccgtcagcaccactctcttggggctggtttggaacagtccgagtggaccggcgagtggctcgaggagaggagcagcagcggttgctgctctaccacacacacctgaggccccggccccgcgcctattacctggagccactgccactgcccccagaagatgaggagccgcctgctcctaccctgctagagcctgagaaaaaggctccagagccccccaaaactgacaaaccgggggctgctccacccagtactgaggaacgcaagaagaagtccaccaagggcaagaaacgcagccagccagctaccaagacagaggactatggaatgggcccgggtcggagcggcccttatggtgtgacagtgcctccggacctcctgcaccacccaaaccctggttctataacacaccttaactacaggcaaggctccataggcctgtacacccagaaccagccactacctgcaggtggccctcgtgtggacccataccgtcctgtgcgcttaccaatgcagaagctgcccacccgaccaacttaccctggagtgctgcccacaaccatgactggcgtcatgggtttagaaccctcctcttataagacctctgtgtaccggcagcagcaacctgcggtgccccaaggacagcgccttcgccaacagctccagcagagtcagggcatgttgggacagtcatctgtccatcagatgactcccagctcttcctacggtttgcagacttcccagggctatactccttatgtttctcatgtgggattgcagcaacacacaggccctgcaggtaccatggtgccccccagctactccagccagccttaccagagcacccacccttctaccaatcctactcttgtagatcctacccgccacctgcaacagcggcccagtggctatgtgcaccagcaggcccccacctatggacatggactgacctccactcaaaggttttcacaccagacactgcagcagacacccatgataagtaccatgactccaatgagtgcccagggcgtccaggcaggcgtccgttcaacagccatcctacctgagcagcagcagcagcagcaacagcagcaacagcaacagcagcagcagcagcaacagcaacagcagcagcagcagcagcagtaccacatccggcagcagcagcagcagcagatcctgcggcagcagcagcaacagcaacagcagcagcagcagcagcagcaacagcaacagcagcagcagcaacagcaacaacagcaacaccagcagcaacagcagcaacaggcggctcctccccaaccccagccccagtcccagccccagttccagcgccaggggcttcagcagacccagcagcagcaacagacagcagctttggtccggcaacttcaacaacagctctctaatacccagccacagcccagtaccaacatatttggacgctactgagccacctggaggaactgcttgtgcactggatgtggccccaccctttcctcttaattcccaatcccattcctgggctagcaccagtagtggttggggccctcccctcaggctccatttttaataagtttttagtatttttgttaatgtgaggcattgagctgttgggttttgtatattatttatatagagaccccagagctgttgcacccaatacacagagcttctttgcaaa

(19) United States
(12) Patent Application Publication
(10) Pub. No.: US 2023/0201166 A1
(43) Pub. Date: Jun. 29, 2023

(54) METHODS FOR TREATING CANCER BY INHIBITING CARM1

(71) Applicant: DANA-FARBER CANCER INSTITUTE, INC., Boston, MA (US)

(72) Inventors: Kai WUCHERPFENNIG, Brookline, MA (US); Sushil KUMAR, Boston, MA (US); Rong En TAY, Boston, MA (US)

(73) Assignee: DANA-FARBER CANCER INSTITUTE, INC., Boston, MA (US)

(21) Appl. No.: 17/910,864

(22) PCT Filed: Mar. 18, 2021
(86) PCT No.: PCT/US2021/023023

§ 371 Date: Sep. 12, 2022

Related U.S. Application Data
(60) Provisional application No. 62/991,479, filed on Mar. 18, 2020.

(51) Int. Cl. A61K 31/422; A61P 35/00; C12N 15/113; A61K 45/06; A61K 31/4409

(52) U.S. Cl. CPC A61K 31/422; A61P 35/00; C12N 15/113; A61K 45/06; A61K 31/4409

(57) ABSTRACT

The present disclosure provides a method of treating a subject having a cancer. The method comprises reducing expression of a Carm 1 gene and/or a Carm 1 effector gene in a cell of the subject, and/or reducing activity of a Carm 1 protein and/or a Carm 1 effector protein in a cell of the subject. The cancer is resistant to immunotherapy and/or checkpoint blockade treatment.

### RELATED APPLICATION

The present application claims priority to U.S. Provisional Application Ser. No. 62/991,479, filed Mar. 18, 2020, the entirety of which is incorporated by reference herein.

### GOVERNMENT SUPPORT

This disclosure was made with government support under grant numbers R01 CA238039 and P01 CA163222 awarded by the National Institutes of Health. The government has certain rights in the disclosure.

### REFERENCE TO APPENDIX [CD ROM/SEQUENCE LISTING]

This application is being filed electronically via EFS-Web and includes an electronically submitted Sequence Listing in .txt format. The .txt file contains a sequence listing entitled “14293_1000_Seq_Listing_ST25” created on Mar. 18, 2021 and is 122,324 bytes in size. The sequence listing contained in this .txt file is part of the specification and hereby incorporated by reference in its entirety.

### FIELD

This disclosure relates to methods of treating cancer, particularly methods of treating cancer resistant to drug treatment, such as immunotherapy, chemotherapy, radiotherapy, and/or checkpoint blockade treatment. Specifically, this disclosure is directed to methods for treating cancer, particularly methods of treating cancer resistant to drug treatment, such as immunotherapy, chemotherapy, radiotherapy, and/or checkpoint blockade treatment, by inhibiting the expression of a Carm1 gene, a Tdrd3 gene, a Med12 gene, and/or other Carm1 effector gene and/or the activity of a Carm1 protein, a Tdrd3 protein, a Med12 protein, and/or other Carm1 effector protein in immune cells, cancer cells, or both.

### BACKGROUND

Many cancer drugs have been developed that induce apoptosis of tumor cells, for example by inducing DNA damage or inhibiting key signaling pathways required for cell proliferation (Bouwman P, Jonkers J. The effects of deregulated DNA damage signalling on cancer chemotherapy response and resistance. Nat Rev Cancer 2012; 12(9):587-98 doi 10.1038/nrc3342). While such drugs can induce substantial tumor shrinkage, recurrence is a major challenge due to outgrowth of drug-resistant tumor cells. The immune system could potentially target residual disease, but many of these tumor cell-targeted drugs also compromise immune cell survival/function or the production of immune cells by the hematopoietic system. For example, chemotherapy drugs not only kill dividing tumor cells, but also rapidly dividing hematopoietic precursors and immune cells (Naito Y, Saito K, Shiiba K, Ohuchi A, Saigenji K, Nagura H, et al. CD8+ T cells infiltrated within cancer cell nests as a prognostic factor in human colorectal cancer. Cancer Res 1998; 58(16):3491-4; Sato E, Olson S H, Ahn J, Bundy B, Nishikawa H, Qian F, et al. Intraepithelial CD8+ tumor-infiltrating lymphocytes and a high CD8+/regulatory T cell ratio are associated with favorable prognosis in ovarian cancer. Proc Natl Acad Sci USA 2005; 102(51):18538-43).

Chemotherapy-induced DNA damage can induce activation of innate immune pathways in tumor cells, including the cGAS-STING pathway (Chen Q, Sun L, Chen Z J. Regulation and function of the cGAS-STING pathway of cytosolic DNA sensing. Nat Immunol 2016; 17(10):1142-9). The cGAS enzyme is activated by cytosolic double-stranded DNA, resulting in the synthesis of the cyclic dinucleotide cGAMP which activates the STING receptor and thereby induces a powerful type 1 interferon response through the IRF3 transcription factor. Importantly, type 1 interferons also induce maturation of dendritic cells, a key step for T cell-mediated immunity (Hervas-Stubbs S, Perez-Gracia J L, Rouzaut A, Sanmamed M F, Le Bon A, Melero I. Direct effects of type I interferons on cells of the immune system. Clin Cancer Res 2011; 17(9):2619-27). Some chemotherapy drugs are being used in combination with immunotherapy agents. For example, the combination of nab-paclitaxel and a PD-L1 blocking mAb was recently approved by the FDA for the treatment of metastatic triple-negative breast cancer (TNBC) but only a small fraction of treated patients benefitted from this combination regimen compared to monotherapy with nab-paclitaxel (Schmid P, Chui S Y, Emens L A. Atezolizumab and Nab-Paclitaxel in Advanced Triple-Negative Breast Cancer. Reply. N Engl J Med 2019; 380(10):987-8). It is important to develop tumor cell-targeted drugs which enhance rather than compromise immune function.

### SUMMARY

In some embodiments, the present disclosure provides a method of treating a subject having a cancer. In some embodiments, the method comprises reducing expression of a Carm1 gene and/or a Carm1 effector gene in a cell of the subject; and/or reducing activity of a Carm1 protein and/or a Carm1 effector protein in a cell of the subject. In some embodiments, the cancer is resistant to immunotherapy and/or checkpoint blockade treatment.

In some embodiments, the Carm1 effector gene is a Tdrd3 gene and the Carm1 effector protein is a Tdrd3 protein. In some embodiments, the Carm1 effector gene is a Med12 gene and the Carm1 effector protein is a Med12 protein.

In some embodiments, the reducing step comprises administering to the subject an inhibiting agent. The inhibiting agent inhibits the expression of the Carm1 gene or the Carm1 effector gene and/or the activity of the Carm1 protein or the Carm1 effector protein in the subject.

In some embodiments, the inhibiting agent is selected from the group consisting of a polynucleotide, a polypeptide, an antibody, a small molecule, a protein degrader, and a combination thereof. In some embodiments, the inhibiting agent comprises EZM2302 or TP-064. In some embodiments, the protein degrader comprises a Carm1 protein degrader, a Tdrd3 protein degrader, and/or a Med12 protein degrader. In some embodiments, the protein degrader comprises a Carm1 protein degrader.

In some embodiments, the reducing step comprises silencing the Carm1 gene or the Carm1 effector gene in the subject by shRNA mediated knockdown of mRNA or inactivation of genes.

In some embodiments, the reducing step comprises modifying the Carm1 gene or the Carm1 effector gene to decrease the expression of the Carm1 gene or the Carm1 effector gene. In some embodiments, the modifying step comprises modifying the Carm1 gene or the Carm1 effector gene by a CRISPR/Cas system.

In some embodiments, the cell is an immune cell.

In some embodiments, the immune cell is an immune effector cell, wherein the reduced expression of the Carm1 gene or the Carm1 effector gene and/or reduced activity of the Carm1 protein or the Carm1 effector protein enhances the cytotoxic function of the immune effector cell and/or reduces exhaustion of the immune effector cell. In some embodiments, the immune effector cell is selected from the group consisting of a cytotoxic T cell, a tumor-infiltrating lymphocyte (TIL), a Natural Killer T cell (NKT), a cytotoxic T lymphocyte (CTL), a dendritic cell, a CD8 T cell and a CD4 T cell.

In some embodiments, the cell is a cancer cell.

In some embodiments, the cancer cell has slowed growth, has reduced metastatic activity, has enhanced sensitivity to killing by CD8 T cells, has increased expression of an interferon response gene (e.g., an IFNα/γ pathway gene, a p53 pathway gene, etc.), has a DNA damage response, or a combination thereof. In some embodiments, the interferon response gene is an IFNα/γ pathway gene and/or a p53 pathway gene.

In some embodiments, the expression or activity is reduced in both an immune cell and a cancer cell of the subject.

In some embodiments, the method further comprises administering to the subject an immune cell having tumor specificity to the cancer and having reduced expression of the Carm1 gene or the Carm1 effector gene and/or reduced activity of the Carm1 protein or the Carm1 effector protein.

In some embodiments, the immune cell has substantially no expression of the Carm1 gene or the Carm1 effector gene.

In some embodiments, the immune cell is a CAR T cell.

In some embodiments, the cancer cell of the subject overexpresses Carm1.

In some embodiments, the cancer is a melanoma, carcinoma, sarcomas, adenocarcinoma, lymphoma, leukemia, kidney, breast, lung, bladder, colon, ovarian, prostate, pancreas, stomach, brain, head and neck, skin, uterine, testicular, glioma, esophagus, or liver cancer.

In some embodiments, the cancer is resistant to checkpoint blockade treatment with a CTLA-4, PD-L1, TIM-3, LAG3, TIGIT, or PD-1 antibody blockade therapy. In some embodiments, checkpoint blockade is selected from a group consisting of Nivolumab, Pembrolizumab, Ipilimumab, Atezolizumab, Avelumab, Durvalumab, Cemiplimab, and a combination thereof.

In some embodiments, the method further comprises administering to the subject a pharmaceutically effective amount of a second therapeutic agent for treating cancer in the subject.

In some embodiments, the second therapeutic agent is selected from the group consisting of a chemotherapy agent, an immunotherapy agent, a checkpoint blockade agent, a toxin, a radiolabel, a siRNA, a cancer vaccine, a small molecule, a peptide, an antibody, a genetically engineered cell, a CAR T cell, a cytokine and a combination thereof. In some embodiments, the second therapeutic agent is an anti-PD1 antibody, an anti-CTLA-4 antibody, an anti-PD-L1 antibody, an anti-TIGIT antibody, an anti-TIM-3 antibody, or an anti-LAG3 antibody.

In some other embodiments, the present disclosure provides a method of treating cancer in a subject. The method comprises reducing expression of a Carm1 gene or a Carm1 effector gene and/or activity of a Carm1 protein or a Carm1 effector protein in a cell of the subject.

In some embodiments, the method further comprises administering to the subject a pharmaceutically effective amount of a second therapeutic agent for treating cancer in the subject.

In some embodiments, the second therapeutic agent is selected from the group consisting of a chemotherapy agent, an immunotherapy agent, a checkpoint blockade agent, a toxin, a radiolabel, a siRNA, a cancer vaccine, a small molecule, a peptide, an antibody, a genetically engineered cell, a CAR T cell, a cytokine and a combination thereof. In some embodiments, the second therapeutic agent is an anti-PD1 antibody, an anti-CTLA-4 antibody, an anti-PD-L1 antibody, an anti-TIGIT antibody, an anti-TIM-3 antibody, or an anti-LAG3 antibody.

In some embodiments, the reducing step comprises administering to the subject an inhibiting agent, wherein the inhibiting agent inhibits the expression of the Carm1 gene or the Carm1 effector gene and/or the activity of the Carm1 protein or the Carm1 effector protein in the subject.

In some embodiments, the reducing step comprises silencing the Carm1 gene or the Carm1 effector gene in the subject by shRNA mediated knockdown of mRNA or inactivation of genes.

In some embodiments, the reducing step comprises modifying the Carm1 gene or the Carm1 effector gene to decrease the expression of the Carm1 gene or the Carm1 effector gene. In some embodiments, the modifying step comprises modifying the Carm1 gene or the Carm1 effector gene by a CRISPR/Cas system.

In some embodiments, the reducing step comprises degrading the Carm1 protein or the Carm1 effector protein.

In yet some embodiments, the present disclosure provides a method of sensitizing a cancer cell to an immune effector cell. The method comprises inhibiting expression and/or activity of a Carm1 gene or protein, or a Carm1 effector gene or protein in a cancer cell by one or more inhibiting agents, wherein the inhibiting sensitizes the cancer cell to an immune cell.

In some embodiments, the inhibiting agent comprises a polynucleotide, a polypeptide, a peptide, an antibody, a small molecule, a protein degrader, or a combination thereof. In some embodiments, the inhibiting agent comprises EZM2302 or TP-064.

In some embodiments, the inhibiting agent comprises a Carm1 protein degrader, a Tdrd3 protein degrader, and/or a Med12 protein degrader. In some embodiments, the inhibiting agent comprises a Carm1 protein degrader.

In some embodiments, the Carm1 effector gene or protein is a Tdrd3 gene or protein. In some embodiments, the Carm1 effector gene or protein is a Med12 gene or protein.

In some embodiments, the immune effector cell is selected from the group consisting of a cytotoxic T cell, a tumor-infiltrating lymphocyte (TIL), a Natural Killer T cell (NKT), a cytotoxic T lymphocyte (CTL), a dendritic cell, a CD8 T cell and a CD4 T cell.

In still some embodiments, the present disclosure provides a method of increasing the anti-tumor function of an immune effector cell. The method comprises reducing expression and/or activity of a Carm1 gene or protein, or a Carm1 effector gene or protein in the immune effector cell, thereby increasing the anti-tumor function of the immune effector cell.

In some embodiments, the Carm1 effector gene or protein is a Tdrd3 gene or protein. In some embodiments, the Carm1 effector gene or protein is a Med12 gene or protein.

In some embodiments, the reducing step comprises inhibiting, by one or more inhibiting agents, the expression and/or activity of the Carm1 gene or protein, or the Carm1 effector gene or protein in the immune cell.

In some embodiments, the inhibiting agent comprises a polynucleotide, a polypeptide, a peptide, an antibody, a small molecule, a protein degrader, a genetically engineered cell, or a combination thereof. In some embodiments, the inhibiting agent comprises EZM2302 or TP-064.

In some embodiments, the inhibiting agent comprises a Carm1 protein degrader, a Tdrd3 protein degrader, and/or a Med12 protein degrader. In some embodiments, the inhibiting agent comprises a Carm1 protein degrader.

In some embodiments, the expression and/or activity of the Carm1 gene or protein, or a Carm1 effector gene or protein is reduced by shRNA mediated knockdown of mRNA or inactivation of gene.

In some embodiments, the reducing step comprises modifying the immune effector cell to reduce or remove the CARM1 gene or Carm1 effector gene in the immune effector cell. In some embodiments, the modifying step comprises modifying the Carm1 gene or the Carm1 effector gene by a CRISPR/Cas system.

In some embodiments, the reducing step comprises silencing the CARM1 gene or the Carm1 effector gene in the immune effector cell.

In some embodiments, the reducing step comprises degrading the Carm1 protein or the Carm1 effector protein in the immune effector cell.

In some embodiments, the immune effector cell is selected from the group consisting of a cytotoxic T cell, a tumor-infiltrating lymphocyte (TIL), a Natural Killer T cell (NKT), a cytotoxic T lymphocyte (CTL), a dendritic cell, a CD8 T cell and a CD4 T cell. In some embodiments, the CD8 T cell expresses a higher level of CD69, CD45.1, granzyme B, IFNγ, Ki67, or a combination thereof.

In some embodiments, the present disclosure provides an immune effector cell. The immune cell has an inhibiting agent of a Carm1 gene/protein or a Carm1 effector gene/protein. The inhibiting agent inhibits expression of the Carm1 gene or Carm1 effector gene, and/or activity of the Carm1 protein or the Carm1 effector protein.

In some embodiments, the Carm1 effector gene is a Tdrd3 gene and the Carm1 effector protein is a Tdrd3 protein. In some embodiments, the Carm1 effector gene is a Med12 gene and the Carm1 effector protein is a Med12 protein.

In some embodiments, the immune effector cell has substantially no expression of the Carm1 gene or the Carm1 effector gene.

In some embodiments, the inhibiting agent is selected from the group consisting of a polynucleotide, a polypeptide, an antibody, a small molecule, a protein degrader, and a combination thereof. In some embodiments, the inhibiting agent comprises EZM2302 or TP-064.

In some embodiments, the inhibiting agent comprises a Carm1 protein degrader, a Tdrd3 protein degrader, and/or a Med12 protein degrader. In some embodiments, the inhibiting agent comprises a Carm1 protein degrader.

In some embodiments, the immune effector cell is selected from the group consisting of a cytotoxic T cell, a tumor-infiltrating lymphocyte (TIL), a Natural Killer T cell (NKT), a cytotoxic T lymphocyte (CTL), a dendritic cell, a CD8 T cell and a CD4T cell.

In some embodiments, the immune effector cell is tumor specific.

In some embodiments, the immune effector cell expresses a tumor-specific T-cell receptor or a chimeric antigen receptor (CAR).

In some embodiments, the immune effector cell further comprises a chimeric antigen receptor (CAR), wherein the CAR comprises an antigen-binding domain, a transmembrane domain, and a stimulatory domain.

In some embodiments, the antigen-binding domain binds a tumor antigen or pathogen antigen.

In some embodiments, the tumor antigen is selected from a group consisting of an antigen present in a cancer cell, a cancer cell, a cancer cell fragment, a tumor antigen, α-galcer, anti-CD3, anti-CD28, anti-IgM, anti-CD40, a pathogen, an attenuated pathogen, and a portion thereof.

In some embodiments, the tumor antigen is associated with a melanoma, carcinoma, sarcomas, adenocarcinoma, lymphoma, leukemia, kidney, breast, lung, bladder, colon, ovarian, prostate, pancreas, stomach, brain, head and neck, skin, uterine, testicular, glioma, esophagus, or liver cancer.

In some embodiments, the tumor antigen is associated with a solid tumor or lymphoid tumor.

In some embodiments, the antigen-binding domain is an antigen-binding fragment of an antibody.

In still some embodiments, the present disclosure provides a composition comprising the immune effector cell described herein and a pharmaceutically acceptable carrier. In some embodiments, the immune effector cell has an inhibiting agent of a Carm1 gene/protein or a Carm1 effector gene/protein. The inhibiting agent inhibits expression of the Carm1 gene or Carm1 effector gene, and/or activity of the Carm1 protein or the Carm1 effector protein.

In some embodiments, the composition further comprises a second therapeutic agent.

In some embodiments, the second therapeutic agent is selected from the group consisting of a chemotherapy agent, an immunotherapy agent, a checkpoint blockade agent, a toxin, a radiolabel, a siRNA, a cancer vaccine, a small molecule, a peptide, an antibody, a genetically engineered cell, a CAR T cell, a cytokine and a combination thereof.

In some embodiments, the second therapeutic agent is an anti-PD1 antibody, an anti-CTLA-4 antibody, an anti-PD-L1 antibody, an anti-TIGIT antibody, an anti-TIM-3 antibody, or an anti-LAG3 antibody.

In some embodiments, the immune effector cell is selected from the group consisting of a cytotoxic T cell, a tumor-infiltrating lymphocyte (TIL), a Natural Killer T cell (NKT), a cytotoxic T lymphocyte (CTL), a dendritic cell, a CD8 T cell and a CD4 T cell.

In yet some embodiments, the present disclosure provides a method of treating cancer in a subject. The method comprises administering to a subject having cancer the immune effector cell described herein or the composition described herein.

In some embodiments, the immune effector cell has an inhibiting agent of a Carm1 gene/protein or a Carm1 effector gene/protein. The inhibiting agent inhibits expression of the Carm1 gene or Carm1 effector gene, and/or activity of the Carm1 protein or the Carm1 effector protein.

In some embodiments, the composition comprises the immune effector cell described herein and a pharmaceutically acceptable carrier. In some embodiments, the immune effector cell has an inhibiting agent of a Carm1 gene/protein or a Carm1 effector gene/protein. The inhibiting agent inhibits expression of the Carm1 gene or Carm1 effector gene, and/or activity of the Carm1 protein or the Carm1 effector protein.

In some embodiments, the immune effector cell is autologous.

In some embodiments, the immune effector cell is specific to a cancer cell of the subject.

In some embodiments, the method further comprises administering to the subject having cancer a second therapeutic agent, or a composition comprising a second therapeutic agent and a pharmaceutically acceptable carrier.

In some embodiments, the second therapeutic agent is selected from the group consisting of a chemotherapy agent, an immunotherapy agent, a checkpoint blockade agent, a toxin, a radiolabel, a siRNA, a cancer vaccine, a small molecule, a peptide, an antibody, a genetically engineered cell, a CAR T cell, a cytokine and a combination thereof.

In some embodiments, the second therapeutic agent is an anti-PD1 antibody, an anti-CTLA-4 antibody, an anti-TIGIT antibody, an anti-TIM-3 antibody, or an anti-LAG3 antibody.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. The disclosures of these publications, patent applications, patents, sequences, database entries, and other references in their entireties are hereby incorporated by reference into this application in order to more fully describe the state of the art as known to those skilled therein as of the date of the disclosure described and claimed herein. In case of conflict, the present specification, including definitions, will control.

Other features and advantages of the disclosure will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1A illustrates that Carm1 is an epigenetic inhibitor in tumor-specific T cells, showing experimental design for in vivo discovery of epigenetic regulators that inhibit CD8 T cell accumulation in tumors.

FIG. 1B illustrates that Carm1 is an epigenetic inhibitor in tumor-specific T cells, showing in vivo CRISPR screen with epigenetic gRNA library in tumor specific CD8 T cells. gRNA quantification in CD8 T cells was compared in tumors (experimental site) and spleens (control organ) (log 2 fold change). Major experimental genes and positive control genes were highlighted in red and blue, respectively.

FIG. 1C illustrates that Carm1 is an epigenetic inhibitor in tumor-specific T cells, showing MAGeCK (Model-based Analysis of Genome-Wide CRIPSR-Cas9 knockout) analysis of in vivo CRISPR screen data; MAGeCK score provides integrated readout for strength of gene effects.

FIG. 1D illustrates that Carm1 is an epigenetic inhibitor in tumor-specific T cells, showing T cell cytotoxicity assay with Carm1-KO and control-KO OT-I CD8 T cells. CD8 T cells were edited by electroporation with Cas9 protein and bound gRNA, and cells were grown in IL-15+IL-7 containing T cell media for 5 days. T cells were co-cultured with B16F10-OVA-ZsGreen tumor cells at indicated effector to target (E:T) ratios (n=8-10/replicates per condition); 24 hours later live GFP-positive tumor cells were counted using a Celigo image cytometer. Data are representative of three experiments and shown as mean±SEM. ****p<0.0001, by unpaired two-sided Mann-Whitney test.

FIG. 1E illustrates that Carm1 is an epigenetic inhibitor in tumor-specific T cells, showing anti-tumor activity of adoptively transferred Carm1-KO or control-KO OT-I CD45.1 CD8 T cells. B16-OVA-ZsGreen tumor cells (0.1×10⁶) were implanted subcutaneously. On day 7 post tumor cell inoculation, edited CD8 T cells (1×10⁶) were transferred via tail vein injection. Tumor size was recorded; n=8-10 mice per group.

FIG. 1F illustrates that Carm1 is an epigenetic inhibitor in tumor-specific T cells, showing tumor weights 7 days following adoptive T cell transfer for experiment shown in FIG. 1E.

FIG. 1G illustrates that Carm1 is an epigenetic inhibitor in tumor-specific T cells, showing flow cytometry analysis of tumor-infiltrating Carm1-KO or control-KO CD8 T cells following adoptive transfer of edited OT-I CD45.1 CD8 T cells (n=10 mice/group) with gating on CD45.1 and CD8 T cell markers. Quantification of CD8 T cell infiltration and expression of effector (granzyme B, IFNγ) as well as proliferation (Ki-67) markers.

FIG. 2A illustrates Carm1 inhibition in CD8 T cells enhances their anti-tumor function, showing RNA-seq analysis of differentially expressed genes in Carm1-KO or control-KO OT-I CD8 T cells co-cultured for 24 hours with B16F10-Ova tumor cells (four biological replicates per condition). Color code represents Z-scores for differential gene expression.

FIG. 2B illustrates Carm1 inhibition in CD8 T cells enhances their anti-tumor function, showing volcano plot of all differentially expressed genes between Carm1-KO and control-KO OT-I CD8 T cells. Statistical significance (log 10 adjusted P value) was plotted against log 2 fold change of gene expression levels (Carm1-KO/control-KO cells).

FIG. 2C illustrates Carm1 inhibition in CD8 T cells enhances their anti-tumor function, showing RT-qPCR analysis of Tcf7, Myb, Bcl6, Btg2, Itgae, Havcr2 and Klrg1 mRNA levels in Carm1-KO and control-KO CD8 T cells (targeting of Carm1 with two different gRNAs, triplicate measurements).

FIG. 2D illustrates Carm1 inhibition in CD8 T cells enhances their anti-tumor function, showing Gene Ontology (GO) analysis of significantly upregulated/downregulated pathways in Carm1-KO versus control-KO T cells.

FIG. 2E illustrates Carm1 inhibition in CD8 T cells enhances their anti-tumor function, showing tumor-infiltrating Carm1-KO or control-KO CD8 T cells following adoptive transfer of edited OT-I CD45.1 CD8 T cells (n=10 mice/group) with gating on CD45.1 and CD8 T cell markers. Quantification of Tcf7+ T cells with high Bcl2 protein levels and Tcf7+ CD8 T cells.

FIG. 2F illustrates Carm1 inhibition in CD8 T cells enhances their anti-tumor function, showing quantification of Bcl2 high tumor-infiltrating Carm1-KO or control-KO CD8 T cells.

FIGS. 2G, 2H and 2I illustrate Carm1 inhibition in CD8 T cells enhances their anti-tumor function, showing tumor-infiltrating Carm1-KO or control-KO CD8 T cells were analyzed 16 or 24 days following adoptive transfer of edited OT-I CD45.1 CD8 T cells (n=8 mice/group) with gating on CD45.1 and CD8 T cell markers. Quantification of CD8 T activation marker (CD69) (FIG. 2G) and markers of T cell exhaustion (FIGS. 2H-2I). In FIGS. 2A-2I, data shown are representative of two experiments. Two-way ANOVA was used to determine statistical significance for time points when all mice were viable for tumor measurement. Graphs shown represent data summarized as mean±S.D. and were analyzed by unpaired two-sided Mann-Whitney test, ****P<0.0001; ***P<0.001; **P<0.01; *P<0.05.

FIG. 3A illustrates that inactivation of Carm1 gene in tumor cells elicits tumor immunity, showing CARM1 mRNA levels in a diverse panel of 1,208 cancer cell lines from the Cancer Cell Line Encyclopedia (CCLE). Tumor cell lines were grouped based on cancer type.

FIG. 3B illustrates that inactivation of Carm1 gene in tumor cells elicits tumor immunity, showing strategy for targeting Carm1 in tumor cells to study impact on T cell-mediated tumor immunity.

FIG. 3C illustrates that inactivation of Carm1 gene in tumor cells elicits tumor immunity, showing Western blot analysis of Carm1 protein in B16F10 melanoma cells following electroporation with RNPs composed of Cas9 protein and bound gRNAs (control, Carm1); two different control and Carm1 gRNAs were evaluated.

FIG. 3D illustrates that inactivation of Carm1 gene in tumor cells elicits tumor immunity, showing growth of Carm1-KO and control-KO B16F10 tumors (left) and survival of tumor bearing mice (right). Mice (n=8-10/group) were treated with CD8 depleting or isotype control antibodies. This in vivo phenotype was confirmed with a second Carm1 gRNA (FIG. 10A).

FIG. 3E illustrates that inactivation of Carm1 gene in tumor cells elicits tumor immunity, showing growth of Carm1-KO tumors in T cell-deficient mice. Carm1-KO and control-KO B16F10 tumor cells (0.2×10⁶) were implanted into immunocompetent or immunodeficient (Tcra KO) mice (n=8-10 mice/group); tumor growth (left) and survival (right) were recorded.

FIG. 3F illustrates that inactivation of Carm1 gene in tumor cells elicits tumor immunity, showing growth of Carm1-KO or control-KO 4T1 tumor cells following implantation into the mammary fat pad (n=8-10 mice/group); tumor growth (left) and survival (right) were recorded

FIG. 3G illustrates that inactivation of Carm1 gene in tumor cells elicits tumor immunity, showing quantification of spontaneous lung metastases formed by Carm1-KO or control-KO 4T1 tumors in immunocompetent mice. Representative images of lung metastases (V, ventral; D, dorsal) (right).

FIG. 311 illustrates that inactivation of Carm1 gene in tumor cells elicits tumor immunity, showing tumor growth (left) and survival (right) following implantation of Carm1-KO and control-KO MC38 tumor cells (n=8-10 mice/group).

FIG. 31 illustrates that inactivation of Carm1 gene in tumor cells elicits tumor immunity, showing T cell cytotoxicity assay with Carm1-KO or control-KO B16F10-OVA-ZsGreen tumor cells. Tumor cells were co-cultured for 24 hours with OT-I CD8 T cells at indicated effector to target (E:T) ratios (n=8-10 replicates per condition).

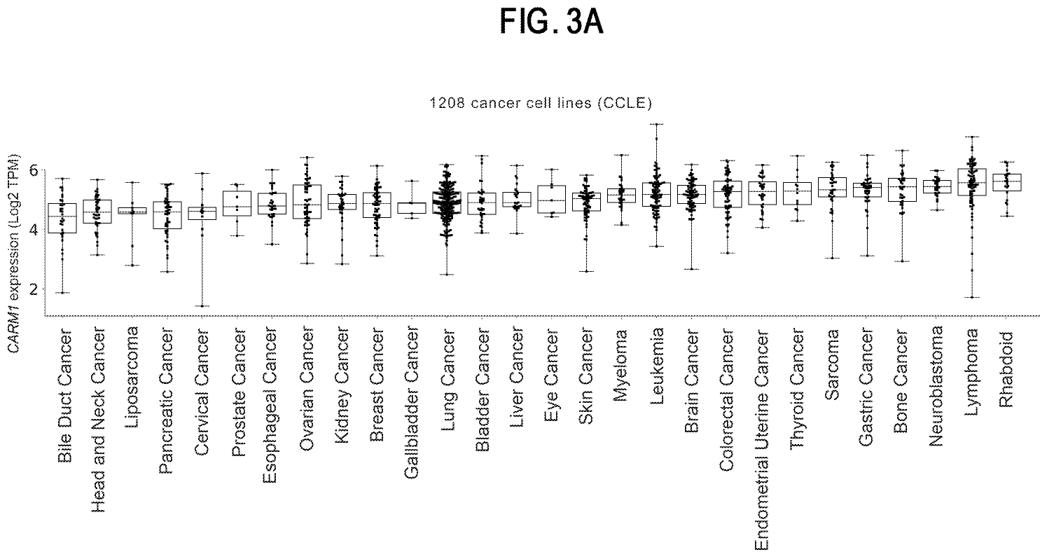

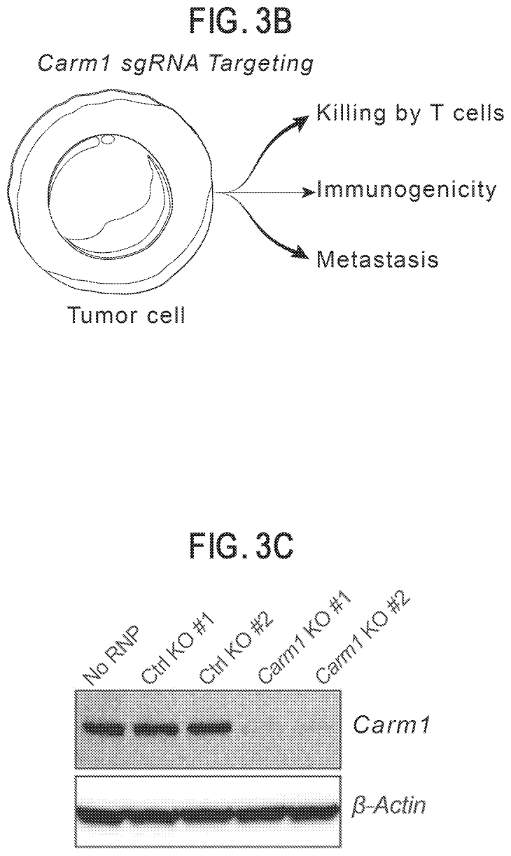

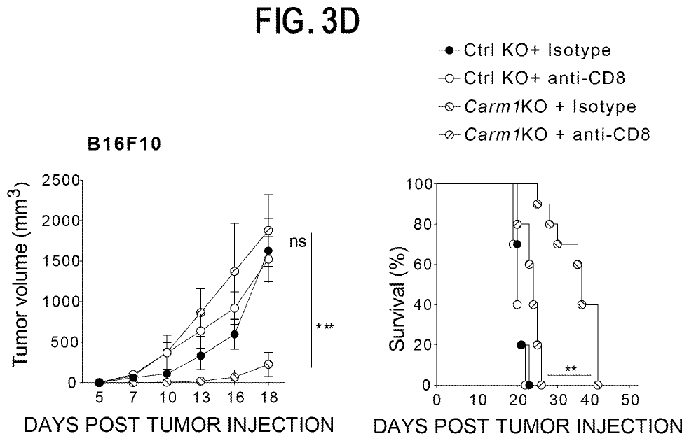

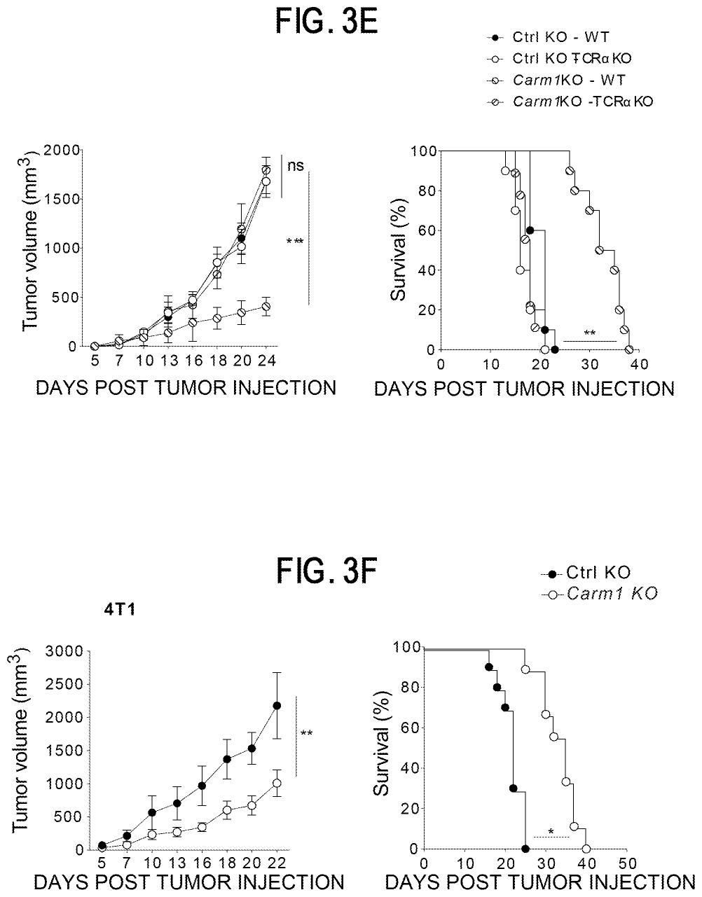

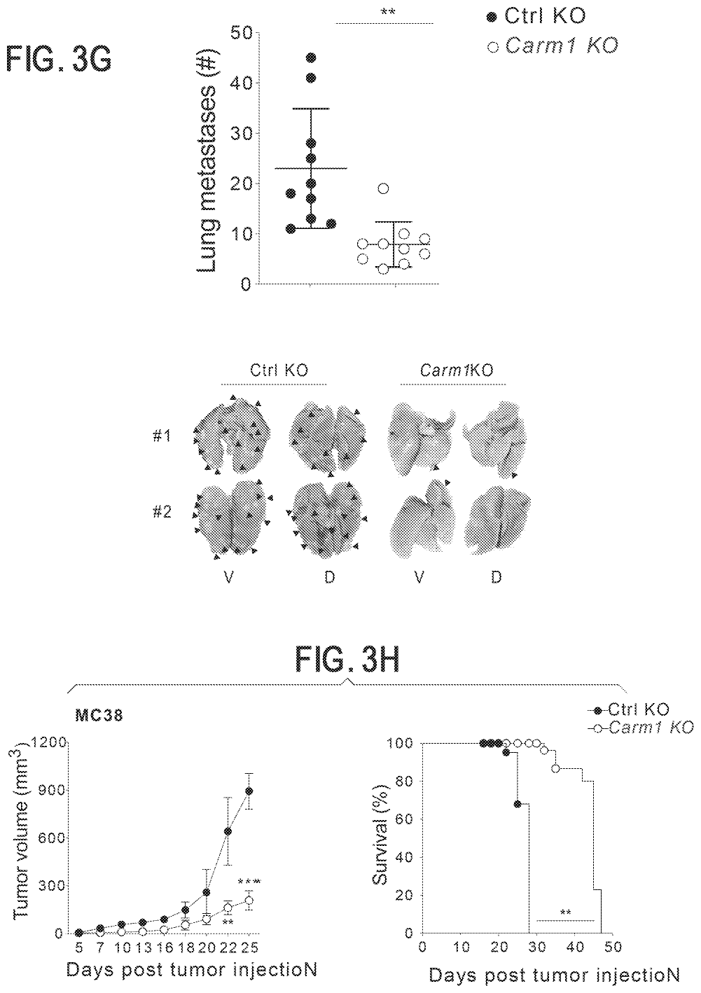

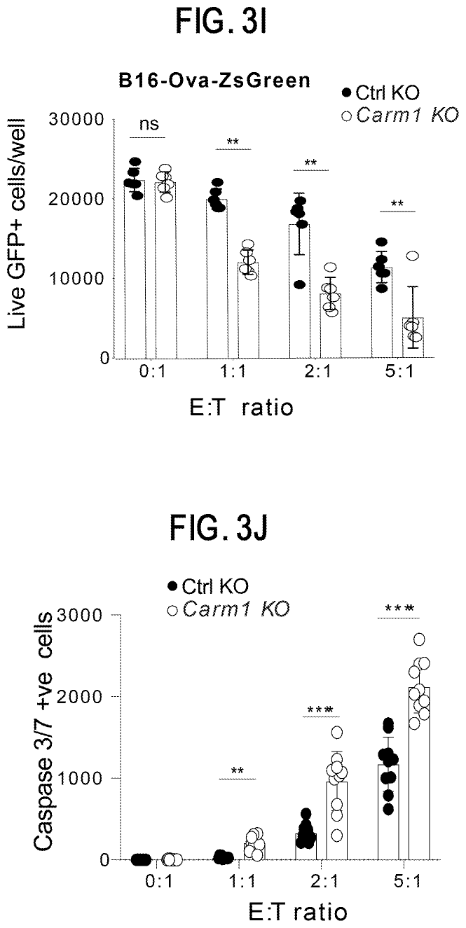

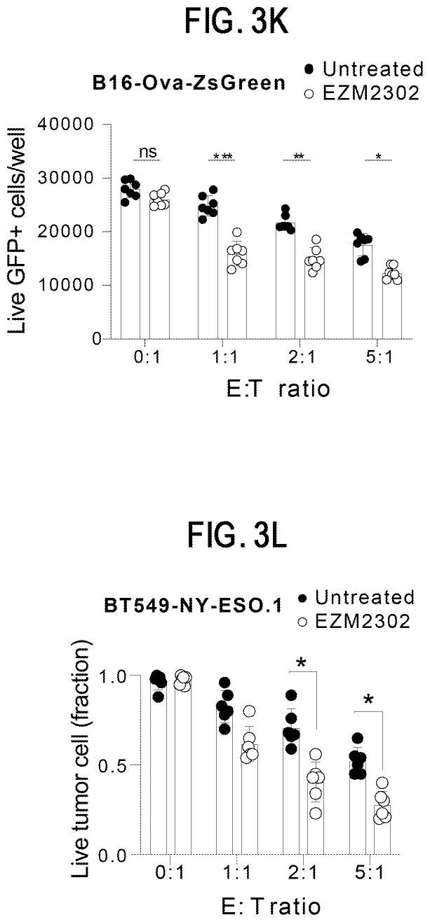

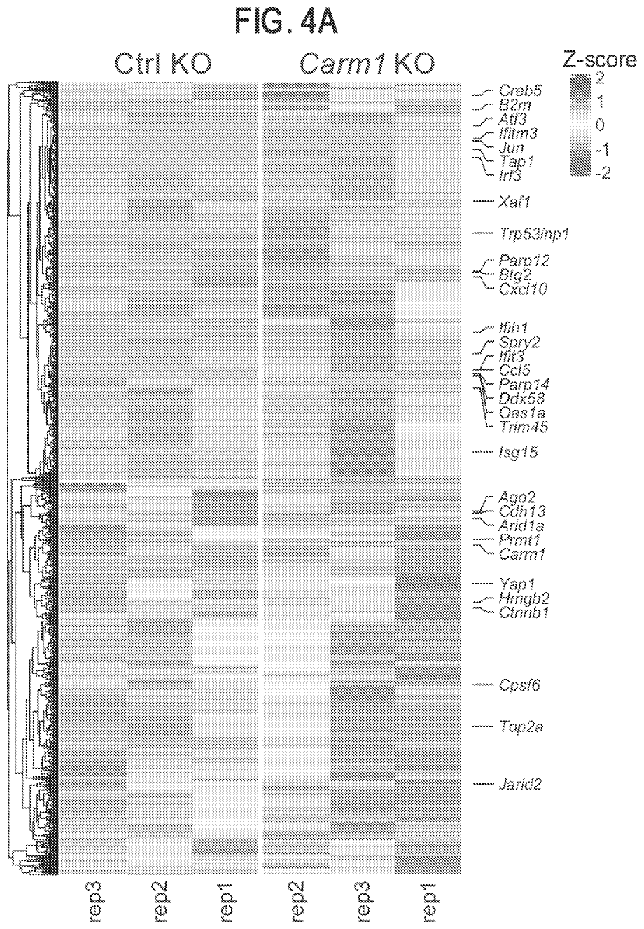

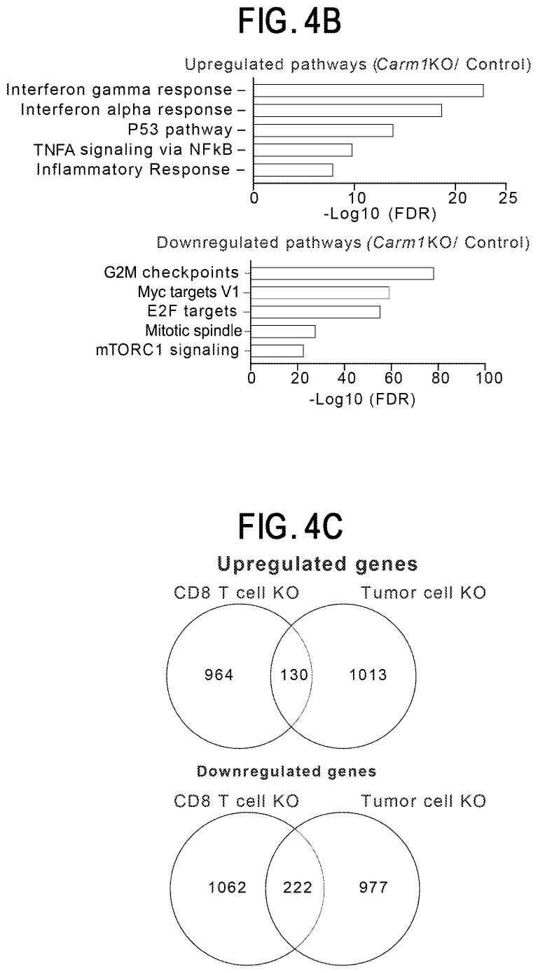

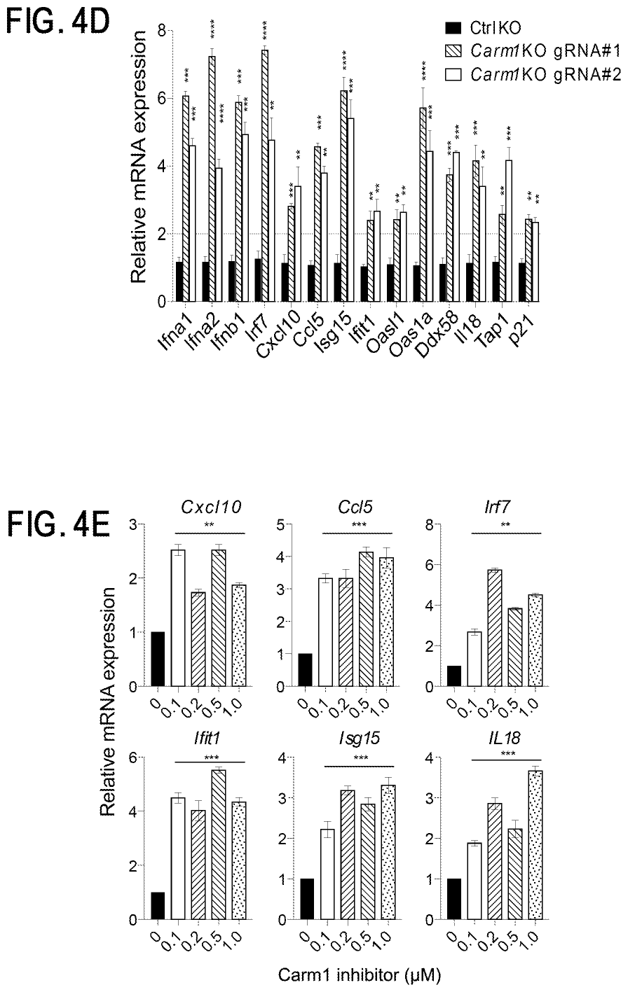

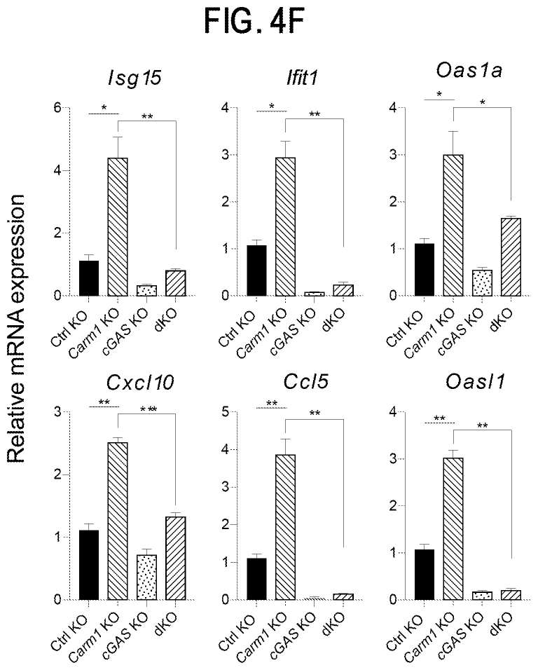

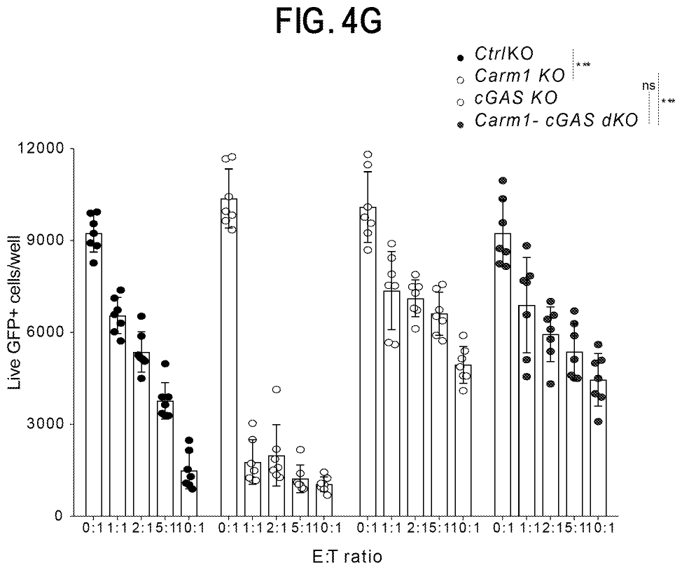

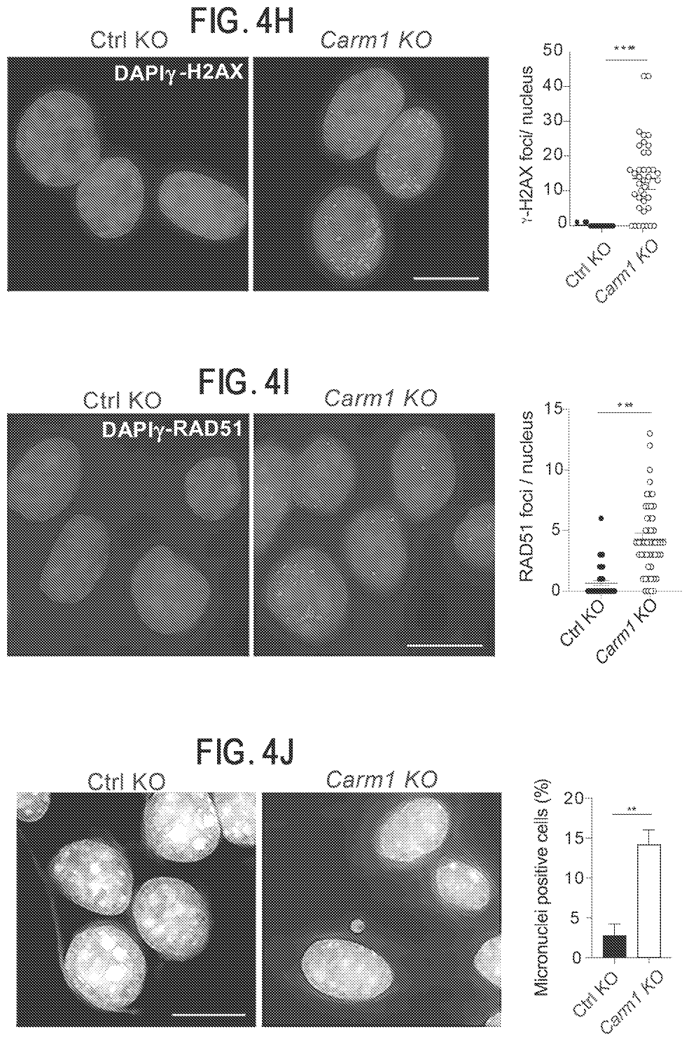

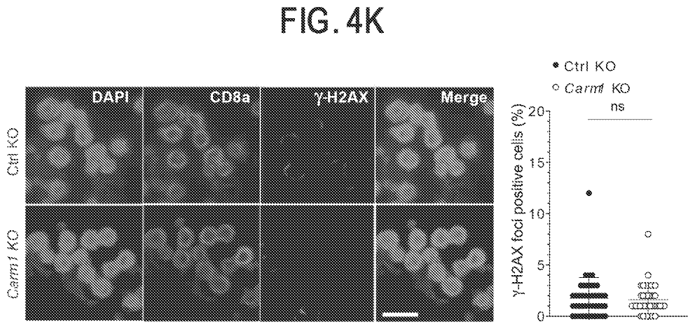

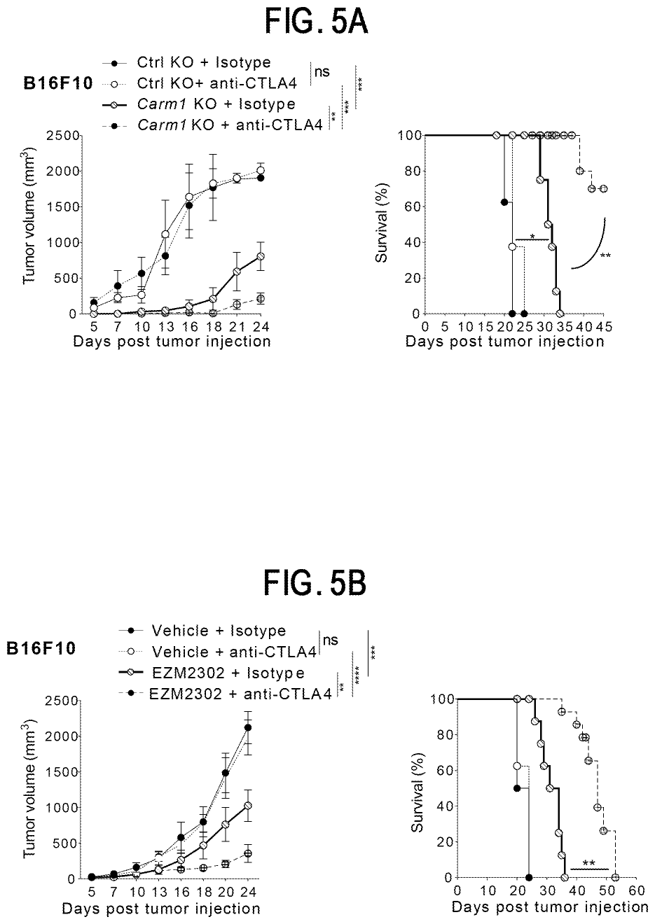

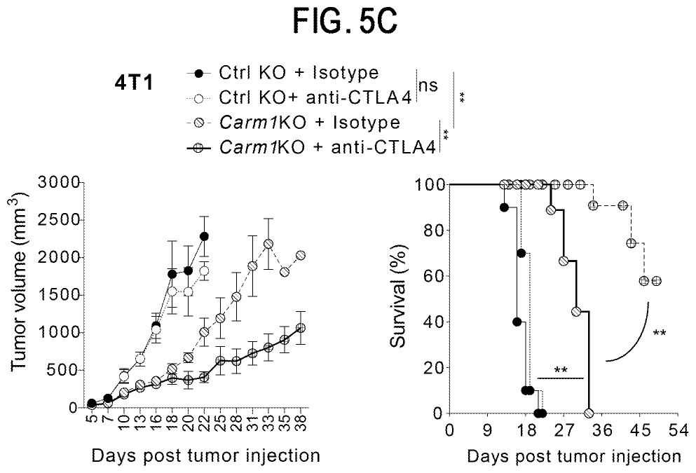

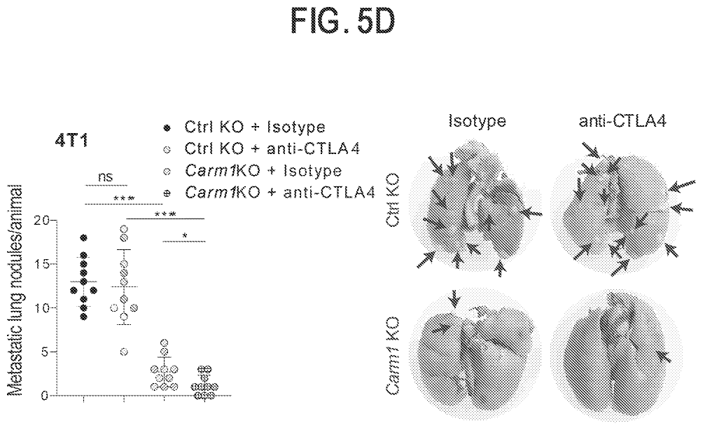

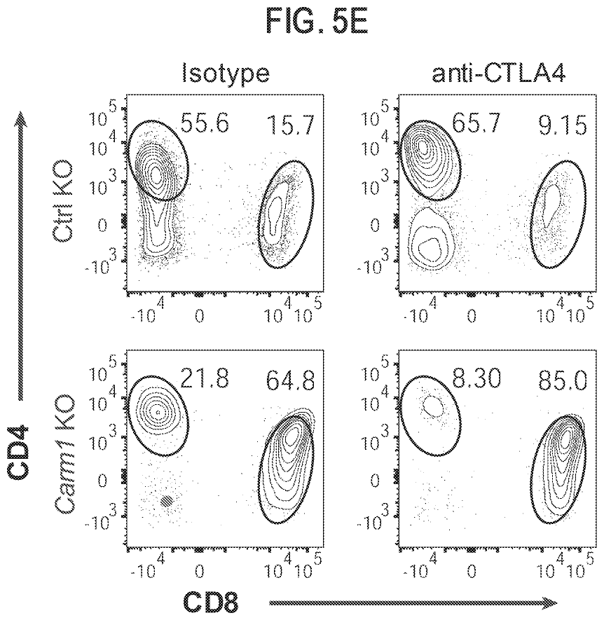

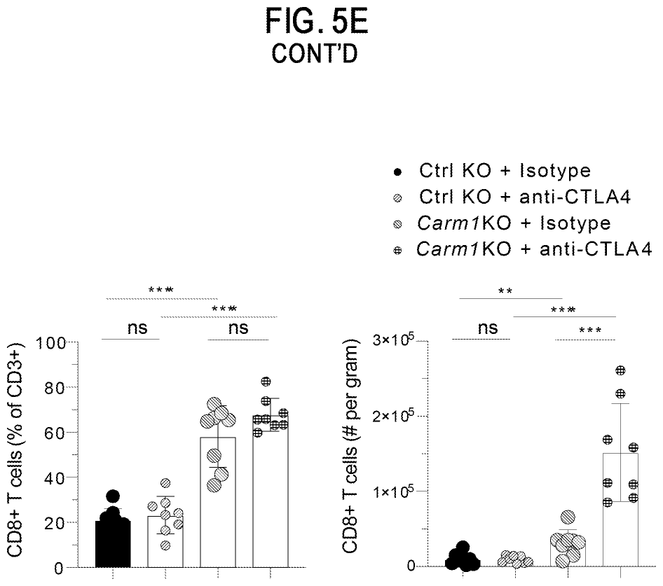

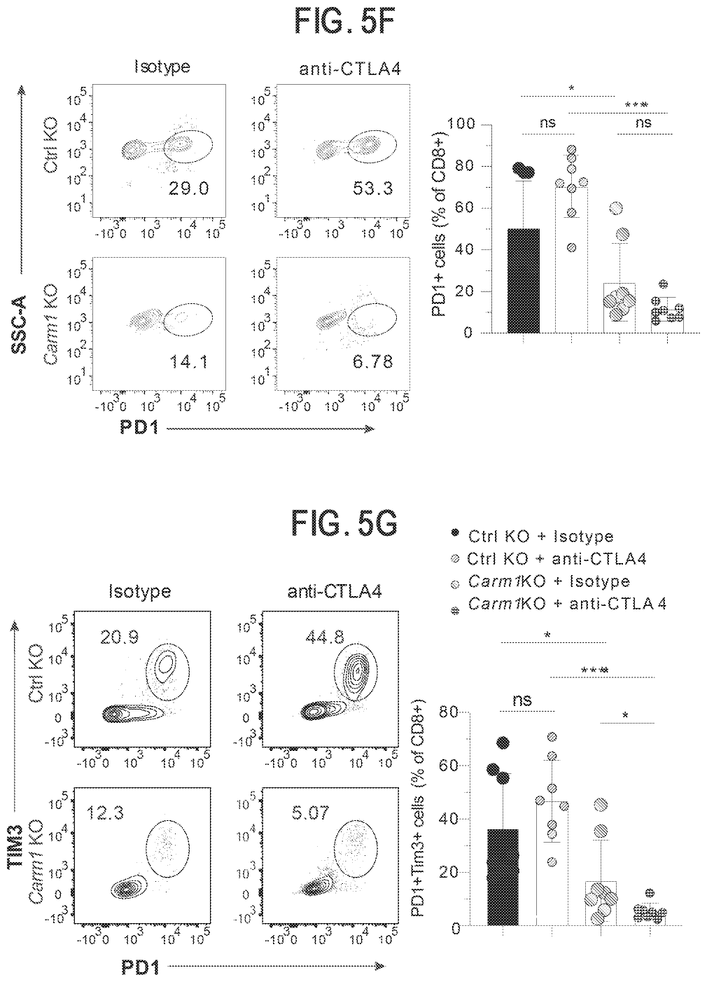

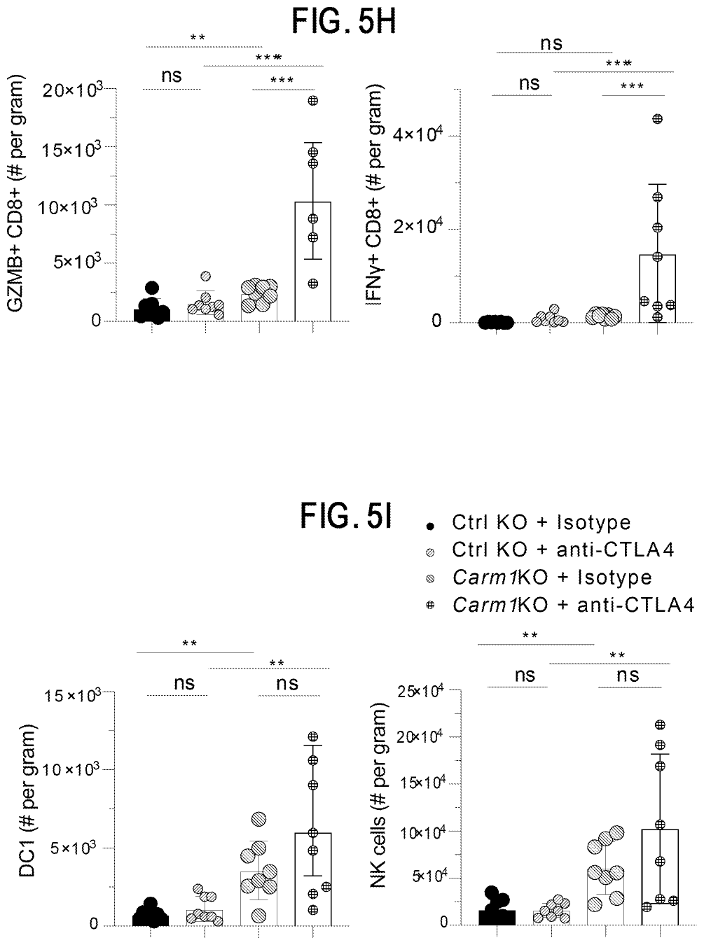

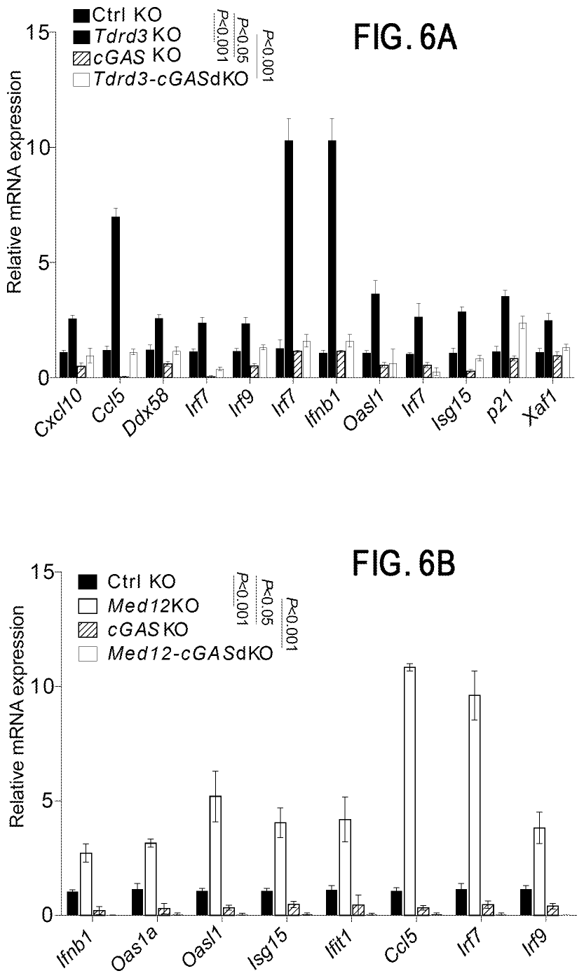

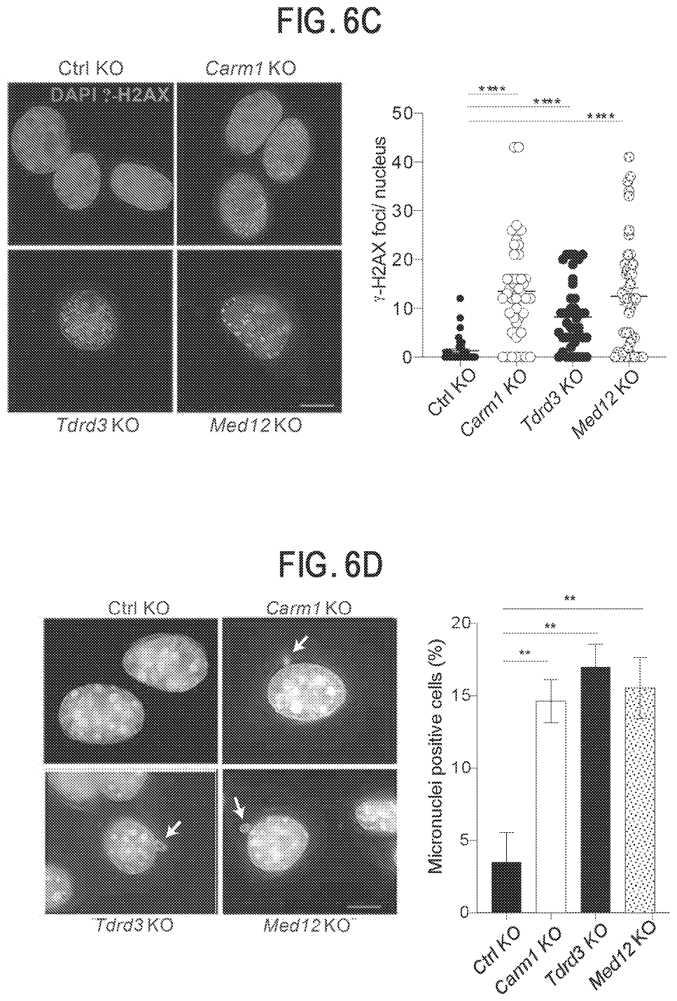

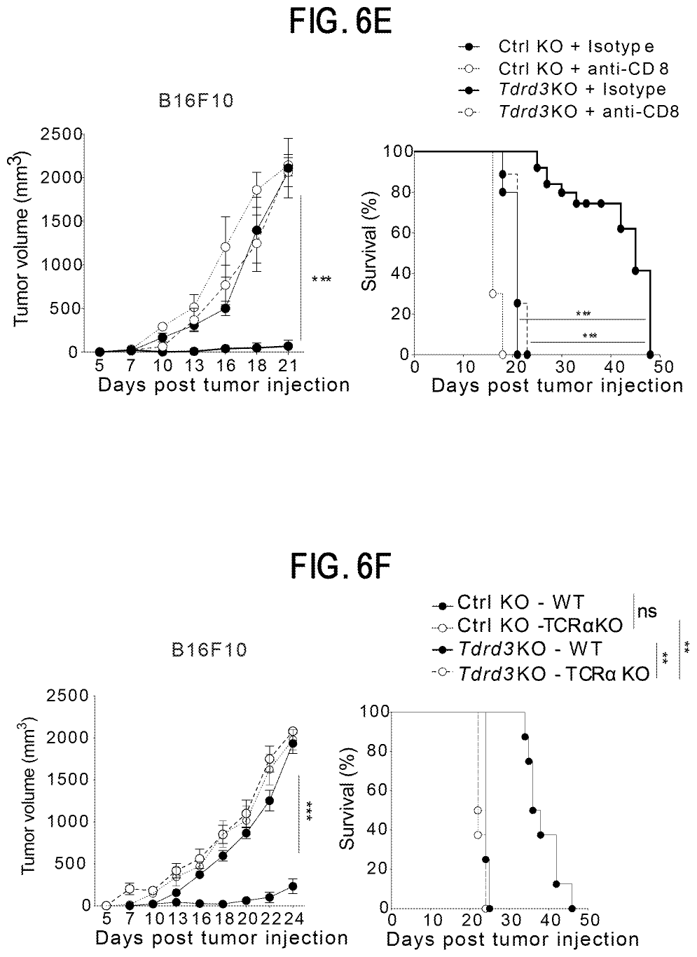

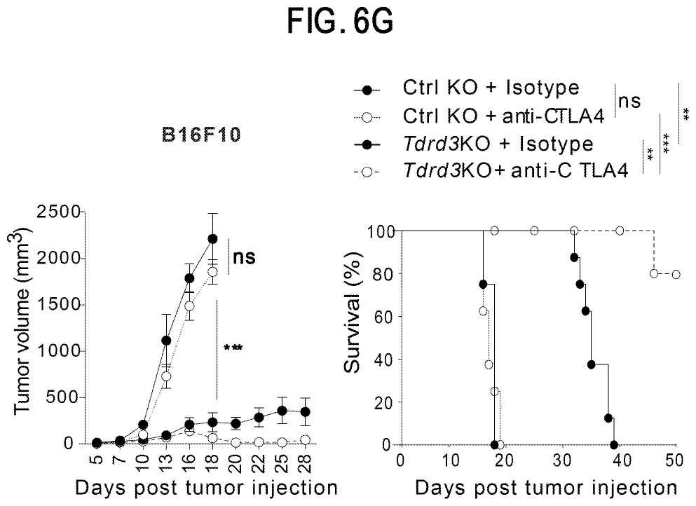

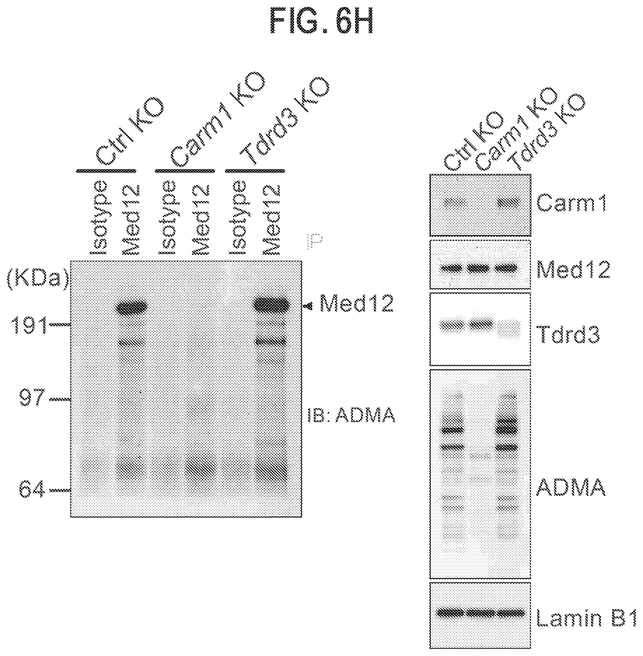

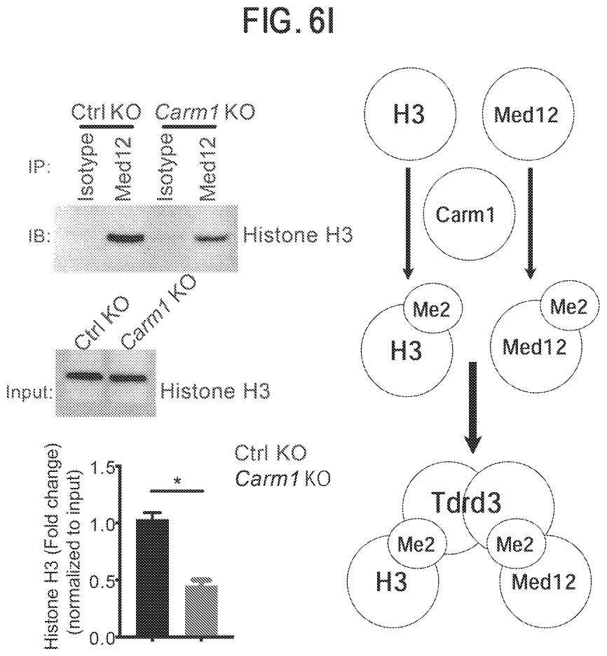

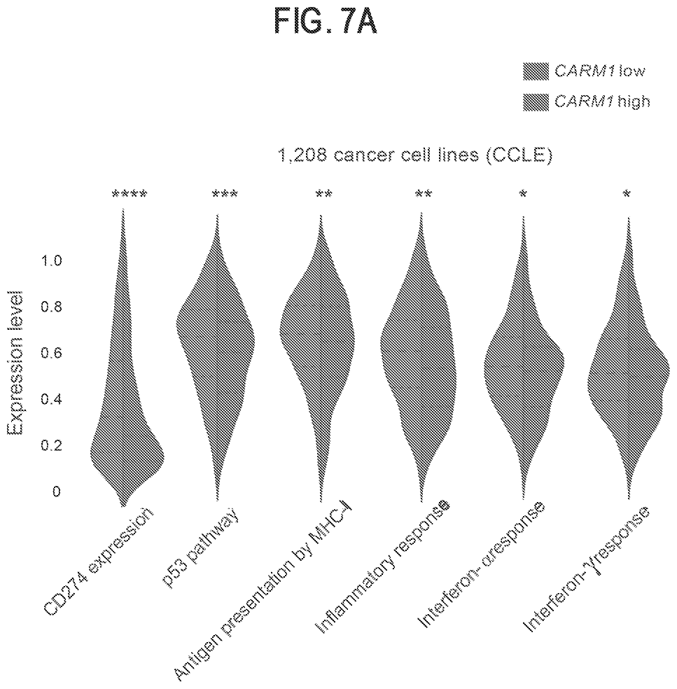

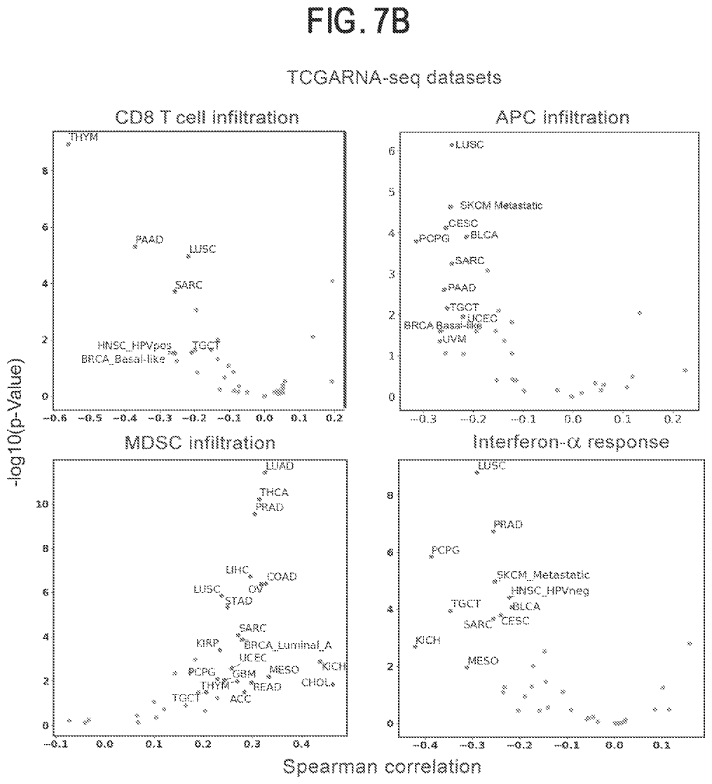

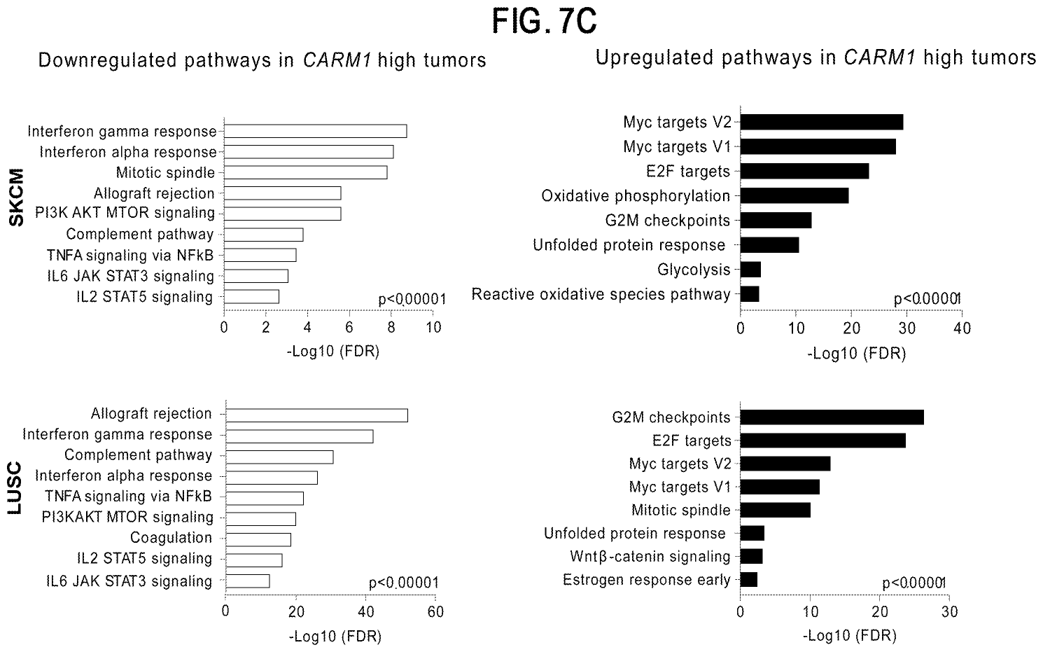

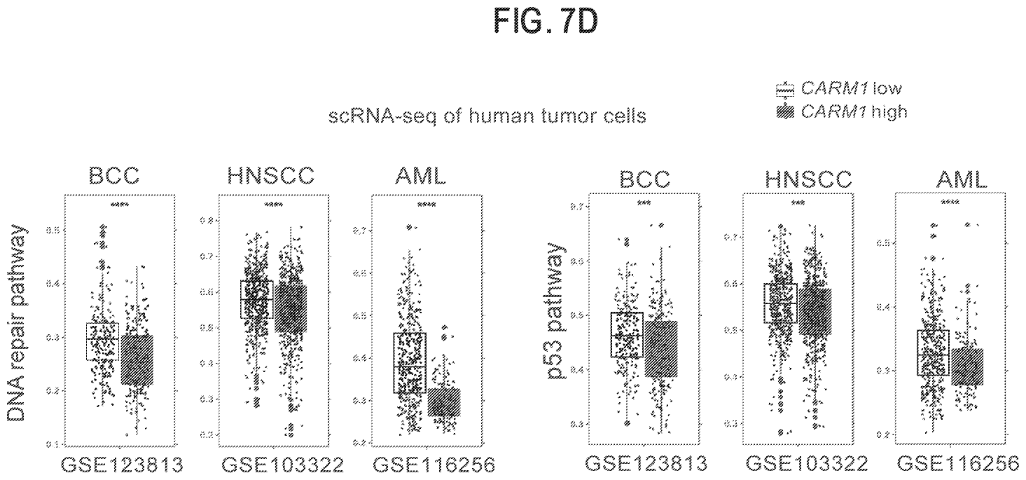

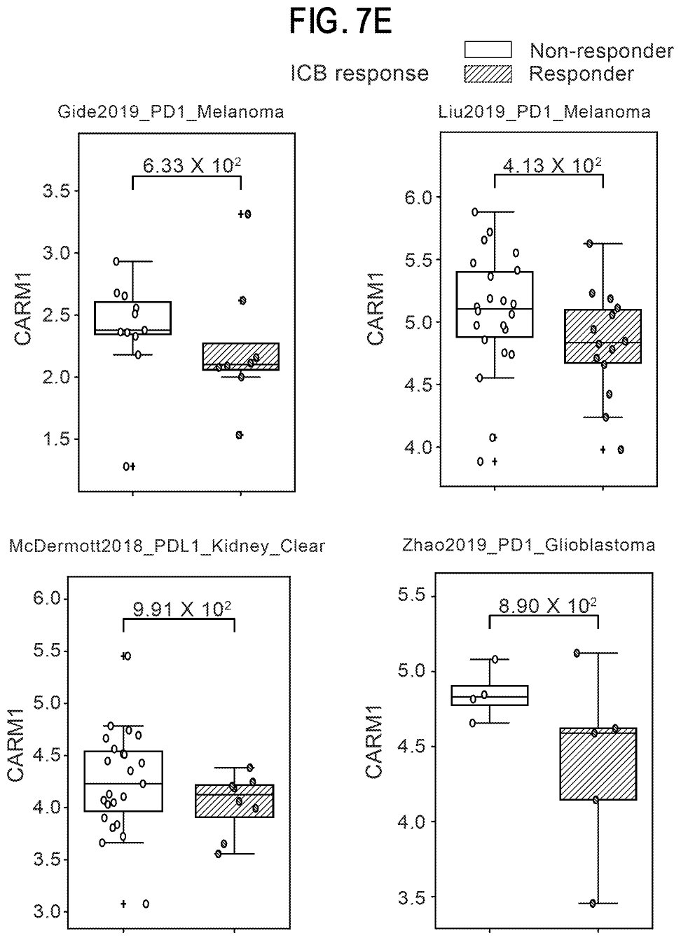

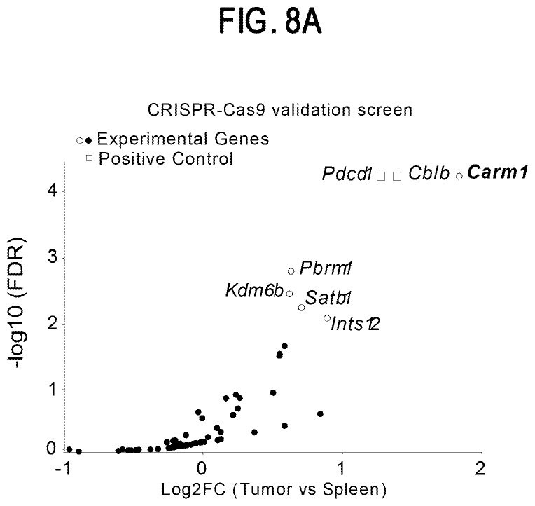

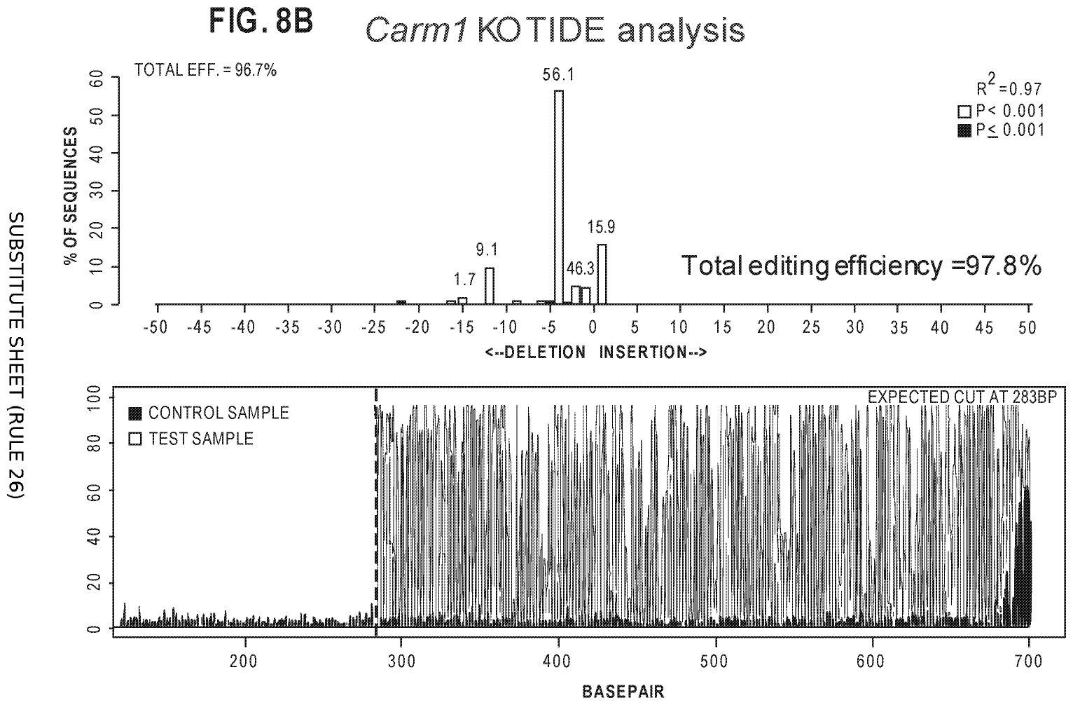

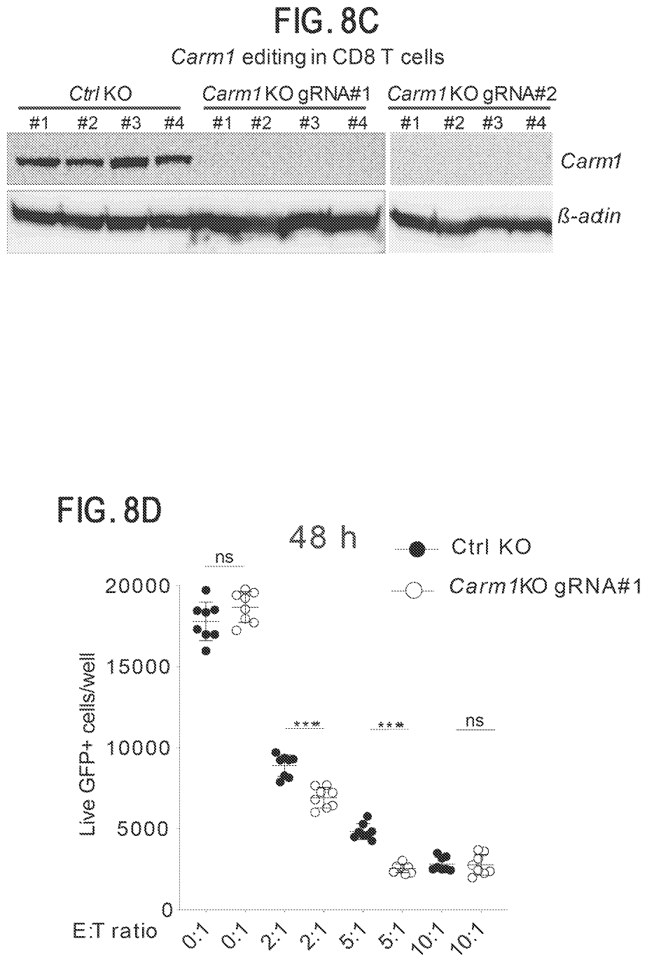

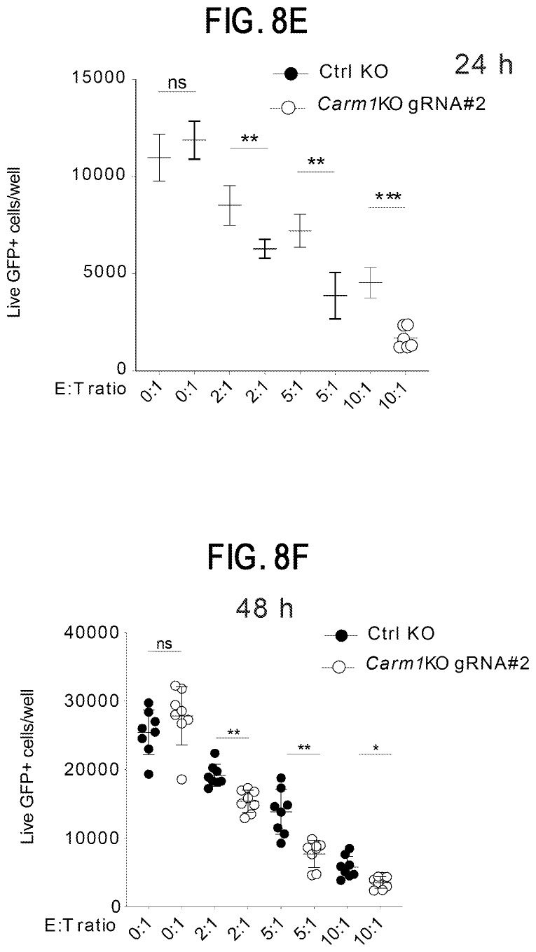

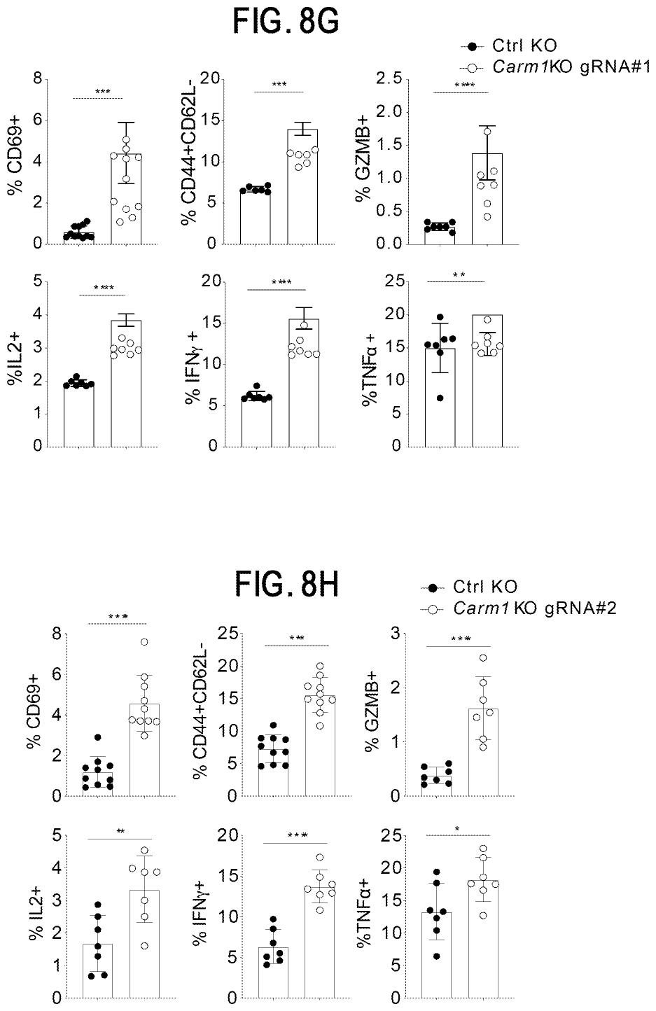

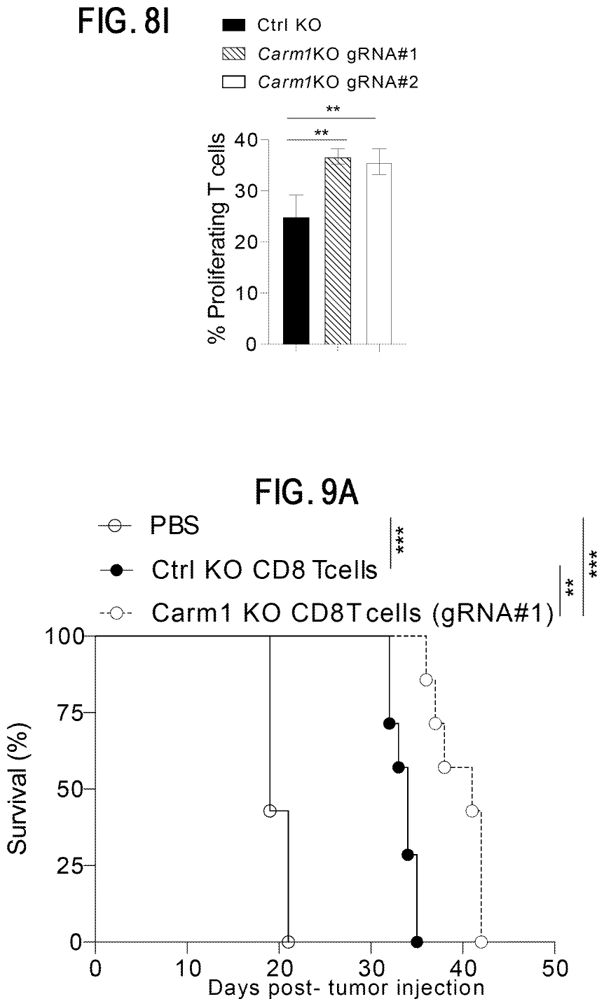

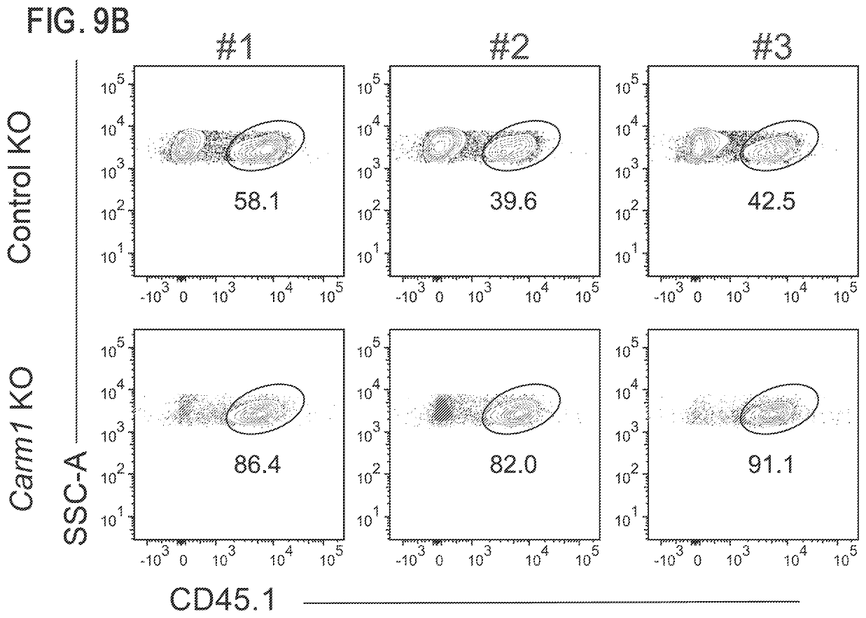

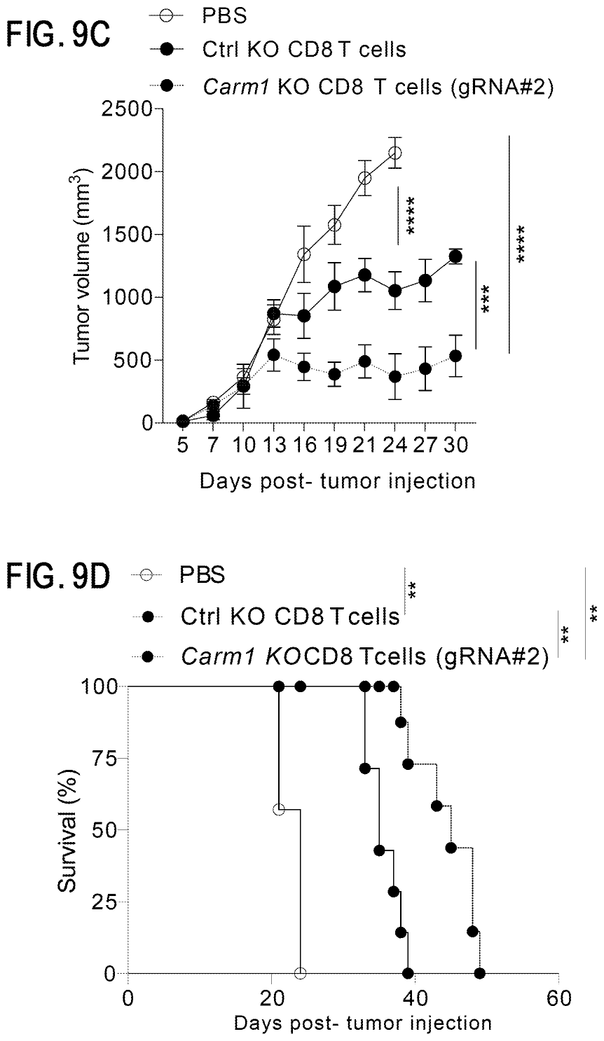

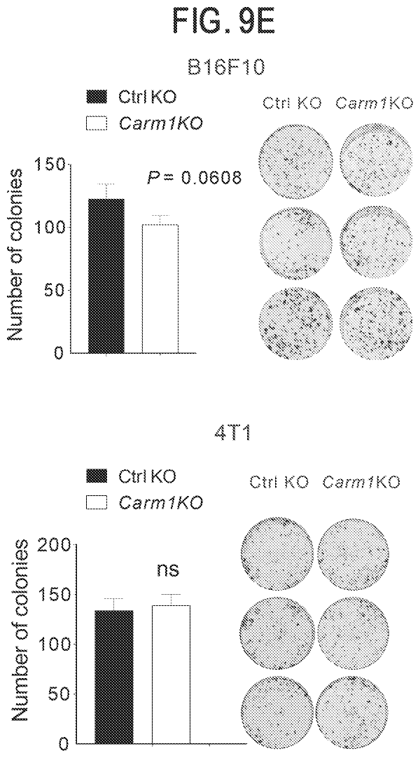

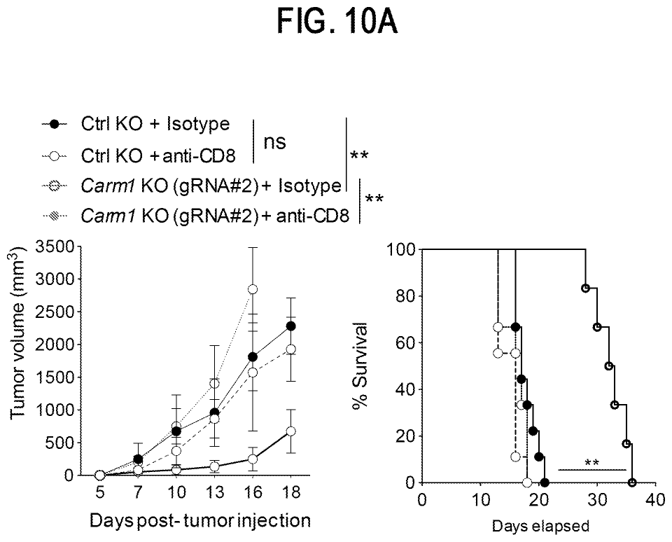

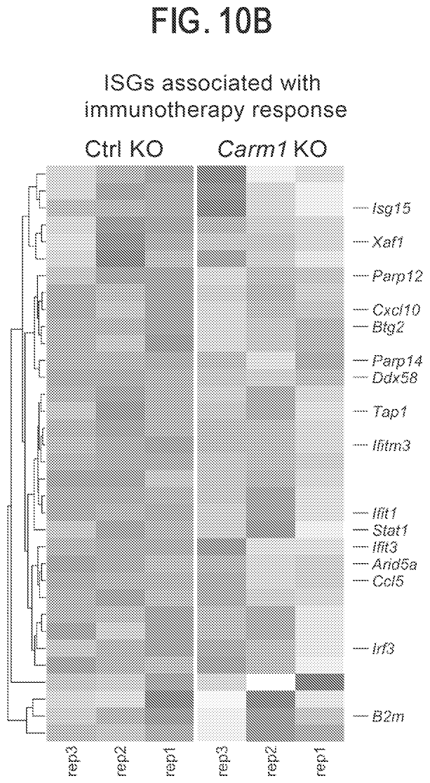

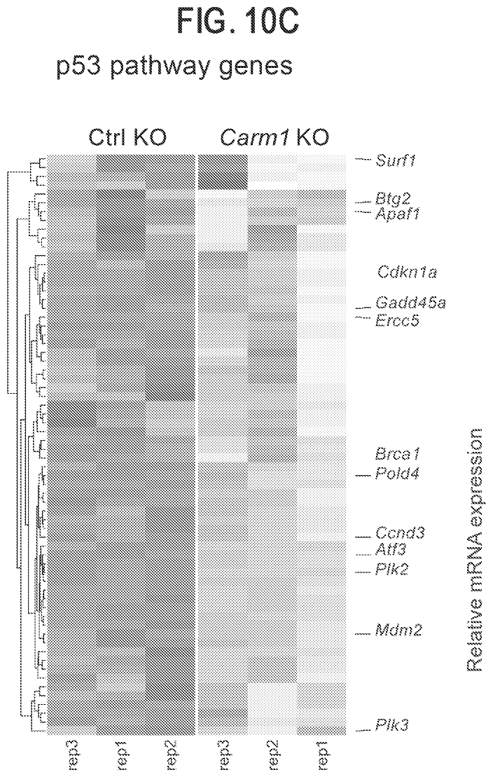

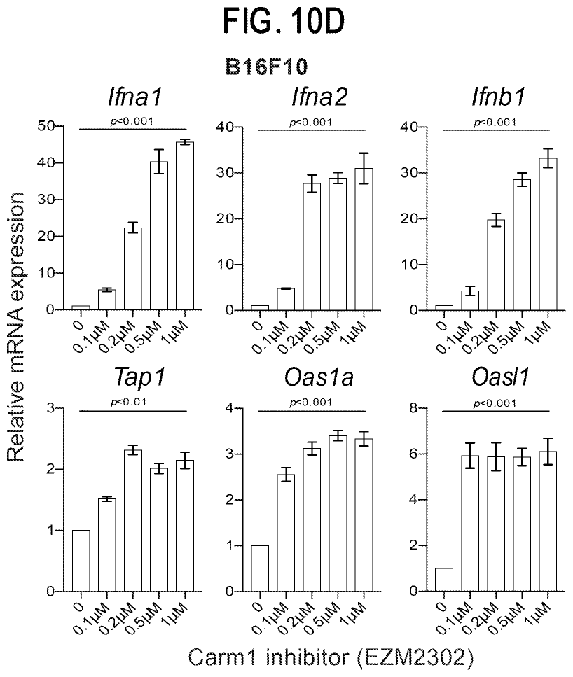

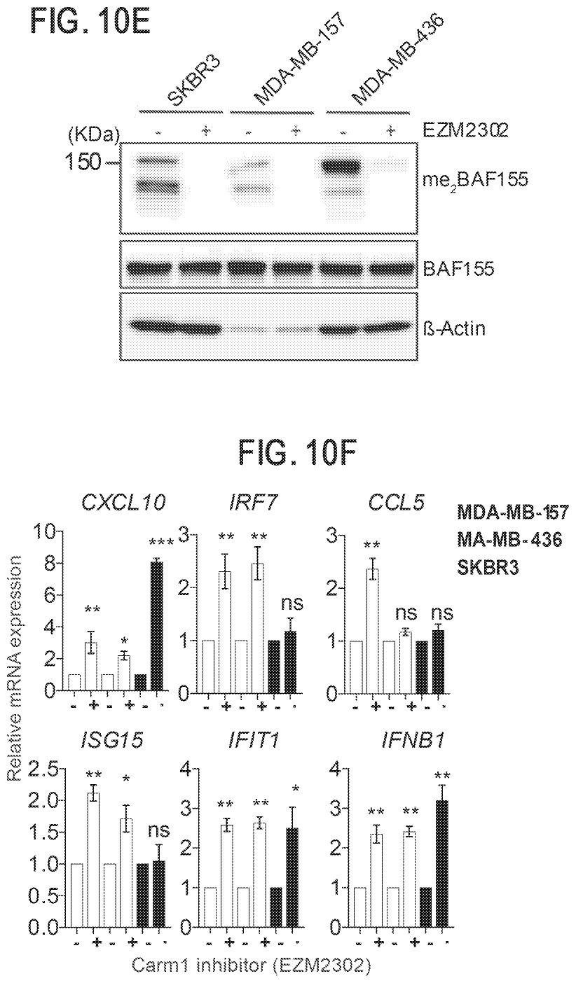

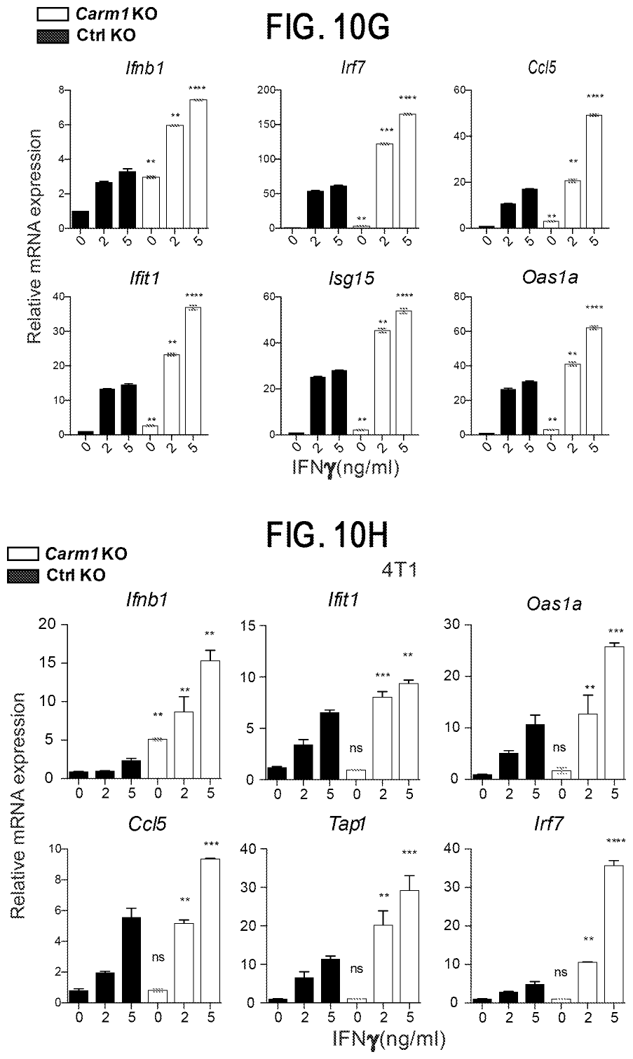

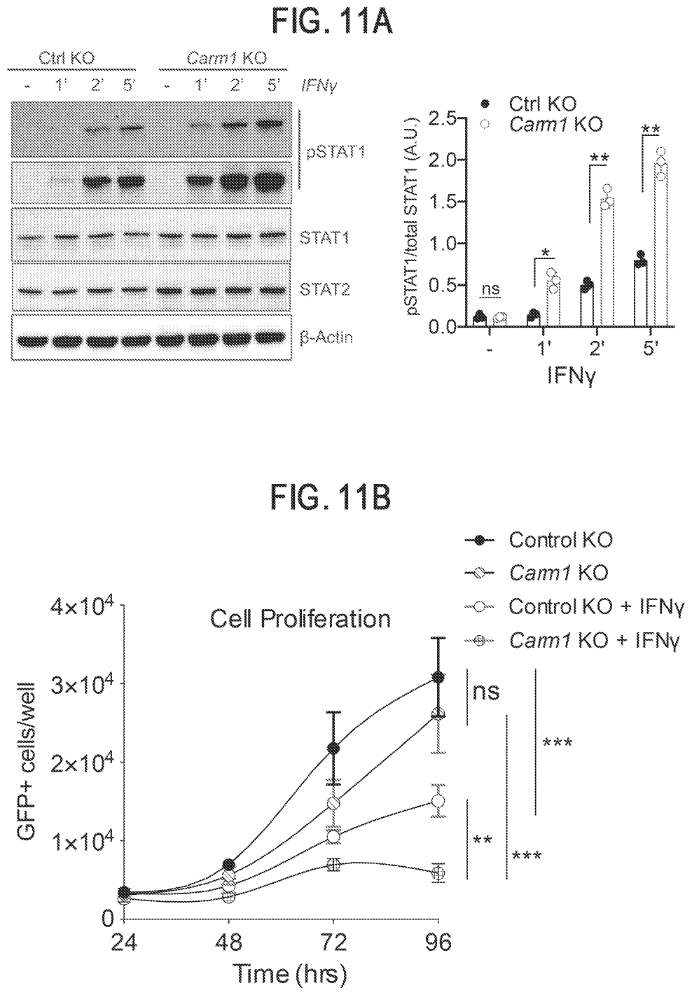

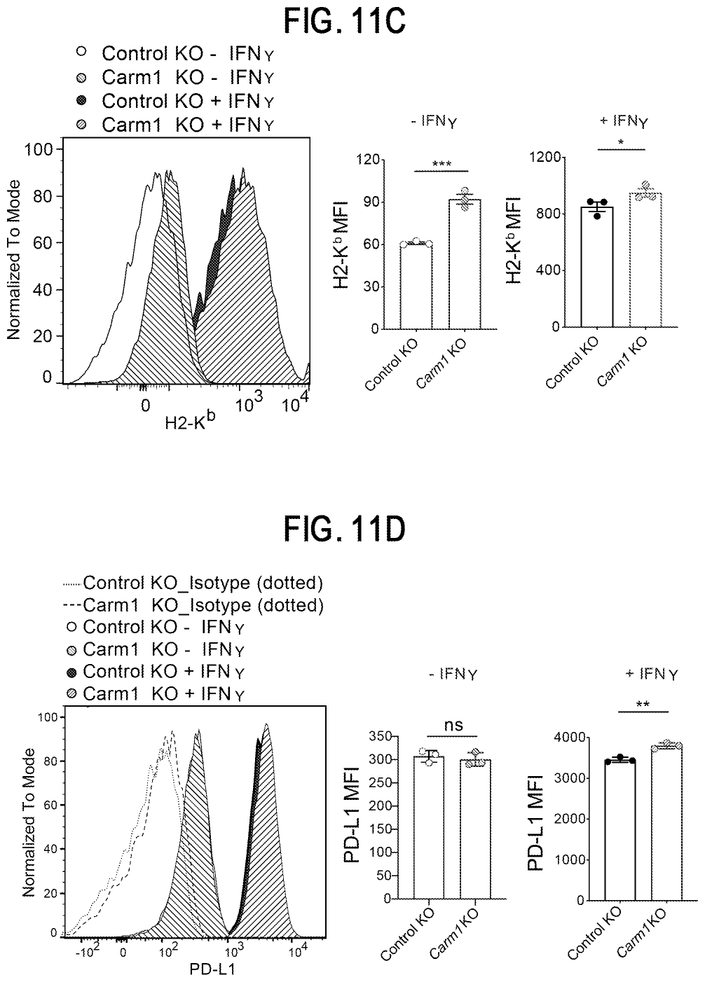

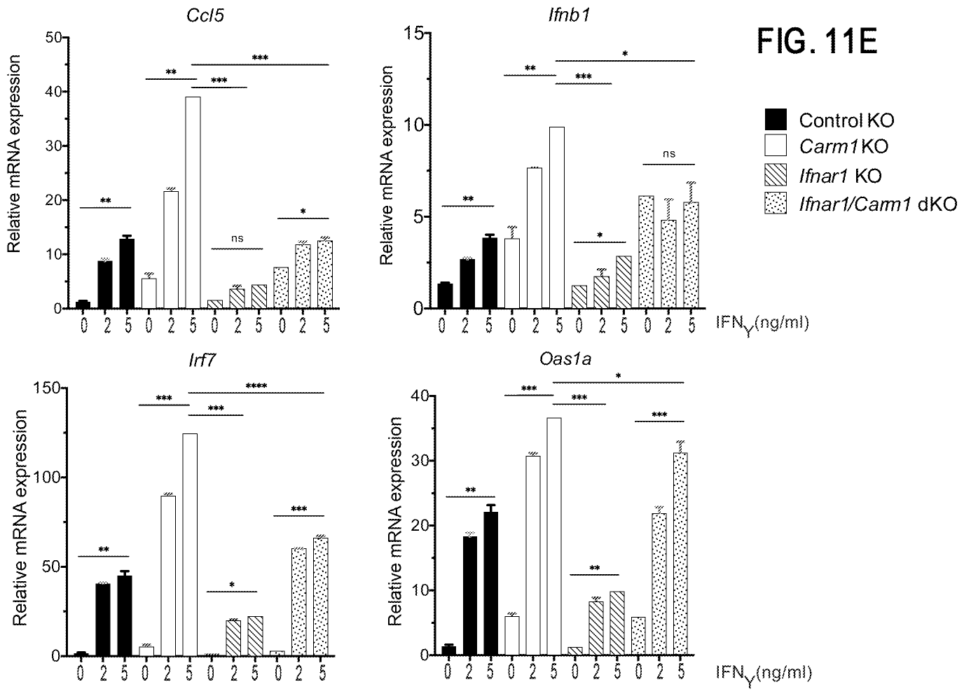

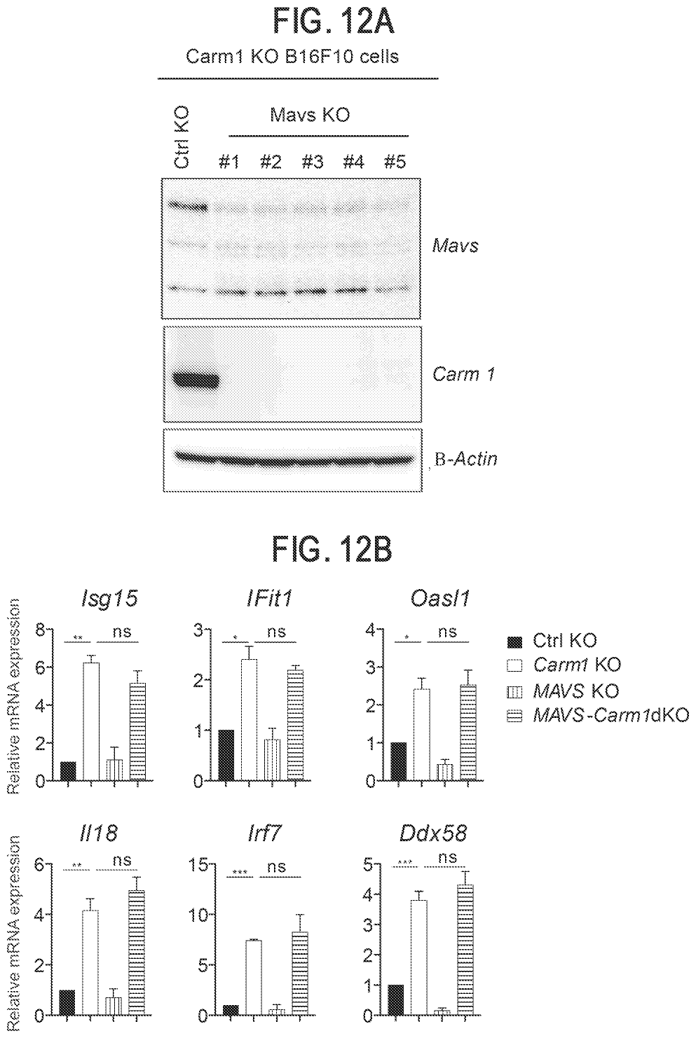

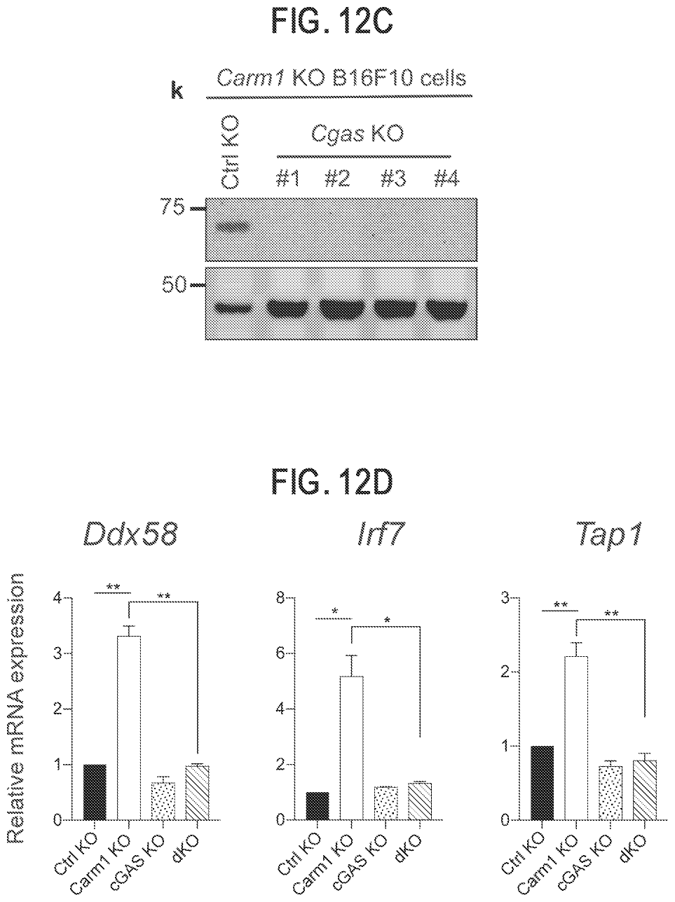

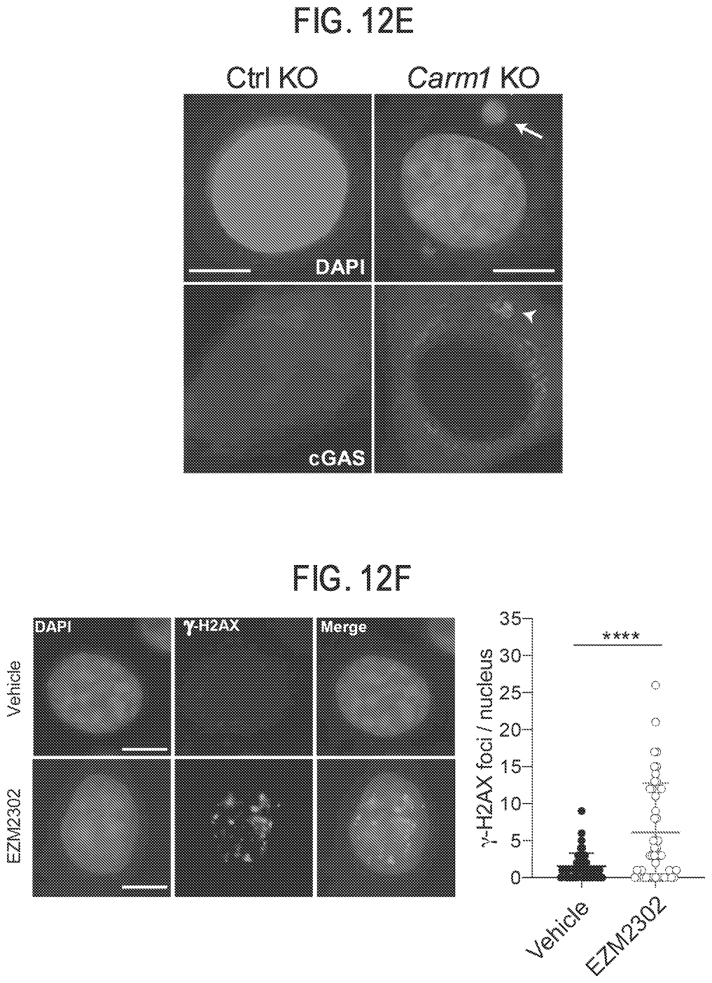

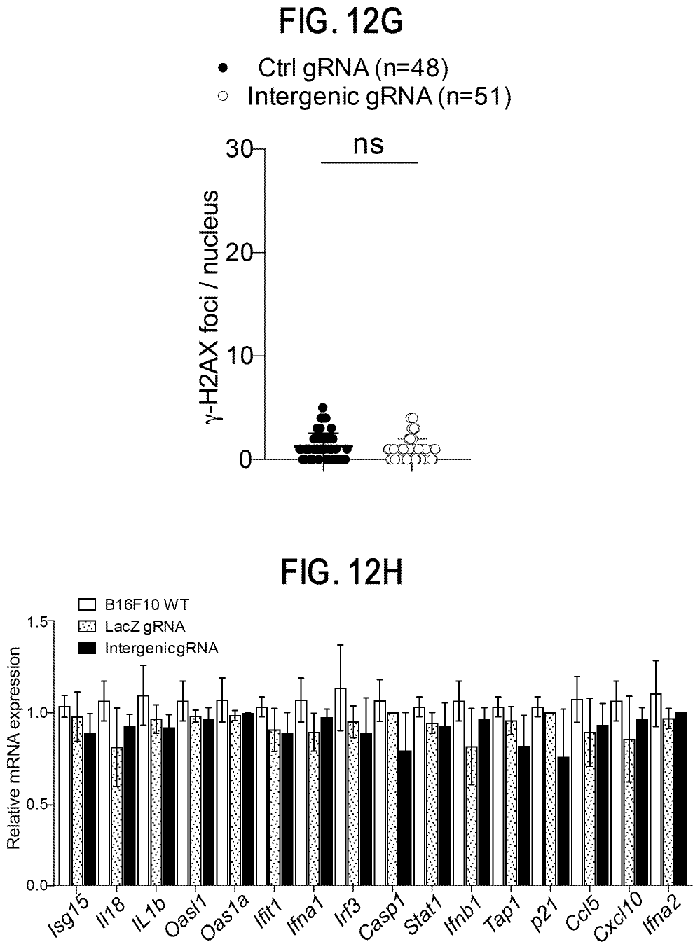

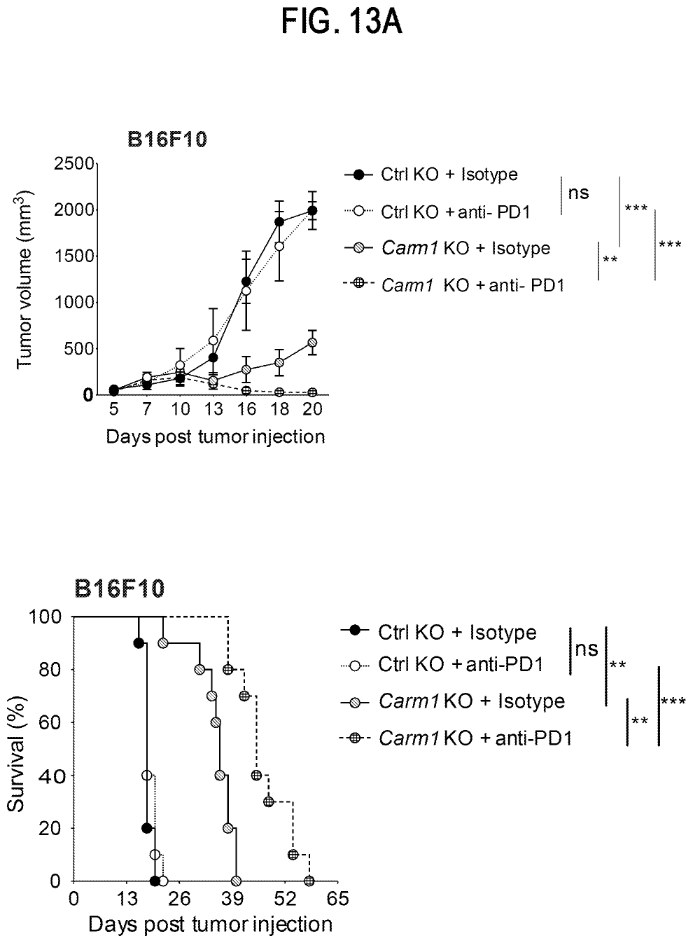

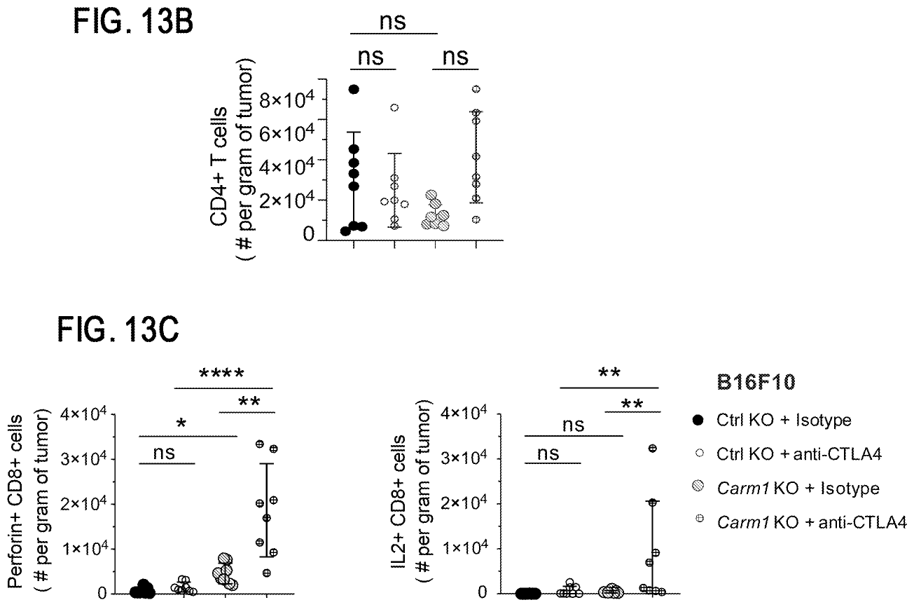

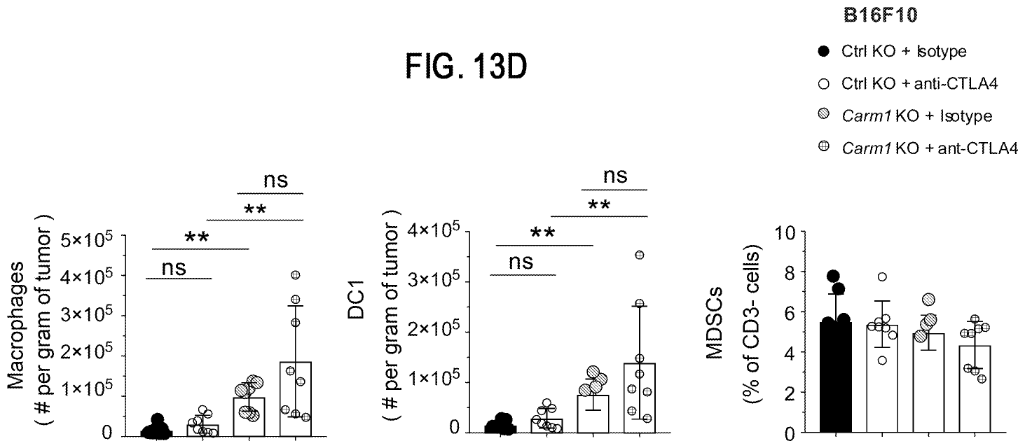

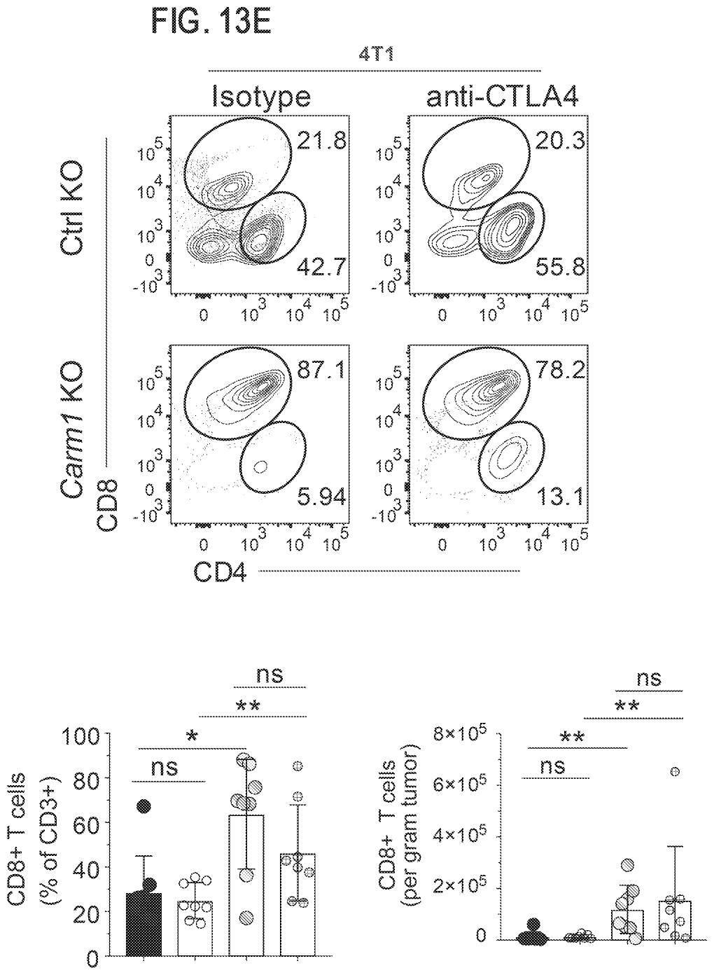

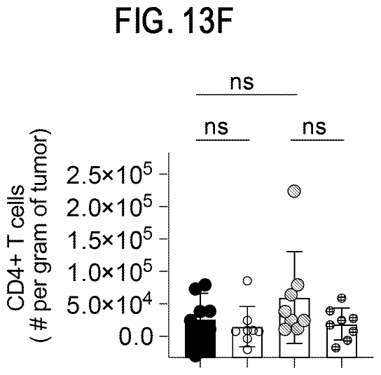

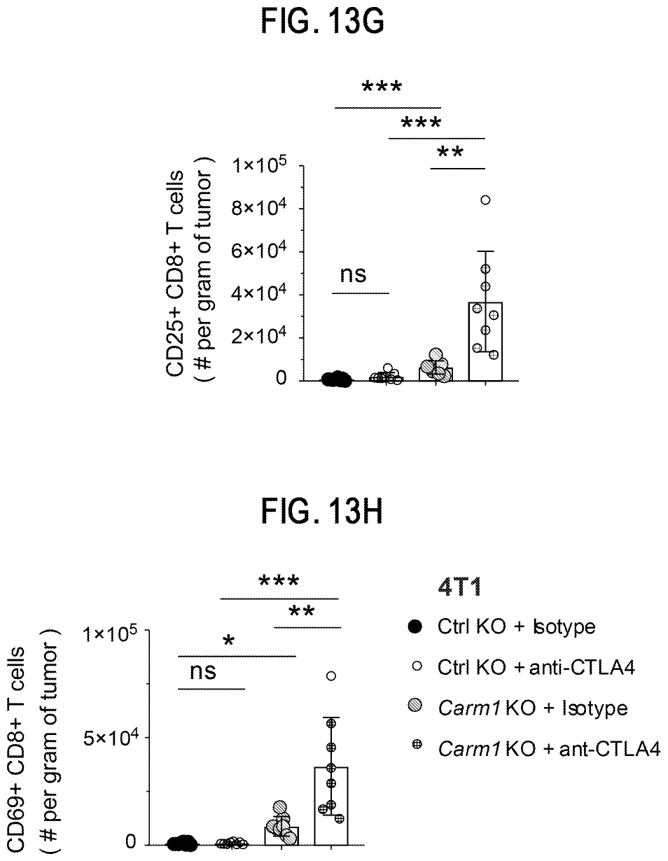

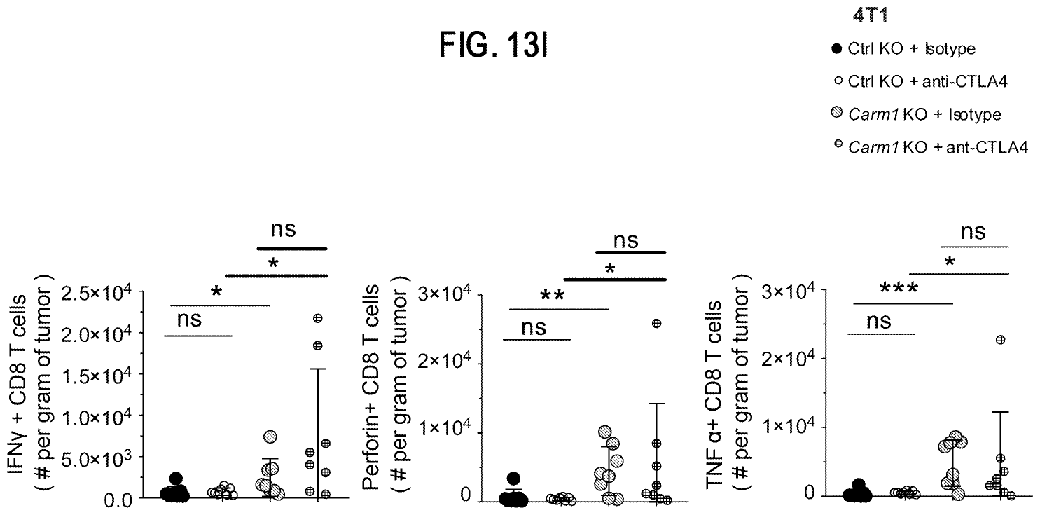

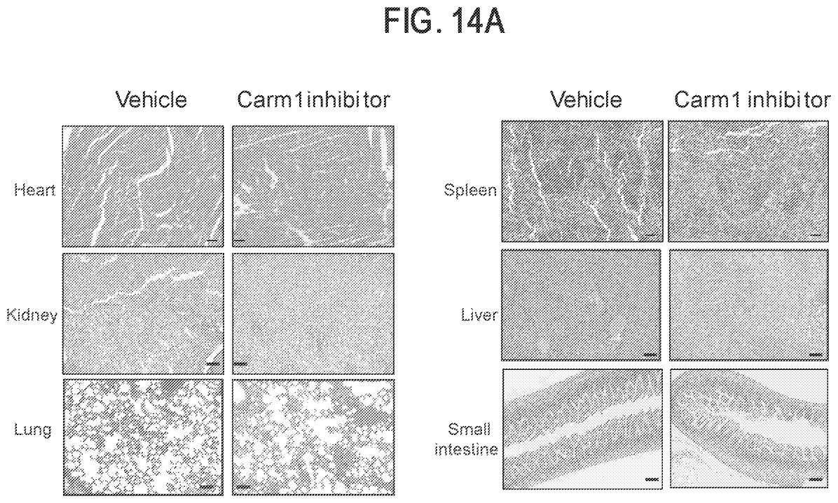

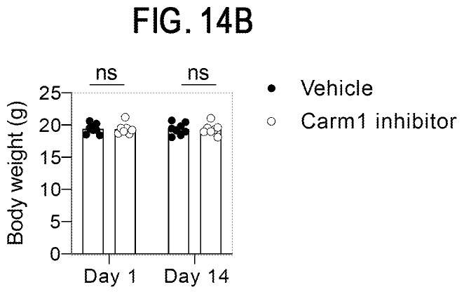

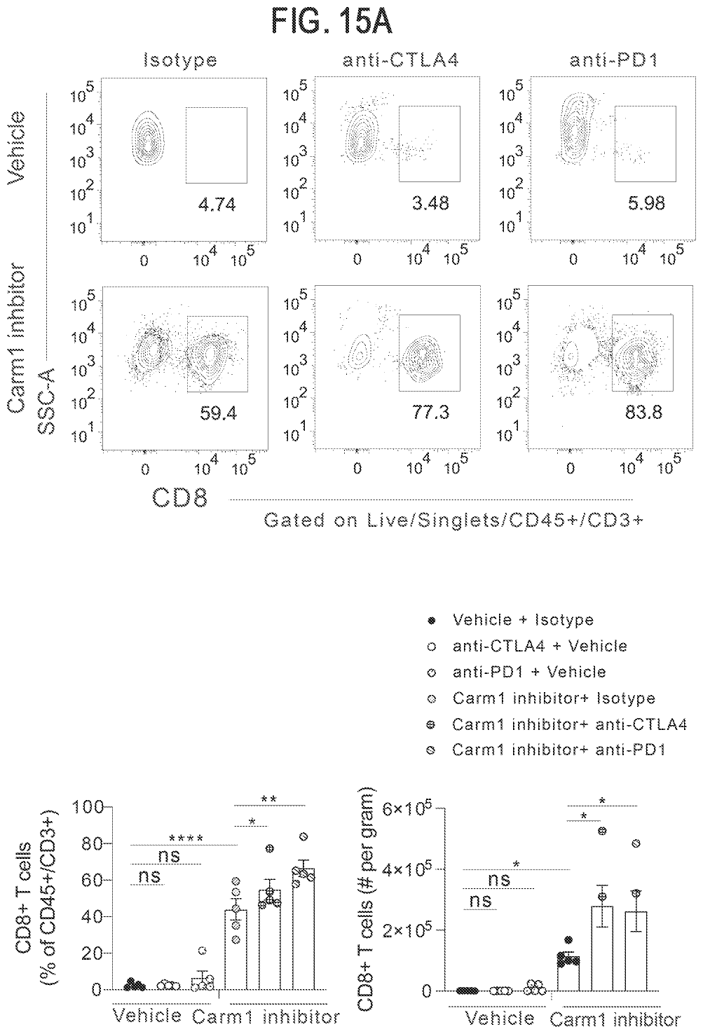

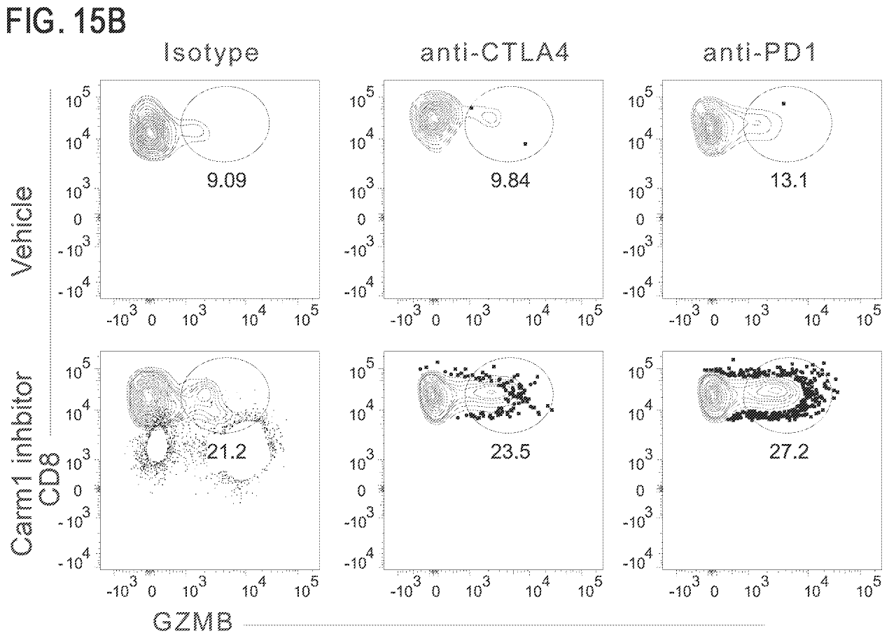

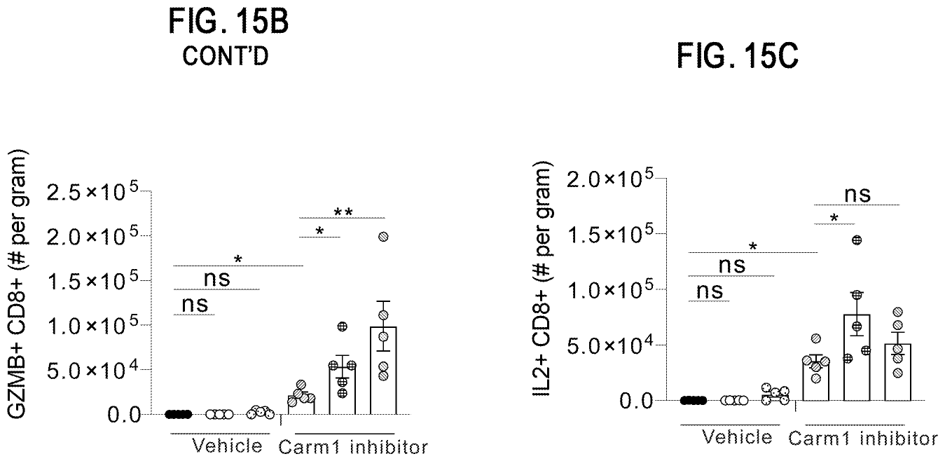

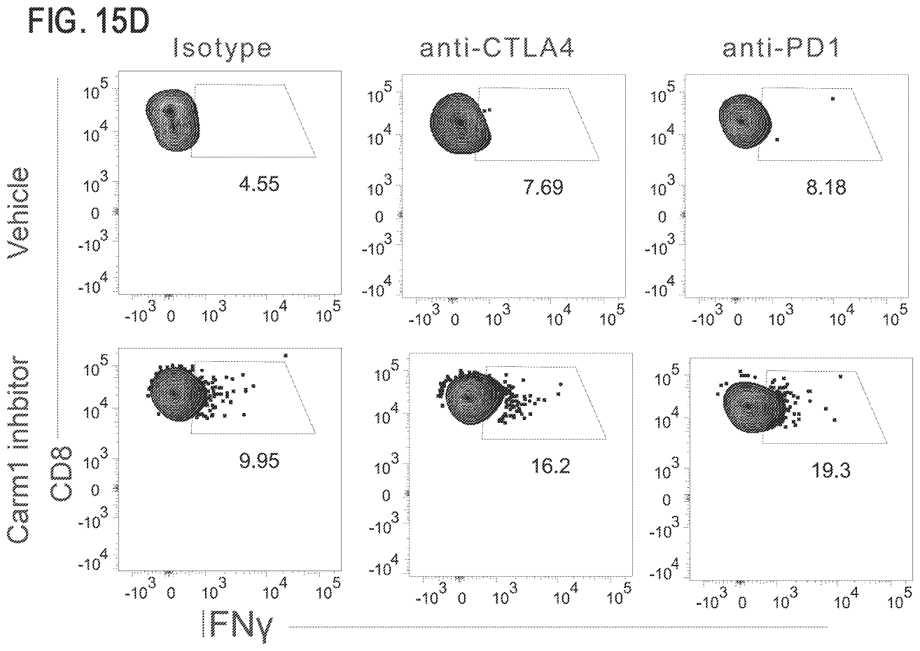

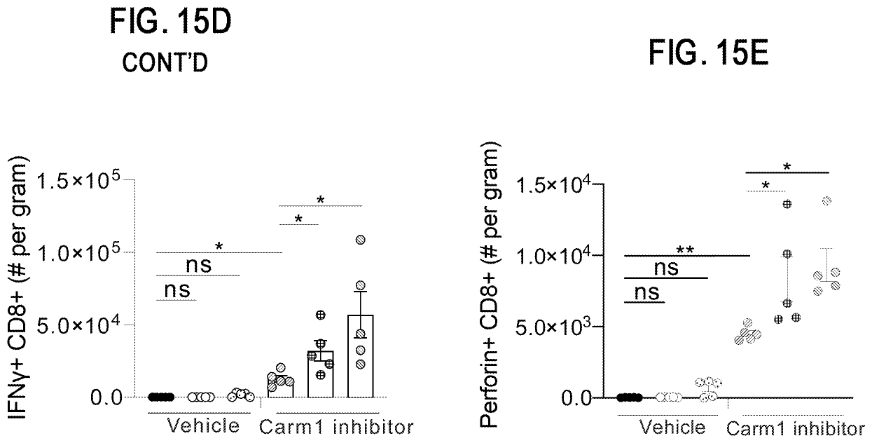

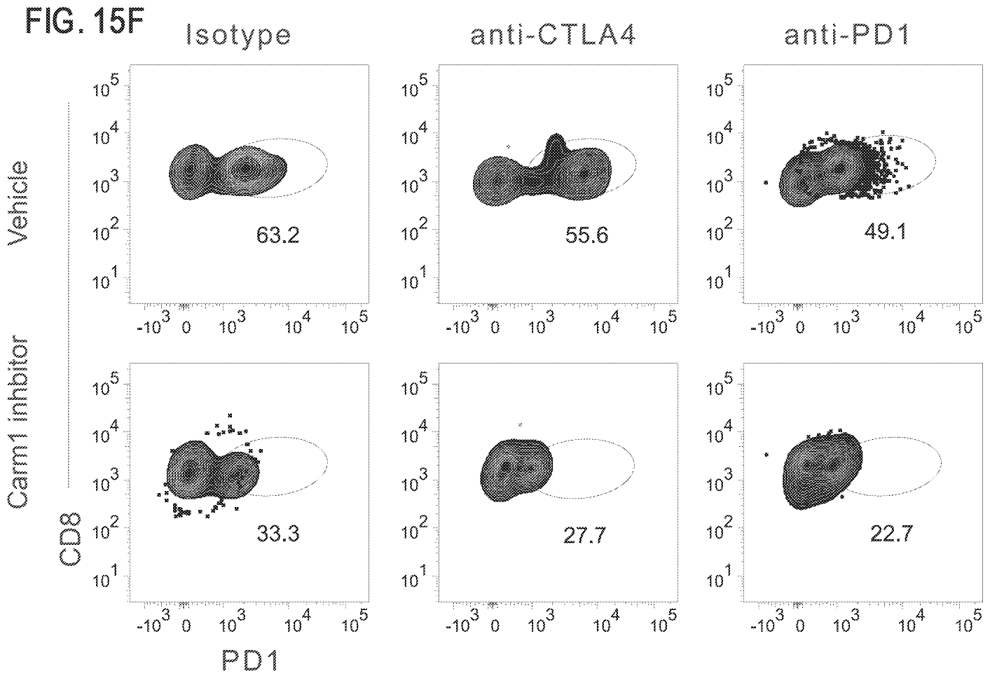

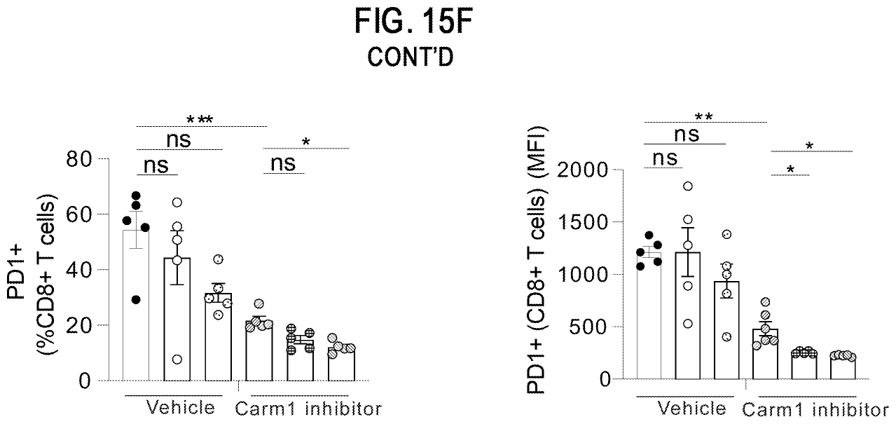

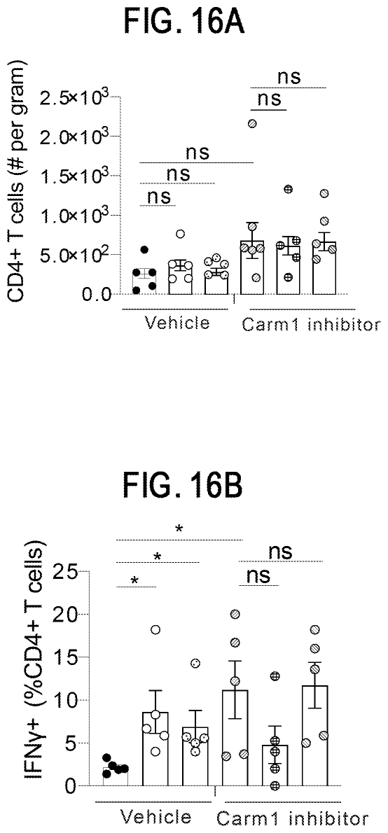

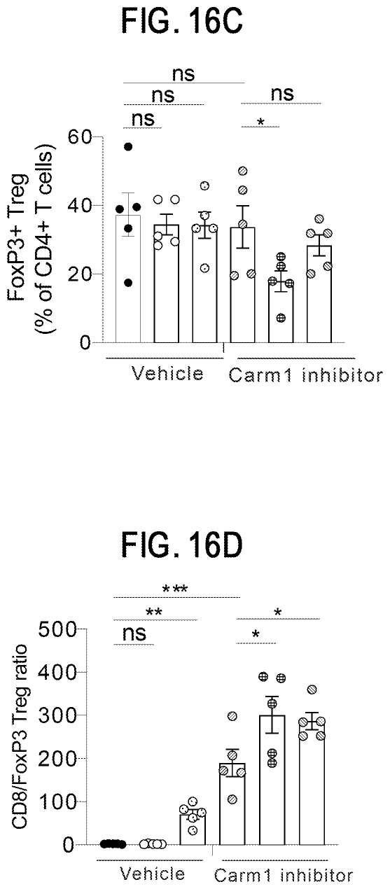

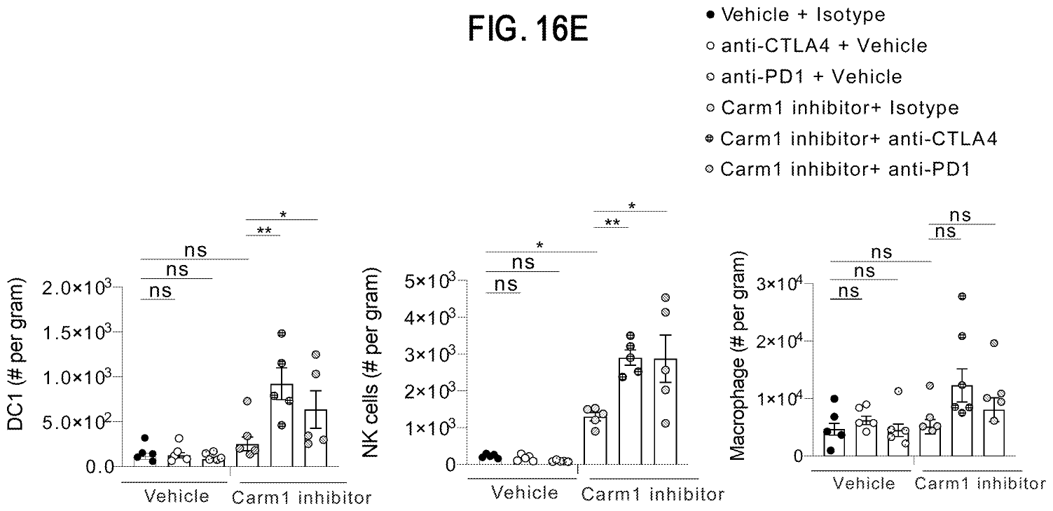

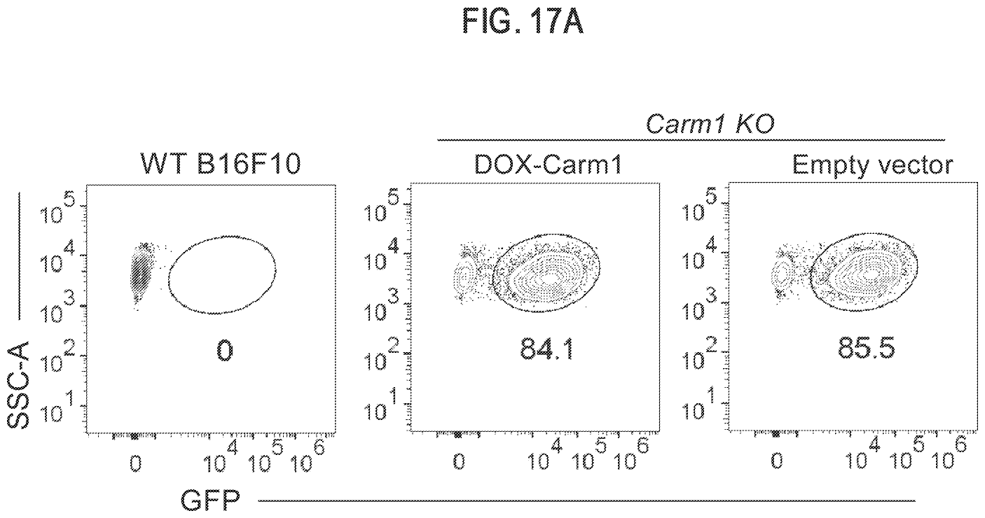

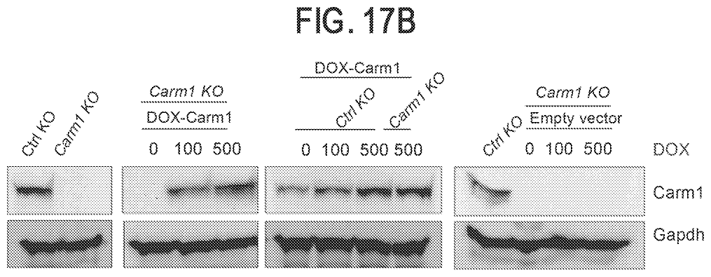

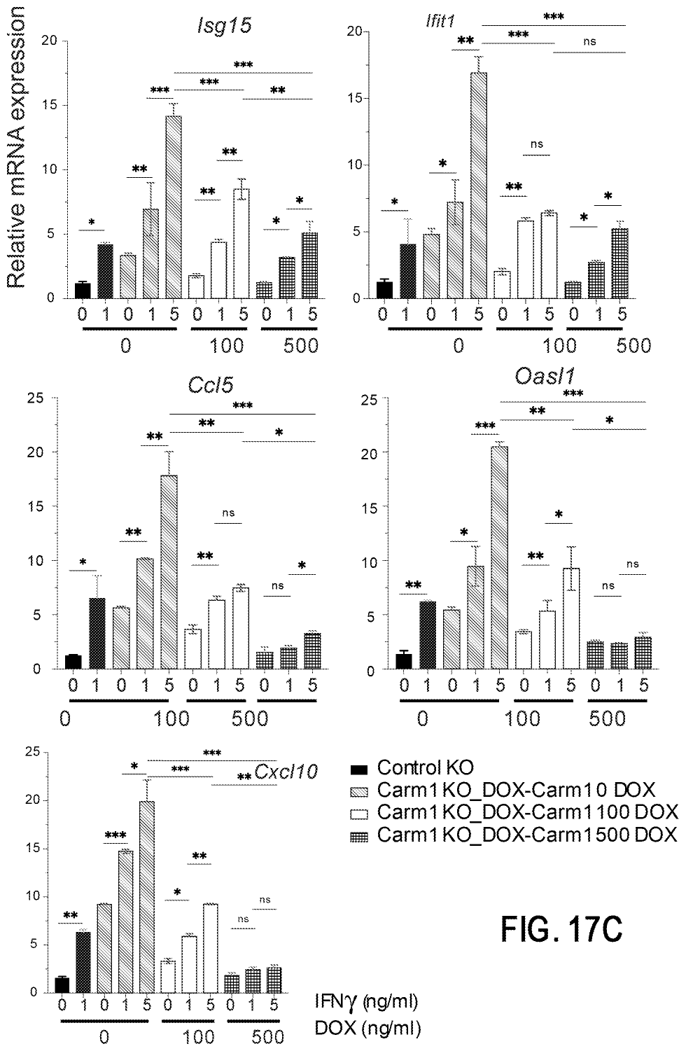

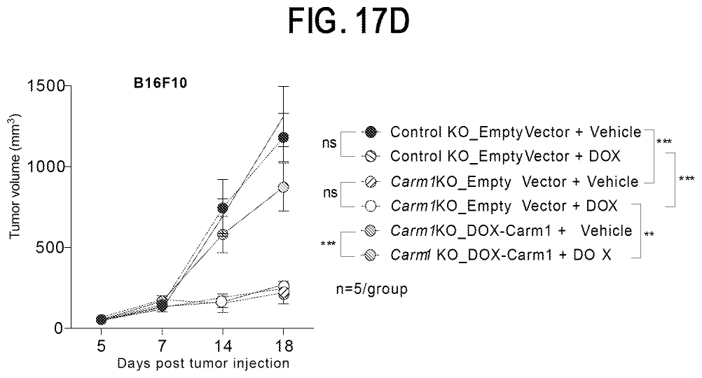

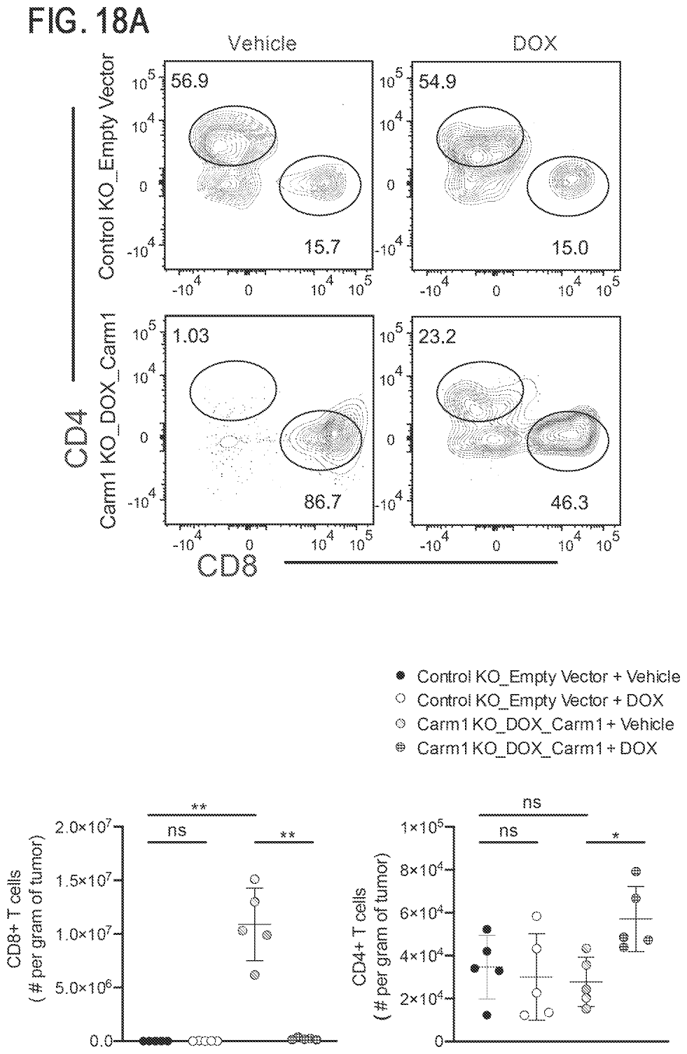

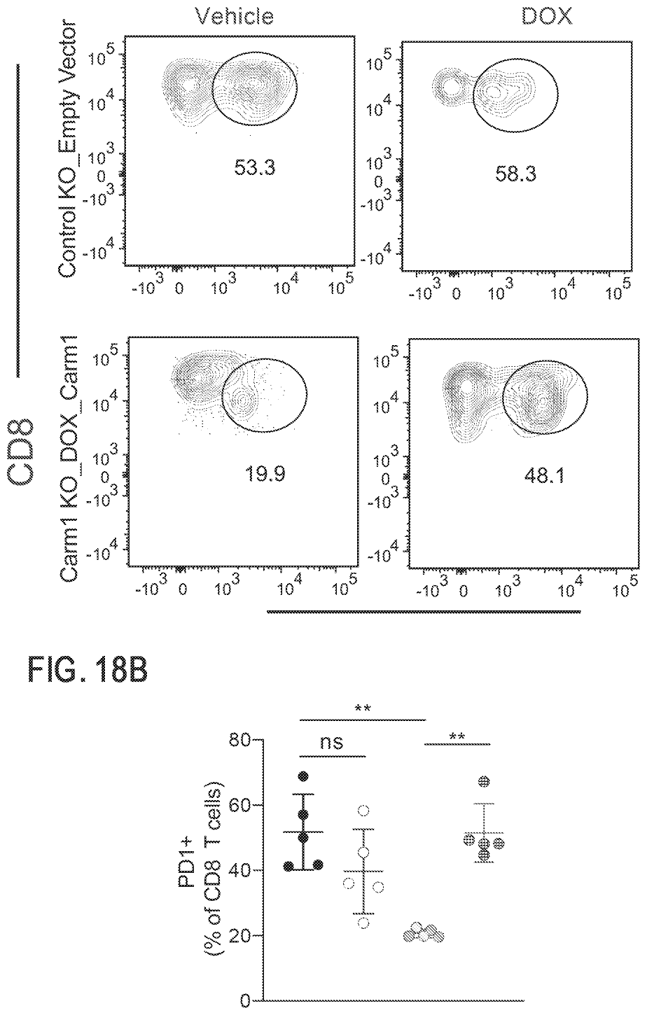

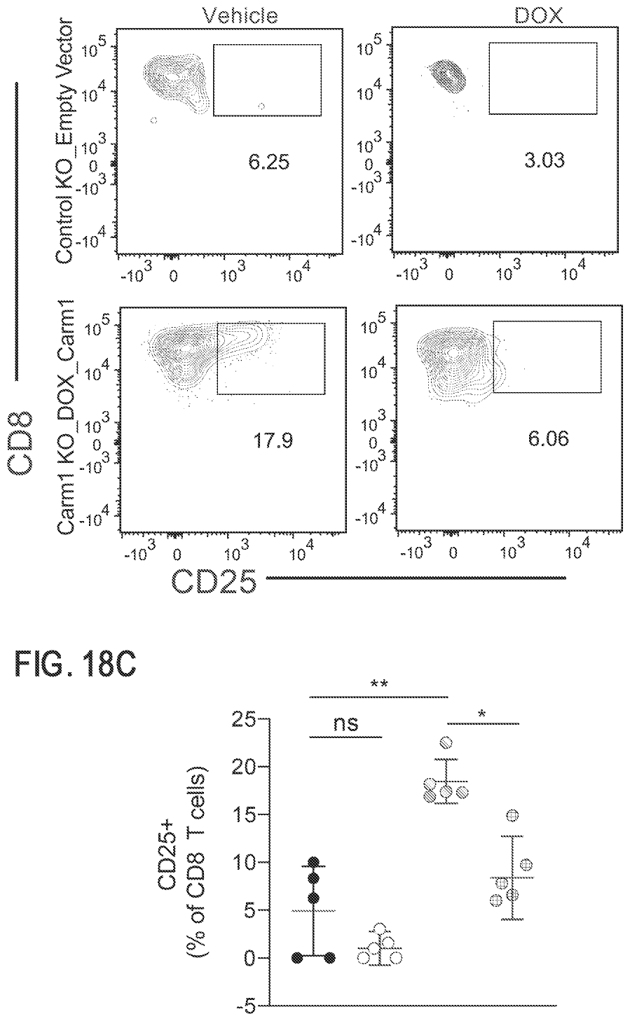

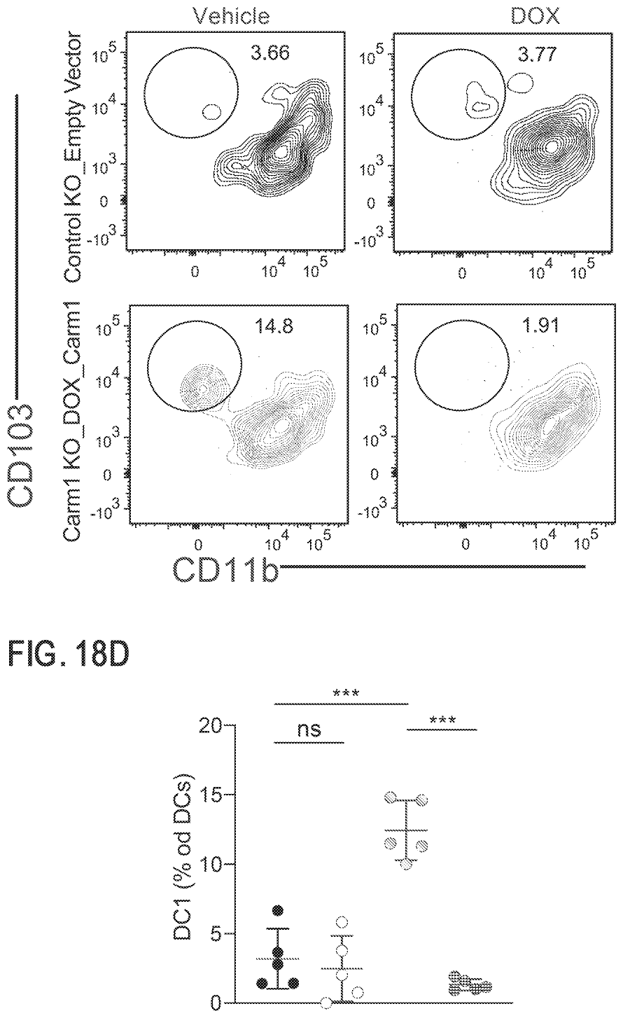

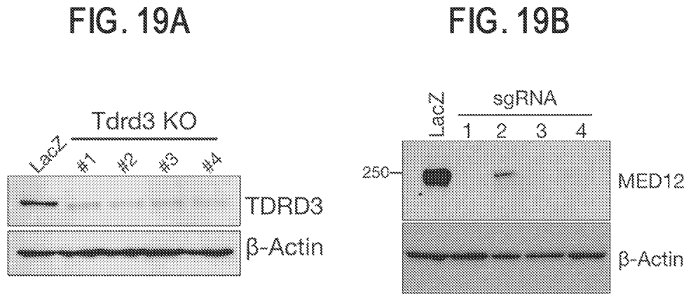

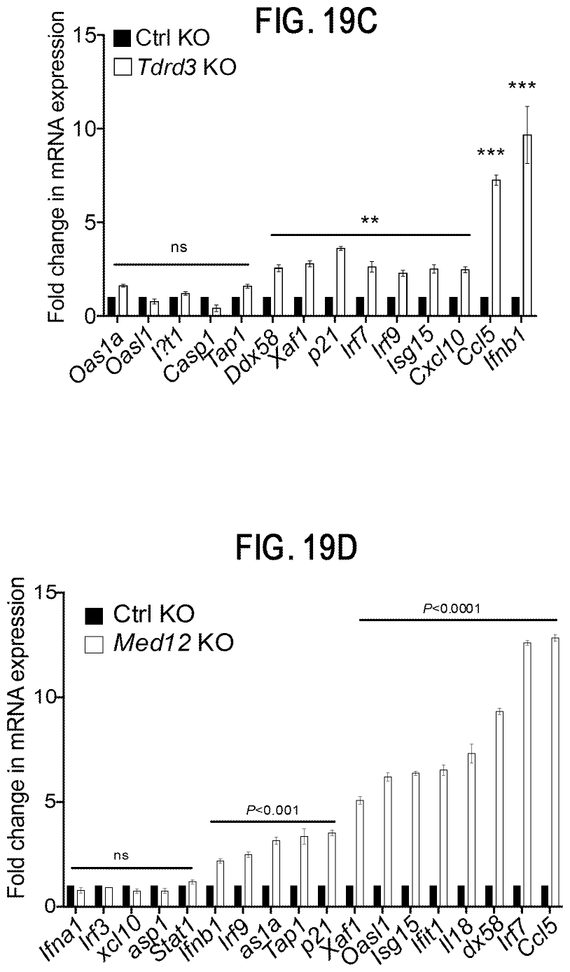

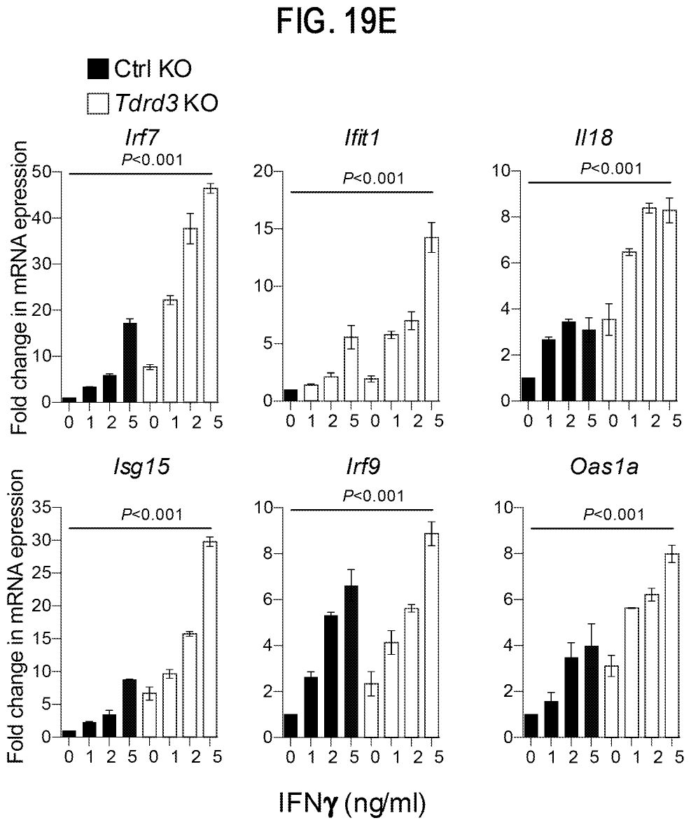

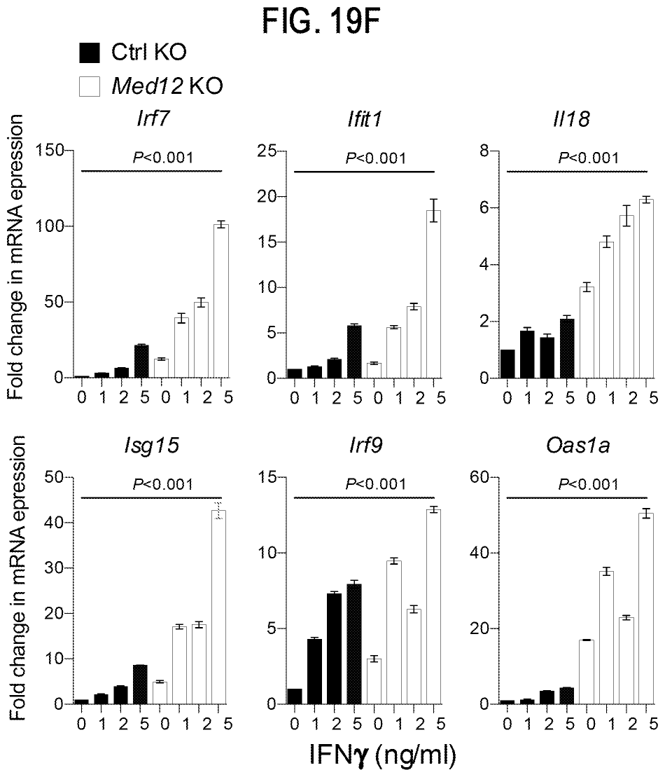

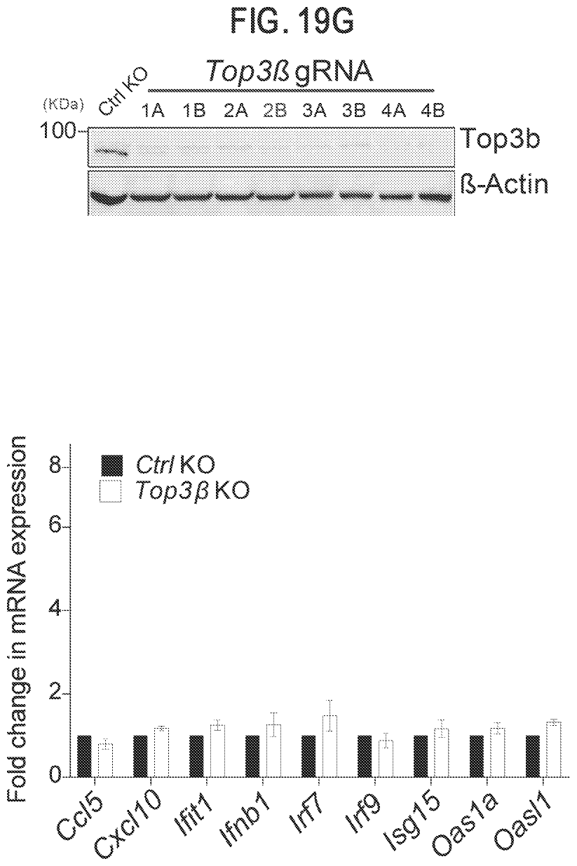

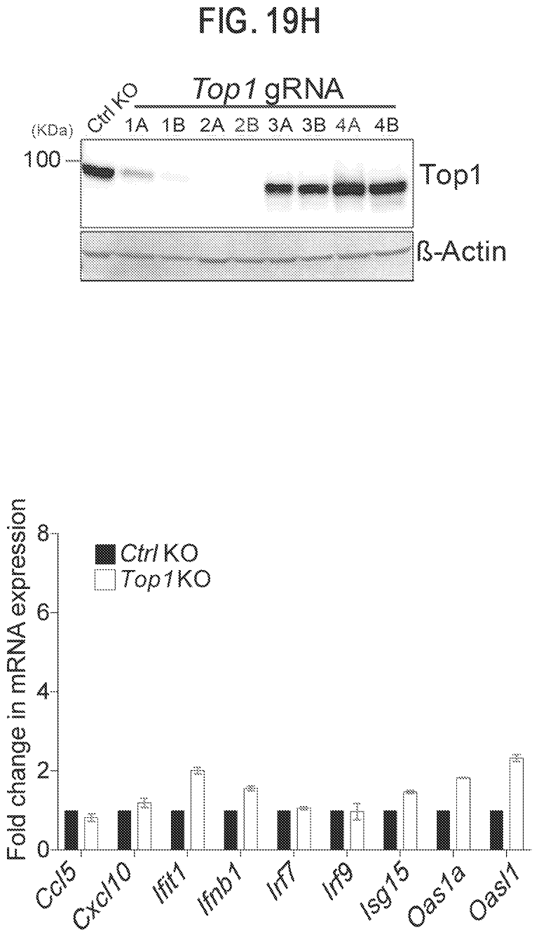

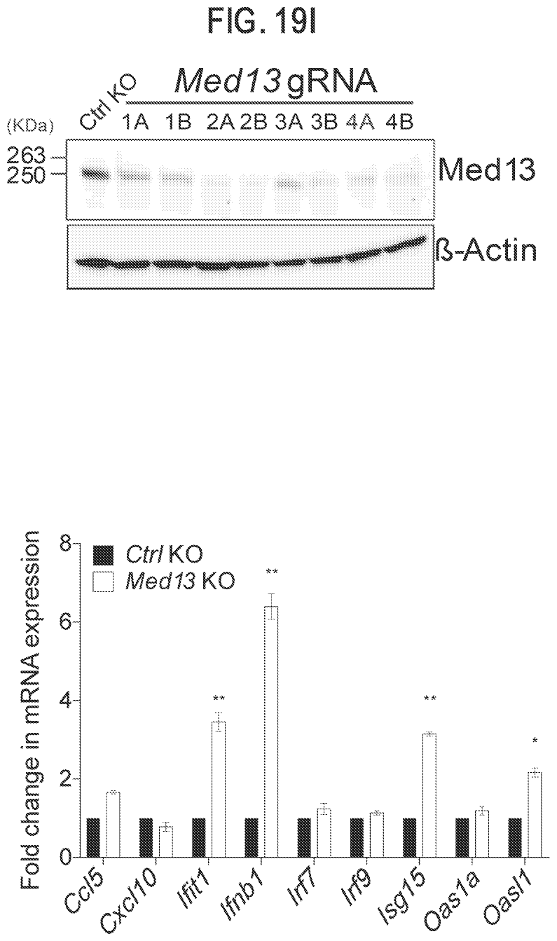

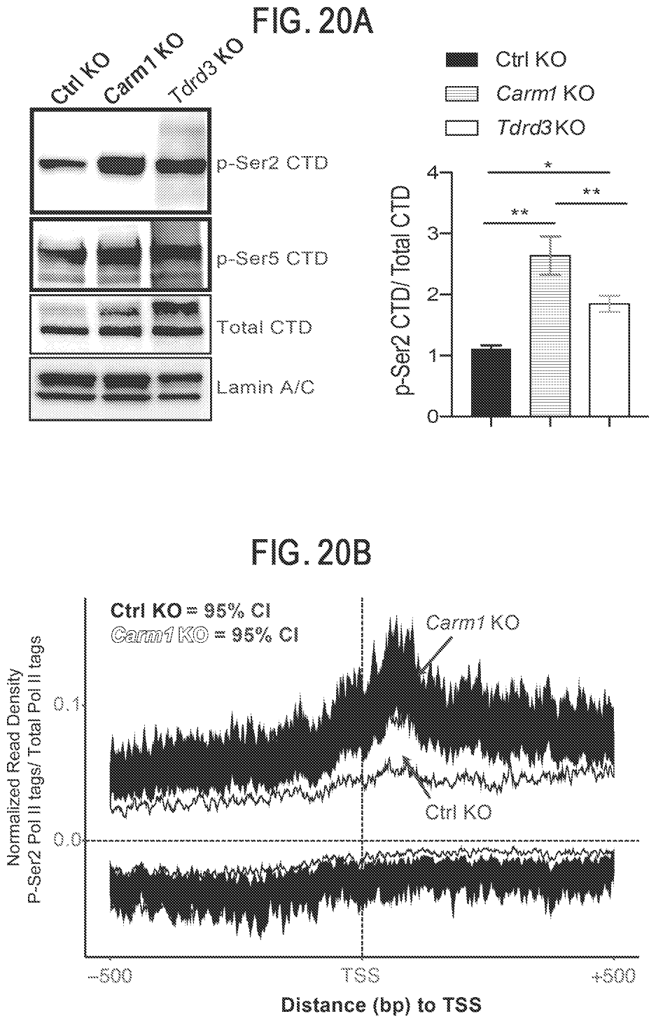

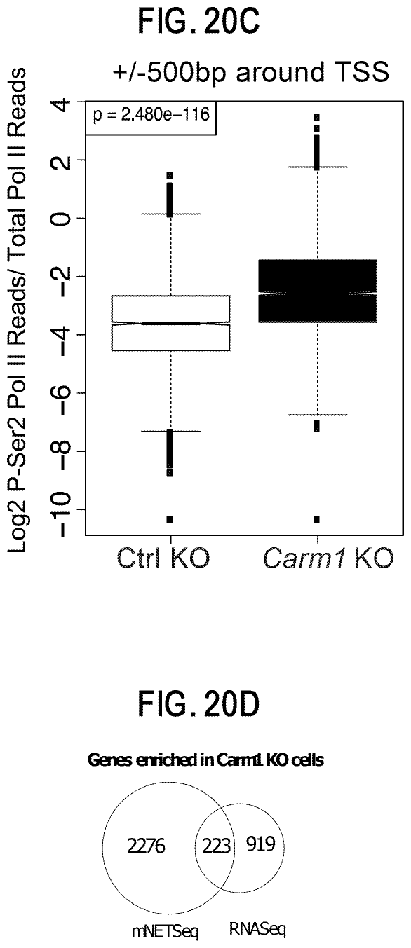

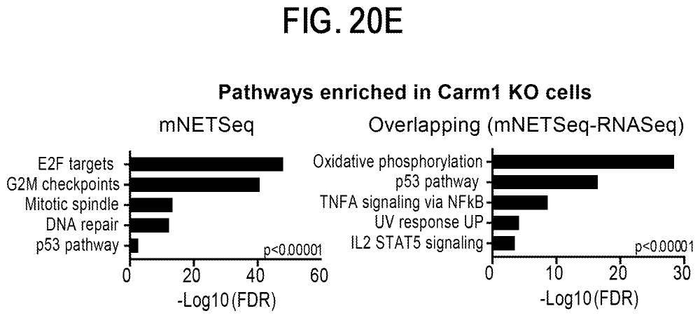

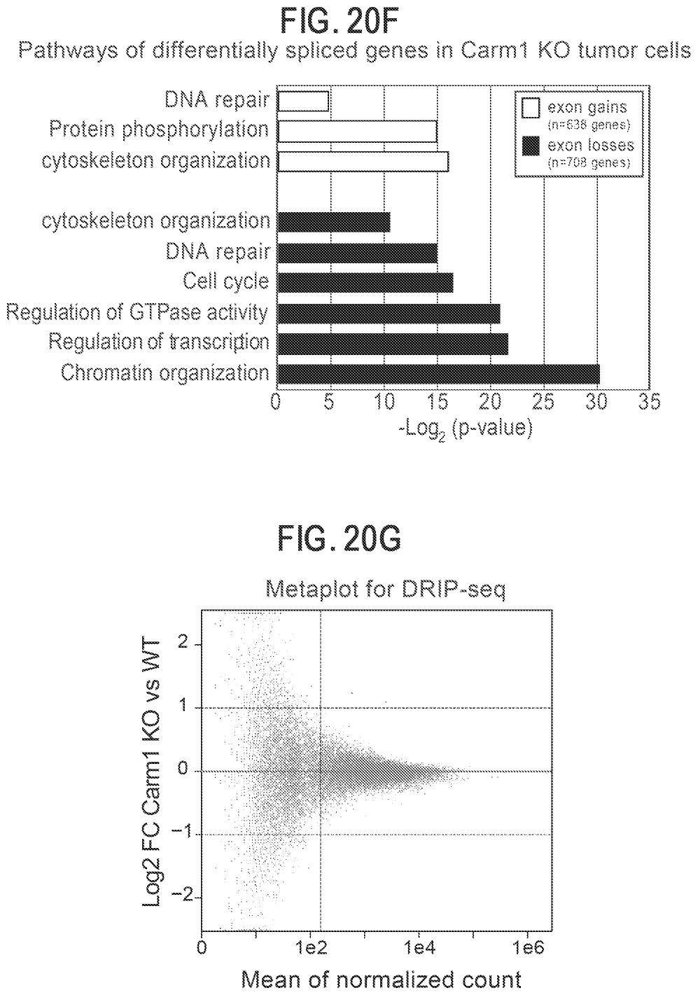

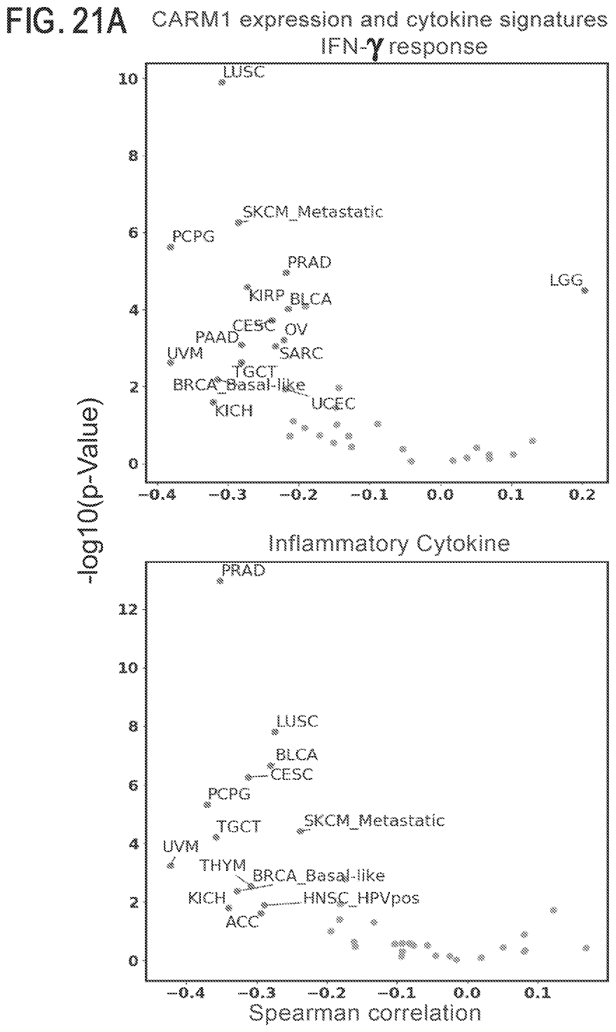

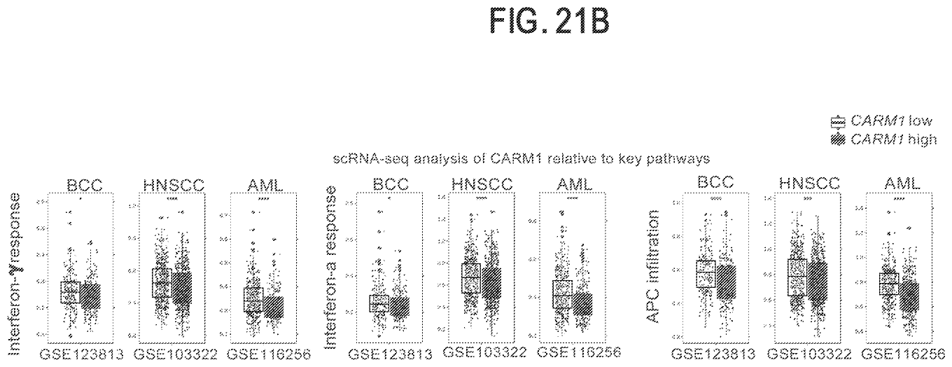

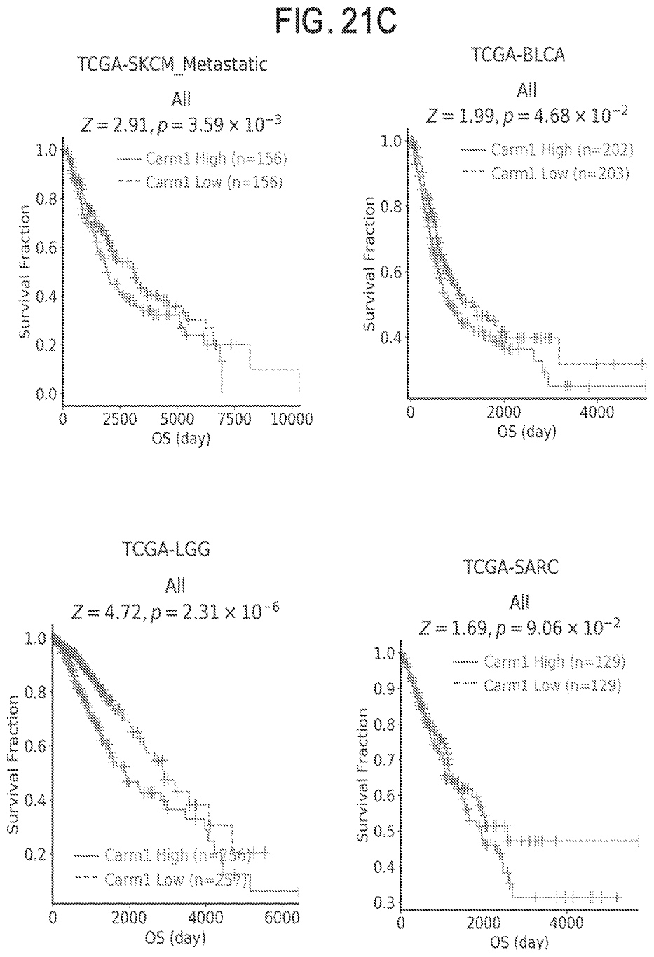

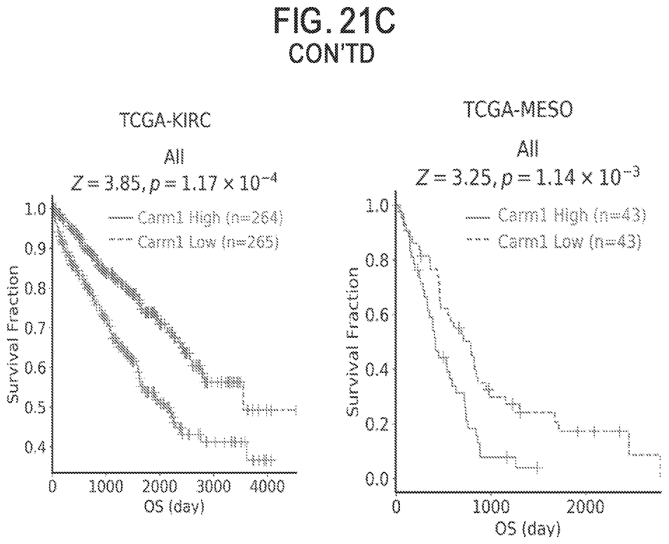

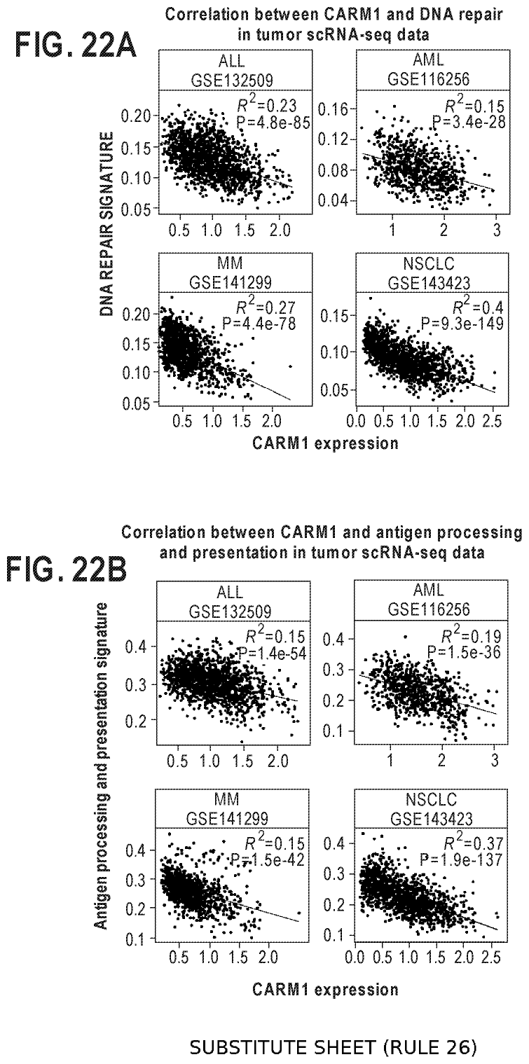

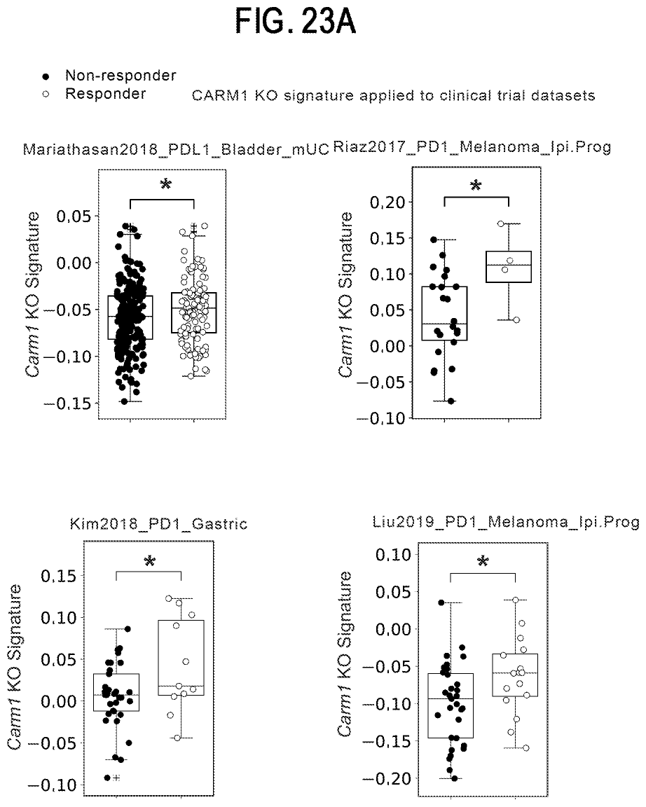

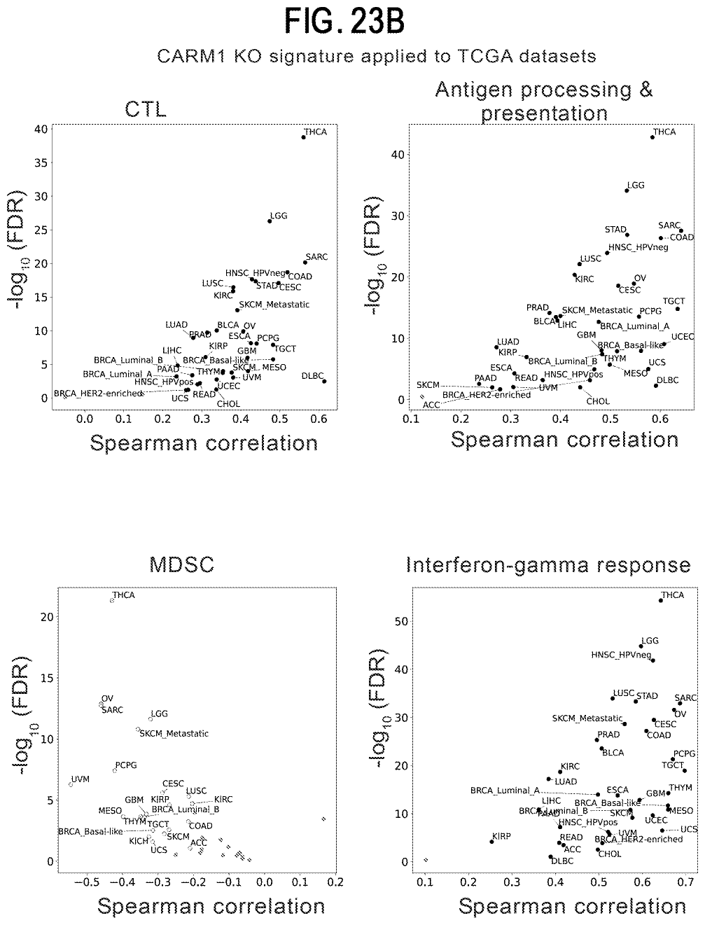

FIG. 3J illustrates that inactivation of Carm1 gene in tumor cells elicits tumor immunity, showing induction of tumor cell apoptosis (Carm1-KO or control-KO B16F10-OVA-ZsGreen cells) by CD8 T cells (as described in FIG. 3I), measured with a Caspase-3/7 dye at different E:T ratios (n=8-10 replicates/group).

FIG. 3K illustrates that inactivation of Carm1 gene in tumor cells elicits tumor immunity, showing sensitization of tumor cells to T cells with a CARM1 inhibitor. B16F10-OVA-ZsGreen tumor cells were pretreated with CARM1 inhibitor (EZM2302, 0.1 μM) for 24 hours. Vehicle or inhibitor treated tumor cells were co-cultured with OT-I CD8 T cells at indicated E:T ratios (n=7-8 replicates/condition).

FIG. 3L illustrates that inactivation of Carm1 gene in tumor cells elicits tumor immunity, showing T cell cytotoxicity assay with human BT549 TNBC and human CD8 T cells that expressed a NY-ESO-1 TCR. Tumor cells were pretreated with CARM1 inhibitor (EZM2302, 0.1 μM) for 24 hours (n=7-10 replicates/group); numbers of surviving tumor cells were quantified after 24 hours of co-culture. In FIGS. 3A-3L, two-way ANOVA was used to determine statistical significance for tumor measurements at time points when all mice were alive. Statistical significance for survival of mice in each treatment group were calculated by Log-rank (Mantel-Cox) test. Bar graphs represent data summarized as mean±S.E.M. and were analyzed by unpaired two-sided Mann-Whitney test. Data shown are representative of three experiments. *p<0.05, **p<0.01, ***p<0.001, ****p<0.0001, ns (non-significant).

FIG. 4A illustrates innate immune activation in Carm1 deficient tumor cells, showing RNA-seq analysis of differentially expressed genes in Carm1-KO and control-KO B16F10 tumor cells (n=3/group). Data are representative of two independent experiments.

FIG. 4B illustrates innate immune activation in Carm1 deficient tumor cells, showing Gene Ontology (GO) analysis of significantly upregulated/downregulated genes in Carm1-KO compared to control-KO B16F10 tumor cells.

FIG. 4C illustrates innate immune activation in Carm1 deficient tumor cells, showing Venn diagram representing number of overlapping differentially expressed genes in Carm1-KO tumor and CD8 T cells.

FIG. 4D illustrates innate immune activation in Carm1 deficient tumor cells, showing validation of interferon inducible genes (ISGs) by RT-qPCR in Carm1-KO compared to control-KO B16F10 cells (n=3/group).

FIG. 4E illustrates innate immune activation in Carm1 deficient tumor cells, showing RT-qPCR analysis of ISG mRNA levels following treatment of B16F10 cells with CARM1 inhibitor EZM2302 (0.1-1 μM) or solvent control for 7 days (n=3/group).

FIG. 4F illustrates innate immune activation in Carm1 deficient tumor cells, showing expression of selected ISGs in control-KO, Carm1-KO, cGAS-KO and Carm1/cGAS double-KO (dKO) B16F10 cells analyzed by RT-qPCR (n=3/group).

FIG. 4G illustrates innate immune activation in Carm1 deficient tumor cells, showing T cell cytotoxicity assay with control-KO, Carm1-KO, cGAS-KO and Carm1/cGAS dKO B16F10 cells. Tumor cells were co-cultured with OT-I CD8 T cells at indicated E:T ratios for 24 hours (n=7-10 replicates/condition); live GFP positive tumor cells were counted using a Celigo image cytometer. Data are shown as mean±SEM. ***p<0.001, by unpaired two-sided Mann-Whitney test.

FIGS. 4H-4I illustrate innate immune activation in Carm1 deficient tumor cells, showing dsDNA damage in Carm1-KO versus control-KO B16F10 tumor cells based on labeling with γH2AX (FIG. 4H) and RAD51 (FIG. 4I) Abs. Representative immunofluorescence images (left) of γH2AX or RAD51 antibody labeling (red); nuclei labeled with DAPI. Quantification of number of □H2AX or RAD51 foci/nucleus (right). Data are shown as mean±SEM, ***p<0.001, by unpaired two-sided Mann-Whitney test. Scale bar—10 μM.

FIG. 4J illustrates innate immune activation in Carm1 deficient tumor cells, showing detection of micronuclei in Carm1-KO and control-KO B16F10 tumor cells. DNA was labeled with DAPI; representative images (left) and quantification of cells positive for micronuclei (right). Data are shown as mean±SEM, **p<0.01, by unpaired two-sided Mann-Whitney test. Scale bar—10 μM.

FIG. 4K illustrates innate immune activation in Carm1 deficient tumor cells, showing analysis of Carm1-KO versus control-KO CD8+ T cells for dsDNA damage. OT-I CD8 T cells were plated 7 days posted editing using CRIPSR-Cas9 as previously described and stained for CD8a, γH2AX and DAPI. Representative immunofluorescence images of CD8a antibody labeling (red), γH2AX antibody labeling (green); nuclei stained with DAPI (blue). Scale bar—20 μM. Data shown in FIGS. 4D-4J are representative of three independent experiments. Bar graphs represent data summarized as mean±S.E.M. and were analyzed by unpaired two-sided Mann-Whitney test. *p<0.05, **p<0.01, ***p<0.001, ****p<0.0001, ns (non-significant). Error bars for all qPCR data represent SD with three replicates per group.

FIG. 5A illustrates that Carm1 inhibition overcomes resistance to checkpoint blockade, showing treatment of Carm1-KO or control-KO B16F10 tumors with CTLA-4 or isotype control antibodies (n=8 mice/group). Tumor growth (left) and survival of tumor bearing mice (right) are shown. Mice bearing comparable tumor volume (˜50 mm³) were randomized into different treatment groups.

FIG. 5B illustrates that Carm1 inhibition overcomes resistance to checkpoint blockade, showing treatment of B16F10 tumors with CARM1 inhibitor (EZM2302) or vehicle control in combination with CTLA-4 or isotype control antibodies (n=8 mice/group). EZM2302 (150 mg/kg b.i.d.) was orally administered for 2 weeks (days 7-21).

FIG. 5C illustrates that Carm1 inhibition overcomes resistance to checkpoint blockade, showing treatment of Carm1-KO or control-KO 4T1 tumors with anti-CTLA-4 or isotype control antibodies (n=8 mice/group).

FIG. 5D illustrates that Carm1 inhibition overcomes resistance to checkpoint blockade, showing change in number of spontaneous lung metastases (left) and representative images of lung metastases formed by 4T1 tumors treated as described in C (right) (n=8 mice/group).

FIG. 5E illustrates that Carm1 inhibition overcomes resistance to checkpoint blockade, showing quantification of tumor-infiltrating CD8 T cells in Carm1-KO and control-KO B16F10 tumors (n=8 mice/group) following treatment with CTLA-4 or isotype control antibodies (day 18 post tumor cell implantation). Representative flow plots (left) and quantification of CD8 T cells as percentage of CD3+ cells and per gram of tumor (middle and right, respectively).

FIG. 5F-5G illustrate that Carm1 inhibition overcomes resistance to checkpoint blockade, showing quantification of PD-1 positive and PD-1/Tim-3 double positive tumor-infiltrating CD8+ T cells for experiment described in FIG. 5E.

FIG. 511-5I illustrate that Carm1 inhibition overcomes resistance to checkpoint blockade, showing H-I. Quantification of CD8 T cells expressing effector markers (GZMB and IFNγ), migratory cross-presenting DCs (CD45+/CD3−/F4/80−/CD11c+/MHC-II^{high}/CD103+/CD11b−) and NK cells (CD45+/CD3−/CD49b+) per gram of tumor for experiment described in FIG. 5E. In FIGS. 5A-5I, data shown are representative of two experiments. Two-way ANOVA was used to determine statistical significance for time points when all mice were viable for tumor measurement. Log-rank (Mantel-Cox) test was used to determine statistical significance for survival of mice. Bar graphs represent data summarized as mean±S.E.M. and were analyzed by unpaired two-sided Mann-Whitney test, ****P<0.0001; ***P<0.001; **P<0.01; *P<0.05; n.s., non-significant.

FIG. 6A illustrates that Tdrd3 and Med12 are downstream effectors of Carm1, showing comparison of ISG expression in control-KO, Tdrd3-KO, cGAS-KO and Tdrd3/cGAS dKO B16F10 cells by RT-qPCR (n=3/group).

FIG. 6B illustrates that Tdrd3 and Med12 are downstream effectors of Carm1, showing comparison of ISG expression in control-KO, Med12-KO, cGAS-KO and Med12/cGAS dKO B16F10 cells by RT-qPCR (n=3/group).

FIG. 6C illustrates that Tdrd3 and Med12 are downstream effectors of Carm1, showing immunofluorescence analysis of dsDNA damage by γH2AX antibody staining (red foci; nuclei labeled with DAPI) in control-KO, Carm1-KO, Tdrd3-KO and Med12-KO B16F10 tumor cells (left). Images for control-KO and Carm1-KO reshown from FIG. 4 to illustrate comparison to other KO tumor cell lines. Quantification of number of γH2AX foci/nucleus for each cell line (right). Scale bar—10 μM.

FIG. 6D illustrates that Tdrd3 and Med12 are downstream effectors of Carm1, showing analysis of micronuclei in control-KO, Carm1-KO, Tdrd3-KO and Med12-KO B16F10 tumor cells using DAPI as a DNA stain. Representative images (left) and quantification of percentage of cells with micronuclei (right). Scale bar—10 μM.

FIG. 6E illustrates that Tdrd3 and Med12 are downstream effectors of Carm1, showing tumor growth and survival of mice bearing control-KO and Tdrd3-KO B16F10 tumors. Mice were treated with CD8 T cell depleting or isotype control antibodies (n=8-10 mice/group).

FIG. 6F illustrates that Tdrd3 and Med12 are downstream effectors of Carm1, showing tumor growth and survival of control-KO and Tdrd3-KO B16F10 tumors in immunocompetent and T cell deficient (Tcra KO) mice (n=8-10 mice/group).

FIG. 6G illustrates that Tdrd3 and Med12 are downstream effectors of Carm1, showing tumor growth and survival of anti-CTLA-4 or isotype control antibody treated of mice bearing Tdrd3-KO or control-KO B16F10 tumors (n=8 mice/group).

FIG. 6H illustrates that Tdrd3 and Med12 are downstream effectors of Carm1, showing arginine methylation of Med12 by Carm1. Immunoprecipitation of Med12 protein from nuclear extracts of control-KO, Carm1-KO or Tdrd3-KO B16F10 tumor cells, followed by Western blot detection with an antibody specific for asymmetric dimethylation of arginine residues (ADMA, left). Western blot analysis of nuclear extracts from the same cell lines with antibodies for the indicated proteins (right).

FIG. 6I illustrates that Tdrd3 and Med12 are downstream effectors of Carm1, showing effect of Carm1 on interaction of Med12 with histone H3. Immunoprecipitation of Med12 protein from control-KO or Carm1-KO B16F10 tumor cells, followed by Western blot detection with histone H3 antibody. Input levels of histone H3 in immunoprecipitated samples is shown (middle); quantification of histone H3 bound to Med12 normalized to total histone H3 (bottom). Graph shows proposed biochemical interactions (right). In FIGS. 6A-6I, two-way ANOVA was used to determine statistical significance for time points when all mice were viable for tumor measurement. Statistical significance for survival of mice in each treatment group was calculated by Log-rank (Mantel-Cox) test. Bar graphs represent data summarized as mean±S.E.M. and were analyzed by unpaired two-sided Mann-Whitney test. Data are representative of three (A-H) and two (I) experiments, *p<0.05, **p<0.01, ***p<0.001, ****p<0.0001, ns (non-significant).

FIG. 7A illustrates relevance of CARM1 in human cancers, showing analysis of indicated pathways across a diverse panel of 1,208 human cancer cell lines (Cancer Cell Line Encyclopedia, CCLE). Symmetric violin plots illustrate stratifications for CARM1 high and low cell lines using median expression levels.

FIG. 7B illustrates relevance of CARM1 in human cancers, showing analysis of TCGA RNA-seq data across human cancer types. Correlation of CARM1 mRNA levels with indicated pathways. Plots show Spearman's correlation and estimated statistical significance for indicated pathways in different cancer types adjusted for tumor purity. Each dot represents a cancer type in TCGA.

FIG. 7C illustrates relevance of CARM1 in human cancers, showing Gene Ontology (GO) analysis of significantly upregulated/downregulated genes in CARM1 high tumor cells in skin cutaneous melanoma (SKCM, N=442 patients) and lung squamous cell carcinoma (LUSC, N=363 patients) datasets (TCGA PanCancer Atlas).

FIG. 7D illustrates relevance of CARM1 in human cancers, showing analysis of scRNA-seq data of malignant cells from three human cancer cohorts (GSE123813: basal cell carcinoma; GSE103322: head and neck cancer; GSE116256: AML). Scores for DNA repair, and p53 pathways are shown. Data were stratified by CARM1 high and low groups using median expression levels. Statistical comparisons were made using two-sided unpaired Mann-Whitney tests.

FIG. 7E illustrates relevance of CARM1 in human cancers, showing correlation of CARM1 mRNA levels with response to ICB (immune checkpoint blockade) in cancer patients treated with PD-1 or PD-L1 mAbs. The analysis is shown for tumors with low (<median) MED12 mRNA levels. CARM1 mRNA levels did not correlate with response to ICB in tumors with high (>median) MED12 mRNA levels. The p-values were inferred by Mann-Whitney U test.

FIG. 8A illustrates identification of CARM1 as a therapeutic target in tumor-infiltrating T cells and tumor cells, showing validation screen with focused epigenetic gRNA library. The library contained gRNAs for the top 31 genes from the primary screen and two positive control genes (Pdcd1 and Cblb); 186 gRNAs were added as controls. OT-I T cells were transduced with the lentiviral gRNA library and injected into mice with subcutaneous B16F10-OVA tumors. On day 10 following T cell transfer, gRNA representation was quantified for T cells isolated from tumors (experimental organ) versus spleens (control organ). Graph shows log 2 fold difference in gRNA representation in tumors versus spleens (X-axis) and statistical significance for indicated genes (Y-axis).

FIG. 8B illustrates identification of CARM1 as a therapeutic target in tumor-infiltrating T cells and tumor cells, showing validation of Carm1 KO in CD8 T cells by CRISPR. TIDE analysis (Tracking of Indels by Decomposition) of genomic DNA sequenced from KO cells showing 97.8% editing efficiency.

FIG. 8C illustrates identification of CARM1 as a therapeutic target in tumor-infiltrating T cells and tumor cells, showing western blot analysis of Carm1 protein in OT-I CD8 T cells edited with control or Carm1 gRNAs (2 different Carm1 gRNAs, 4 technical replicates in each group). CD8 T cells were electroporated with RNPs composed of Cas9 protein with bound gRNAs; Carm1 protein levels were analyzed on day 7 following electroporation.

FIG. 8D illustrates identification of CARM1 as a therapeutic target in tumor-infiltrating T cells and tumor cells, showing T cell cytotoxicity assay with Carm1-KO (Carm1 gRNA #1) and control-KO OT-I CD8 T cells. T cells were co-cultured with B16F10-OVA-ZsGreen tumor cells at indicated effector to target (E:T) ratios (n=7-10/replicates per condition); 48 hours later live GFP-positive tumor cells were counted using a Celigo image cytometer. Data are representative of three experiments and shown as mean±SEM. ****p<0.0001, by unpaired two-sided Mann-Whitney test.

FIGS. 8E-8F illustrate identification of CARM1 as a therapeutic target in tumor-infiltrating T cells and tumor cells, showing T cell cytotoxicity assay with Carm1-KO (Carm1 gRNA #2) and control-KO OT-I CD8 T cells. 24 (FIG. 8E) and 48 (FIG. 8F) hours later live GFP-positive tumor cells were counted using a Celigo image cytometer.

FIGS. 8G-8H illustrate identification of CARM1 as a therapeutic target in tumor-infiltrating T cells and tumor cells, showing flow cytometry analysis of Carm1-KO CD8 T cells generated using gRNA #1 (FIG. 8G), gRNA #2 (FIG. 8H) and control-KO OT-I CD8 T cells following co-culture with tumor cells. Edited T cells were co-cultured with B16-OVA-Zsgreen tumor cells at a 1:2 ratio for 24 hours followed by flow cytometric analysis of indicated markers. Data are representative of two experiments and shown as mean±SEM, ** p<0.01, ***p<0.001, ****p<0.0001 by unpaired two-sided Mann-Whitney test.

FIG. 8I illustrates identification of CARM1 as a therapeutic target in tumor-infiltrating T cells and tumor cells, showing assay to examine antigen-dependent proliferation of control-KO or Carm1-KO CD8 T cells. OT-I CD8 T cells were edited with control or Carm1 gRNAs (gRNA #1 or #2) and cultured for 5 days in the presence of IL-15 and IL-7. T cells were then co-cultured for 4 days with B16-OVA-ZsGreen cells at a 5:1 (E:T) ratio, and CTV dilution was assessed by flow cytometry. Data were summarized as mean±S.E.M. and analyzed by unpaired two-sided Mann-Whitney test. **p<0.01, ****p<0.0001.

FIG. 9A illustrates that inactivation of Carm1 in CD8 T cells enhances their anti-tumor function, showing survival of mice with B16F10-Ova melanomas following adoptive transfer of Carm1-KO (generated using gRNA #1) or control-KO OT-I CD45.1 CD8 T cells.

FIG. 9B illustrates that inactivation of Carm1 in CD8 T cells enhances their anti-tumor function, showing representative flow plots of tumor infiltrating CD45.1 CD8 T cells following adoptive transfer of Carm1-KO or control-KO OT-I CD45.1 CD8 T cells. Gated on Live/singlets/CD45+ cells.

FIG. 9C illustrates that inactivation of Carm1 in CD8 T cells enhances their anti-tumor function, showing anti-tumor activity of adoptively transferred Carm1-KO (generated using gRNA #2) or control-KO OT-I CD45.1 CD8 T cells. B16-OVA-ZsGreen tumor cells (0.1×10⁶) were implanted subcutaneously. On day 7 following tumor cell inoculation, edited CD8 T cells (1×10⁶) were transferred via tail vein injection. Tumor size was recorded; n=8-10 mice per group.

FIG. 9D illustrates that inactivation of Carm1 in CD8 T cells enhances their anti-tumor function, showing survival of mice with B16F10-Ova melanomas following adoptive transfer of Carm1-KO (generated using gRNA #2) or control-KO OT-I CD45.1 CD8 T cells.

FIG. 9E illustrates that inactivation of Carm1 in CD8 T cells enhances their anti-tumor function, showing growth of Carm1-KO and control-KO B16F10 melanoma cells (left) and 4T1 breast cancer cells (right) in a colony formation assay (500 input cells/well, 6-well plates for 5 days). Quantification of number of colonies in each group and representative images of plates of colonies are shown for each condition. Data are representative of two independent experiments.

FIG. 10A illustrates that targeting of Carm1 induces a type I interferon response in human and murine tumor cells, showing growth of Carm1-KO (Carm1 gRNA #2) and control-KO B16F10 tumors (left) and survival of tumor bearing mice (right). Mice (n=8-10/group) were treated with CD8 depleting or isotype control antibodies.

FIG. 10B illustrates that targeting of Carm1 induces a type I interferon response in human and murine tumor cells, showing heatmap of differentially expressed ISGs in Carm1-KO and control-KO B16F10 tumor cells (n=3/group) that were previously found to be associated with immunotherapy response in human melanoma (Benci et al., 2019). Data are representative of two independent experiments.

FIG. 10C illustrates that targeting of Carm1 induces a type I interferon response in human and murine tumor cells, showing heatmap of differentially expressed p53 pathway genes in Carm1-KO versus control-KO B16F10 tumor cells (n=3/group). Data are representative of two independent experiments.

FIG. 10D illustrates that targeting of Carm1 induces a type I interferon response in human and murine tumor cells, showing RT-qPCR analysis of indicated ISGs in B16F10 cells treated with CARM1 inhibitor EZM2302 (0-1 μM) for 7 days (n=3/group).

FIG. 10E illustrates that targeting of Carm1 induces a type I interferon response in human and murine tumor cells, showing western blot analysis to validate activity of CARM1 inhibitor (EZM2302) in human tumor cells. BAF155 is a well-validated target of CARM1. SKBR3, MDA-MB-157 and MDA-MB-436 cells were treated with EZM2302 (0.1 μM) or solvent control for 24 hours. Western blots were probed with Abs specific for di-methylated BAF155 protein (me2BAF155), total BAF155 protein and β-actin (loading control).

FIG. 10F illustrates that targeting of Carm1 induces a type I interferon response in human and murine tumor cells, showing analysis of ISGs in human tumor cells treated with CARM1 inhibitor (EZM2302). RT-qPCR analysis of selected ISGs and IFNs in human SKBR3, MDA-MB-157 and MDA-MB-436 cells treated with vehicle or CARM1 inhibitor (2 μM) for 7 days (n=3/group).

FIG. 10G illustrates that targeting of Carm1 induces a type I interferon response in human and murine tumor cells, showing responsiveness of Carm1-KO compared to control-KO B16F10 cells to IFNγ treatment. Cells were treated overnight with IFNγ (0, 2 or 5 ng/ml), and mRNA levels of ISGs were analyzed by RT-qPCR (n=3/group).

FIG. 10H illustrates that targeting of Carm1 induces a type I interferon response in human and murine tumor cells, showing responsiveness of Carm1-KO compared to control-KO 4T1 cells to IFNγ treatment. Cells were treated overnight with IFNγ (0, 2 or 5 ng/ml), and mRNA levels of ISGs were analyzed by RT-qPCR (n=3/group).

FIG. 11A illustrates that targeting of Carm1 enhances sensitivity of tumor cells to IFNγ, showing analysis of IFNγ signaling in Carm1-KO and control-KO B16F10 cells. B16F10 cells were stimulated with IFNγ for 0-5 minutes, and levels of phosphorylated STAT1 (pSTAT1) as well as total STAT1, STAT2 and β-actin were analyzed by Western blotting. Long (5 min) and short (1 min) exposures of same blot are shown for pSTAT1 (left). Quantification of pSTAT1 blots using ImageJ from three separate experiments shown on right.

FIG. 11B illustrates that targeting of Carm1 enhances sensitivity of tumor cells to IFNγ, showing analysis of cell proliferation of Carm1-KO and control-KO B16F10 cells in the presence of IFNγ. Equal numbers of GFP+ B16F10 cells were cultured in complete media supplemented with IFNγ for 4 days, and GFP+ live cells were counted each day using a Celigo image cytometer (n=4/group).

FIG. 11C illustrates that targeting of Carm1 enhances sensitivity of tumor cells to IFNγ, showing analysis of H2-K^{b} expression+/− IFNγ treatment of Carm1-KO and control-KO B16F10 cells.

FIG. 11D illustrates that targeting of Carm1 enhances sensitivity of tumor cells to IFNγ, showing analysis of PD-L1 expression+/− IFNγ treatment of Carm1-KO and control-KO B 16F10 cells.

FIG. 11E illustrates that targeting of Carm1 enhances sensitivity of tumor cells to IFNγ, showing RT-qPCR analysis of indicated ISGs following stimulation with the indicated concentrations of IFNγ in control-KO, Carm1-KO, Ifnar 1-KO and Ifnar 1/Carm1 dKO B16F10 cells (n=3/group).

FIG. 12A illustrates that inactivation of Carm1 induces a cGAS-mediated type I interferon response in human and murine tumor cells, showing Mavs protein levels in Carm1-KO B16F10 cells edited with Mavs or control gRNAs. Replicates of different lines edited with the same gRNA are shown. β-actin is shown as a loading control.

FIG. 12B illustrates that inactivation of Carm1 induces a cGAS-mediated type I interferon response in human and murine tumor cells, showing RT-qPCR analysis of selected ISGs and IFNs in control, Carm1-KO, Mavs-KO and Carm1/Mavs dKO B16F10 cells (n=3/group).

FIG. 12C illustrates that inactivation of Carm1 induces a cGAS-mediated type I interferon response in human and murine tumor cells, showing cGas protein levels in Carm1-KO B16F10 cells edited with Cgas or control gRNAs. Replicates of different lines edited with the same Cgas gRNA or a control gRNA are shown. β-actin is shown as a loading control.

FIG. 12D illustrates that inactivation of Carm1 induces a cGAS-mediated type I interferon response in human and murine tumor cells, showing RT-qPCR analysis of selected ISGs in control, Carm1-KO, Cgas-KO and Carm1/Cgas dKO B16F10 cells (n=3/group).

FIG. 12E illustrates that inactivation of Carm1 induces a cGAS-mediated type I interferon response in human and murine tumor cells, showing analysis of micronuclei in Carm1-KO and control-KO B16F10 cells. Tumor cells were transduced with a HA epitope tagged Cgas cDNA; ZsGreen was expressed downstream of an IRES by the same lentiviral vector. Representative immunofluorescence for HA epitope tagged cGAS (purple) and DAPI (blue); DAPI labeling was used to identify nuclei and micronuclei. Scale bar—10 μM.

FIG. 12F illustrates that inactivation of Carm1 induces a cGAS-mediated type I interferon response in human and murine tumor cells, showing dsDNA damage in CARM1 inhibitor (EZM2302) versus vehicle (5% Dextrose) treated B16F10 tumor cells based on labeling with γH2AX antibody. Representative immunofluorescence images (left) of γH2AX antibody labeling (purple); nuclei labeled with DAPI. Quantification of number of γH2AX foci/nucleus (right). Data are shown as mean±SEM, ***p<0.001, by unpaired two-sided Mann-Whitney test. Scale bar—10 μM.

FIG. 12G illustrates that inactivation of Carm1 induces a cGAS-mediated type I interferon response in human and murine tumor cells, showing Quantification of the percentage of B16F10 tumor cells with γH2AX foci following editing with two different control gRNAs (LacZ control gRNA, intergenic gRNA).

FIG. 12H illustrates that inactivation of Carm1 induces a cGAS-mediated type I interferon response in human and murine tumor cells, showing RT-qPCR analysis of ISGs in B16F10 cells without editing (WT) and following editing with two different control gRNAs (LacZ, intergenic). ISG levels were analyzed >7 days following editing. Data shown in FIGS. 12B, 12D, 12F and 12G are summarized as mean±S.D. and were analyzed by unpaired two-sided Mann-Whitney test. Data are representative of three experiments, *p<0.05, **p<0.01, ***p<0.001, ns (non-significant).

FIG. 13A illustrates that Carm1 inactivation sensitizes resistant tumors to treatment with CTLA-4 or PD-1 mAbs, showing treatment of Carm1-KO or control-KO B16F10 tumors with PD-1 or isotype control antibodies (n=8 mice/group). Tumor growth (left) and survival of tumor bearing mice (right) are shown.

FIG. 13B illustrates that Carm1 inactivation sensitizes resistant tumors to treatment with CTLA-4 or PD-1 mAbs, showing quantification of CD4 T cells (number per gram of tumor) in Carm1-KO and control-KO B16F10 tumors following treatment with anti-CTLA4 or control mAbs, n=8/group.

FIG. 13C illustrates that Carm1 inactivation sensitizes resistant tumors to treatment with CTLA-4 or PD-1 mAbs, showing number of intra-tumoral perforin+CD8+(left) and IL2+CD8+(right) T cells (per gram of tumor) in Carm1-KO and control-KO B16F10 tumors following treatment with anti-CTLA4 or control mAbs, n=8/group.

FIG. 13D illustrates that Carm1 inactivation sensitizes resistant tumors to treatment with CTLA-4 or PD-1 mAbs, showing number of intra-tumoral macrophages (live, singlet, CD45+CD3− F4/80+) (left) and dendritic cells (DCs) (live, singlet, CD45+CD3− F4/80− CD11c+ MHCII^{hi}) (middle) calculated per gram of tumor. Quantification of myeloid derived suppressor (MDSCs) (CD45+/CD3−/F4/80−/Gr1+, as percentage of CD3− cells) (right). Myeloid cells were analyzed in Carm1-KO and control-KO B16 tumors following treatment with anti-CTLA4 or control mAbs, n=8/group.

FIG. 13E illustrates that Carm1 inactivation sensitizes resistant tumors to treatment with CTLA-4 or PD-1 mAbs, showing analysis of Carm1-KO and control-KO 4T1 tumors following treatment with anti-CTLA4 or control mAbs. Contour plot show percentage of CD4 and CD8 positive intra-tumoral T cells (left). Quantification of percentage (middle) and number (right) of CD8+ T cells for indicated treatment groups.

FIG. 13F illustrates that Carm1 inactivation sensitizes resistant tumors to treatment with CTLA-4 or PD-1 mAbs, showing quantification of CD4+ T cells (number per gram of tumor) for indicated treatment groups in 4T1 tumor model.

FIG. 13G illustrates that Carm1 inactivation sensitizes resistant tumors to treatment with CTLA-4 or PD-1 mAbs, showing quantification of CD25+ intra-tumoral CD8+ T cells for indicated treatment groups in the 4T1 tumor model.

FIG. 13H illustrates that Carm1 inactivation sensitizes resistant tumors to treatment with CTLA-4 or PD-1 mAbs, showing quantification of CD69+ intra-tumoral CD8+ T cells for indicated treatment groups in the 4T1 tumor model.

FIG. 13I illustrates that Carm1 inactivation sensitizes resistant tumors to treatment with CTLA-4 or PD-1 mAbs, showing quantification of IFNγ (left), perforin (middle) and TNFα (right) positive intra-tumoral CD8+ T cells for indicated treatment groups in the 4T1 tumor model. In FIGS. 13A-13I, data shown are representative of two independent experiments with 8 mice per group. No outliers were removed. Bar graphs represent data summarized as mean±S.E.M and were analyzed by unpaired two-sided Mann-Whitney test, ****P<0.0001; ***P<0.001; **P<0.01; *P<0.05; n.s., non-significant.

FIG. 14A illustrates evaluation of potential toxicity of Carm1 inhibitor (EZM2302) in C57Bl/6 mouse model, showing histopathological evaluation of major organs for assessment of potential toxicity of Carm1 inhibitor. Sex and age matched C57Bl/6 mice were randomized into two groups treated twice daily with either CARM1 inhibitor (150 mg/kg) or vehicle via oral gavage for 14 days. Major organs including heart, spleen, kidney, liver, lung and small intestine were harvested for pathological assessment. Representative images of histopathological images (H&E stain) from vehicle or Carm1 inhibitor treated mice are shown (n=8/group). Scale bar=50 μM.

FIG. 14B illustrates evaluation of potential toxicity of Carm1 inhibitor (EZM2302) in C57Bl/6 mouse model, showing analysis of body weight of sex and age matched C57Bl/6 mice treated twice daily with CARM1 inhibitor (150 mg/kg) or vehicle via oral gavage for 14 days (n=8/group).

FIG. 15A illustrates changes in tumor microenvironment induced by monotherapy or combination therapy with CARM1 inhibitor, showing treatment of mice with B16F10 melanomas with CARM1 inhibitor or vehicle control. Mice also received isotype control, CTLA-4 or PD-1 mAbs, as indicated (n=5 mice/group). Representative flow plots of CD8 T cells gated on live/singlets/CD45+/CD3+ cells from indicated groups are shown on left. Quantification of CD8 T cells is shown as percentage of CD3+ T cells (middle) or number per gram of tumor (right) for the indicated treatment groups.

FIG. 15B illustrates changes in tumor microenvironment induced by monotherapy or combination therapy with CARM1 inhibitor, showing number of intra-tumoral granzyme B+CD8+ T cells per gram of tumor for the indicated treatment groups (n=5 mice/group). Representative flow plots are shown on left.

FIG. 15C illustrates changes in tumor microenvironment induced by monotherapy or combination therapy with CARM1 inhibitor, showing number of intra-tumoral IL2+CD8+ T cells per gram of tumor for the indicated treatment groups (n=5 mice/group).

FIG. 15D illustrates changes in tumor microenvironment induced by monotherapy or combination therapy with CARM1 inhibitor, showing number of intra-tumoral IFNγ+CD8+ T cells per gram of tumor for the indicated treatment groups (n=5 mice/group). Representative flow plots are shown on left.

FIG. 15E illustrates changes in tumor microenvironment induced by monotherapy or combination therapy with CARM1 inhibitor, showing number of intra-tumoral perforin+CD8+ T cells per gram of tumor for the indicated treatment groups (n=5 mice/group).

FIG. 15F illustrates changes in tumor microenvironment induced by monotherapy or combination therapy with CARM1 inhibitor, showing analysis of intra-tumoral PD1+CD8+ T cells (quantified as percentage of CD8+ T cells) for the indicated treatment groups (n=5 mice/group). Representative flow plots (left) and quantification of PD-1 expression as percentage of CD8 T cells (middle) or MFI (right) are shown.

FIG. 16A illustrates changes in tumor microenvironment induced by monotherapy or combination therapy with CARM1 inhibitor, showing treatment of mice with B16F10 melanomas with CARM1 inhibitor or vehicle control. Mice also received isotype control, CTLA-4 or PD-1 mAbs, as indicated (n=5 mice/group). Quantification of CD4+ T cells (number per gram of tumor) for the indicated treatment groups.

FIG. 16B illustrates changes in tumor microenvironment induced by monotherapy or combination therapy with CARM1 inhibitor, showing quantification of IFNγ+ cells (as percentage of CD4+ T cells) for the indicated treatment groups.

FIG. 16C illustrates changes in tumor microenvironment induced by monotherapy or combination therapy with CARM1 inhibitor, showing quantification of FoxP3+ Treg cells (as percentage of CD4+ T cells) for the indicated treatment groups.

FIG. 16D illustrates changes in tumor microenvironment induced by monotherapy or combination therapy with CARM1 inhibitor, showing quantification of the CD8/FoxP3 Treg ratio for the indicated treatment groups.

FIG. 16E illustrates changes in tumor microenvironment induced by monotherapy or combination therapy with CARM1 inhibitor, showing number of intra-tumoral NK cells (left), dendritic cells (live, singlet, CD45+CD3−F4/80-CD11c+ MHCII^{hi}) (middle) and macrophages (live, singlet, CD45+CD3−F4/80+) (right) calculated per gram of tumor.

FIG. 17A illustrates reconstitution of Carm1 gene expression using a doxycycline-inducible promoter reverses the Carm1 knockout phenotype, showing transduction of Carm1-KO B16F10 cells with a lentiviral vector (DOX-Carm1) driving expression of a Carm1 cDNA under the control of a doxycycline (DOX) inducible promoter. Representative flow plots are shown following sorting of GFP+ cells for tumor cells transduced with DOX-Carm1 or empty vectors.

FIG. 17B illustrates reconstitution of Carm1 gene expression using a doxycycline-inducible promoter reverses the Carm1 knockout phenotype, showing western blot validation of Carm1 protein expression following DOX induction for 7 days in the indicated cell populations. Gapdh is shown as loading control.

FIG. 17C illustrates reconstitution of Carm1 gene expression using a doxycycline-inducible promoter reverses the Carm1 knockout phenotype, showing RT-qPCR analysis of selected ISGs. Carm1-KO cells expressing the DOX-inducible Carm1 cDNA were treated with doxycycline (0, 100 or 500 ng/ml) for 7 days and then treated overnight with IFNγ (0, 1 or 5 ng/ml) (n=3/group). Control-KO cells not treated with IFNγ were included for comparison.

FIG. 17D illustrates reconstitution of Carm1 gene expression using a doxycycline-inducible promoter reverses the Carm1 knockout phenotype, showing growth of B16F10 melanomas was compared for the following conditions: control-KO tumor cells transduced with the empty vector, Carm1-KO tumor cells transduced with the empty vector and Carm1 KO tumor cells transduced with DOX-Carm1 vector. For each of these groups, mice were fed a regular diet or a doxycycline-containing diet (625 ppm, Envigo Teclad) post tumor cell injection until the experimental endpoint (18 days).

FIG. 18A illustrates that reconstitution of Carm1 gene expression using a doxycycline-inducible promoter reverses the favorable changes in the tumor microenvironment, showing control-KO B16F10 tumor cells transduced with the empty lentiviral vector or Carm1-KO tumor cells expressing the DOX-Carm1 cDNA were implanted into C57Bl/6 mice. Mice with either type of tumor cells were fed a regular diet or a doxycycline-containing diet (625 ppm, Envigo Teclad) post tumor cell injection until the experimental endpoint (18 days). CD8 and CD4 T cell infiltration was analyzed for the indicated treatment groups (n=5 mice/group). Contour plots show percentage of CD4+ and CD8+ intra-tumoral T cells (left). Summary graphs show quantification of CD8 and CD4 T cells as number per gram of tumor (right).

FIG. 18B illustrates that reconstitution of Carm1 gene expression using a doxycycline-inducible promoter reverses the favorable changes in the tumor microenvironment, showing quantification of PD-1+ intra-tumoral CD8+ T cells for the indicated treatment groups (right). Contour plots (left) show percentage of CD8+PD-1+ positive intra-tumoral T cells, n=5/group.

FIG. 18C illustrates that reconstitution of Carm1 gene expression using a doxycycline-inducible promoter reverses the favorable changes in the tumor microenvironment, showing quantification of CD25+ intra-tumoral CD8+ T cells for the indicated treatment groups (right). Contour plots (left) show percentage of CD8+ T cells that are CD25 positive, n=5/group.

FIG. 18D illustrates that reconstitution of Carm1 gene expression using a doxycycline-inducible promoter reverses the favorable changes in the tumor microenvironment, showing characterization of intra-tumoral cDC1 cells. Contour plot show percentage of CD103+CD11b− cDC1s (gated on live, singlet, CD45+CD3−F4/80− CD11c+ MHCII^{hi}) (left). Summary plot shows percentage of cDC1 (CD103+CD11b−) cells as percentage of total DCs (right), n=5/group.

FIG. 19A illustrates that inactivation of Tdrd3 and Med12 genes results in a similar phenotype as inactivation of Carm1 gene, showing Tdrd3 protein levels in B16F10 cells edited with Tdrd3 or control (Ctrl) gRNAs. Replicates of different lines edited with same gRNA are shown. Cell line highlighted in red was used for experiments.

FIG. 19B illustrates that inactivation of Tdrd3 and Med12 genes results in a similar phenotype as inactivation of Carm1 gene, showing Med12 protein levels in B16F10 cells edited with Med12 or control gRNAs. Replicates of different lines edited with the same gRNA are shown.

FIG. 19C illustrates that inactivation of Tdrd3 and Med12 genes results in a similar phenotype as inactivation of Carm1 gene, showing RT-qPCR analysis of selected ISGs and IFNs in Tdrd3-KO and control-KO B16F10 cells (n=4/group).

FIG. 19D illustrates that inactivation of Tdrd3 and Med12 genes results in a similar phenotype as inactivation of Carm1 gene, showing RT-qPCR analysis of transcripts of selected ISGs and IFNs in Med12-KO and control-KO B16F10 cells (n=4/group).

FIG. 19E illustrates that inactivation of Tdrd3 and Med12 genes results in a similar phenotype as inactivation of Carm1 gene, showing response of Tdrd3-KO and control-KO B16F10 cells to IFNγ stimulation. RT-qPCR analysis of transcripts of selected ISGs and IFNs following overnight stimulation with the indicated concentrations of IFNγ (n=4/group).

FIG. 19F illustrates that inactivation of Tdrd3 and Med12 genes results in a similar phenotype as inactivation of Carm1 gene, showing response of Med12-KO and control-KO B16F10 cells to IFNγ stimulation. RT-qPCR analysis of transcripts of selected ISGs and IFNs following overnight stimulation with the indicated concentrations of IFNγ (n=4/group).

FIGS. 19G-19I illustrate that inactivation of Tdrd3 and Med12 genes results in a similar phenotype as inactivation of Carm1 gene, showing western blot validation of Top3b (FIG. 19G), Top1 (FIG. 19H) and Med13 (FIG. 19I) editing in B16F10 cells. RT-qPCR analysis was performed for selected ISGs and IFNs in indicated cell lines (bottom) (n=4/group). In FIGS. 19A-19I, data shown are representative of two independent experiments with 4 replicates/group. Mann-Whitney test was used to determine significance, ***P<0.001; **P<0.01; *P<0.05; n.s., not significant.

FIG. 20A illustrates alterations in transcription in Carm1-KO tumor cells, showing western blot analysis of nuclear lysates from control-KO, Carm1-KO or Tdrd3-KO B16F10 cells. Equal quantities of nuclear protein extracts from indicated cell lines were probed using antibodies specific for phosphorylated (p-Ser2 CTD and p-Ser5) or non-phosphorylated (total) C-terminal domain of RNA Pol II (CTD). Lamin A/C was used as loading control (left). Fraction of phosphorylated RNA Pol II was estimated by normalizing p-Ser2 CTD to total CTD levels (right). Data shown are representative of three experiments.

FIG. 20B illustrates alterations in transcription in Carm1-KO tumor cells, showing ratio of normalized P-Ser2 Pol II and total Pol II mNET-Seq signals within 500 bp of transcription start sites (TSS) of expressed protein-encoding genes in Carm1-KO (red) and control-KO (blue) cells. Shaded region indicates a 95% confidence interval.

FIG. 20C illustrates alterations in transcription in Carm1-KO tumor cells, showing Boxplots representing the ratio of P-Ser2 Pol II to total Pol II mNET-Seq reads within 500 bp of TSS in Carm1-KO (red) and in control-KO (blue) cells.

FIG. 20D illustrates alterations in transcription in Carm1-KO tumor cells, showing Venn diagram showing overlap between genes with increased normalized P-Ser2 RNA Pol II reads (shown as mNETSeq, blue) and genes with higher expression (shown as RNASeq, red) in Carm1-KO relative to control-KO B16F10 cells.

FIG. 20E illustrates alterations in transcription in Carm1-KO tumor cells, showing top enriched pathways from GSEA analysis of genes with increased normalized P-Ser2 RNA Pol II reads (all 2,837 mNETSeq genes, blue) (left) in Carm1-KO compared to control-KO B16F10 cells. GSEA analysis for 275 overlapping genes between mNETSeq and RNASeq (right).

FIG. 20F illustrates alterations in transcription in Carm1-KO tumor cells, showing pathway analysis of differentially spliced genes in Carm1-KO tumor cells. Differential splicing analysis was conducted using DESeq2 using log 2(FC) greater than 2-fold and adjusted p-value<0.05) as statistical thresholds. Enriched gene ontologies were identified using the String Database. Number of exon gains and losses are shown in inset.

FIG. 20G illustrates alterations in transcription in Carm1-KO tumor cells, showing metaplot for DRIPseq (DNA-RNA immunoprecipitation analysis) representing the log 2 fold change in mean normalized count of peaks in Carm1-KO and control-KO cells.

FIG. 21A illustrates expression of CARM1 in human cancers, showing analysis of TCGA RNA-seq data across human cancer types. Correlation of CARM1 mRNA levels with indicated pathways. Plots show Spearman's correlation and estimated statistical significance for indicated pathways in different cancer types adjusted for tumor purity. Each dot represents a cancer type in TCGA.

FIG. 21B illustrates expression of CARM1 in human cancers, showing analysis of scRNA-seq data of malignant cells from three human cancer cohorts (GSE123813: basal cell carcinoma; GSE103322: head and neck cancer; GSE116256: AML). Scores for IFN-γ response, IFN-α response and APC (antigen presentation cell infiltration) pathways are shown. Data were stratified by CARM1 high and low groups using median expression levels. Statistical comparisons were made using two-sided unpaired Mann-Whitney tests.

FIG. 21C illustrates expression of CARM1 in human cancers, showing association of CARM1 mRNA levels with survival in metastatic melanoma (SKCM), bladder urothelial carcinoma (BLCA), Low Grade Glioma (LGG), sarcoma (SARC), Kidney Renal Clear Cell Carcinoma (KIRC), Mesothelioma (MESO). A total of 12 TCGA datasets were analyzed. Statistical analysis was performed using TIMER2.0; shown are all cancer types in which CARM1 mRNA levels correlated with survival.

FIGS. 22A-22B illustrate single cell analysis of human tumor cells for correlation between CARM1 mRNA expression and DNA repair as well as antigen presentation pathways, showing single-cell RNA-seq data of malignant cells were investigated for correlation between CARM1 mRNA expression and DNA repair pathway hallmark genes (msigdb/hallmark: DNA Repair) (FIG. 22A) as well as antigen processing and presentation pathway (KEGG: https://www.genome.jp/kegg-bin/show_pathway?hsa04612) (FIG. 22B). Data are shown for the following human scRNA-seq datasets: ALL (Acute Lymphoblastic Leukemia), AML (Acute Myeloid Leukemia), MM (Multiple Myeloma) and NSCLC (Non-Small Cell Lung Cancer).

FIG. 23A illustrates analysis of CARM1 KO gene expression signature for ICB response in clinical trials and immune-related pathways, showing CARM1 KO gene signature levels in indicated ICB (immune checkpoint blockade) cohorts of responder and non-responder patients enrolled in clinical trials evaluating PD-1 or PD-L1 blocking mAbs. In each subgroup, predictive power of ICB response was evaluated by comparing CARM1 KO gene signature high and low groups. The p-values were inferred by Mann-Whitney U test. *p-value<0.1; NS (non-significant). Ipi (Ipilimumab).

FIG. 23B illustrates analysis of CARM1 KO gene expression signature for ICB response in clinical trials and immune-related pathways, showing analysis of CARM1 KO signature in TCGA RNA-seq datasets across human cancer types. Correlation of CARM1 KO signature with indicated immune-related pathways. Plots show Spearman's correlation and estimated statistical significance for indicated pathways in different cancer types adjusted for tumor purity. Each dot represents a cancer type in TCGA.

### DETAILED DESCRIPTION

Detailed descriptions of one or more preferred embodiments are provided herein. It is to be understood, however, that the present disclosure may be embodied in various forms. Therefore, specific details disclosed herein are not to be interpreted as limiting, but rather as a basis for the claims and as a representative basis for teaching one skilled in the art to employ the present disclosure in any appropriate manner.

### Definitions

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be non-limiting.

The term “substantially” allows for deviations from the descriptor that do not negatively impact the intended purpose. Descriptive terms are understood to be modified by the term “substantially” even if the word “substantially” is not explicitly recited.

As used herein the term “about” is used herein to mean approximately, roughly, around, or in the region of. When the term “about” is used in conjunction with a numerical range, it modifies that range by extending the boundaries above and below the numerical values set forth.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., at least 85%, 90%, 91%, 92%, 93%, 94%, 95%, 96%, 97%, 98%, 99% or 100% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. The disclosure provides polypeptides or polynucleotides that are substantially identical to the polypeptides or polynucleotides, respectively, exemplified herein. The identity exists over a region that is at least about 15, 25 or 50 nucleotides in length, or more preferably over a region that is 100 to 500 or 1000 or more nucleotides in length, or over the full length of the reference sequence. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, substantial identity exists when one or two amino acid residues are conservatively substituted, according to the conservative substitutions defined herein.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The terms “subject,” “patient,” and “individual” interchangeably refer to a mammal, for example, a human or a non-human primate mammal. The mammal can also be a laboratory mammal, e.g., mouse, rat, rabbit, hamster. In some embodiments, the mammal can be an agricultural mammal (e.g., equine, ovine, bovine, porcine, camelid) or domestic mammal (e.g., canine, feline).

As used herein, the terms “treat,” “treating,” or “treatment” of any disease or disorder refer in one embodiment, to ameliorating the disease or disorder (i.e., slowing or arresting or reducing the development of the disease or at least one of the clinical symptoms thereof). In another embodiment, “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically, (e.g., stabilization of a discernible symptom), physiologically, (e.g., stabilization of a physical parameter), or both. In yet another embodiment, “treat,” “treating,” or “treatment” refers to preventing or delaying the onset or development or progression of a disease or disorder.

The terms “cancer” and “cancerous” can refer to or describe the physiological condition in mammals that is typically characterized by unregulated cell growth.

As used herein, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas) and Hodgkin's lymphoma, leukemia (including AML, ALL, and CML), and multiple myeloma.

The terms “cancerous cell” or “cancer cell”, used either in the singular or plural form, can refer to cells that have undergone a malignant transformation that makes them pathological to the host organism. Malignant transformation is a single- or multi-step process, which involves in part an alteration in the genetic makeup of the cell and/or the expression profile. Malignant transformation may occur either spontaneously, or via an event or combination of events such as drug or chemical treatment, radiation, fusion with other cells, viral infection, or activation or inactivation of particular genes. Malignant transformation may occur in vivo or in vitro, and can if necessary be experimentally induced.

The term “Carm1 effector” as used herein refers to a gene or a protein which is part of the Carm1 pathway. For example, a Carm1 effector can be a protein methylated by Carm1 (such as Med12) or a gene expressing such protein, or a protein which recognizes Carm1-methylation (such as Tdrd3) or a gene expressing such protein. In some embodiments, a Carm1 effector may be a component of the Mediator complex.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

As used herein, the terms “engineered” cells or host cells, such as “genetically-engineered cells”, can refer to a cell into which an exogenous nucleic acid sequence, such as, for example, a vector, has been introduced. Therefore, recombinant or engineered cells are distinguishable from naturally occurring cells which do not contain a recombinantly introduced nucleic acid. In embodiments of the disclosure, a host cell is a T cell, including a cytotoxic T cell (also known as TC, Cytotoxic T Lymphocyte, CTL, T-Killer cell, cytolytic T cell, CD8+ T-cells or killer T cell); NK cells and NKT cells are also encompassed in the disclosure. In embodiments, the genetically engineered cell can comprise a CAR T cell, or an armed CAR T cell. See, for example, PCT/US2015/067225.

The term “inhibit” can refer to any degree of inhibition, reduction, dampen or suppression, and is not limited for these purposes to only total inhibition. Thus, any degree of partial inhibition or relative reduction is intended to be included within the scope of the term “inhibit”. For example, the term inhibit can refer to the inhibition of a biological activity or process, or the inhibition of a condition, symptom, or disease, by about 10%, about 20%, about 30%, about 40%, about 50%, about 60%, about 70%, about 80%, about 90%, about 100%, or 100% when compared to a control sample. The term “inhibit” can refer to the inhibition, reduction, dampen or suppression of a given biological activity or process, such as the expression of a gene or the activity of a protein. The term “inhibit” can also refer to the inhibition, reduction, dampen or suppression of a given condition, symptom, or disease. For example, embodiments of the disclosure can inhibit tumor growth (i.e., reduce tumor growth). The term “inhibit” can be used interchangeably with the term “suppress”, “reduce” and the like.

“Inhibiting agent” refers to any agent that can inhibit or reduce the expression or activity of a protein. For example, the inhibiting agent can inhibit or reduce the activity of the CARM1 protein, or degrade the CARM1 protein, by about 10%, about 20%, about 30%, about 40%, about 50%, about 60%, about 70%, about 80%, about 90%, about 100%, or 100% when compared to a control sample. In another example, the inhibiting agent can inhibit or reduce the activity of a Carm1 effector protein, or degrade a CARM1 effector protein, such as the Med 12 or Tdrd3 protein, by about 10%, about 20%, about 30%, about 40%, about 50%, about 60%, about 70%, about 80%, about 90%, about 100%, or 100% when compared to a control sample. The term “agent” or “compound” can be used interchangeably. In some embodiments, the inhibiting agent can inhibit or reduce the expression of a Carm1 gene, by about 10%, about 20%, about 30%, about 40%, about 50%, about 60%, about 70%, about 80%, about 90%, about 100%, or 100% when compared to a control sample. In some embodiments, the inhibiting agent can inhibit or reduce the expression of a Carm1 effector gene, such as Med12 or Tdrd3, etc., by about 10%, about 20%, about 30%, about 40%, about 50%, about 60%, about 70%, about 80%, about 90%, about 100%, or 100% when compared to a control sample.

As used herein, “gene silencing” induced by RNA interference refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without introduction of RNA interference. In one preferred embodiment, the mRNA levels are decreased by at least about 70%, about 80%, about 90%, about 95%, about 99%, about 100%.

“RNA interference (RNAi)” is a process whereby the expression or introduction of RNA of a sequence that is identical or highly similar to a target gene results in the sequence specific degradation or PTGS of messenger RNA (mRNA) transcribed from that targeted gene, thereby inhibiting expression of the target gene. This process has been described in plants, invertebrates, and mammalian cells. RNAi can also be initiated by introducing nucleic acid molecules, e.g., synthetic siRNAs or RNA interfering agents, to inhibit or silence the expression of target genes. As used herein, “inhibition of target gene expression” or “inhibition of marker gene expression” includes any decrease in expression or protein activity or level of the target gene (e.g., a marker gene of the disclosure) or protein encoded by the target gene, e.g., a marker protein of the disclosure. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene. These are the effector molecules for inducing RNAi, leading to posttranscriptional gene silencing with RNA-induced silencing complex (RISC). In addition to siRNA, which can be chemically synthesized, various other systems in the form of potential effector molecules for posttranscriptional gene silencing are available, including short hairpin RNAs (shRNAs), long dsRNAs, short temporal RNAs, and micro RNAs (miRNAs). These effector molecules either are processed into siRNA, such as in the case of shRNA, or directly aid gene silencing, as in the case of miRNA. The present disclosure thus encompasses the use of shRNA as well as any other suitable form of RNA to effect posttranscriptional gene silencing by RNAi. Use of shRNA has the advantage over use of chemically synthesized siRNA in that the suppression of the target gene is typically long-term and stable. An siRNA may be chemically synthesized, may be produced by in vitro by transcription, or may be produced within a host cell from expressed shRNA.

The term “small molecule” can refer to a non-peptidic, non-oligomeric organic compound either synthesized or found in nature. Small molecules, as used herein, can refer to compounds that are “natural product-like”, however, the term “small molecule” is not limited to “natural product-like” compounds. Rather, a small molecule is typically characterized in that it possesses one or more of the following characteristics including having several carbon-carbon bonds, having multiple stereocenters, having multiple functional groups, and having at least two different types of functional groups, although this characterization is not intended to be limiting for the purposes of the present disclosure.

The term “subject afflicted with a cancer” can refer to a subject who has been tested and found to have cancerous cells. The words “afflicted” and “diagnosed with” can be used interchangeably.

The term “therapeutically effective amount” can refer to those amounts that, when administered to a particular subject in view of the nature and severity of that subject's disease or condition, will have a desired therapeutic effect, e.g., an amount which will cure, prevent, inhibit, or at least partially arrest or partially prevent a target disease or condition. In some embodiments, the term “therapeutically effective amount” or “effective amount” can refer to an amount of a therapeutic agent that when administered alone or in combination with an additional therapeutic agent to a cell, tissue, or subject is effective to prevent or ameliorate the disease or condition, such as checkpoint blockade resistant cancers, or the progression of the disease or condition. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

When used herein, the term “solid tumor” can refer to an abnormal growth or abnormal mass of tissue that usually does not contain bubbles or regions filled with fluid. Solid tumors may be benign (noncancerous) or malignant. Different types of solid tumors are called cells of the type that form them. Examples of solid tumors are sarcomas, carcinomas and lymphomas. Leukemias (cancers of the blood) generally do not form solid tumors (National Cancer Institute, Dictionary of Cancer Terms).

### CARM1

Several cancer drugs activate innate immune pathways in tumor cells but unfortunately compromise anti-tumor immune function. It was surprisingly discovered that inhibition of Carm1, an epigenetic enzyme, elicited beneficial effects in both cytotoxic T cells and tumor cells. Inactivation of Carm1 enhanced transcription in both T cells and tumor cells but with distinct cellular consequences. Carm1 inactivation in cytotoxic T cells enhanced their activation state and anti-tumor function. In contrast, Carm1 inhibition in tumor cells resulted in accumulation of R-loops, genomic damage and activation of the cGAS-STING pathway. Inactivation of Med12, a regulatory component of the Mediator complex, elicited the same tumor cell phenotype, thereby connecting Carm1 to regulation of transcription. Inhibition of Carm1 induced potent T cell mediated tumor immunity and sensitized resistant, highly aggressive tumor cells to checkpoint blockade. The Carm1—Med12 pathway thus offers an opportunity to enhance anti-tumor immunity while simultaneously sensitizing resistant tumor cells to immune attack.

In some embodiments, a genetic screen in tumor-infiltrating T cells was performed and it was discovered that inactivation of the CARM1 gene enhanced T cell function against tumors. Interestingly, CARM1 was the top hit in this screen and scored higher than the gene encoding the PD-1 inhibitory receptor. Functional validation demonstrated that inactivation of this gene improved T cell-mediated cytotoxicity against tumor cells. CARM1 is therefore an interesting target for enhancing the anti-tumor function of cytotoxic T cells.

The CARM1 gene in tumor cells was also inactivated and it was discovered that CARM1 deficient tumor cells elicited a substantial anti-tumor immune response mediated by T cells. Inactivation of CARMI elicited such anti-tumor immune responses even in tumor models that are refractory to checkpoint blockade with PD-I or PD-1 plus CTLA-4 antibodies. It has been shown that an available small molecule inhibitor of CARM1 has significant therapeutic activity, again in an immunotherapy refractory model. Mechanistic studies demonstrated that inactivation of CARMI induced an innate immune response in tumor cells (type 1 interferon signaling). This innate immune response was lost when the gene encoding cGAS, a sensor for cytosolic DNA, was inactivated. Inactivation of CARM1 thus leads to a DNA damage response in tumor cells and production or cytosolic DNA that inactivates an innate immune response in tumor cells. Targeting CARM1 represents a novel therapeutic strategy for cancers that fail to respond to checkpoint blockade with PD-I or CTLA-4 antibodies.

In some embodiments, the present disclosure is directed to Carm1, an arginine methyltransferase that introduces asymmetric methylation of arginine residues in histone H3 and other chromatin-associated proteins. Asymmetric methylation refers to a highly specific modification in which two methyl groups are attached to one of the two nitrogen atoms of the arginine side chain (Wysocka J, Allis C D, Coonrod S. Histone arginine methylation and its dynamic regulation. Frontiers in bioscience: a journal and virtual library 2006; 11:344-55). CARM1 acts as a transcriptional co-activator for nuclear hormone receptors and other transcription factors. It is recruited to chromatin by a member of the p160 family of proteins which also recruits the p300/CBP histone acetyltransferases. Following recruitment, CARM1 enhances the activity of this co-activation complex by methylation of arginine residues in p160, p300/CBP and histone H3 (on residues H3R17 and H3R26) (Daujat S, Bauer U M, Shah V, Turner B, Berger S, Kouzarides T. Crosstalk between CARM1 methylation and CBP acetylation on histone H3. Curr Biol 2002; 12 (24): 2090-7; Chen D, Ma H, Hong H, Koh S S, Huang S M, Schurter B T, et al. Regulation of transcription by a protein methyltransferase. Science 1999; 284 (5423); 2174-7; Chen D, Huang S M, Stallcup M R. Synergistic, p160 coactivator-dependent enhancement of estrogen receptor function by CARM1 and p300. The Journal of biological chemistry 2000; 275 (52): 40810-6). Overexpression of CARM1 mRNA has been reported for many human cancer types, and in breast and prostate cancers it serves as a co-activator of transcription for estrogen alpha and androgen receptors (Kim Y R, Lee B K, Park R Y, Nguyen N T, Bae J A, Kwon D D, et al. Differential CARM1 expression in prostate and colorectal cancers. BMC Cancer 2010; 10:197; Al-Dhaheri M, Wu J, Skliris G P, Li J, Higashimato K, Wang Y, et al. CARM1 is an important determinant of ERalpha-dependent breast cancer cell differentiation and proliferation in breast cancer cells. Cancer Res 2011; 71 (6): 2118-28).

Using an in vivo CRISPR/Cas9 screen, it was discovered that inactivation of the Carm1 gene in T cells enhanced their anti-tumor function and increased the pool of tumor-infiltrating memory-like T cells which are known to be required for sustained immunity. Recent work demonstrated that effector T cell populations are only maintained in tumors when sufficient numbers of tumor-specific memory-like cells are present in the microenvironment (Jansen C S, Prokhnevska N, Master V A, Sanda M G, Carlisle J W, Bilen M A, et al. An intra-tumoral niche maintains and differentiates stem-like CD8 T cells. Nature 2019; 576 (7787): 465-70; Siddiqui I, Schaeuble K, Chennupati V, Fuertes Marraco S A, Calderon-Copete S, Pais Ferreira D, et al. Intratumoral Tcf1(+)PD-1(+)CD8(+) T Cells with Stem-like Properties Promote Tumor Control in Response to Vaccination and Checkpoint Blockade Immunotherapy. Immunity 2019; 50(1): 195-211). Inactivation of Carm1 in tumor cells elicited a potent T cell-dependent immune attack associated with greatly increased infiltration of tumors by CD8 T cells and dendritic cells. These data demonstrate that targeting of Carm1 induces potent tumor immunity by sensitizing resistant tumors to immune attack and enhancing anti-tumor T cell function.

In some embodiments, the present disclosure provides a novel approach for immunotherapy of tumors resistant to checkpoint blockade. Many human cancers fail to respond to PD-1 and/or CTLA-4 antibodies, and these resistant tumors frequently lack significant infiltration by CD8 T cells (‘cold’ tumors) (Gajewski T F, Schreiber H, Fu Y X. Innate and adaptive immune cells in the tumor microenvironment. Nat Immunol 2013; 14(10): 1014-22). Without being bound by theory, such tumors are not sufficiently immunogenic to elicit a spontaneous T cell response that can be enhanced by checkpoint blockade. Priming of a tumor-specific cytotoxic T cell response requires recruitment of cross-presenting dendritic cells (cDC1) into tumors, followed by activation and migration of dendritic cells into tumor-draining lymph nodes where they prime naïve CD8 T cells (Wculek S K, Cueto F J, Mujal A M, Melero I, Krummel M F, Sancho D. Dendritic cells in cancer immunology and immunotherapy. Nat Rev Immunol 2020; 20(1): 7-24). These cellular events require activation of innate immune signals that induce production of key chemokines and cytokines, including type 1 interferons that activate dendritic cells. It is shown in the disclosure that this barrier to effective cancer immunotherapy can be overcome by inhibition of Carm1, an epigenetic regulator. Interestingly, Carm1 inhibition in either tumor cells or T cells had major beneficial effects on T cell-mediated tumor immunity. Inactivation of Carm1 in tumor cells induced a type 1 interferon response and resulted in substantially increased numbers of tumor infiltrating CD8 T cells, NK cells and dendritic cells. Also, these tumor-infiltrating CD8 T cells showed higher functionality and lower expression of exhaustion markers. Inactivation of Carm1 in T cells preserved a substantial pool of tumor-infiltrating memory-like CD8 T cells with enhanced anti-tumor function. These findings are surprisingly significant because T cell exhaustion and loss of tumor-infiltrating memory populations are considered to represent major barriers to protective tumor immunity. The present disclosure provides substantial evidence that this pathway is relevant in human cancers. Analysis of TCGA data demonstrated high CARM1 mRNA levels across a wide range of human cancer types, including human cancers that have thus far been largely resistant to checkpoint blockade. CARM1 mRNA levels were negatively correlated with gene expression signatures of key immune pathways, including the MHC class I antigen presentation, type 1 interferon and IFNγ pathways. These human data are consistent with previous publications which demonstrated that a type 1 gene expression signature is associated with T cell inflamed (‘hot’) as opposed to non-T cell inflamed (‘cold’) tumors (Gajewski T F, Schreiber H, Fu Y X. Innate and adaptive immune cells in the tumor microenvironment. Nat Immunol 2013; 14(10): 1014-22).

It was surprisingly found that inhibition of Carm1 elicits such distinct responses in T cells versus tumor cells. Without being bound by theory, inactivation of Carm1 in T cells greatly increased the CD8 T cell accumulation in tumors. RNA-seq analysis of Carm1-KO compared to control-KO T cells indicated that Carm1 inactivation reduced terminal effector differentiation (reduced expression of Klrg1) which is known to impair T cell-mediated tumor immunity. Rather, Carm1-KO T cells expressed higher levels of transcription factors critical for differentiation, self-renewal and persistence of memory T cells, including Tcf7 and Myb (Raghu D, Xue H H, Mielke L A. Control of Lymphocyte Fate, Infection, and Tumor Immunity by TCF-1. Trends in immunology 2019; 40(12): 1149-62). A recent single-cell RNA-seq analysis of CD8 T cells in human melanomas demonstrated that higher expression of TCF7 by CD8 T cells predicted a positive outcome in patients treated with checkpoint blockade (Sade-Feldman M, Yizhak K, Bjorgaard S L, Ray J P, de Boer C G, Jenkins R W, et al. Defining T Cell States Associated with Response to Checkpoint Immunotherapy in Melanoma. Cell 2018; 175(4): 998-1013). Carm1-KO T cells expressed higher levels of Myb, which encodes a transcription factor that promotes memory T cell formation by transcriptional activation of Tcf7 and repression of Zeb2. Myb overexpression was previously shown to enhance CD8 T cell memory formation, poly-functionality and promote protective anti-tumor immunity (Gautam S, Fioravanti J, Zhu W, Le Gall J B, Brohawn P, Lacey N E, et al. The transcription factor c-Myb regulates CD8(+) T cell stemness and antitumor immunity. Nat Immunol 2019; 20(3): 337-49). These data are consistent with the hypothesis that Carm1 acts as a co-transcriptional activator that promotes terminal differentiation of tumor-infiltrating T cells.

In contrast, inactivation of Carm1, Tdrd3 and Med12 in tumor cells resulted in induction of a type 1 interferon response that sensitized tumors to T cell-mediated attack. This type 1 interferon response was accompanied by DNA damage, formation of micronuclei and cGAS-STING activation. These findings are consistent with the previous finding that CARM1 cooperates with BRCA1 and p53 to induce expression of the cell cycle inhibitor p21^{CIP1} (CDKN1A) (21). Cell cycle progression despite the presence of double-stranded DNA breaks can result in chromosome mis-segregation during mitosis and the formation of micronuclei that activate the cGAS-STING pathway (Harding S M, Benci J L, Irianto J, Discher D E, Minn A J, Greenberg R A. Mitotic progression following DNA damage enables pattern recognition within micronuclei. Nature 2017; 548 (7668): 466-70). Interestingly, no DNA damage was observed in T cells, even though these cells can undergo rapid proliferation following triggering of the T cell receptor. The possibility that the DNA damage phenotype in tumor cells was caused by accumulation of genic R-loops was considered but DRIP-seq did not demonstrate increased formation of R-loops in Carm1-KO compared to control-KO tumor cells. Many genes involved in the DNA damage response represent tumor suppressors and are inactivated in tumor cells due to mutations or epigenetic mechanisms. Without being bound by theory, it is hypothesized that Carm1 inactivation in tumor cells amplifies pre-existing DNA damage by interfering with p53-induced inhibition of cell cycle progression. This hypothesis could explain why Carm1 inhibition induces cGAS-STING activation in tumor cells but not T cells.

Some chemotherapy drugs can induce activation of the cGAS-STING pathway in tumor cells but targeting of the cell cycle is detrimental to hematopoietic precursors and proliferating tumor-specific T cells. Also, a number of small molecule STING agonists have been developed which are delivered by intra-tumoral injection (Corrales L, Glickman L H, McWhirter S M, Kanne D B, Sivick K E, Katibah G E, et al. Direct Activation of STING in the Tumor Microenvironment Leads to Potent and Systemic Tumor Regression and Immunity. Cell reports 2015; 11(7): 1018-30). The approach presented in the present disclosure may be particularly relevant in the setting of metastatic disease resistant to checkpoint blockade by sensitizing tumor cells to T cells and improving persistence of cytotoxic T cells. Inactivation of the Carm1 gene in tumor cells combined with CTLA-4 blockade induced a substantial survival benefit in B16F10 melanoma and 4T1 TNBC models. Importantly, a small molecule inhibitor of Carm1 also showed synergy with a CTLA-4 blocking mAb in the B16F10 melanoma model that is resistant even to the combination of PD-1 and CTLA-4 mAbs. This inhibitor greatly increased tumor infiltration by CD8 T cells, NK cells and cross-presenting dendritic cells. These data provide the rationale for targeting of CARM1 in human cancers resistant to current immunotherapies. This approach may not only be useful for checkpoint blockade therapy, as illustrated here, but also other immunotherapy approaches in which T cells serve as key effector cells, including neoantigen-based cancer vaccines and CAR T cell therapies for solid tumor indications. In adoptive T cell therapies, CARM1 inhibition may not only sensitize resistant solid tumors to cytotoxic T cells but also enhance T cell memory and persistence, which are critical for sustained clinical benefit with such cellular therapies (Busch D H, Frassle S P, Sommermeyer D, Buchholz V R, Riddell S R. Role of memory T cell subsets for adoptive immunotherapy. Semin Immunol 2016; 28(1): 28-34).

A number of cancer drugs activate innate immune pathways in tumor cells but unfortunately also compromise anti-tumor immune function. In some embodiments, the present disclosure provides that inhibition of Carm1, an epigenetic enzyme and co-transcriptional activator, elicited beneficial anti-tumor activity in both cytotoxic T cells and tumor cells. In T cells, Carm1 inactivation substantially enhanced their anti-tumor function and preserved memory-like populations required for sustained anti-tumor immunity. In tumor cells, Carm1 inactivation induced a potent type 1 interferon response that sensitized resistant tumors to cytotoxic T cells. Substantially increased numbers of dendritic cells, CD8 T cells and NK cells were present in Carm1-deficient tumors, and infiltrating CD8 T cells expressed low levels of exhaustion markers. Targeting of Carm1 with a small molecule elicited potent anti-tumor immunity and sensitized resistant tumors to checkpoint blockade. Targeting of this co-transcriptional regulator thus offers an opportunity to enhance immune function while simultaneously sensitizing resistant tumor cells to immune attack.

Resistance to cancer immunotherapy remains a major challenge. Targeting of CARM1 enables immunotherapy of resistant tumors by enhancing T cell functionality and preserving memory-like T cell populations within tumors. CARM1 inhibition also sensitizes resistant tumor cells to immune attack by inducing a tumor cell-intrinsic type I interferon response.

### Methods of Treating Cancer by Inhibiting Carm1

In some embodiments, the present disclosure provides a method of treating a subject having a cancer. In some embodiments, the method comprises reducing expression of a Carm1 gene and/or a Carm1 effector gene in a cell of the subject; and/or reducing activity of a Carm1 protein and/or a Carm1 effector protein in a cell of the subject. In some embodiments, the cancer is resistant to immunotherapy and/or checkpoint blockade treatment. In some embodiments, the cancer is resistant to drug treatment, such as immunotherapy, chemotherapy, radiotherapy, and/or checkpoint blockade treatment.

In some embodiments, the Carm1 effector gene is a Tdrd3 gene and the Carm1 effector protein is a Tdrd3 protein. In some embodiments, the Carm1 effector gene is a Med12 gene and the Carm1 effector protein is a Med12 protein.

In some embodiments, the method comprises reducing expression of both the Carm1 gene and the Carm1 effector gene in the subject. In some embodiments, the method comprises reducing activity of both the Carm1 protein and the Carm1 effector protein in the subject.

In some embodiments, the reducing step described above comprises administering to the subject an inhibiting agent, wherein the inhibiting agent inhibits the expression of the Carm1 gene or the Carm1 effector gene and/or the activity of the Carm1 protein or the Carm1 effector protein in the subject.

In some embodiments, the inhibiting agent is selected from the group consisting of a polynucleotide, a polypeptide, an antibody, a small molecule, a protein degrader, and a combination thereof. In some other embodiment, the inhibiting agent comprises any CARM1 inhibitor or CARM1 effector inhibitor such as EZM2302, TP-064, a CARM1 degrader, or a combination thereof. In some embodiments, the inhibiting agent comprises EZM2302 or TP-064.

In some embodiments, the inhibiting agent comprises a Carm1 protein degrader, a Tdrd3 protein degrader, a Med12 protein degrader, a CARM1 effector protein degrader, or a combination thereof. In some embodiments, the inhibiting agent comprises a Carm1 protein degrader.

In some embodiments, the reducing step described above comprises silencing the Carm1 gene or the Carm1 effector gene in the subject by shRNA mediated knockdown of mRNA or inactivation of genes.

In some embodiments, the reducing step described herein comprises modifying the Carm1 gene or the Carm1 effector gene to decrease the expression of the Carm1 gene or the Carm1 effector gene. In some embodiments, the modifying step comprises modifying the Carm1 gene or the Carm1 effector gene by A CRISPR/Cas system.

In some embodiments described herein, the expression or activity is reduced in an immune cell of the subject. In some embodiments, the immune cell is an immune effector cell. In some embodiments, the reduced expression of the Carm1 gene or the Carm1 effector gene and/or reduced activity of the Carm1 protein or the Carm1 effector protein enhances the cytotoxic function of the immune effector cell and/or reduces exhaustion of the immune effector cell. In some embodiments, the immune effector cell is selected from the group consisting of a cytotoxic T cell, a tumor-infiltrating lymphocyte (TIL), a Natural Killer T cell (NKT), a cytotoxic T lymphocyte (CTL), a dendritic cell, a CD8 T cell and a CD4 T cell.

In some embodiments, the expression or activity is reduced in a cancer cell of the subject. In some embodiments, the expression or activity is reduced in both an immune cell and a cancer cell of the subject.

In some embodiments, the cancer cell has slowed growth, has reduced metastatic activity, has enhanced sensitivity to killing by CD8 T cells, has increased expression of an interferon response gene, has a DNA damage response, or a combination thereof. In some embodiments, the interferon response gene is an IFNα/γ pathway gene and/or a p53 pathway gene.

In some embodiments, the reducing step described above comprises degrading the CARM1 protein and/or the CARM1 effector protein.

In some embodiments, the method of treating cancer further comprises administering to the subject the reduced expression of the Carm1 gene or the Carm1 effector gene and/or reduced activity of the Carm1 protein or the Carm1 effector protein enhances the cytotoxic function and reduce exhaustion of the immune effector cell.

In some embodiments, the immune cell has substantially no expression of the Carm1 gene or the Carm1 effector gene. In some embodiments, the immune cell is a CAR T cell.

In some embodiments, the cancer cell of the subject overexpresses Carm1. In some embodiments, the cancer is a melanoma, carcinoma, sarcomas, adenocarcinoma, lymphoma, leukemia, kidney, breast, lung, bladder, colon, ovarian, prostate, pancreas, stomach, brain, head and neck, skin, uterine, testicular, glioma, esophagus, or liver cancer.

In some embodiments, the cancer is resistant to checkpoint blockade treatment with a CTLA-4, PD-L1, TIM-3, LAG3, TIGIT, or PD-1 antibody blockade therapy. In some embodiments, the checkpoint blockade is selected from a group consisting of Nivolumab, Pembrolizumab, Ipilimumab, Atezolizumab, Avelumab, Durvalumab, Cemiplimab, and a combination thereof.

In some embodiments, the method further comprises administering to the subject a pharmaceutically effective amount of a second therapeutic agent for treating cancer in the subject.

In some embodiments, the second therapeutic agent is selected from the group consisting of a chemotherapy agent, an immunotherapy agent, a checkpoint blockade agent, a toxin, a radiolabel, a siRNA, a cancer vaccine, a small molecule, a peptide, an antibody, a genetically engineered cell, a CAR T cell, a cytokine and a combination thereof. In some embodiments, the second therapeutic agent is an anti-PD1 antibody, an anti-CTLA-4 antibody, an anti-PD-L1 antibody, an anti-TIGIT antibody, an anti-TIM-3 antibody, or an anti-LAG3 antibody.

According to another aspect of the present disclosure, a method of treating cancer in a subject comprises reducing expression of a Carm1 gene or a Carm1 effector gene and/or activity of a Carm1 protein or a Carm1 effector protein in a cell of the subject.

In some embodiments, the method further comprise administering to the subject a pharmaceutically effective amount of a second therapeutic agent for treating cancer in the subject.

In some embodiments, the second therapeutic agent is selected from the group consisting of a chemotherapy agent, an immunotherapy agent, a checkpoint blockade agent, a toxin, a radiolabel, a siRNA, a cancer vaccine, a small molecule, a peptide, an antibody, a genetically engineered cell, a CAR T cell, a cytokine and a combination thereof. In some embodiments, the second therapeutic agent is a checkpoint blockade agent. In some embodiments, the checkpoint blocker is selected from a group consisting of Nivolumab, Pembrolizumab, Ipilimumab, Atezolizumab, Avelumab, Durvalumab, Cemiplimab, and a combination thereof. In some embodiments, the second therapeutic agent is a CAR T cell.

In some embodiments, the second therapeutic agent is an anti-PD1 antibody, an anti-CTLA-4 antibody, an anti-PD-L1 antibody, an anti-TIGIT antibody, an anti-TIM-3 antibody, or an anti-LAG3 antibody. In some embodiments, the second therapeutic agent is an anti-PD1 antibody. In some embodiments, the second therapeutic agent is an anti-CTLA-4 antibody. In some embodiments, the second therapeutic agent is PD-1 inhibitor. In some embodiments, the second therapeutic agent is CTLA-4 inhibitor.

In some embodiments, the Carm1 effector gene is a Tdrd3 gene and the Carm1 effector protein is a Tdrd3 protein. In some embodiments, the Carm1 effector gene is a Med12 gene and the Carm1 effector protein is a Med12 protein.

In some embodiments, the method comprises reducing expression of both the Carm1 gene and the Carm1 effector gene in the subject. In some embodiments, the method comprises reducing activity of both the Carm1 protein and the Carm1 effector protein in the subject.

In some embodiments, the reducing step described herein comprises administering to the subject an inhibiting agent, wherein the inhibiting agent inhibits the expression of the Carm1 gene or the Carm1 effector gene and/or the activity of the Carm1 protein or the Carm1 effector protein in the subject.

In some embodiments, the inhibiting agent is selected from the group consisting of a polynucleotide, a polypeptide, an antibody, a small molecule, a protein degrader, and a combination thereof. In some other embodiment, wherein the inhibiting agent comprises any CARM1 inhibitor or CARM1 effector inhibitor such as EZM2302, TP-064, a CARM1 degrader, or a combination thereof.

In some embodiments, the inhibiting agent comprises a Carm1 protein degrader, a Tdrd3 protein degrader, a Med12 protein degrader, a Carm1 effector protein inhibitor, or a combination thereof. In some embodiments, the inhibiting agent comprises a Carm1 protein degrader.

In some embodiments, the reducing step described herein comprises silencing the Carm1 gene or the Carm1 effector gene in the subject by shRNA mediated knockdown of mRNA or inactivation of genes.

In some embodiments, the reducing step described herein comprises modifying the Carm1 gene or the Carm1 effector gene to decrease the expression of the Carm1 gene or the Carm1 effector gene. In some embodiments, the modifying step comprises modifying the Carm1 gene or the Carm1 effector gene by A CRISPR/Cas system.

In some embodiments, the reducing step described herein comprises degrading the Carm1 protein or the Carm1 effector protein.

In some embodiments described herein, the expression or activity is reduced in an immune cell of the subject. In some embodiments, the immune cell is an immune effector cell. In some embodiments, the reduced expression of the Carm1 gene and/or Carm1 effector gene, and/or activity of the Carm1 protein or Carm1 effector protein enhances the cytotoxic function and reduce exhaustion of the immune effector cell. In some embodiments, the immune effector cell is selected from the group consisting of a cytotoxic T cell, a tumor-infiltrating lymphocyte (TIL), a Natural Killer T cell (NKT), a cytotoxic T lymphocyte (CTL), a dendritic cell, a CD8 T cell and a CD4 T cell.

In some embodiments, the expression or activity is reduced in a cancer cell of the subject. In some embodiments, the expression or activity is reduced in both an immune cell and a cancer cell of the subject.

In some embodiments, the method of treating cancer further comprises administering to the subject an immune cell having tumor specificity to the cancer and having reduced expression of the Carm1 gene or the Carm1 effector gene and/or reduced activity of the Carm1 protein or the Carm1 effector protein.

In some embodiments, the immune cell has substantially no expression of the Carm1 gene or the Carm1 effector gene. In some embodiments, the cancer cell of the subject overexpresses Carm1. In some embodiments, the cancer is a melanoma, carcinoma, sarcomas, adenocarcinoma, lymphoma, leukemia, kidney, breast, lung, bladder, colon, ovarian, prostate, pancreas, stomach, brain, head and neck, skin, uterine, testicular, glioma, esophagus, or liver cancer.

According to another aspect of the present disclosure, a method of sensitizing a cancer cell to an immune cell comprises inhibiting expression and/or activity of a Carm1 gene or protein, or a Carm1 effector gene or protein in a cancer cell by one or more inhibiting agents, wherein the inhibiting sensitizes the cancer cell to an immune cell.

In some embodiments, the inhibiting agent comprises a polynucleotide, a polypeptide, a peptide, an antibody, a small molecule, a protein degrader, or a combination thereof.

In some other embodiments, the inhibiting agent comprises any CARM1 inhibitor or CARM1 effector inhibitor such as EZM2302, TP-064, a CARM1 degrader, or a combination thereof. In some embodiments, the inhibiting agent comprises EZM2302 or TP-064.

In some embodiments, the inhibiting agent comprises a Carm1 protein degrader, a Tdrd3 protein degrader, a Med12 protein degrader, a Carm1 effector protein degrader, or a combination thereof. In some embodiments, the inhibiting agent comprises a Carm1 protein degrader.

In some embodiments, the Carm1 effector gene or protein is a Tdrd3 gene or protein. In some embodiments, the Carm1 effector gene or protein is a Med12 gene or protein.

In some embodiments, the immune cell is a cytotoxic T cell.

### Methods of Treating Cancer by Inhibiting Carm1 in an Immune Effector Cell

According to another aspect of the present disclosure, provided herein is a method of increasing the anti-tumor function of an immune effector cell. In some embodiments, the method comprises reducing expression and/or activity of a Carm1 gene or protein, or a Carm1 effector gene or protein in the immune effector cell, thereby increasing the anti-tumor function of the immune effector cell.

In some embodiments, the Carm1 effector gene or protein is a Tdrd3 gene or protein. In some embodiments, the Carm1 effector gene or protein is a Med12 gene or protein.

In some embodiments, the method described herein comprises reducing expression of both a Carm1 gene and a Carm1 effector gene in an immune effector cell. In some embodiments, the method described herein comprises reducing activity of both a Carm1 protein and a Carm1 effector protein in an immune effector cell.

In some embodiments, the reducing step described above comprises inhibiting, by one or more inhibiting agents, the expression and/or activity of the Carm1 gene or protein, or the Carm1 effector gene or protein in the immune effector cell. In some embodiments, the inhibiting agent comprises a polynucleotide, a polypeptide, a peptide, an antibody, a small molecule, a protein degrader, a genetically engineered cell, or a combination thereof. In some other embodiment, the inhibiting agent comprises any CARM1 inhibitor or a CARM1 effector protein inhibitor such as EZM2302, TP-064, a CARM1 degrader, or a combination thereof. In some embodiments, the inhibiting agent comprises EZM2302 or TP-064.

In some embodiments, the inhibiting agent comprises a Carm1 protein degrader, a Tdrd3 protein degrader, a Med12 protein degrader, a Carm1 effector protein degrader, or a combination thereof. In some embodiments, the inhibiting agent comprises a Carm1 protein degrader.

In some embodiments, the expression and/or activity of the Carm1 gene or protein, or a Carm1 effector gene or protein is reduced by shRNA mediated knockdown of mRNA or inactivation of gene.

In some embodiments, the reducing step comprises modifying the immune effector cell to reduce or remove the CARM1 gene or Carm1 effector gene in the immune effector cell. In some embodiments, the modifying step comprises modifying the Carm1 gene or the Carm1 effector gene by A CRISPR/Cas SYSTEM.

In some embodiments, the reducing step comprises silencing the CARM1 gene or the Carm1 effector gene in the immune effector cell.

In some embodiments, the reducing step comprises degrading the Carm1 protein or the Carm1 effector protein in the immune effector cell.

In some embodiments, the immune effector cell is a T cell. In some embodiments, the immune effector cell is selected from the group consisting of a cytotoxic T cell, a tumor-infiltrating lymphocyte (TIL), a Natural Killer T cell (NKT), a cytotoxic T lymphocyte (CTL), a dendritic cell, a CD8 T cell and a CD4 T cell. In some embodiments, the immune effector cell is a cytotoxic T cell. In some embodiments, the immune effector cell is Natural Killer T cell. In some embodiments, the CD8 T cell expresses a higher level of CD69, CD45.1, granzyme B, IFNγ, Ki67, or a combination thereof.

### Immune Effector Cells Comprising an Inhibitor of Carm1

According to another aspect of the present disclosure, provided herein is an immune effector cell having an inhibiting agent of a Carm1 gene/protein or a Carm1 effector gene/protein, wherein the inhibiting agent inhibits expression of the Carm1 gene or Carm1 effector gene, and/or activity of the Carm1 protein or the Carm1 effector protein in the immune effector cell.

In some embodiments, the Carm1 effector gene is a Tdrd3 gene and the Carm1 effector protein is a Tdrd3 protein. In some embodiments, the Carm1 effector gene is a Med12 gene and the Carm1 effector protein is a Med12 protein.

In some embodiments, the immune effector cell has substantially no expression of the Carm1 gene or the Carm1 effector gene.

In some embodiments, the inhibiting agent is selected from the group consisting of a polynucleotide, a polypeptide, an antibody, a small molecule, a protein degrader, and a combination thereof. In some other embodiment, wherein the inhibiting agent comprises any CARM1 inhibitor or CARM1 effector protein inhibitor such as EZM2302, TP-064, a CARM1 degrader, or a combination thereof. In some embodiments, the inhibiting agent comprises EZM2302 or TP-064.

In some embodiments, the inhibiting agent comprises a Carm1 protein degrader, a Tdrd3 protein degrader, a Med12 protein degrader, a Carm1 effector protein degrader, or a combination thereof. In some embodiments, the inhibiting agent comprises a Carm1 protein degrader.

In some embodiments, the immune effector cell has reduced expression of both a CARM1 gene and a CARM1 effector gene. In some embodiments, the immune effector cell has reduced activity of both a CARM1 protein and a CARM1 effector protein.

In some embodiments, the immune effector cell is a T cell. In some embodiments, the immune effector cell is selected from the group consisting of a cytotoxic T cell, a tumor-infiltrating lymphocyte (TIL), a Natural Killer T cell (NKT), a cytotoxic T lymphocyte (CTL), a dendritic cell, a CD8 T cell and a CD4 T cell. In some embodiments, the immune effector cell is a cytotoxic T cell. In some embodiments, the immune effector cell is Natural Killer T cell.

In some embodiments, the immune effector cell is tumor specific. In some embodiments, the immune effector cell expresses a tumor-specific T-cell receptor or a chimeric antigen receptor (CAR).

In some embodiments, the immune effector cell further comprises a chimeric antigen receptor (CAR), wherein the CAR comprises an antigen-binding domain, a transmembrane domain, and a stimulatory domain. In some embodiments, the antigen-binding domain binds a tumor antigen or pathogen antigen.

In some embodiments, the tumor antigen is selected from a group consisting of an antigen present in a cancer cell, a cancer cell, a cancer cell fragment, a tumor antigen, α-galcer, anti-CD3, anti-CD28, anti-IgM, anti-CD40, a pathogen, an attenuated pathogen, and a portion thereof. In some embodiments, the tumor antigen is a cancer cell, a cancer cell fragment, or a tumor antigen.

In some embodiments, the tumor antigen is associated with a melanoma, carcinoma, sarcomas, adenocarcinoma, lymphoma, leukemia, kidney, breast, lung, bladder, colon, ovarian, prostate, pancreas, stomach, brain, head and neck, skin, uterine, testicular, glioma, esophagus, or liver cancer.

In some embodiments, the tumor antigen is associated with a solid tumor or lymphoid tumor.

In some embodiments, the antigen-binding domain is an antigen-binding fragment of an antibody.

According to another aspect of the present disclosure, provided herein is a composition comprising an immune effector cell described herein and a pharmaceutically acceptable carrier.

In some embodiments, the immune effector cell has an inhibiting agent of a Carm1 gene/protein or a Carm1 effector gene/protein. The inhibiting agent inhibits expression of the Carm1 gene or Carm1 effector gene, and/or activity of the Carm1 protein or the Carm1 effector protein.

In some embodiments, the immune effector cell has substantially no expression of the Carm1 gene or the Carm1 effector gene.

In some embodiments, the inhibiting agent is selected from the group consisting of a polynucleotide, a polypeptide, an antibody, a small molecule, a protein degrader, and a combination thereof. In some other embodiment, wherein the inhibiting agent comprises any CARM1 inhibitor or CARM1 effector protein inhibitor such as EZM2302, TP-064, a CARM1 degrader, or a combination thereof. In some embodiments, the inhibiting agent comprises EZM2302 or TP-064.

In some embodiments, the inhibiting agent comprises a Carm1 protein degrader, a Tdrd3 protein degrader, a Med12 protein degrader, a CARM1 effector protein degrader, or a combination thereof. In some embodiments, the inhibiting agent comprises a Carm1 protein degrader.

In some embodiments, the immune effector cell has reduced expression of both the CARM1 gene and the CARM1 effector gene. In some embodiments, the immune effector cell has reduced activity of both the CARM1 protein and the CARM1 effector protein.

In some embodiments, the immune effector cell is a T cell. In some embodiments, the immune effector cell is selected from the group consisting of a cytotoxic T cell, a tumor-infiltrating lymphocyte (TIL), a Natural Killer T cell (NKT), a cytotoxic T lymphocyte (CTL), a dendritic cell, a CD8 T cell and a CD4T cell. In some embodiments, the immune effector cell is a cytotoxic T cell. In some embodiments, the immune effector cell is Natural Killer T cell.

In some embodiments, the immune effector cell is tumor specific. In some embodiments, the immune effector cell expresses a tumor-specific T-cell receptor or a chimeric antigen receptor (CAR).

In some embodiments, the immune effector cell further comprises a chimeric antigen receptor (CAR), wherein the CAR comprises an antigen-binding domain, a transmembrane domain, and a stimulatory domain. In some embodiments, the antigen-binding domain binds a tumor antigen or pathogen antigen.

In some embodiments, the tumor antigen is selected from a group consisting of an antigen present in a cancer cell, a cancer cell, a cancer cell fragment, a tumor antigen, α-galcer, anti-CD3, anti-CD28, anti-IgM, anti-CD40, a pathogen, an attenuated pathogen, and a portion thereof. In some embodiments, the tumor antigen is a cancer cell, a cancer cell fragment, or a tumor antigen.

In some embodiments, the tumor antigen is associated with a melanoma, carcinoma, sarcomas, adenocarcinoma, lymphoma, leukemia, kidney, breast, lung, bladder, colon, ovarian, prostate, pancreas, stomach, brain, head and neck, skin, uterine, testicular, glioma, esophagus, or liver cancer.

In some embodiments, the tumor antigen is associated with a solid tumor or lymphoid tumor.

In some embodiments, the antigen-binding domain is an antigen-binding fragment of an antibody.

In some embodiments, the composition further comprises a second therapeutic agent. In some embodiments, the second therapeutic agent is selected from the group consisting of a chemotherapy agent, an immunotherapy agent, a checkpoint blockade agent, a toxin, a radiolabel, a siRNA, a cancer vaccine, a small molecule, a peptide, an antibody, a genetically engineered cell, a CAR T cell, a cytokine and a combination thereof. In some embodiments, the second therapeutic agent is a checkpoint blockade agent. In some embodiments, the second therapeutic agent is a CAR T cell.

In some embodiments, the second therapeutic agent is an anti-PD1 antibody, an anti-CTLA-4 antibody, an anti-PD-L1 antibody, an anti-TIGIT antibody, an anti-TIM-3 antibody, or an anti-LAG3 antibody. In some embodiments, the second therapeutic agent is an anti-PD1 antibody. In some embodiments, the second therapeutic agent is an anti-CTLA-4 antibody. In some embodiments, the second therapeutic agent is a PD1 inhibitor. In some embodiments, the second therapeutic agent is a CTLA-4 inhibitor.

In some embodiments, the immune effector cell is a T cell. In some embodiments, the immune effector cell is selected from the group consisting of a cytotoxic T cell, a tumor-infiltrating lymphocyte (TIL), a Natural Killer T cell (NKT), a cytotoxic T lymphocyte (CTL), a dendritic cell, a CD8 T cell and a CD4T cell. In some embodiments, the immune effector cell is a cytotoxic T cell. In some embodiments, the immune effector cell is a Natural Killer T cell.

### Methods of Treating Cancer by Using an Immune Effector Cell

According to another aspect of the present disclosure, provided herein is a method of treating cancer in a subject. In some embodiments, the method comprises administering to a subject having cancer an immune effector cell or a composition described herein.

In some embodiments, the immune effector cell has an inhibiting agent of a Carm1 gene/protein or a Carm1 effector gene/protein. The inhibiting agent inhibits expression of the Carm1 gene or Carm1 effector gene, and/or activity of the Carm1 protein or the Carm1 effector protein.

In some embodiments, the composition comprises the immune effector cell described herein and a pharmaceutically acceptable carrier. In some embodiments, the immune effector cell has an inhibiting agent of a Carm1 gene/protein or a Carm1 effector gene/protein. The inhibiting agent inhibits expression of the Carm1 gene or Carm1 effector gene, and/or activity of the Carm1 protein or the Carm1 effector protein.

In some embodiments, the immune effector cell is autologous. In some embodiments, the immune effector cell is specific to a cancer cell of the subject.

In some embodiments, the method further comprises administering to the subject having cancer a second therapeutic agent, or a composition comprising a second therapeutic agent and a pharmaceutically acceptable carrier.

In some embodiments, the second therapeutic agent is selected from the group consisting of a chemotherapy agent, an immunotherapy agent, a checkpoint blockade agent, a toxin, a radiolabel, a siRNA, a cancer vaccine, a small molecule, a peptide, an antibody, a genetically engineered cell, a CAR T cell, a cytokine and a combination thereof. In some embodiments, the second therapeutic agent is a checkpoint blockade agent. In some embodiments, the second therapeutic agent is a CAR T cell.

In some embodiments, the second therapeutic agent is an anti-PD1 antibody, an anti-CTLA-4 antibody, an anti-PD-L1 antibody, an anti-TIGIT antibody, an anti-TIM-3 antibody, or an anti-LAG3 antibody. In some embodiments, the second therapeutic agent is an anti-PD1 antibody. In some embodiments, the second therapeutic agent is an anti-CTLA-4 antibody. In some embodiments, the second therapeutic agent is PD-1 inhibitor. In some embodiments, the second therapeutic agent is CTLA-4 inhibitor.

In some embodiments, the cancer is a carcinoma, sarcomas, adenocarcinoma, lymphoma, leukemia, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas) and Hodgkin's lymphoma, leukemia (including AML, ALL, and CIVIL), and multiple myeloma. In some embodiments, the cancer is melanoma. In some embodiments, the cancer is a plasma cell malignancy, for example, multiple myeloma (MM) or pre-malignant condition of plasma cells. In some embodiments the subject has been diagnosed as having a cancer or as being predisposed to cancer.

In some instances, treatments methods can include a single administration, multiple administrations, and repeating administration as required for the prophylaxis or treatment of the disease or condition from which the subject is suffering. In some instances, treatment methods can include assessing a level of disease in the subject prior to treatment, during treatment, and/or after treatment. In some instances, treatment can continue until a decrease in the level of disease in the subject is detected.

Following administration, the subject can be evaluated to detect, assess, or determine their level of disease. In some instances, treatment can continue until a change (e.g., reduction) in the level of disease in the subject is detected.

Upon improvement of a patient's condition (e.g., a change (e.g., decrease) in the level of disease in the subject), a maintenance dose of a compound, composition or combination of this disclosure may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

It is also within the scope of the present disclosure to combine any of the methods and any of the compositions disclosed herein with one or more therapeutic agents. A therapeutic agent includes, but is not limited to, small molecules, peptides, antibodies, ribozymes, antisense oligonucleotides, chemotherapeutic agents and radiation.

It is also within the scope of the present disclosure to combine any of the methods and any of the compositions disclosed herein with conventional cancer therapies and various drugs in order to enhance the efficacy of such therapies through either reducing the doses/toxicity of conventional therapies and/or to increase the sensitivity of conventional therapies. One conventional therapy is the use of radiation therapy. Another conventional therapy is the use of chemotherapeutic drugs that can be divided into: alkylating agents, antimetabolites, anthracyclines, plant alkaloids, topoisomerase inhibitors, and antitumour agents. All of these drugs affect cell division or DNA synthesis and function in some way. Other conventional cancer therapies are agents that do not directly interfere with DNA. Examples of such agents for which to combine with the present disclosure may include for example “small-molecule” drugs that block specific enzymes involved in cancer cell growth. Monoclonal antibodies, cancer vaccines, angiogenesis inhibitors, and gene therapy are targeted therapies that can also be combined with the compositions and methods disclosed herein because they also interfere with the growth of cancer cells.

A frequent feature of cancer cells is the tendency to grow in a manner that is uncontrollable by the host. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells.

In some embodiments, the inhibiting agent causes degradation of the protein, and thus inhibits or reduces the activity of the protein. For example, the inhibiting agent can be targeted with a small molecule degrader which causes degradation of the protein, and thus inhibit or reduce the activity of the protein.

In some embodiments, the inhibiting agent inhibits the expression of the protein, such as with shRNA or by gene inactivation (such as with A CRISPR/Cas sy stem).

In some embodiments, the inhibiting agent can be a CARM1 inhibiting agent.

In some embodiments, the CARM1 inhibiting agent is a small molecule.

Small molecules typically have a molecular weight that is less than 10 kD, typically less than 2 kD, and preferably less than 1 kD. Small molecules include, without limitation, inorganic molecules, organic molecules, organic molecules containing an inorganic component, molecules comprising a radioactive atom, synthetic molecules, peptide mimetics, and antibody mimetics. As a small molecule therapeutic agent can better penetrate into the cell, it may be less sensitive to disintegration and less capable of eliciting an immune response than large molecules.

For example, several CARM1 inhibiting agents are shown in Tables 1 and 2 below. See, Drew, A. E., Moradei, O., Jacques, S. L. et al., Identification of a CARM1 Inhibitor with Potent In Vitro and In Vivo Activity in Preclinical Models of Multiple Myeloma, Scientific Reports, 7, 17993 (2017); Nakayama K. et al., TP-064, A Potent And Selective Small Molecule Inhibitor Of PRMT4 For Multiple Myeloma, Oncotarget, 2018, 9, pp. 18480-18493, both of which are incorporated herein by reference.

TABLE 1

| | | CARM1 | CARM1 |
|---|---|---|---|
| | | Biochemical | ICW |
| | | IC₅₀ | IC₅₀ |
| # | Structure | (nM) | (uM) |
| 1 | 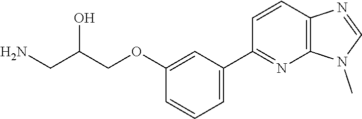 [SMILES: Cn1cnc2ccc(-c3cccc(OCC(O)CN)c3)nc21] | 2,300 | >20 |
| 2 | 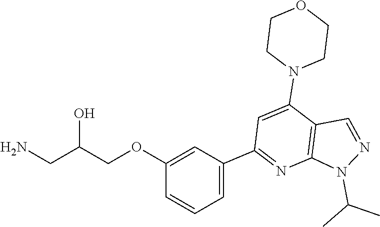 [SMILES: CC(C)n1ncc2c(N3CCOCC3)cc(-c3cccc(OCC(O)CN)c3)nc21] | 46 | 19 |
| EPZ025654 GSK35336023 | 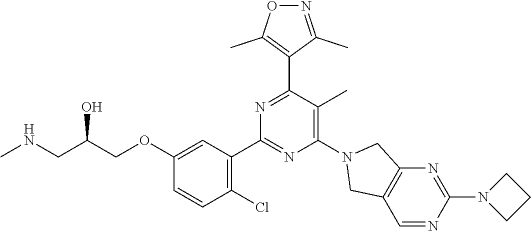 [SMILES: CNC[C@@H](O)COc1ccc(Cl)c(-c2nc(-c3c(C)noc3C)c(C)c(N3Cc4cnc(N5CCC5)nc4C3)n2)c1] | 3 | .011 |
| EZM2302 GSK3359088 | 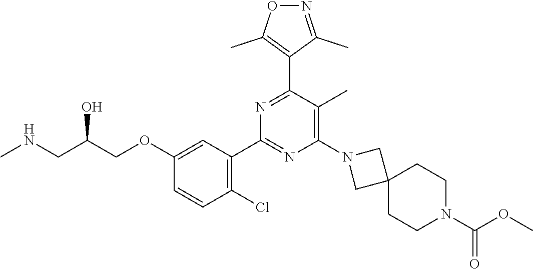 | 6 | 0.055 |
| 3 | 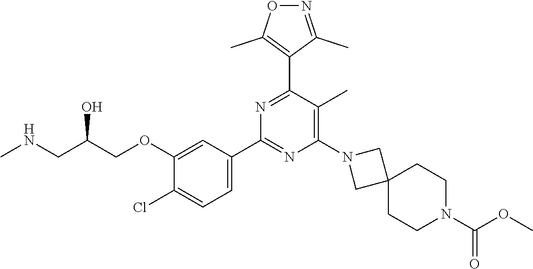 | >1000 | >6.5 |

TABLE 2

| | |
|---|---|
| 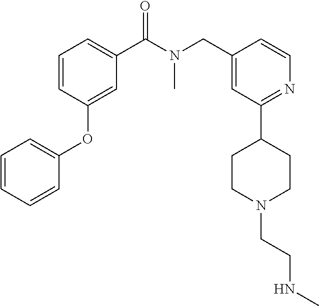 [SMILES: CNCCN1CCC(c2cc(CN(C)C(=O)c3cccc(Oc4ccccc4)c3)ccn2)CC1] | TP-064 |

The CARM1 inhibiting agent can be EZM2302 (GSK3359088). EZM2302 is a potent and selective inhibitor of CARM1 enzymatic activity that exhibits anti-proliferative effects both in vitro and in vivo, in biochemical assays (IC₅₀=6 nm) with broad selectivity against histone methyltransferases.

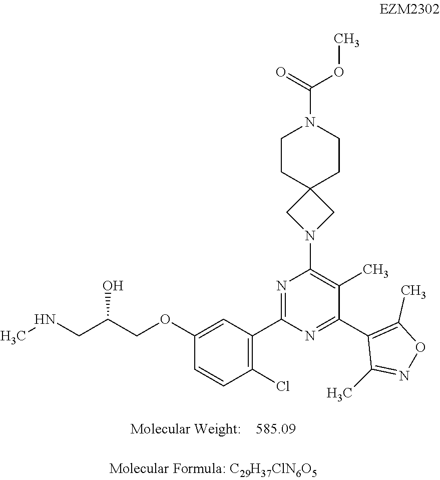 [SMILES: CNC[C@H](O)COc1ccc(Cl)c(-c2nc(-c3c(C)noc3C)c(C)c(N3CC4(CCN(C(=O)OC)CC4)C3)n2)c1]

Treatment of malignant melanoma cell lines with EZM2302 leads to inhibition of PABP1 and SMB methylation and cell stasis with IC50 values in the nanomolar range. Oral dosing of EZM2302 demonstrates dose-dependent in vivo CARM1 inhibition and anti-tumor activity in a malignant melanoma xenograft model. EZM2302 (GSK3359088) is a potent and selective inhibitor of CARM1 enzymatic activity that exhibits anti-proliferative effects both in vitro and in vivo, in biochemical assays (IC50=6 nM) with broad selectivity against other histone methyltransferases.

For example, EZM2302 at sub-micromolar levels can suppress CARM1 activity in tumor cells. Further, interferon stimulated gene expression in B16F10 cells upon Carm1 inhibitor EZM2302 treatment was investigated. Specifically, EZM2302 treatment induced up regulation of the ISGs expression mimicking genetic ablation of Carm1 phenotype in B16F10 cells. EZM2302 also significantly suppresses B16F10 tumor growth when administered via oral save to B16F10 tumor bearing mice at 150 mg/kg/day.

The CARM1 inhibiting agent can be the arginine methyltransferase inhibitor TP-064. TP-064 is a potent, selective, and cell-active chemical probe of human CARM1. TP-064 inhibited the methyltransferase activity of CARM1 with high potency (half-maximal inhibitory concentration, IC₅₀<10 nM) and selectivity over other CARM family proteins.

The skilled artisan will recognize that other small molecule inhibitors of CARM1 gene/protein and/or Carm1 effector gene/protein can be used in the methods provided herein. For example, other small molecule inhibitors of CARM1 can be used interchangeably with EZM2302.

Thus, some aspects of the disclosure can comprise administering to a subject a composition comprising a therapeutically effective amount of a small molecule inhibitor.

In some embodiments, the therapeutically effective amount of the inhibiting agent is at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 3500 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight.

The inhibiting agent(s) or composition comprising the inhibiting agent(s) can be administered to the subject one time (e.g., as a single injection, bolus, or deposition). Alternatively, administration can be once daily, twice daily, or more than twice daily to a subject in need thereof for a period of time, such as from about 2 to about 28 days, or from about 7 to about 10 days, or from about 7 to about 15 days. It can also be administered once daily, twice daily, or more than twice daily to a subject for a period of 1, 2, 3, 4, 5, 6, 7, 8, 9, 10, 11, 12 times per year, or a combination thereof.

The dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

A therapeutically effective dose can depend upon a number of factors known to those of ordinary skill in the art. The dose(s) can vary, for example, depending upon the identity, size, and condition of the subject or sample being treated, further depending upon the route by which the composition is to be administered, if applicable, and the effect which the practitioner desires. These amounts can be readily determined by the skilled artisan.

Tudor domain containing protein 3 (TDRD3) is a modular protein identified based on its ability to recognize methylated arginine motifs through its Tudor domain. TDRD3 localizes to cytoplasmic stress granules, a structure shown to promote survival upon treatment with chemotherapeutic drugs in cancer cells. TDRD3 regulates cell proliferation and invasion in breast cancer cells. TDRD3 depletion in cancer cells inhibits tumor formation and metastasis to the lung in vivo. Furthermore, TDRD3 regulates the expression of a number of key genes associated with promotion of breast cancer tumorigenesis and disease progression. See Morettin, Alan, et al. “Tudor domain containing protein 3 promotes tumorigenesis and invasive capacity of breast cancer cells.” Scientific reports 7.1 (2017): 5153.

Genetic inactivation of TDRD3 mimics the CARM1-inactivation phenotype. Thus, the inhibiting agent as referenced in some embodiments of the disclosure can also comprise a TDRD3 inhibiting agent. In some embodiments, the inhibiting agent can inhibit the activation and/or activity of TDRD3, and/or can degrade the TDRD3 protein. For example, the inhibiting agent can be a small molecule inhibitor that binds the active site of TDRD3 and inhibits the activation of and/or activity of TDRD3, or a small molecule degrader of TDRD3.

Some aspects of the disclosure relate to methods of treating a subject afflicted with cancer in a subject. In an embodiment, the method comprises administering to the subject a composition comprising one or more inhibiting agent(s) and a pharmaceutically acceptable excipient, wherein the inhibiting agent(s) inhibit expression and/or activity of CARM1, TDRD3, or a combination thereof.

The cancer can be diagnosed using any suitable method, including but not limited to, biopsy, x-ray, blood test, and the diagnostic methods of the present disclosure.

In some embodiments of the disclosure, the cancer can comprise a solid tumor.

In other embodiments, the cancer can comprise a hematologic malignancy, such as those cancers that begin in the hematopoiestic stem and progenitor cells in the bone marrow. For example, the hematologic malignancies can comprise a leukemia, a lymphoma, or a myeloma.

Non-limiting examples of cancers that pertain to aspects of the disclosure include, but are not limited to, carcinoma, lymphoma, blastoma, sarcoma, and leukemia. More particular examples of such cancers include squamous cell cancer, small-cell lung cancer, non-small cell lung cancer, adenocarcinoma (for example, adenocarcinoma of the lung), squamous carcinoma (for example, squamous carcinoma of the lung), cancer of the peritoneum, hepatocellular cancer, gastrointestinal cancer, gastric cancer, stomach cancer, melanoma, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer. Additional examples include cancers in which Carm1 is overexpressed. In exemplary embodiments, the cancer is breast cancer. In another exemplary embodiment, the cancer is prostate cancer. For example, CARM1 is a co-activator of the estrogen and androgen hormone receptors, respectively, that drive growth of these tumors.

Some embodiments of the disclosure also apply to cancers with mutations in one or more tumor suppressor genes, non-limiting examples of which include p53, adenomatous polyposis coli (APC), retinoblastoma-associated protein (RB), Von-Hippel-Lindau (VHL), BRCA1, and BRCA2 mutations.

p53 is a transcription factor that regulates several intracellular pathways, including those involved in cell survival, DNA-repair, apoptosis and senescence. p53 preserves DNA integrity in response to a number of stimuli, such as ionizing radiations, genotoxic insults and oxidative stress. p53 is often mutated in cancer. See, for example, Perri, Francesco, Salvatore Pisconti, and Giuseppina Della Vittoria Scarpati. “P53 mutations and cancer: a tight linkage.” Annals of translational medicine 4.24 (2016). Most p53 mutations in human cancer are missense mutations, which result in the production of full-length mutant p53 proteins. In fact, only 10-15% of TP53 mutations are defined as “disruptive mutations”, namely those leading to an inactive or truncated protein, while the remaining 85-90% often lead to the synthesis of functioning proteins. Missense mutations are often clustered in the 4-9 exons of the TP53 gene, which correspond to a particular sequence of the gene, representing the p53 DNA-binding-domain. As a consequence, missense p53 mutations are able to modify its ordinary function of transcriptional factor. Interestingly, several studies have demonstrated that many mutant p53 proteins, not only lose their tumor suppression functions, but also gain new oncogenic functions. This phenomenon is termed “the gain of function of mutant p53”. More specifically, mutant p53 interacts with proteins that normally partner with wild type p53. This new association deprive them of their anticancer activities and in place, they are corrupted to act as cancerogenesis promoters.

In some embodiments, the cancer is characterized by over expression of a gene encoding CARM1, a gene encoding Tdrd3, or a combination thereof.

In some embodiments, the cancer is characterized by increased activation of TGF-beta or a mutation in a DNA repair pathway, such as the p53 signaling pathway.

Tumor cell resistance to chemotherapy and radiotherapy agents represents a major problem in clinical oncology. One goal of current cancer research is to find ways to improve the efficacy of chemo- and radiotherapy by combining it with other therapies. In the context of the present disclosure, it is contemplated that inhibiting agents, such as CARM1 inhibiting agents or Tdrd3 inhibiting agents, could be used similarly in conjunction with chemotherapeutic, radiotherapeutic, or immunotherapeutic intervention, as well as pro-apoptotic or cell cycle regulating agents.

Alternatively, the present disclosure therapy may precede or follow the other agent treatment by intervals ranging from minutes to weeks. In embodiments where the other agent and present disclosure are applied separately to the individual, one would generally ensure that a significant period of time did not expire between the time of each delivery, such that the agent and inventive therapy would still be able to exert an advantageously combined effect on the cancer cell. In such instances, it is contemplated that one may contact the cancer cell with both modalities within about 12-24 h of each other and, more preferably, within about 6-12 h of each other. In some situations, the time period can be extended for treatment significantly, however, where several days (for example, 2, 3, 4, 5, 6 or 7 days) to several weeks (e.g., 1, 2, 3, 4, 5, 6, 7 or 8 weeks) lapse between the respective administrations.

It is expected that the treatment cycles would be repeated as necessary. It also is contemplated that various standard therapies, as well as surgical intervention, may be applied in combination with the inventive cell therapy.

Some aspects of the disclosure are also directed towards methods of treating drug-resistant cancers. Drug-resistant cancers can also be referred to as “refractory” (i.e., resistant) to treatment. In one embodiment “drug-resistant cancer” can refer to cancer cells that acquire resistance to an anti-cancer therapy, such as immunotherapy and/or chemotherapy. Cancer cells can acquire resistance to a therapy by a range of mechanisms, including the mutation or overexpression of the drug target, inactivation of the drug, or elimination of the drug from the cell. Tumors that recur after an initial response to a therapy may be resistant to multiple drugs (they are multidrug resistant). In the conventional view of drug resistance, one or several cells in the tumor population acquire genetic changes that confer drug resistance. Accordingly, the reasons for drug resistance, inter alia, are: a) some of the cells that are not killed by the therapy mutate (change) and become resistant to the drug. Once they multiply, there may be more resistant cells than cells that are sensitive to the therapy; b) Gene amplification. A cancer cell may produce hundreds of copies of a particular gene. This gene triggers an overproduction of protein that renders the anticancer drug ineffective; c) cancer cells may pump the drug out of the cell as fast as it is going in; d) cancer cells may stop taking in the drugs because the protein that transports the drug across the cell wall stops working; e) the cancer cells may learn how to repair the DNA breaks caused by some anti-cancer drugs; f) cancer cells may develop a mechanism that inactivates the drug. Thus, the resistance to anticancer agents used is the main cause of treatment failure in malignant disorders, provoking tumors to become resistant. Drug resistance is the major cause of cancer therapy failure.

In one embodiment, “resistant cancer” refers to drug-resistant cancer as described herein above. In another embodiment, “resistant cancer” refers to cancer cells that acquire resistance to any treatment such as chemotherapy, immunotherapy, radiotherapy or biological therapy.

For example, immune checkpoint blockades, such as inhibitors against programmed death 1 (PD-1) and its ligand (PD-L1), have received extensive attention in the past decade because of their dramatic clinical outcomes in advanced malignancies. However, both primary and acquired resistance becomes one of the major obstacles, which greatly limits the long-lasting effects and wide application of PD-1/PD-L1 blockade therapy. See Bai, Jie, et al. “Regulation of PD-1/PD-L1 pathway and resistance to PD-1/PD-L1 blockade.” Oncotarget 8.66 (2017): 110693.

Similarly, antibodies blocking the immune checkpoint receptor CTLA-4 (such as ipilimumab) has resulted in an increase in patient survival in a number of studies when compared to convention anti-cancer therapies. See Seidel, Judith A., Atsushi Otsuka, and Kenji Kabashima. “Anti-PD-1 and anti-CTLA-4 therapies in cancer: mechanisms of action, efficacy, and limitations.” Frontiers in oncology 8 (2018): 86. However, as with PD-1, patients will eventually relapse and develop tumor progression.

The selection pressure caused by checkpoint inhibitor treatment may give rise to tumor cells that can evade immunomediated recognition and deletion through new pathways. Tumor cells from patients refractory to anti-PD-1 treatment, for example, acquire mutations making them less susceptible to T cell-mediated killing via loss of IFN-γ response elements or MHC class I. Anti-PD-1 or anti-CTLA-4 treatment may also cause upregulation of other inhibitory receptors.

Inactivation or reduced expression of a CARM1 gene/protein and/or a CARM1 effector gene/protein elicited anti-tumor immune responses even in tumor models that are refractory to checkpoint blockade, such as with PD-1 or PD-1 plus CTLA-4 antibodies. In some embodiments, the B16F10 melanoma model and the 4T1 breast cancer model are both refractory to checkpoint blockade. Thus, targeting of CARM1 gene/protein and/or CARM1 effector gene/protein is a therapeutic strategy for refractory (or drug-resistant) cancers, including those that fail to respond to checkpoint blockade, such as with PD1 or CTLA-4 antibodies.

In some embodiments, an inhibiting agent, such as a CARM1 inhibiting agent, is administered to a subject. The administration of the inhibiting agent according to the present disclosure can be carried out in any convenient manner, including by aerosol inhalation, injection, ingestion, transfusion, implantation or transplantation. The compositions described herein can be administered to a patient subcutaneously, intradermally, intratumorally, intranodally, intramedullary, intramuscularly, by intravenous or intralymphatic injection, or intraperitoneally. In one embodiment, the compositions of the present disclosure are preferably administered by intravenous injection.

While individual needs vary, determination of optimal ranges of effective amounts of a given composition for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

Embodiments can also comprise an anti-cancer treatment comprising combinations of one or more therapies against cancer. For example, the one or more therapies can be selected from the group comprising antibody therapy (for example, anti-PD1 antibody, such as Nivolumab or Pembrolizumab, an anti-CTLA-4 antibody, such as Ipilimumab, or both), cancer vaccine, adoptive T cell therapy (for example, CAR T cells, TCR T cells), chemotherapy, cytokines therapy, toxin, a radiolabel, a siRNA, a small molecule, a peptide, an antibody, a genetically engineered cell, a cytokine, dendritic cell therapy, gene therapy, hormone therapy, laser light therapy and radiation therapy. Thus, embodiments can comprise administering to the subject at least one additional therapeutic agent (i.e., a second inhibitor agent and/or an anti-cancer agent). In some embodiments, inactivation of Carm1 in the tumor synergizes with checkpoint blockade therapy (i.e., anti-PD1).

Further, some embodiments comprise methods of cancer stratification. The term “stratify” or “stratifying” refers to sorting patients into those who are more (or less) likely to benefit from an anti-cancer therapy which is based on a CARM1, Med12 and/or TDRD3 inhibitor than others. The methods of present disclosure may thus be employed for stratifying cancer patients with regard to their susceptibility to treatment with a CARM1 inhibitor, a Med12 inhibitor and/or a TDRD3 inhibitor.

Specifically, a “patient who may benefit” from anti-cancer therapy with a CARM1 inhibitor, a Med12 inhibitor and/or a TDRD3 inhibitor is a patient in which the inhibitor has a higher likelihood to have a therapeutic effect. The likelihood that (a) cancer and/or a cancer patient may or may not respond favorably is dependent on whether CARM1, Med12 or TDRD3 are overexpressed in the cancer. Also, the likelihood that (a) cancer and/or cancer patient may or may not respond favorably can be dependent on whether the cancer is resistant to checkpoint blockade.

Correspondingly, a “patient who may not benefit” from anti-cancer therapy with a CARM1 inhibitor, a Med12 inhibitor and/or a TDRD3 inhibitor is a patient in which the inhibitor does not have a higher likelihood to have a therapeutic effect.

Aspects of the disclosure are also directed towards methods of sensitizing a cancer cell to an immune effector cell, e.g., a cytotoxic T cell.

In one embodiment, the method comprises suppressing expression and/or activity of one or more of CARM1, Med12, Tdrd3 in the cancer cell by contacting the cancer cell with one or more inhibiting agents, wherein suppression of expression and/or activity of one or more of CARM1, Med12, Tdrd3 in the cancer cell sensitizes the cancer cell to an immune effector cell, e.g., a cytotoxic T cell.

Other aspects of the disclosure are directed towards methods increasing the anti-tumor function of an immune effector cell, e.g., a cytotoxic T cell.

In one embodiment, the method comprises decreasing expression and/or activity of one or more of CARM1, Med12, Tdrd3 in an immune effector cell, e.g., a cytotoxic T cell, wherein the expression and/or activity of one or more of CARM1, Med12, Tdrd3 is suppressed by one or more inhibiting agents, and wherein decreasing expression and/or activity of one or more of CARM1, Med12, Tdrd3 in the T cell increases the anti-tumor function of an immune effector cell, e.g., a cytotoxic T cell.

Yet further aspects of the disclosure are directed towards methods of decreasing tumor growth in a subject.

In one embodiment, the method comprises administering to the subject a composition comprising one or more inhibiting agents and a pharmaceutically acceptable excipient, wherein the inhibiting agents inhibit expression and/or activity of one or more of CARM1, Med12, Tdrd3 in a tumor cell and/or an immune effector cell, e.g., a cytotoxic T cell; wherein suppression of the expression and/or activity of one or more of CARM1, Med12, Tdrd3 in a tumor cell and/or an immune effector cell, e.g., a cytotoxic T-cell, suppresses tumor growth in the subject.

Tumor growth can refer to cellular proliferation, invasiveness, angiogenesis, or metastasis in mammals. The skilled artisan will recognize that there are various techniques in the art to indicate tumor growth, for example, tumor volume, tumor size, cell number, and the like.

In one embodiment, tumor volume is indicative of tumor growth. In some embodiments, CARM1 knockout in B16 melanoma cells suppresses tumor growth in vivo as indicated by reduced tumor volume. Likewise, CARM1 knockout in B16-Ova cells suppresses tumor growth, and CARM1 inhibition with a small molecule inhibiting suppresses tumor growth in B16F10 tumor bearing mice.

Any of the therapeutic applications described herein can be applied to any subject in need of such therapy, including, for example, a mammal such as a mouse, a rat, a dog, a cat, a cow, a horse, a rabbit, a monkey, a pig, a sheep, a goat, or a human. In some embodiments, the subject is a mouse, rat or human. In some embodiments, the subject is a mouse. In some embodiments, the subject is a rat. In some embodiments, the subject is a human.

### Therapeutic Compositions

As described herein, some aspects of the disclosure refer to administering compositions to a subject to prevent or treat a cancer, for example preventing tumor growth and/or preventing tumor metastasis.

In some embodiments, the composition can comprise an inhibiting agent and a pharmaceutically acceptable carrier. According to the disclosure, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

Non-limiting examples of pharmaceutically acceptable carriers comprise solid or liquid fillers, diluents, and encapsulating substances, including but not limited to lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. The dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

A therapeutically effective dose can depend upon a number of factors known to those of ordinary skill in the art. The dose(s) can vary, for example, depending upon the identity, size, and condition of the subject or sample being treated, further depending upon the route by which the composition is to be administered, if applicable, and the effect which the practitioner is looking for. These amounts can be readily determined by the skilled artisan.

The inhibiting agent(s) can be incorporated into pharmaceutical compositions suitable for administration. Such compositions can comprise the inhibiting agent(s) and a pharmaceutically acceptable carrier. Thus, in some embodiments, the compounds of the disclosure are present in a pharmaceutical composition.

Embodiments can further comprise combination compositions, that is compositions comprise two or more agents that prevent and/or treat a cancer. The term “combination” can refer to either a fixed combination in one dosage unit form, or a kit of parts for the combined administration where a compound and a combination partner (e.g., another drug, also referred to as “therapeutic agent” or “co-agent”) may be administered independently at the same time or separately within time intervals, especially where these time intervals allow that the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

For example, the combination composition can comprise an inhibitor agent and at least one additional therapeutic agent (i.e., a second inhibitor agent and/or an anti-cancer agent). For example, the one additional therapeutic agent can be a toxin, a chemotherapy, a radiolabel, a siRNA, a small molecule, a peptide, an antibody, a genetically engineered cell, or a cytokine.

In some embodiments, the at least one additional therapeutic agent is selected from the group consisting of a chemotherapy agent, an immunotherapy agent, a checkpoint blockade agent, a toxin, a radiolabel, a siRNA, a cancer vaccine, a small molecule, a peptide, an antibody, a genetically engineered cell, a cytokine and a combination thereof. In an exemplary embodiment, the at least one additional therapeutic agent can comprise a checkpoint inhibitor, such as that which targets key regulators of a subject's immune system.

A subject's immune system protects the subject from disease, including cancer. One main type of immune cell that does this is called a T cell. T cells have proteins on them that turn on an immune response and other proteins that turn it off. These are called checkpoints. Some checkpoint proteins help tell T cells to become active. But if T cells are active for too long, or react to things they shouldn't, they can start to destroy healthy cells and tissues. So other checkpoints help tell T cells to switch off. Some cancer cells make high levels of proteins that can switch off T cells, when the T cells should be attacking the cancer cells. By inhibiting the immune system, the cancer cells are causing the T cells to no longer recognize and kill cancer cells. In an exemplary embodiment, the at least one additional therapeutic agent can be T cells. In another exemplary embodiment, the at least one additional therapeutic agent can be CAR T cells.

Agents that block checkpoint proteins are called checkpoint inhibitors. Such agents can stop the proteins on the cancer cells from inhibiting the subject's immune system. This turns the immune system back on, and the T cells are able to find and attack the cancer cells.

Key checkpoint proteins include CTLA-4 (found on T cells), PD-1 (found on T cells), and PD-L1 (found on cancer cells). Thus, some aspects of the disclosure can comprise administering to a subject a composition comprising a checkpoint inhibitor, such as a checkpoint inhibitor antibody. For example, the antibody can be an anti-PD1 antibody, an anti-CTLA-4 antibody, an anti-PD-L1 antibody, or a combination thereof (see Table 3):

TABLE 3
Checkpoint Inhibitors

| ANTIBODY NAME | TARGET | |
|---|---|---|
| Nivolumab | PD-1 | fully human monoclonal |
| | | IgG4 antibody |
| Pembrolizumab | PD-1 | humanized IgG4 antibody |
| Ipilimumab | CTLA-4 | human antibody |
| Atezolizumab | PD-L1 | humanized IgG1 antibody |
| Avelumab | PD-L1 | fully human monoclonal |
| | | antibody |
| Durvalumab | PD-L1 | human IgG1 monoclonal |
| | | antibody |
| Cemiplimab | PD-1 | human antibody |
| See, for example, He, | TIM-3 | TIM-3 plays an important |
| Yayi, et al. | | role in T-cell exhaustion |
| OncoTargets and therapy 11 | | |
| (2018): 7005 | | |
| See, for example, Andrews, | LAG3 | LAG3 plays a role in T-cell |
| Lawrence P., et al. | | exhaustion |
| Immunological reviews 276.1 | | |
| (2017): 80-96 | | |
| Solomon, Benjamin L., and | TIGIT | TIGIT plays a role in |
| Ignacio Garrido-Laguna. Cancer | | immunosurveillance, and |
| Immunology, Immunotherapy | | regulates T-cell mediated |
| 67.11 (2018): 1659-1667 | | immunity. |

The skilled artisan will recognize that any checkpoint inhibitor can be utilized in embodiments described herein. In some embodiments, the synergistic effect on tumor growth suppression of the combination therapy of inactivation of Carm1 and checkpoint blockade therapy, anti-PD-1 antibody. In other embodiments, the synergistic effect on tumor growth suppression of the combination therapy of inactivation of Carm1 and checkpoint blockade therapy, anti-CTLA-4 antibody.

The inhibiting agent and the one or more additional therapeutic agent can be referred to as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

In certain embodiments, compositions and methods of the present disclosure can also include a variety of combination therapies with both chemical and radiation based treatments. For example, combination chemotherapies can include, for example, abraxane, altretamine, docetaxel, herceptin, methotrexate, novantrone, zoladex, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristin, vinblastin and methotrexate, or any analog or derivative variant of the foregoing and also combinations thereof.

In specific embodiments, chemotherapy for the individual is employed in conjunction with the methods described herein, for example before, during and/or after administration of the methods described herein.

Other factors that cause DNA damage and have been used extensively include what are commonly known as gamma-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy other than the inventive therapy described herein could thus be used as part of a combined therapy, in conjunction with the present therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present disclosure. Common tumor markers include PD-1, PD-L1, CTLA4, carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

In yet another embodiment, the secondary treatment is a gene therapy in which a therapeutic polynucleotide is administered before, after, or at the same time as the present disclosure clinical embodiments. A variety of expression products are encompassed within the disclosure, including inducers of cellular proliferation, inhibitors of cellular proliferation, or regulators of programmed cell death.

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and controlled surgery (Mohs' surgery). It is further contemplated that the present disclosure may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

It is contemplated that other agents may be used in combination with the present disclosure to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-lbeta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present disclosure to improve the anti-hyperproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

### Kit

Some aspects of the disclosure also encompasses kits, such as for treating and/or diagnosing cancer.

In one embodiment, the kit can comprise one or more compositions as described herein, such as inhibiting agents, for example.

For example, the kit can comprise one or more reagents useful for detection of the level of CARM1, Med12, TDRD3, or other proteins in a sample. The one or more reagents can be immobilized to a solid support. Non-limiting examples of the composition of the solid support structure comprise plastic, cardboard, glass, plexiglass, tin, paper, or a combination thereof. The solid support can also comprise a dip stick, spoon, scoopula, filter paper or swab.

The reagents can comprise a labeled compound or agent capable of detecting a cancer or tumor cell (e.g., an scFv or monoclonal antibody) in a biological sample; means for determining the amount of gene expression in the sample; and means for comparing the amount of gene expression in the sample with a standard, such as a control sample or threshold. The standard is, in some embodiments, a non-cancer cell or cell extract thereof. The compound or agent can be packaged in a suitable container. The kit can further comprise instructions for using the kit to detect cancer in a sample.

In embodiments, the kit can also include primers for amplifying an mRNA transcribed from a gene that encodes the polypeptide and/or control samples for testing the primers. For example, the control samples can comprise nucleic acids that hybridize to the primers.

The kit can also comprise a sample collection apparatus such as those devices used for collecting a biological fluid or tumor biopsy.

In an embodiment, the kit can include a container that contains the one or more reagents and, optionally informational material. The informational material can be descriptive, instructional, marketing or other material that relates to the methods described herein and/or the use of the agents for diagnostic purposes. In one embodiment, the kit includes also includes one or more anti-cancer therapeutics.

The informational material of the kits is not limited in its form. In one embodiment, the informational material can include information about production of the components of the kit, such as molecular weight, concentration, date of expiration, batch or production site information, and so forth. In one embodiment, the informational material relates to methods of using the components of the kit. The information can be provided in a variety of formats, include printed text, computer readable material, video recording, or audio recording, or information that provides a link or address to substantive material.

The kit can include other ingredients, such as solvents or buffers, a stabilizer, or a preservative. Optionally, the kit can comprise therapeutic agents that can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

### Immune Effector Cells

In some embodiments, the disclosure provides immune effector cells, including T cells, cytotoxic T cells, tumor-infiltrating lymphocytes (TIL), regulatory (CD4) T cells, and Natural Killer (NKT) cells, expressing at least one of an antigen-recognizing receptor. In any aspect, the immune effector cells express at least one tumor specific antigen-recognizing receptor. In some aspects, tumor cell antigen specific T cells, NKT cells, TIL, CTL cells or other immune effector cells are used. Non-limiting examples of immune effector cells include T cells, such as, for example, αβ-TCR+ T cells (e.g., CD8+ T cells or CD4+ T cells) γδ-TCR+ T cells, tumor-infiltrating lymphocytes (TIL), Natural Killer T cells (NKT), a cytotoxic T lymphocytes (CTL), and a CD4 T cells.

In some embodiments, the immune effector cells disclosed herein comprise a chimeric antigen receptor (CAR).

### Antibodies

In some embodiments, the inhibiting agent disclosed herein is an antibody, such as an antagonistic antibody, or antigen-binding fragment thereof. For example, the inhibiting agent may be a CARM1 antibody, a Med12 antibody, a TDRD3 antibody, or antigen-binding fragment thereof.

An antibody of the present disclosure binds at least one specified epitope specific to CARM1, Med12, or TDRD3 protein, subunit, fragment, portion of the invention, or any combination thereof. The epitope can comprise an antibody binding region that comprises at least one portion of the amino acid sequence of CARM1, Med12, or TDRD3 (e.g., SEQ ID NOs: 1-9), which epitope is preferably comprised of at least 1-5 amino acids of the sequences. The antibody can include or be derived from any mammal, such as, but not limited to, a human, a mouse, a rabbit, a rat, a rodent, a primate, or any combination thereof, and the like.

An anti-CARM1, anti-Med12, or anti-TDRD3 antibody, as described herein, has at least one activity, such as, but not limited to inhibiting CARM1, Med12, or TDRD3 activity. An anti-CARM1, anti-Med12, or anti-TDRD3 antibody can thus be screened for a corresponding activity according to known methods, such as but not limited to, at least one biological activity of human CARM1, Med12, or TDRD3.

As used herein, an “antibody,” “antibody portion,” or “antibody fragment” and/or “antibody variant” and the like include any protein or polypeptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof. Such antibody optionally further affects a specific ligand, such as but not limited to, where such antibody modulates, decreases, increases, antagonizes, angonizes, mitigates, alleviates, blocks, inhibits, abrogates and/or interferes with at least one CARM1, Med12, or TDRD3 activity or binding, in vitro, in situ and/or in vivo. As a non-limiting example, a suitable anti-CARM1 antibody, specified portion or variant of the present invention can bind at least one CARM1 protein or polypeptide of the invention, or specified portions, variants or domains thereof. A suitable anti-CARM1 antibody, specified portion, or variant can also optionally affect at least one of CARM1 activity or function, such as but not limited to, CARM1 signaling, CARM1 activity, CARM1 production and/or synthesis.

An “antibody” refers to a molecule of the immunoglobulin family comprising a tetrameric structural unit. Each tetramer is composed of two identical pairs of polypeptide chains, each pair having one “light” chain (about 25 kD) and one “heavy” chain (about 50-70 kD), connected through a disulfide bond. Recognized immunoglobulin genes include the κ, λ, α, γ, δ, ε, and μ constant region genes, as well as the myriad immunoglobulin variable region genes. Light chains are classified as either κ or λ. Heavy chains are classified as γ, α, δ, or ε, which in turn define the immunoglobulin classes, IgG, IgM, IgA, IgD, and IgE, respectively. Antibodies can be of any isotype/class (e.g., IgG, IgM, IgA, IgD, and IgE), or any subclass (e.g., IgG1, IgG2, IgG3, IgG4, IgA1, IgA2).

The term “antibody” is further intended to encompass antibodies, digestion fragments, specified portions and variants thereof, including antibody mimetics or portions of antibodies that mimic the structure and/or function of an antibody or specified fragment or portion thereof, including single chain antibodies and fragments thereof. Functional fragments include antigen-binding fragments that bind to a mammalian CARM1, Med12, or TDRD3. For example, antibody fragments capable of binding to CARM1, Med12, or TDRD3 or portions thereof, including, but not limited to, Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Both the light and heavy chains are divided into regions of structural and functional homology. The terms “constant” and “variable” are used structurally and functionally. The N-terminus of each chain defines a variable (V) region or domain of about 100 to 110 or more amino acids primarily responsible for antigen recognition. The terms variable light chain (VL) and variable heavy chain (VH) refer to these regions of light and heavy chains respectively. The pairing of a VH and VL together forms a single antigen-binding site. In addition to V regions, both heavy chains and light chains contain a constant (C) region or domain. A secreted form of an immunoglobulin C region is made up of three C domains, CH1, CH2, CH3, optionally CH4 (Cμ), and a hinge region. A membrane-bound form of an immunoglobulin C region also has membrane and intracellular domains. Each light chain has a VL at the N-terminus followed by a constant domain (C) at its other end. The constant domains of the light chain (CL) and the heavy chain (CH1, CH2 or CH3) confer important biological properties such as secretion, transplacental mobility, Fc receptor binding, complement binding, and the like. By convention, the numbering of the constant region domains increases as they become more distal from the antigen binding site or amino-terminus of the antibody. The N-terminus is a variable region and at the C-terminus is a constant region; the CH3 and CL domains actually comprise the carboxy-terminal domains of the heavy and light chain, respectively. The VL is aligned with the VH and the CL is aligned with the first constant domain of the heavy chain (CH1). As used herein, an “antibody” encompasses conventional antibody structures and variations of antibodies. Thus, within the scope of this concept are full length antibodies, chimeric antibodies, humanized antibodies, human antibodies, and fragments thereof.

Antibodies exist as intact immunoglobulin chains or as a number of well-characterized antibody fragments produced by digestion with various peptidases. The term “antibody fragment,” as used herein, refers to one or more portions of an antibody that retain the ability to specifically interact with (e.g., by binding, steric hindrance, stabilizing/destabilizing, spatial distribution) an epitope of an antigen. Thus, for example, pepsin digests an antibody below the disulfide linkages in the hinge region to produce F(ab)′2, a dimer of Fab′ which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (Paul, Fundamental Immunology 3d ed. (1993)). While various antibody fragments are defined in terms of the digestion of an intact antibody, one of skill will appreciate that such fragments may be synthesized de novo either chemically or by using recombinant DNA methodology. As used herein, an “antibody fragment” refers to one or more portions of an antibody, either produced by the modification of whole antibodies, or those synthesized de novo using recombinant DNA methodologies, that retain binding specificity and functional activity. Examples of antibody fragments include Fv fragments, single chain antibodies (ScFv), Fab, Fab′, Fd (Vh and CH1 domains), dAb (Vh and an isolated CDR); and multimeric versions of these fragments (e.g., F(ab′)2,) with the same binding specificity.

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CH1 domain and/or hinge region of the heavy chain. The various portions of antibodies can be joined together chemically by conventional techniques, or can be prepared as a contiguous protein using genetic engineering techniques.

A “Fab” domain as used in the context comprises a heavy chain variable domain, a constant region CH1 domain, a light chain variable domain, and a light chain constant region CL domain. The interaction of the domains is stabilized by a disulfide bond between the CH1 and CL domains. In some embodiments, the heavy chain domains of the Fab are in the order, from N-terminus to C-terminus, VH—CH and the light chain domains of a Fab are in the order, from N-terminus to C-terminus, VL-CL. In some embodiments, the heavy chain domains of the Fab are in the order, from N-terminus to C-terminus, CH—VH and the light chain domains of the Fab are in the order CL-VL. Although the Fab fragment was historically identified by papain digestion of an intact immunoglobulin, in the context of this disclosure, a “Fab” is typically produced recombinantly by any method. Each Fab fragment is monovalent with respect to antigen binding, i.e., it has a single antigen-binding site.

“Complementarity-determining domains” or “complementarity-determining regions” (“CDRs”) interchangeably refer to the hypervariable regions of VL and VH. CDRs are the target protein-binding site of antibody chains that harbors specificity for such target protein. There are three CDRs (CDR1-3, numbered sequentially from the N-terminus) in each human VL or VH, constituting about 15-20% of the variable domains. CDRs are structurally complementary to the epitope of the target protein and are thus directly responsible for the binding specificity. The remaining stretches of the VL or VH, the so-called framework regions (FR), exhibit less variation in amino acid sequence (Kuby, Immunology, 4th ed., Chapter 4. W.H. Freeman & Co., New York, 2000).

Positions of CDRs and framework regions can be determined using various well known definitions in the art, e.g., Kabat, Chothia, and AbM (see, e.g., Kabat et al. 1991 Sequences of Proteins of Immunological Interest, Fifth Edition, U.S. Department of Health and Human Services, NIH Publication No. 91-3242, Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997)). Definitions of antigen combining sites are also described in the following: Ruiz et al., Nucleic Acids Res., 28:219-221 (2000); and Lefranc, M. P., Nucleic Acids Res., 29:207-209 (2001); (ImMunoGenTics (IMGT) numbering) Lefranc, M.-P., The Immunologist, 7, 132-136 (1999); Lefranc, M.-P. et al., Dev. Comp. Immunol., 27, 55-77 (2003); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996).

Under Kabat, CDR amino acid residues in the VH are numbered 31-35 (HCDR1), 50-65 (HCDR2), and 95-102 (HCDR3); and the CDR amino acid residues in the VL are numbered 24-34 (LCDR1), 50-56 (LCDR2), and 89-97 (LCDR3). Under Chothia, CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

An “antibody variable light chain” or an “antibody variable heavy chain” as used herein refers to a polypeptide comprising the VL or VH, respectively. The endogenous VL is encoded by the gene segments V (variable) and J (junctional), and the endogenous VH by V, D (diversity), and J. Each of VL or VH includes the CDRs as well as the framework regions (FR). The term “variable region” or “V-region” interchangeably refer to a heavy or light chain comprising FR1-CDR1-FR2-CDR2-FR3-CDR3-FR4. A V-region can be naturally occurring, recombinant or synthetic. In this application, antibody light chains and/or antibody heavy chains may, from time to time, be collectively referred to as “antibody chains.”

The C-terminal portion of an immunoglobulin heavy chain herein, comprising, e.g., CH2 and CH3 domains, is the “Fc” domain. An “Fc region” as used herein refers to the constant region of an antibody excluding the first constant region (CH1) immunoglobulin domain. Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains CH2 and CH3 and the hinge between CH1 and CL. It is understood in the art that boundaries of the Fc region may vary, however, the human IgG heavy chain Fc region is usually defined to comprise residues C226 or P230 to its carboxyl-terminus, using the numbering is according to the EU index as in Kabat et al. (1991, NIH Publication 91-3242, National Technical Information Service, Springfield, Va.). “Fc region” may refer to this region in isolation or this region in the context of an antibody or antibody fragment. “Fc region” includes naturally occurring allelic variants of the Fc region, e.g., in the CH2 and CH3 region, including, e.g., modifications that modulate effector function. Fc regions also include variants that don't result in alterations to biological function. For example, one or more amino acids are deleted from the N-terminus or C-terminus of the Fc region of an immunoglobulin without substantial loss of biological function. For example, in certain embodiments a C-terminal lysine is modified replaced or removed. In particular embodiments one or more C-terminal residues in the Fc region is altered or removed. In certain embodiments one or more C-terminal residues in the Fc (e.g., a terminal lysine) is deleted. In certain other embodiments one or more C-terminal residues in the Fc is substituted with an alternate amino acid (e.g., a terminal lysine is replaced). Such variants are selected according to general rules known in the art so as to have minimal effect on activity (see, e.g., Bowie, et al., Science 247:306-1310, 1990). The Fc domain is the portion of the immunoglobulin (Ig) recognized by cell receptors, such as the FcR, and to which the complement-activating protein, Cl q, binds. The lower hinge region, which is encoded in the 5′ portion of the CH2 exon, provides flexibility within the antibody for binding to FcR receptors.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, and drug; or (b) the variable region, or a portion thereof, is altered, replaced or exchanged with a variable region having a different or altered antigen specificity.

A “humanized” antibody is an antibody that retains the reactivity (e.g., binding specificity, activity) of a non-human antibody while being less immunogenic in humans. This can be achieved, for instance, by retaining non-human CDR regions and replacing remaining parts of an antibody with human counterparts (see, e.g., Morrison et al., Proc. Natl. Acad. Sci. USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994)).

A “human antibody” includes antibodies having variable regions in which both the framework and CDR regions are derived from sequences of human origin. Furthermore, if an antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000. Human antibodies may include amino acid residues not encoded by human sequences (e.g., mutations introduced by random or site-specific mutagenesis in vitro or by somatic mutation in vivo, or a conservative substitution to promote stability or manufacturing).

The term “corresponding human germline sequence” refers to a nucleic acid sequence encoding a human variable region amino acid sequence or subsequence that shares the highest determined amino acid sequence identity with a reference variable region amino acid sequence or subsequence in comparison to all other all other known variable region amino acid sequences encoded by human germline immunoglobulin variable region sequences. A corresponding human germline sequence can also refer to the human variable region amino acid sequence or subsequence with the highest amino acid sequence identity with a reference variable region amino acid sequence or subsequence in comparison to all other evaluated variable region amino acid sequences. A corresponding human germline sequence can be framework regions only, complementary determining regions only, framework and complementary determining regions, a variable segment (as defined above), or other combinations of sequences or sub-sequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 92%, 93%, 94%, 95%, 96%, 97%, 98%, 99%, or 100% sequence identity with the reference variable region nucleic acid or amino acid sequence.

The term “valency” as used herein refers to the number of potential target binding sites in a polypeptide. Each target binding site specifically binds one target molecule or a specific site on a target molecule. When a polypeptide comprises more than one target binding site, each target binding site may specifically bind the same or different molecules (e.g., may bind to different molecules, e.g., different antigens, or different epitopes on the same molecule). A conventional antibody, for example, has two binding sites and is bivalent; “trivalent” and “tetravalent” refer to the presence of three binding sites and four binding sites, respectively, in an antibody molecule.

The phrase “specifically binds” when used in the context of describing the interaction between a target (e.g., a protein) and an antibody, refers to a binding reaction that is determinative of the presence of the target in a heterogeneous population of proteins and other biologics, e.g., in a biological sample, e.g., a blood, serum, plasma or tissue sample. Thus, under certain designated conditions, an antibody with a particular binding specificity binds to a particular target at least two times the background and do not substantially bind in a significant amount to other targets present in the sample. In one embodiment, under designated conditions, an antibody with a particular binding specificity binds to a particular antigen at least ten (10) times the background and does not substantially bind in a significant amount to other targets present in the sample. Specific binding to an antibody under such conditions may require an antibody to have been selected for its specificity for a particular protein. A variety of formats may be used to select antibodies that are specifically reactive with a particular target antigen protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least than 10 to 100 times over the background.

The term “equilibrium dissociation constant (KD, M)” refers to the dissociation rate constant (kd, time⁻¹) divided by the association rate constant (ka, time⁻¹, M⁻¹). Equilibrium dissociation constants can be measured using any known method in the art. The antibody generally will have an equilibrium dissociation constant of less than about 10⁻⁷ or 10⁻⁸M, for example, less than about 10⁻⁹M or 10⁻¹⁰ M, in some embodiments, less than about 10⁻¹¹ M, 10⁻¹² M or 10⁻¹³ M.

At least one antibody of the invention binds at least one specified epitope specific to CARM1, Med12, or TDRD3 protein, subunit, fragment, portion or any combination thereof, as described herein (e.g., SEQ ID NOs: 1-9). The at least one epitope can comprise at least one antibody binding region that comprises at least one portion of the protein sequences corresponding to the peptide sequences from the receptor binding region of human CARM1, Med12, or TDRD3, as described herein, which epitope is preferably comprised of at least one extracellular, soluble, hydrophilic, external or cytoplasmic portion of the protein.

Generally, the human antibody or antigen-binding fragment of the present invention will comprise an antigen-binding region that comprises at least one human complementarity determining region (CDR1, CDR2 and CDR3) or variant of at least one heavy chain variable region and at least one human complementarity determining region (CDR1, CDR2 and CDR3) or variant of at least one light chain variable region.

Anti-CARM1, anti-Med12, or anti-TDRD3 antibodies useful in the methods and compositions of the present invention can optionally be characterized by high affinity binding to a CARM1, Med12, or TDRD3 protein and, optionally and preferably, having low toxicity. In particular, an antibody, specified fragment or variant of the invention, where the individual components, such as the variable region, constant region and framework, individually and/or collectively, optionally and preferably, possess low immunogenicity, is useful in the present invention. The antibodies that can be used in the invention are optionally characterized by their ability to treat patients for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably, less than about 100 measured with a double antigen enzyme immunoassay) (Elliott et al., Lancet 344:1125-1127 (1994), entirely incorporated herein by reference).

In another aspect, the invention relates to human antibodies and antigen-binding fragments, as described herein, which are modified by the covalent attachment of an organic moiety. Such modification can produce an antibody or antigen-binding fragment with improved pharmacokinetic properties (e.g., increased in vivo serum half-life). The organic moiety can be a linear or branched hydrophilic polymeric group, fatty acid group, or fatty acid ester group. Lipid molecules such as a disteroylphosphatidyl ethanolamine moiety, either alone or covalently bonded to a hydrophilic polymer, are useful. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified antibodies and antigen-binding fragments of the invention can comprise one or more organic moieties that are covalently bonded, directly or indirectly, to the antibody. Each organic moiety that is bonded to an antibody or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an antibody modified by the covalent attachment of polylysine is encompassed by the invention. Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the antibody of the invention has a molecular weight of about 800 to about 150,000 Daltons as a separate molecular entity. For example, PEG5000 and PEG20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Fatty acids that are suitable for modifying antibodies of the invention include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-Δ9-octadecanoate (C18, oleate), all cis-Δ5,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified human antibodies and antigen-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups such as tosylate, mesylate, halo (chloro, bromo, fluoro, iodo), N-hydroxysuccinimidyl esters (HS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom such as oxygen, nitrogen or sulfur. Suitable linker moieties include, for example, tetraethylene glycol, —(CH2)3-, —NH—(CH2)6-NH—, (CH2)2-NH— and —CH2-O—CH2-CH2-O—CH2—CH2-O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified antibodies of the invention can be produced by reacting a human antibody or antigen-binding fragment with a modifying agent. For example, the organic moieties can be bonded to the antibody in a non-site specific manner by employing an amine-reactive modifying agent, for example, a NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

Monoclonal antibodies of this invention may be raised by traditional immunization and hybridoma technology. After immunization of mice with human CARM1, Med12, or TDRD3 antigenic compositions comprising the polypeptides of the invention, spleen cells or lymphocytes from lymph node tissue from immunized animals are recovered and immortalized by fusion with myeloma cells or by Epstein-Barr (EB)-virus transformation. Monoclonal antibodies are obtained by screening for clones expressing the desired antibody. While mice are frequently employed as the test model, it is contemplated that any mammalian subject, including human subjects or antibody-producing cells, can be manipulated according to the processes of this invention to serve as the basis for production of mammalian, including human and hybrid cell lines. Techniques for cloning recombinant DNA of antibody molecule directly from an antibody-expressing B cell are within the scope of this invention. Such B cells can be isolated by the fluorescence activated cell sorter.

While routinely, mouse monoclonal antibodies are generated, the invention is not so limited. For therapeutic applications, human or humanized antibodies are desired. Such antibodies can be obtained by using human hybridomas or by generating humanized antibodies. Humanized antibodies can be developed by replacing the specific segments of a non-human antibody with corresponding segments of a human antibody gene. This process retains most or all of CDR regions of the light and heavy chain variable regions of parental antibody and largely replaces the framework regions with human sequences (EP Patent No. 184187; EP Patent No. 171496; EP Patent No. 173494 and WO Patent No. 86/01533). Human monoclonal antibodies are also generated in transgenic mice that contain genes or gene segments encoding human antibodies in their genome (U.S. Pat. No. 6,162,963; WO Patent No. 93/12227; U.S. Pat. Nos. 58,775,397; 5,874,299; 5,814,318; 5,789,650; 5,770,429; 5,661,016; 5,625,126; 5,569,825; 5,545,806; and WO Patent No. 91/10741).

Human monoclonal antibodies are also obtained from recombinant antibody libraries, generated in vitro or in vivo, using phage display, ribosome display, or related screening or selection techniques. Examples of procedures for generating antibody libraries, primarily of human origin, are disclosed by A. Knappik and others (U.S. Pat. Nos. 6,291,158; 6,291,159; 6,291,160 and 6,291,161). Examples of methods for selections of human antibodies to specific antigen targets from such libraries are disclosed by B. Krebs and others (U.S. Pat. Nos. 5,955,341; 5,759,817; 5,658,727; 6,235,469; 5,969,108; 5,886,793)].

At least one anti-CARM1, anti-Med12, or anti-TDRD3 antibody of the present invention can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2nd Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001), each entirely incorporated herein by reference.

Human antibodies that are specific for human CARM1, Med12, or TDRD3 (as described herein), variants, or fragments thereof can be raised against an appropriate immunogenic antigen as described herein, such as the isolated and/or CARM1, Med12, or TDRD3, variants, or portions thereof (including synthetic molecules, such as synthetic polypeptides) as described herein. Other specific or general mammalian antibodies can be similarly raised. Preparation of immunogenic antigens, and monoclonal antibody production can be performed using any suitable technique.

In one approach, a hybridoma is produced by fusing a suitable immortal cell line, e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMAIWA, NEURO 2A, or the like, heteromylomas, fusion products thereof, any cell or fusion cell derived therefrom, or any other suitable cell line as known in the art, which can be found, for example at ATCC, with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid molecule, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, chapter 2, entirely incorporated herein by reference.

Antibody producing cells can also be obtained from the peripheral blood or, preferably the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells that produce antibodies with the desired specificity can be selected by a suitable assay (e.g., ELISA).

Other suitable methods of producing or isolating antibodies of the requisite specificity can be used, including, but not limited to, methods that select recombinant antibody from a polypeptide or protein display library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, Del.; Biovation, Aberdeen, Scotland, UK; BioInvent, Lund, Sweden; Dyax Corp., Enzon, Affymax/Biosite; Xoma, Berkeley, Calif.; Ixsys. See, e.g., EP 368,684, PCT/GB91/01134; PCT/GB92/01755; PCT/GB92/002240; PCT/GB92/00883; PCT/GB93/00605; U.S. Ser. No. 08/350,260 (May 12, 1994); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); WO96/13583, WO97/08320 (MorphoSys); WO95/16027 (BioInvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins—U.S. Pat. Nos. 5,723,323, 5,763,192, 5,814,476, 5,817,483, 5,824,514, 5,976,862, WO 86/05803, EP 590 689 (Ixsys, now Applied Molecular Evolution (AME), each entirely incorporated herein by reference) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated by reference as well as related patents and applications) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (November 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, Mass.; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Methods for engineering or humanizing non-human or human antibodies can also be used and are well known in the art. Generally, a humanized or engineered antibody has one or more amino acid residues from a source that is non-human, e.g., but not limited to, mouse, rat, rabbit, non-human primate or other mammal. These human amino acid residues are often referred to as “import” residues, which are typically taken from an “import” variable, constant or other domain of a known human sequence. Known human Ig sequences are disclosed, e.g., at
www.ncbi.nlm.nih.gov/entrez/query.fcgi; www.atcc.org/phage/hdb.html;
www.sciquest.com/; www.abcam.com/;
www.antibodyresource.com/onlinecomp.html;
www.public.iastate.edu/\tilde{ }pedro/research_tools.html; www.mgen.uni-heidelberg.de/SD/IT/IT.html; www.whfreeman.com/immunology/CH05/kuby05.htm;
www.library.thinkquest.org/12429/Immune/Antibody.html;
www.hhmi.org/grants/lectures/1996/vlab/;
www.path.cam.ac.uk/\tilde{ }mrc7/mikeimages.html; www.antibodyresource.com/;
mcb.harvard.edu/BioLinks/Immunology.html.www.immunologylink.com;
pathbox.wustl.edu/\tilde{ }hcenter/index.html; www.biotech.ufl.edu/\tilde{ }hcl/;
www.pebio.com/pa/340913/340913.html; www.nal.usda.gov/awic/pubs/antibody/;
www.m.ehime-u.ac.jp/\tilde{ }yasuhito/Elisa.html; www.biodesign.com/table.asp;
www.icnet.uk/axp/facs/davies/links.html; www.biotech.ufl.edu/\tilde{ }fccl/protocol.html;
www.isac-net.org/sites_geo.html; aximtl.imt.uni-marburg.de/\tilde{ }rek/AEPStart.html;
baserv.uci.kun.nl/\tilde{ }jraats/linksl.html; www.recab.uni-hd.de/immuno.bme.nwu.edu/;
www.mrc-cpe.cam.ac.uk/imt-doc/public/INTRO.html;
www.ibt.unam.mx/vir/V_mice.html; imgt.cnusc.fr:8104/;
www.biochem.ucl.ac.uk/\hat{ }martin/abs/index.html; antibody.bath.ac.uk/;
abgen.cvm.tamu.edu/lab/wwwabgen.html;
www.unizh.ch/\tilde{ }honegger/AHOseminar/Slide01.html; www.cryst.bbk.ac.uk/\tilde{ }ubcg07s/;
www.nimr.mrc.ac.uk/CC/ccaewg/ccaewg.htm;
www.path.cam.ac.uk/\tilde{ }mrc7/humanisation/TAHHP.html;
www.ibt.unam.mx/vir/structure/stat_aim.html; www.biosci.missouri. edu/smithgp/index.html; www.cryst.bioc.cam.ac.uk/\tilde{ }fmolina/Web-pages/Pept/spottech.html; www.jerini.de/fr_products.htm;
www.patents.ibm.com/ibm.html. Kabat et al., Sequences of Proteins of Immunological Interest, U.S. Dept. Health (1983), each entirely incorporated herein by reference.

Such imported sequences can be used to reduce immunogenicity or reduce, enhance or modify binding, affinity, on-rate, off-rate, avidity, specificity, half-life, or any other suitable characteristic, as known in the art. Generally part or all of the non-human or human CDR sequences are maintained while the non-human sequences of the variable and constant regions are replaced with human or other amino acids. Antibodies can also optionally be humanized with retention of high affinity for the antigen and other favorable biological properties. To achieve this goal, humanized antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Humanization or engineering of antibodies of the present invention can be performed using any known method, such as but not limited to, those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; and 4,816,567, PCT/: US98/16280, US96/18978, US91/09630, US91/05939, US94/01234, GB89/01334, GB91/01134, GB92/01755; WO90/14443, WO90/14424, WO90/14430, and EP 229246, each entirely incorporated herein by reference, included references cited therein.

The anti-CARM1, anti-Med12, or anti-TDRD3 antibody can also be optionally generated by immunization of a transgenic animal (e.g., mouse, rat, hamster, non-human primate, and the like) capable of producing a repertoire of human antibodies, as described herein and/or as known in the art. Cells that produce a human anti-CARM1, anti-Med12, or anti-TDRD3 antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 Bl, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Screening antibodies for specific binding to similar proteins or fragments can be conveniently achieved using peptide display libraries. This method involves the screening of large collections of peptides for individual members having the desired function or structure. Antibody screening of peptide display libraries is well known in the art. The displayed peptide sequences can be from 3 to 5,000 or more amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278. Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754 and 5,643,768. Peptide display libraries, vector, and screening kits are commercially available from such suppliers as Invitrogen (Carlsbad, Calif.), and Cambridge antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, U.S. Pat. Nos. 5,427,908, 5,580,717, assigned to Affymax; U.S. Pat. No. 5,885,793, assigned to Cambridge antibody Technologies; U.S. Pat. No. 5,750,373, assigned to Genentech, U.S. Pat. Nos. 5,618,920, 5,595,898, 5,576,195, 5,698,435, 5,693,493, and 5,698,417, assigned to Xoma, Colligan, supra; Ausubel, supra; or Sambrook, supra, each of the above patents and publications entirely incorporated herein by reference.

Antibodies of the present invention can also be prepared in milk by administering at least one anti-CARM1, anti-Med12, or anti-TDRD3 antibody encoding nucleic acid to transgenic animals or mammals, such as goats, cows, horses, sheep, and the like, that produce antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

Antibodies of the present invention can additionally be prepared using at least one anti-CARM1, anti-Med12, or anti-TDRD3 antibody encoding nucleic acid to provide transgenic plants and cultured plant cells (e.g., but not limited to, tobacco and maize) that produce such antibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., Curr. Top. Microbol. Immunol. 240:95-118 (1999) and references cited therein. Also, transgenic maize has been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. Antibodies have also been produced in large amounts from transgenic plant seeds including antibody fragments, such as single chain antibodies (scFv's), including tobacco seeds and potato tubers. See, e.g., Conrad et al., Plant Mol. Biol. 38:101-109 (1998) and reference cited therein. Thus, antibodies of the present invention can also be produced using transgenic plants, according to known methods. See also, e.g., Fischer et al., Biotechnol. Appl. Biochem. 30:99-108 (October, 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein. Each of the above references is entirely incorporated herein by reference.

The antibodies of the invention can bind human CARM1, Med12, or TDRD3 with a wide range of affinities (KD). In a preferred embodiment, at least one human mAb of the present invention can optionally bind human CARM1, Med12, or TDRD3 with high affinity. For example, a human mAb can bind human CARM1, Med12, or TDRD3 with a KD equal to or less than about 10⁻⁷M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³ or any range or value therein.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., KD, Ka, Kd) are preferably made with standardized solutions of antibody and antigen, and a standardized buffer, such as the buffer described herein.

An anti-CARM1, anti-Med12, or anti-TDRD3 antibody can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Antibodies of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody of the present invention can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

A typical mammalian expression vector contains at least one promoter element, which mediates the initiation of transcription of mRNA, the antibody coding sequence, and signals required for the termination of transcription and polyadenylation of the transcript. Additional elements include enhancers, Kozak sequences, and intervening sequences flanked by donor and acceptor sites for RNA splicing. Highly efficient transcription can be achieved with the early and late promoters from SV40, the long terminal repeats (LTRS) from Retroviruses, e.g., GAS-6, HTLVI, HIVI and the early promoter of the cytomegalovirus (CMV). However, cellular elements can also be used (e.g., the human actin promoter). Suitable expression vectors for use in practicing the present invention include, for example, vectors such as pIRES1neo, pRetro-Off, pRetro-On, PLXSN, or pLNCX (Clonetech Labs, Palo Alto, Calif.), pcDNA3.1 (+/−), pcDNA/Zeo (+/−) or pcDNA3.1/Hygro (+/−) (Invitrogen), PSVL and PMSG (Pharmacia, Uppsala, Sweden), pGAS-6cat (ATCC 37152), pSV2dhfr (ATCC 37146) and pBC12MI (ATCC 67109). Mammalian host cells that could be used include human Hela 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded antibody. The DHFR (dihydrofolate reductase) marker is useful to develop cell lines that carry several hundred or even several thousand copies of the gene of interest. Another useful selection marker is the enzyme glutamine synthase (GS) (Murphy, et al., Biochem. J. 227:277-279 (1991); Bebbington, et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of antibodies.

The expression vectors pC1 and pC4 contain the strong promoter (LTR) of the Rous Sarcoma Virus (Cullen, et al., Molec. Cell. Biol. 5:438-447 (1985)) plus a fragment of the CMV-enhancer (Boshart, et al., Cell 41:521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

### RNA Interference

One of the most important recent discoveries in biomedical research is the RNA interference (RNAi) pathway, which is used by cells to regulate the activity of many genes. The principles of RNAi have opened many new possibilities for the identification of therapeutic targets. RNA interference (RNAi) is an effective tool for genome-scale, high throughput analysis of gene function. The term “RNA interference” (RNAi), also called post transcriptional gene silencing (PTGS), refers to the biological process in which RNA molecules inhibit gene expression. An “RNA interfering agent” as used herein, is defined as any agent that interferes with or inhibits expression of a target gene, e.g., a target gene of the disclosure, by RNA interference (RNAi). Such RNA interfering agents include, but are not limited to, nucleic acid molecules including RNA molecules which are homologous to the target gene, e.g., a target gene of the disclosure, or a fragment thereof, short interfering RNA (siRNA), short hairpin RNA (shRNA), and small molecules which interfere with or inhibit expression of a target gene by RNA interference (RNAi).

In one embodiment, a siRNA is a small hairpin (also called stem loop) RNA (shRNA). These shRNAs are composed of a short (e.g., 19-25 nucleotides) antisense strand, followed by a 5-9 nucleotide loop, and the complementary sense strand. Alternatively, the sense strand may precede the nucleotide loop structure and the antisense strand may follow. These shRNAs may be contained in plasmids, retroviruses, and lentiviruses.

### CRISPR

By “CRISPR” is meant a set of clustered regularly interspaced short palindromic repeats, or a system comprising such a set of repeats. By “Cas”, as used herein, is meant a CRISPR-associated protein. By “CRISPR/Cas” system is meant a system derived from CRISPR and Cas which can be used to silence, enhance or mutate the CARM1 or CARM1 effector genes.

Naturally-occurring CRISPR/Cas systems are found in approximately 40% of sequenced eubacteria genomes and 90% of sequenced archaea (Grissa et al. 2007. BMC Bioinformatics 8: 172). This system is a type of prokaryotic immune system that confers resistance to foreign genetic elements such as plasmids and phages and provides a form of acquired immunity (Barrangou et al. 2007. Science 315: 1709-1712; Marragini et al. 2008 Science 322: 1843-1845).

The CRISPR/Cas system has been modified for use in gene editing (silencing, enhancing or changing specific genes) in eukaryotes such as mice or primates (Wiedenheft et al. 2012. Nature 482: 331-8). This is accomplished by introducing into the eukaryotic cell a plasmid containing a specifically designed CRISPR and one or more appropriate Cas.

The CRISPR sequence, sometimes called a CRISPR locus, comprises alternating repeats and spacers. In a naturally-occurring CRISPR, the spacers usually comprise sequences foreign to the bacterium such as a plasmid or phage sequence; in the CRISPR/Cas system, the spacers are derived from the CARM1 or CARM1 effector gene sequence. The repeats generally show some dyad symmetry, and may form a secondary structure such as a hairpin, and may or may not be palindromic.

RNA from the CRISPR locus is constitutively expressed and processed by Cas proteins into small RNAs. These processed RNAs comprise a spacer flanked by a repeat sequence. The RNAs guide other Cas proteins to silence exogenous genetic elements at the RNA or DNA level (Horvath et al. 2010. Science 327: 167-170; Makarova et al. 2006 Biology Direct 1: 7). The spacers thus serve as templates for RNA molecules, analogously to siRNAs (Pennisi 2013. Science 341: 833-836).

As these naturally occur in many different types of bacteria, the exact arrangements of the CRISPR and structure, function and number of Cas genes and their product differ somewhat from species to species (Haft et al. 2005 PLoS Comput. Biol. 1: e60; Kunin et al. 2007. Genome Biol. 8: R61; Mojica et al. 2005. J. Mol. Evol. 60: 174-182; Bolotin et al. 2005. Microbiol. 151: 2551-2561; Pourcel et al. 2005. Microbiol. 151: 653-663; and Stern et al. 2010. Trends. Genet. 28: 335-340). For example, the Cse (Cas subtype, E. coli) proteins (e.g., CasA) form a functional complex, Cascade, that processes CRISPR RNA transcripts into spacer-repeat units that Cascade retains (Brouns et al. 2008. Science 321: 960-964). In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing (Pennisi 2013. Science 341: 833-836).

The CRISPR/Cas system can thus be used to edit the CARM1 or CARM1 effector gene (adding or deleting a basepair), e.g., repairing a damaged CARM1 or CARM1 effector gene (e.g., if the damage to CARM1 or CARM1 effector gene results in high or low post-translational modification, production, expression, level, stability or activity of CARM1 or CARM1 effector), or introducing a premature stop which thus decreases expression of an over-expressed CARM1 or CARM1 effector gene. The CRISPR/Cas system can alternatively be used like RNA interference, turning off the CARM1 or CARM1 effector gene in a reversible fashion. In a mammalian cell, for example, the RNA can guide the Cas protein to the CARM1 or CARM1 effector gene promoter, sterically blocking RNA polymerases.

Artificial CRISPR systems can be generated which inhibit CARM1 or CARM1 effector gene, using technology known in the art, e.g., that described in U.S. patent application Ser. No. 13/842,859 (published as US 20140068797). Such CARM1 or CARM1 effector gene-inhibitory CRISPR system can include a guide RNA (gRNA) comprising a CARM1 or CARM1 effector gene-targeting domain, i.e., a nucleotide sequence that is complementary to a CARM1 or CARM1 effector DNA strand, and a second domain that interacts with an RNA-directed nuclease, e.g., cpf1 or Cas molecule, e.g., Cas9 molecule.

In some embodiments, the ability of an RNA-directed nuclease, e.g., cpf1 or Cas molecule, e.g., Cas9 molecule, to interact with and cleave a target nucleic acid is Protospacer Adjacent Motif (PAM) sequence dependent. A PAM sequence is a sequence in the target nucleic acid. In some embodiments, cleavage of the target nucleic acid occurs upstream from the PAM sequence. RNA-directed nuclease molecules, e.g., cpf1 or Cas molecules, e.g., Cas9 molecules, from different bacterial species can recognize different sequence motifs (e.g., PAM sequences). In addition to recognizing different PAM sequences, RNA-directed nucleases, e.g., cpf1 or Cas molecules, e.g., Cas9 molecules, from different species may be directed to different target sequences (e.g., target sequences adjacent, e.g., immediately upstream, to the PAM sequence) by gRNA molecules comprising targeting domains capable of hybridizing to said target sequences and a tracr sequence that binds to said RNA-directed nuclease, e.g., cpf1 or Cas molecule, e.g., Cas9 molecule.

In some embodiments, the CRISPR system comprises a gRNA molecule and a Cas9 molecule from S. pyogenes. A Cas9 molecule of S. pyogenes recognizes the sequence motif NGG and directs cleavage of a target nucleic acid sequence 1 to 10, e.g., 3 to 5, base pairs upstream from that sequence. A gRNA molecule useful with S. pyogenes-based CRISPR systems may include a CARM1 or CARM1 effector gene-targeting sequence, and a tracr sequence known to interact with S. Pyogenes (see, e.g., Mali el al, SCIENCE 2013; 339(6121): 823-826).

In some embodiments, the CRISPR system comprises a gRNA molecule and a Cas9 molecule from S. thermophilus. A Cas9 molecule of S. thermophilus recognizes the sequence motif NGGNG and NNAGAAW (W=A or T) and directs cleavage of a core target nucleic acid sequence 1 to 10, e.g., 3 to 5, base pairs upstream from these sequences. A gRNA molecule useful with S. thermophilus-based CRISPR systems may include a CARM1 or CARM1 effector gene-targeting sequence, and a tracr sequence known to interact with S. thermophilus (see, e.g., Horvath et al., SCIENCE 2010; 327(5962): 167-170, and Deveau et al., J BACTERIOL 2008; 190(4): 1390-1400).

In some embodiments, the CRISPR system comprises a gRNA molecule and a Cas9 molecule from S. aureus. A Cas9 molecule of S. aureus recognizes the sequence motif NNGRR (R=A or G) and directs cleavage of a target nucleic acid sequence 1 to 10, e.g., 3 to 5, base pairs upstream from that sequence. A gRNA molecule useful with S. aureus-based CRISPR systems may include a CARM1 or CARM1 effector gene-targeting sequence, and a tracr sequence known to interact with S. aureus (see, e.g., Ran F. et al., NATURE, vol. 520, 2015, pp. 186-191).

In some embodiments, the CRISPR system comprises a gRNA molecule and a RNA-directed nuclease, e.g., cpf1 molecule, e.g., a cpf1 molecule from Lachnospiraceae bacterium or a cpf1 molecule from Acidaminococcus sp. A cpf1 molecule, e.g., a cpf1 molecule from Lachnospiraceae bacterium or a cpf1 molecule from Acidaminococcus sp., recognizes the sequence motive of TTN (where N=A, T, G or C) or preferably TTTN (where N=A, T, G or C), and directs cleavage of a target nucleic acid sequence 1-25 base pairs upstream of the PAM sequence, e.g., 18-19 base pairs upstream from the PAM sequence on the same strand as the PAM and 23 base pairs upstream of the PAM sequence on the opposite strand as the PAM, creating a sticky end break. A gRNA molecule useful with cpf1-based CRISPR systems (e.g., those utilizing cpf1 molecules from Lachnospiraceae bacterium or Acidaminococcus sp.) may include a CARM1 or CARM1 effector gene-targeting sequence, and a tracr sequence which interacts with cpf1 (see, e.g., Zetsche B. et al., CELL, vol. 163:3, October 2015, 759-771).

### Nucleic Acid and Amino Acid Compositions

Provided herein are nucleic acid sequences for human CARM1, and CARM1 effector genes such as MED12 and TDRD3 (SEQ ID NOs: 10-16). Table 4 provides a list of amino acid sequences for human CARM1, and CARM1 effector genes such as MED12 and TDRD3.

TABLE 4

| Gene | Human Amino Acid Sequence |
|---|---|
| CARM1 | MAAAAAAVGPGAGGAGSAVPGGAGPCATVSVFPGARLLTIGDANGEIQRHAEQQA |
| Isoform 1 | LRLEVRAGPDSAGIALYSHEDVCVFKCSVSRETECSRVGKQSFIITLGCNSVLIQFAT |
| (SEQ ID | PNDFCSFYNILKTCRGHTLERSVFSERTEESSAVQYFQFYGYLSQQQNMMQDYVRT |
| NO. 1) | GTYQRAILQNHTDFKDKIVLDVGCGSGILSFFAAQAGARKIYAVEASTMAQHAEVL |
| | VKSNNLTDRIVVIPGKVEEVSLPEQVDIIISEPMGYMLFNERMLESYLHAKKYLKPS |
| | GNMFPTIGDVHLAPFTDEQLYMEQFTKANFWYQPSFHGVDLSALRGAAVDEYFRQ |
| | PVVDTFDIRILMAKSVKYTVNFLEAKEGDLHRIEIPFKFHMLHSGLVHGLAFWFDV |
| | AFIGSIMTVWLSTAPTEPLTHWYQVRCLFQSPLFAKAGDTLSGTCLLIANKRQSYDI |
| | SIVAQVDQTGSKSSNLLDLKNPFFRYTGTTPSPPPGSHYTSPSENMWNTGSTYNLSS |
| | GMAVAGMPTAYDLSSVIASGSSVGHNNLIPLGSSGAQGSGGGSTSAHYAVNSQFTM |
| | GGPAISMASPMSIPTNTMHYGS (SEQ ID NO. 1) |
| CARM1 | MAAAAAAVGPGAGGAGSAVPGGAGPCATVSVFPGARLLTIGDANGEIQRHAEQQA |
| Isoform 2 | LRLEVRAGPDSAGIALYSHEDVCVFKCSVSRETECSRVGKQSFIITLGCNSVLIQFAT |
| (SEQ ID | PNDFCSFYNILKTCRGHTLERSVFSERTEESSAVQYFQFYGYLSQQQNMMQDYVRT |
| NO. 2) | GTYQRAILQNHTDFKDKIVLDVGCGSGILSFFAAQAGARKIYAVEASTMAQHAEVL |
| | VKSNNLTDRIVVIPGKVEEVSLPEQVDIIISEPMGYMLFNERMLESYLHAKKYLKPS |
| | GNMFPTIGDVHLAPFTDEQLYMEQFTKANFWYQPSFHGVDLSALRGAAVDEYFRQ |
| | PVVDTFDIRILMAKSVKYTVNFLEAKEGDLHSACLASPAATALCLPG (SEQ ID NO. |
| | 2) |
| CARM1 | MAAAAAAVGPGAGGAGSAVPGGAGPCATVSVFPGARLLTIGDANGEIQRHAEQQA |
| Isoform 3 | LRLEVRAGPDSAGIALYSHEDVCVFKCSVSRETECSRVGKQSFIITLGCNSVLIQFAT |
| (SEQ ID | PNDFCSFYNILKTCRGHTLERSVFSERTEESSAVQYFQFYGYLSQQQNMMQDYVRT |
| NO. 3) | GTYQRAILQNHTDFKDKIVLDVGCGSGILSFFAAQAGARKIYAVEASTMAQHAEVL |
| | VKSNNLTDRIVVIPGKVEEVSLPEQVDIIISEPMGYMLFNERMLESYLHAKKYLKPS |
| | GNMFPTIGDVHLAPFTDEQLYMEQFTKANFWYQPSFHGVDLSALRGAAVDEYFRQ |
| | PVVDTFDIRILMAKSVKYTVNFLEAKEGDLHRIEIPFKFHMLHSGLVHGLAFWFDV |
| | AFIGSIMTVWLSTAPTEPLTHWYQVRCLFQSPLFAKAGDTLSGTCLLIANKRQSYDI |
| | SIVAQVDQTGSKSSNLLDLKNPFFRYTGTTPSPPPGSHYTSPSENMWNTGSTYNLSS |
| | GMAVAGMPTAYDLSSVIASGSSVGHNNLIPLANTGIVNHTHSRMGSIMSTGIVQGSS |
| | GAQGSGGGSTSAHYAVNSQFTMGGPAISMASPMSIPTNTMHYGS (SEQ ID NO. 3) |
| TDRD3 | MLRLQMTDGHISCTAVEFSYMSKISLNTPPGTKVKLSGIVDIKNGFLLLNDSNTTVL |
| Isoform 1 | GGEVEHLIEKWELQRSLSKHNRSNIGTEGGPPPFVPFGQKCVSHVQVDSRELDRRK |
| (SEQ ID | TLQVTMPVKPTNDNDEFEKQRTAAIAEVAKSKETKTFGGGGGGARSNLNMNAAG |
| NO. 4) | NRNREVLQKEKSTKSEGKHEGVYRELVDEKALKHITEMGFSKEASRQALMDNGN |
| | NLEAALNVLLTSNKQKPVMGPPLRGRGKGRGRIRSEDEEDLGNARPSAPSTLFDFL |
| | ESKMGTLNVEEPKSQPQQLHQGQYRSSNTEQNGVKDNNHLRHPPRNDTRQPRNE |
| | KPPRFQRDSQNSKSVLEGSGLPRNRGSERPSTSSVSEVWAEDRIKCDRPYSRYDRTK |
| | DTSYPLGSQHSDGAFKKRDNSMQSRSGKGPSFAEAKENPLPQGSVDYNNQKRGKR |
| | ESQTSIPDYFYDRKSQTINNEAFSGIKIEKHFNVNTDYQNPVRSNSFIGVPNGEVEM |
| | PLKGRRIGPIKPAGPVTAVPCDDKIFYNSGPKRRSGPIKPEKILESSIPMEYAKMWKP |
| | GDECFALYWEDNKFYRAEVEALHSSGMTAVVKFIDYGNYEEVLLSNIKPIQTEAWE |
| | EEGTYDQTLEFRRGGDGQPRRSTRPTQQFYQPPRARN (SEQ ID NO. 4) |
| TDRD3 | MLRLQMTDGHISCTAVEFSYMSKISLNTPPGTKVKLSGIVDIKNGFLLLNDSNTTVL |
| Isoform 2 | GGEVEHLIEKWELQRSLSKHNRSNIGTEGGPPPFVPFGQCVSHVQVDSRELDRRKT |
| (SEQ ID | LQVTMPVKPTNDNDEFEKQRTAAIAEVAKSKETKTFGGGGGGARSNLNMNAAGN |
| NO. 5) | RNREVLQKEKSTKSEGKHEGVYRELVDEKALKHITEMGFSKEASRQALMDNGNN |
| | LEAALNVLLTSNKQKPVMGPPLRGRGKGRGRIRSEDEEDLGNARPSAPSTLFDFLE |
| | SKMGTLNVEEPKSQPQQLHQGQYRSSNTEQNGVKDNNHLRHPPRNDTRQPRNEK |
| | PPRFQRDSQNSKSVLEGSGLPRNRGSERPSTSSVSEVWAEDRIKCDRPYSRYDRTKD |
| | TSYPLGSQHSDGAFKKRDNSMQSRSGKGPSFAEAKENPLPQGSVDYNNQKRGKRE |
| | SQTSIPDYFYDRKSQTINNEAFSGIKIEKHFNVNTDYQNPVRSNSFIGVPNGEVEMP |
| | LKGRRIGPIKPAGPVTAVPCDDKIFYNSGPKRRSGPIKPEKILESSIPMEYAKMWKPG |
| | DECFALYWEDNKFYRAEVEALHSSGMTAVVKFIDYGNYEEVLLSNIKPIQTEAWEE |
| | EGTYDQTLEFRRGGDGQPRRSTRPTQQFYQPPRARN (SEQ ID NO. 5) |
| TDRD3 | MAQVAGAALSQAGWYLSDEGIEACTSSPDKVNVNDIILIALNTDLRTIGKKFLPSDI |
| Isoform 3 | NSGKVEKLEGPCVLQIQKIRNVAAPKDNEESQAAPRMLRLQMTDGHISCTAVEFSY |
| (SEQ ID | MSKISLNTPPGTKVKLSGIVDIKNGFLLLNDSNTTVLGGEVEHLIEKWELQRSLSKH |
| NO. 6) | NRSNIGTEGGPPPFVPFGQKCVSHVQVDSRELDRRKTLQVTMPVKPTNDNDEFEK |
| | QRTAAIAEVAKSKETKTFGGGGGGARSNLNMNAAGNRNREVLQKEKSTKSEGKH |
| | EGVYRELVDEKALKHITEMGFSKEASRQALMDNGNNLEAALNVLLTSNKQKPVM |
| | GPPLRGRGKGRGRIRSEDEEDLGNARPSAPSTLFDFLESKMGTLNVEEPKSQPQQL |
| | HQGQYRSSNTEQNGVKDNNHLRHPPRNDTRQPRNEKPPRFQRDSQNSKSVLEGSG |
| | LPRNRGSERPSTSSVSEVWAEDRIKCDRPYSRYDRTKDTSYPLGSQHSDGAFKKRD |
| | NSMQSRSGKGPSFAEAKENPLPQGSVDYNNQKRGKRESQTSIPDYFYDRKSQTINN |
| | EAFSGIKIEKHFNVNTDYQNPVRSNSFIGVPNGEVEMPLKGRRIGPIKPAGPVTAVPC |
| | DDKIFYNSGPKRRSGPIKPEKILESSIPMEYAKMWKPGDECFALYWEDNKFYRAEV |
| | EALHSSGMTAVVKFIDYGNYEEVLLSNIKPIQTEAWEEEGTYDQTLEFRRGGDGQP |
| | RRSTRPTQQFYQPPRARN (SEQ ID NO. 6) |
| MED12 | MAAFGILSYEHRPLKRPRLGPPDVYPQDPKQKEDELTALNVKQGFNNQPAVSGDE |
| Isoform 1 | HGSAKNVSFNPAKISSNFSSIIAEKLRCNTLPDTGRRKPQVNQKDNFWLVTARSQSA |
| (MED12 | INTWFTDLAGTKPLTQLAKKVPIFSKKEEVFGYLAKYTVPVMRAAWLIKMTCAYY |
| SEQ ID | AAISETKVKKRHVDPFMEWTQIITKYLWEQLQKMAEYYRPGPAGSGGCGSTIGPLP |
| NO. 7) | HDVEVAIRQWDYTEKLAMFMFQDGMLDRHEFLTWVLECFEKIRPGEDELLKLLLP |
| | LLLRYSGEFVQSAYLSRRLAYFCTRRLALQLDGVSSHSSHVISAQSTSTLPTTPAPQP |
| | PTSSTPSTPFSDLLMCPQHRPLVFGLSCILQTILLCCPSALVWHYSLTDSRIKTGSPLD |
| | HLPIAPSNLPMPEGNSAFTQQVRAKLREIEQQIKERGQAVEVRWSFDKCQEATAGF |
| | TIGRVLHTLEVLDSHSFERSDFSNSLDSLCNRIFGLGPSKDGHEISSDDDAVVSLLCE |
| | WAVSCKRSGRHRAMVVAKLLEKRQAEIEAERCGESEAADEKGSIASGSLSAPSAPIF |
| | QDVLLQFLDTQAPMLTDPRSESERVEFFNLVLLFCELIRHDVFSHNMYTCTLISRGD |
| | LAFGAPGPRPPSPFDDPADDPEHKEAEGSSSSKLEDPGLSESMDIDPSSSVLFEDME |
| | KPDFSLFSPTMPCEGKGSPSPEKPDVEKEVKPPPKEKIEGTLGVLYDQPRHVQYATH |
| | FPIPQEESCSHECNQRLVVLFGVGKQRDDARHAIKKITKDILKVLNRKGTAETDQL |
| | APIVPLNPGDLTFLGGEDGQKRRRNRPEAFPTAEDIFAKFQHLSHYDQHQVTAQVS |
| | RNVLEQITSFALGMSYHLPLVQHVQFIFDLMEYSLSISGLIDFAIQLLNELSVVEAEL |
| | LLKSSDLVGSYTTSLCLCIVAVLRHYHACLILNQDQMAQVFEGLCGVVKHGMNRS |
| | DGSSAERCILAYLYDLYTSCSHLKNKFGELFSDFCSKVKNTIYCNVEPSESNMRWAP |
| | EFMIDTLENPAAHTFTYTGLGKSLSENPANRYSFVCNALMHVCVGHHDPDRVNDI |
| | AILCAELTGYCKSLSAEWLGVLKALCCSSNNGTCGFNDLLCNVDVSDLSFHDSLAT |
| | FVAILIARQCLLLEDLIRCAAIPSLLNAACSEQDSEPGARLTCRILLHLFKTPQLNPCQ |
| | SDGNKPTVGIRSSCDRHLLAASQNRIVDGAVFAVLKAVFVLGDAELKGSGFTVTGG |
| | TEELPEEEGGGGSGGRRQGGRNISVETASLDVYAKYVLRSICQQEWVGERCLKSLC |
| | EDSNDLQDPVLSSAQAQRLMQLICYPHRLLDNEDGENPQRQRIKRILQNLDQWTM |
| | RQSSLELQLMIKQTPNNEMNSLLENIAKATIEVFQQSAETGSSSGSTASNMPSSSKT |
| | KPVLSSLERSGVWLVAPLIAKLPTSVQGHVLKAAGEELEKGQHLGSSSRKERDRQK |
| | QKSMSLLSQQPFLSLVLTCLKGQDEQREGLLTSLYSQVHQIVNNWRDDQYLDDCK |
| | PKQLMHEALKLRLNLVGGMFDTVQRSTQQTTEWAMLLLEIIISGTVDMQSNNELF |
| | TTVLDMLSVLINGTLAADMSSISQGSMEENKRAYMNLAKKLQKELGERQSDSLEK |
| | VRQLLPLPKQTRDVITCEPQGSLIDTKGNKIAGFDSIFKKEGLQVSTKQKISPWDLF |
| | EGLKPSAPLSWGWFGTVRVDRRVARGEEQQRLLLYHTHLRPRPRAYYLEPLPLPPE |
| | DEEPPAPTLLEPEKKAPEPPKTDKPGAAPPSTEERKKKSTKGKKRSQPATKTEDYG |
| | MGPGRSGPYGVTVPPDLLHHPNPGSITHLNYRQGSIGLYTQNQPLPAGGPRVDPYR |
| | PVRLPMQKLPTRPTYPGVLPTTMTGVMGLEPSSYKTSVYRQQQPAVPQGQRLRQQ |
| | LQQSQGMLGQSSVHQMTPSSSYGLQTSQGYTPYVSHVGLQQHTGPAGTMVPPSYS |
| | SQPYQSTHPSTNPTLVDPTRHLQQRPSGYVHQQAPTYGHGLTSTQRFSHQTLQQTP |
| | MISTMTPMSAQGVQAGVRSTAILPEQQQQQQQQQQQQQQQQQQQQQQQQQQYH |
| | IRQQQQQQILRQQQQ0QQQQQQQQQQ0QQQQQQQQQQQHQQQQQQQAAPPQPQP |
| | QSQPQFQRQGLQQTQQQQQTAALVRQLQQQLSNTQPQPSTNIFGRY (SEQ ID NO. |
| | 7) |
| MED12 | MAAFGILSYEHRPLKRPRLGPPDVYPQDPKQKEDELTALNVKQGFNNQPAVSGDE |
| Isoform 2 | HGSAKNVSFNPAKISSNFSSIIAEKLRCNTLPDTGRRKPQVNQKDNFWLVTARSQSA |
| (MED12 | INTWFTDLAGTKPLTQLAKKVPIFSKKEEVFGYLAKYTVPVMRAAWLIKMTCAYY |
| SEQ ID | AAISETKVKKRHVDPFMEWTQIITKYLWEQLQKMAEYYRPGPAGSGGCGSTIGPLP |
| NO. 8) | HDVEVAIRQWDYTEKLAMFMFQDGMLDRHEFLTWVLECFEKIRPGEDELLKLLLP |
| | LLLRYSGEFVQSAYLSRRLAYFCTRRLALQLDGVSSHSSHVISAQSTSTLPTTPAPQP |
| | PTSSTPSTPFSDLLMCPQHRPLVFGLSCILQTILLCCPSALVWHYSLTDSRIKTGSPLD |
| | HLPIAPSNLPMPEGNSAFTQQVRAKLREIEQQIKERGQAVEVRWSFDKCQEATAGF |
| | TIGRVLHTLEVLDSHSFERSDFSNSLDSLCNRIFGLGPSKDGHEISSDDDAVVSLLCE |
| | WAVSCKRSGRHRAMVVAKLLEKRQAEIEAERCGESEAADEKGSIASGSLSAPSAPIF |
| | QDVLLQFLDTQAPMLTDPRSESERVEFFNLVLLFCELIRHDVFSHNMYTCTLISRGD |
| | LAFGAPGPRPPSPFDDPADDPEHKEAEGSSSSKLEDPGLSESMDIDPSSSVLFEDME |
| | KPDFSLFSPTMPCEGKGSPSPEKPDVEKEVKPPPKEKIEGTLGVLYDQPRHVQYATH |
| | FPIPQEESCSHECNQRLVVLFGVGKQRDDARHAIKKITKDILKVLNRKGTAETDQL |
| | APIVPLNPGDLTFLGGEDGQKRRRNRPEAFPTAEDIFAKFQHLSHYDQHQVTAQVS |
| | RNVLEQITSFALGMSYHLPLVQHVQFIFDLMEYSLSISGLIDFAIQLLNELSVVEAEL |
| | LLKSSDLVGSYTTSLCLCIVAVLRHYHACLILNQDQMAQVFEGLCGVVKHGMNRS |
| | DGSSAERCILAYLYDLYTSCSHLKNKFGELFSDFCSKVKNTIYCNVEPSESNMRWAP |
| | EFMIDTLENPAAHTFTYTGLGKSLSENPANRYSFVCNALMHVCVGHHDPDRVNDI |
| | AILCAELTGYCKSLSAEWLGVLKALCCSSNNGTCGFNDLLCNVDVSDLSFHDSLAT |
| | FVAILIARQCLLLEDLIRCAAIPSLLNAACSEQDSEPGARLTCRILLHLFKTPQLNPCQ |
| | SDGNKPTVGIRSSCDRHLLAASQNRIVDGAVFAVLKAVFVLGDAELKGSGFTVTGG |
| | TEELPEEEGGGGSGGRRQGGRNISVETASLDVYAKYVLRSICQQEWVGERCLKSLC |
| | EDSNDLQDPVLSSAQAQRLMQLICYPHRLLDNEDGENPQRQRIKRILQNLDQWTM |
| | RQSSLELQLMIKQTPNNEMNSLLENIAKATIEVFQQSAETGSSSGSTASNMPSSSKT |
| | KPVLSSLERSGVWLVAPLIAKLPTSVQGHVLKAAGEELEKGQHLGSSSRKERDRQK |
| | QKSMSLLSQQPFLSLVLTCLKGQDEQREGLLTSLYSQVHQIVNNWRDDQYLDDCK |
| | PKQLMHEALKLRLNLVGGMFDTVQRSTQQTTEWAMLLLEIIISGTVDMQSNNELF |
| | TTVLDMLSVLINGTLAADMSSISQGSMEENKRAYMNLAKKLQKELGERQSDSLEK |
| | VRQLLPLPKQTRDVITCEPQGSLIDTKGNKIAGFDSIFKKEGLQVSTKQKISPWDLF |
| | EGLKPSAPLSWGWFGTVRVDRRVARGEEQQRLLLYHTHLRPRPRAYYLEPLPLPPE |
| | DEEPPAPTLLEPEKKAPEPPKTDKPGAAPPSTEERKKKSTKGKKRSQPATKTEDYG |
| | MGPGRSGPYGVTVPPDLLHHPNPGSITHLNYRQGSIGLYTQNQPLPAGGPRVDPYR |
| | PVRLPMQKLPTRPTYPGVLPTTMTGVMGLEPSSYKTSVYRQQQPAVPQGQRLRQQ |
| | LQAKIQSQGMLGQSSVHQMTPSSSYGLQTSQGYTPYVSHVGLQQHTGPAGTMVPP |
| | SYSSQPYQSTHPSTNPTLVDPTRHLQQRPSGYVHQQAPTYGHGLTSTQRFSHQTLQ |
| | QTPMISTMTPMSAQGVQAGVRSTAILPEQQQQQQQQQQQQQQQQQQQQQQQQQ |
| | QYHIRQQQQQQILRQQQQQQQQQQQQQQQQQQQQQQQQQQHQQQQQQQAAPP |
| | QPQPQSQPQFQRQGLQQTQQQQQTAALVRQLQQQLSNTQPQPSTNIFGRY (SEQ ID |
| | NO. 8) |
| MED12 | MAAFGILSYEHRPLKRPRLGPPDVYPQDPKQKEDELTALNVKQGFNNQPAVSGDE |
| Isoform 3 | HGSAKNVSFNPAKISSNFSSIIAEKLRCNTLPDTGRRKPQVNQKDNFWLVTARSQSA |
| (MED12 | INTWFTDLAGTKPLTQLAKKVPIFSKKEEVFGYLAKYTVPVMRAAWLIKMTCAYY |
| SEQ ID | AAISETKVKKRHVDPFMEWTQIITKYLWEQLQKMAEYYRPGPAGSGGCGSTIGPLP |
| NO. 9) | HDVEVAIRQWDYTEKLAMFMFQDGMLDRHEFLTWVLECFEKIRPGEDELLKLLLP |
| | LLLRYSGEFVQSAYLSRRLAYFCTRRLALQLDGVSSHSSHVISAQSTSTLPTTPAPQP |
| | PTSSTPSTPFSDLLMCPQHRPLVFGLSCILQTILLCCPSALVWHYSLTDSRIKTGSPLD |
| | HLPIAPSNLPMPEGNSAFTQQVRAKLREIEQQIKERGQAVEVRWSFDKCQEATAGF |
| | TIGRVLHTLEVLDSHSFERSDFSNSLDSLCNRIFGLGPSKDGHEISSDDDAVVSLLCE |
| | WAVSCKRSGRHRAMVVAKLLEKRQAEIEAERCGESEAADEKGSIASGSLSAPSAPIF |
| | QDVLLQFLDTQAPMLTDPRSESERVEFFNLVLLFCELIRHDVFSHNMYTCTLISRGD |
| | LAFGAPGPRPPSPFDDPADDPEHKEAEGSSSSKLEDPGLSESMDIDPSSSVLFEDME |
| | KPDFSLFSPTMPCEGKGSPSPEKPDVEKEVKPPPKEKIEGTLGVLYDQPRHVQYATH |
| | FPIPQEESCSHECNQRLVVLFGVGKQRDDARHAIKKITKDILKVLNRKGTAETDQL |
| | APIVPLNPGDLTFLGGEDGQKRRRNRPEAFPTAEDIFAKFQHLSHYDQHQVTAQVS |
| | RNVLEQITSFALGMSYHLPLVQHVQFIFDLMEYSLSISGLIDFAIQLLNELSVVEAEL |
| | LLKSSDLVGSYTTSLCLCIVAVLRHYHACLILNQDQMAQVFEGLCGVVKHGMNRS |
| | DGSSAERCILAYLYDLYTSCSHLKNKFGELFSDFCSKVKNTIYCNVEPSESNMRWAP |
| | EFMIDTLENPAAHTFTYTGLGKSLSENPANRYSFVCNALMHVCVGHHDPDRVNDI |
| | AILCAELTGYCKSLSAEWLGVLKALCCSSNNGTCGFNDLLCNVDVSDLSFHDSLAT |
| | FVAILIARQCLLLEDLIRCAAIPSLLNAACSEQDSEPGARLTCRILLHLFKTPQLNPCQ |
| | SDGNKPTVGIRSSCDRHLLAASQNRIVDGAVFAVLKAVFVLGDAELKGSGFTVTGG |
| | TEELPEEEGGGGSGGRRQGGRNISVETASLDVYAKYVLRSICQQEWVGERCLKSLC |
| | EDSNDLQDPVLSSAQAQRLMQLICYPHRLLDNEDGENPQRQRIKRILQNLDQWTM |
| | RQSSLELQLMIKQTPNNEMNSLLENIAKATIEVFQQSAETGSSSGSTASNMPSSSKT |
| | KPVLSSLERSGVWLVAPLIAKLPTSVQGHVLKAAGEELEKGQHLGSSSRKERDRQK |
| | QKSMSLLSQQPFLSLVLTCLKGQDEQREGLLTSLYSQVHQIVNNWRDDQYLDDCK |
| | PKQLMHEALKLRLNLVGGMFDTVQRSTQQTTEWAMLLLEIIISGTVDMQSNNELF |
| | TTVLDMLSVLINGTLAADMSSISQGSMEENKRAYMNLAKKLQKELGERQSDSLEK |
| | VRQLLPLPKQTRDVITCEPQGSLIDTKGNKIAGFDSIFKKEGLQVSTKQKISPWDLF |
| | EGLKPSAPLSWGWFGTVRVDRRVARGEEQQRLLLYHTHLRPRPRAYYLEPLPLPPE |
| | DEEPPAPTLLEPEKKAPEPPKTDKPGAAPPSTEERKKKSTKGKKRSQPATKTEDYG |
| | MGPGRSGPYGVTVPPDLLHHPNPGSITHLNYRQGSIGLYTQNQPLPAGGPRVDPYR |
| | PVRLPMQKLPTRPTYPGVLPTTMTGVMGLEPSSYKTSVYRQQQPAVPQGQRLRQQ |
| | LQSQGMLGQSSVHQMTPSSSYGLQTSQGYTPYVSHVGLQQHTGPAGTMVPPSYSS |
| | QPYQSTHPSTNPTLVDPTRHLQQRPSGYVHQQAPTYGHGLTSTQRFSHQTLQQTPM |
| | ISTMTPMSAQGVQAGVRSTAILPEQQQQQQQQQQQQQQQQQQQQQQQQQQYHIR |
| | QQQQQQILRQQQQQQQQQQQQQQQQQQQQQQQQQQHQQQQQQQAAPPQPQPQ |
| | SQPQFQRQGLQQTQQQQQTAALVRQLQQQLSNTQPQPSTNIFGRY (SEQ ID NO. 9) |

### Chimeric Antigen Receptors

In some instances, the present disclosure provides chimeric antigen receptors (CARs) comprising an antigen binding domain directed to a tumor cell antigen. A CAR is an artificially constructed hybrid protein or polypeptide containing an extracellular portion that recognizes a tumor cell antigen (e.g., the antigen binding domains of an antibody (scFv) and a cytoplasmic signaling domain derived from the T cell receptor and costimulatory domain. (Kalos M, et al., Sci Transl Med. 2011 Aug. 10; 3(95)). Kalos et al. describes the generation of CAR T cells that target CD19 and demonstrates the CAR modified T-cells mediated potent antitumor effect in chronic lymphocytic leukemia patients. Characteristics of CARs include their ability to redirect T-cell specificity and reactivity toward a selected target in a non-MHC-restricted manner, exploiting the antigen-binding properties of monoclonal antibodies. The CAR-modified T-cells have the potential to replicate in vivo and long-term persistence allows for sustained tumor control and obviate the need for repeated infusions of antibody. (Kalos M, et al., Sci Transl Med. 2011 Aug. 10; 3(95)). The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains. CAR-modified T cells are described in detail in WO2014/201021, WO2012/079000 and WO2012/09999 and in Milone et al. 2009 Mol. Ther. 17:1453, which are hereby incorporated by reference.

A CAR combines the binding site of a molecule that recognizes an antigen being targeted (i.e., an “antigen binding domain”) with one or more domains of conventional immune receptors responsible for initiating signal transduction that leads to lymphocyte activation (e.g., the “stimulatory domain” or “signaling domain”).

In some embodiments, the binding portion used is derived from the structure of the Fab (antigen binding) fragment of a monoclonal antibody (mAb) that has high affinity for the tumor antigen being targeted. Because the Fab is the product of two genes, the corresponding sequences are usually combined via a short linker fragment that allows the heavy-chain to fold over the light-chain derived peptides into their native configuration, creating a single-chain fragment variable (scFv) region.

Fv or (scFv) antibody fragments comprise the VH and VL domains of antibody, wherein these domains are present in a single polypeptide chain. Generally the Fv polypeptide further comprises a polypeptide linker between the VH and VL domains, which enables the scFv to form the desired structure for antigen binding.

In some embodiments, the binding portion used is derived from a cytoplasmic signaling domain derived from T cell receptor and costimulatory molecules.

In some embodiments, the signaling portion of CARs contains usually the intracellular domains of the zeta (ζ) chain of the TCR/CD3 complex or, less commonly, of the gamma (γ) chain of the immunoglobulin receptor FcεRI, or the CD3-epsilon (ε) chain, with the transmembrane region being derived from the same molecule.

In some aspects, the CARs comprise an antigen binding domain, a transmembrane domain, a stimulatory domain, and a co-stimulatory domain. Further embodiments of the disclosure provide related nucleic acids, recombinant expression vectors, host cells, populations of cells, antibodies, or antigen binding portions thereof, and pharmaceutical compositions relating to the CARs of the disclosure.

In one aspect, the antigen binding domain binds to a tumor cell antigen. The term “tumor cell antigen” or “tumor antigen” as used herein refers to any polypeptide expressed by a tumor that is capable of inducing an immune response. Non-limiting examples of tumor antigens include, for example, prostate-specific membrane antigen (PSMA), Carcinoembryonic Antigen (CEA), CD19, CD20, CD22, ROR1, mesothelin, CD333/IL3Ra, c-Met, Glycolipid F77, EGFRvIII, GD-2, NY-ESO-1 TCR, ERBB2, BIRC5, CEACAM5, WDR46, BAGE, CSAG2, DCT, MAGED4, GAGE1, GAGE2, GAGE3, GAGE4, GAGE5, GAGE6, GAGE7, GAGE8, IL13RA2, MAGEA1, MAGEA2, MAGEA3, MAGEA4, MAGEA6, MAGEA9, MAGEA10, MAGEA12, MAGEB1, MAGEB2, MAGEC2, TP53, TYR, TYRP1, SAGE1, SYCP1, SSX2, SSX4, KRAS, PRAME, NRAS, ACTN4, CTNNB1, CASP8, CDC27, CDK4, EEF2, FN1, HSPA1B, LPGAT1, ME1, HHAT, TRAPPC1, MUM3, MYO1B, PAPOLG, OS9, PTPRK, TPI1, ADFP, AFP, AIM2, ANXA2, ART4, CLCA2, CPSF1, PPIB, EPHA2, EPHA3, FGF5, CA9, TERT, MGAT5, CEL, F4.2, CAN, ETV6, BIRC7, CSF1, OGT, MUC1, MUC2, MUM1, CTAG1A, CTAG2, CTAG, MRPL28, FOLH1, RAGE, SFMBT1, KAAG1, SART1, TSPYL1, SART3, SOX10, TRG, WT1, TACSTD1, SILV, SCGB2A2, MC1R, MLANA, GPR143, OCA2, KLK3, SUPT7L, ARTC1, BRAF, CASP5, CDKN2A, UBXD5, EFTUD2, GPNMB, NFYC, PRDX5, ZUBR1, SIRT2, SNRPD1, HERV-K-MEL, CXorf61, CCDC110, VENTXP1, SPA17, KLK4, ANKRD30A, RAB38, CCND1, CYP1B1, MDM2, MMP2, ZNF395, RNF43, SCRN1, STEAP1, 707-AP, TGFBR2, PXDNL, AKAP13, PRTN3, PSCA, RHAMM, ACPP, ACRBP, LCK, RCVRN, RPS2, RPL10A, SLC45A3, BCL2L1, DKK1, ENAH, CSPG4, RGS5, BCR, BCR-ABL, ABL-BCR, DEK, DEK-CAN, ETV6-AML1, LDLR-FUT, NPM1-ALK1, PML-RARA, SYT-SSX1, SYT-SSX2, FLT3, ABL1, AML1, LDLR, FUT1, NPM1, ALK, PML1, RARA, SYT, SSX1, MSLN, UBE2V1, HNRPL, WHSC2, EIF4EBP1, WNK2, OAS3, BCL-2, MCL1, CTSH, ABCC3, BST2, MFGE8, TPBG, FMOD, XAGE1, RPSA, COTL1, CALR3, PA2G4, EZH2, FMNL1, HPSE, APC, UBE2A, BCAP31, TOP2A, TOP2B, ITGB8, RPA1, ABI2, CCNI, CDC2, SEPT2, STAT1, LRP1, ADAM17, JUP, DDR1, ITPR2, HMOX1, TPM4, BAAT, DNAJC8, TAPBP, LGALS3BP, PAGE4, PAK2, CDKN1A, PTHLH, SOX2, SOX11, TRPM8, TYMS, ATIC, PGK1, SOX4, TOR3A, TRGC2, BTBD2, SLBP, EGFR, IER3, TTK, LY6K, IGF2BP3, GPC3, SLC35A4, HSMD, H3F3A, ALDH1A1, MFI2, MMP14, SDCBP, PARP12, MET, CCNB1, PAX3-FKHR, PAX3, FOXO1, XBP1, SYND1, ETV5, HSPA1A, HMHA1, TRIM68 and any combination thereof.

T cells expressing a CAR are generally referred to as CAR T cells. T cells expressing a CAR are referred to herein as CAR T cells or CAR modified T cells. In some embodiments, the cell can be genetically modified to stably express an antibody binding domain on its surface, conferring novel antigen specificity to various cancer cells. In some instances, the T cell is genetically modified to stably express a CAR that combines an antigen recognition domain of a specific antibody with an intracellular stimulatory domain (e.g., signaling domain). Thus, in addition to an antigen binding domain the CAR can include the intracellular domains of the zeta (ζ) chain of the TCR/CD3 complex, the gamma (γ) chain of the immunoglobulin receptor FcεRI26, 27 or the CD3-epsilon (ε) chain, the CAR can also include a transmembrane region being from the same molecules or other type I transmembrane proteins such as CD4, CD8 and CD28.

In one embodiment, the CAR of the disclosure comprises an extracellular domain having an antigen recognition domain, a transmembrane domain, and a cytoplasmic domain.

In one embodiment, the transmembrane domain that naturally is associated with one of the domains in the CAR is used. In another embodiment, the cytoplasmic domain can be designed to comprise a stimulatory domain and a costimulatory domain.

A CAR can include intracytoplasmatic portion of co-stimulatory molecules, such as CD28, CD134/OX40, CD137/4-1BB, Lck, ICOS or DAP10.

The present disclosure also relates to a strategy of Adoptive cell therapy (ACT). ACT is a procedure in which therapeutic lymphocytes are administered to patients in order to treat cancer. This approach entails the ex vivo generation of tumor specific T cell lymphocytes and infusing them to patients. In addition to the lymphocyte infusion, the host may be manipulated in other ways which support the take of the T cells and their immune response, for example, preconditioning the host (with radiation or chemotherapy) and administration of lymphocyte growth factors (such as IL-2). One method for generating such tumor specific lymphocytes involves the expansion of antigen specific T cells. In some embodiments, the antigen can be an antigen present in a cancer cell, a cancer cell, a cancer cell fragment, a tumor antigen, α-galcer, anti-CD3, anti-CD28, anti-IgM, anti-CD40, a pathogen, an attenuated pathogen, or a portion thereof.

In one embodiment, the present disclosure provides generating T cells with reduced expression of Carm1 gene and/or activity of Carm1 protein as described herein and a desired CAR directed to a tumor antigen. The modified T cells can be generated by introducing a vector (e.g., plasmid, lentiviral vector, retroviral vector, adenoviral vector, adeno-associated viral vector) encoding a desired CAR into the cells. In another embodiment, T cells include one or more inhibiting agents that inhibit the expression of Carm1 gene and/or activity of Carm1 protein, and a desired CAR directed to a tumor antigen. The modified T cells are able to replicate in vivo resulting in long term persistence that can lead to tumor control.

In one aspect, the present disclosure provides methods of treating cancer comprising administering a composition capable of silencing genes that inhibit T cell function. In one embodiment, the methods relate to administering T cell with reduced expression of Carm1 gene and/or activity of Carm1 protein and a desired CAR directed to a tumor antigen.

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

The disclosure will be further described in the following examples, which do not limit the scope of the invention described in the claims.

### EXAMPLES

### Example 1. Discovery of Carm1 As A Negative Regulator Of Tumor-Infiltrating T Cells

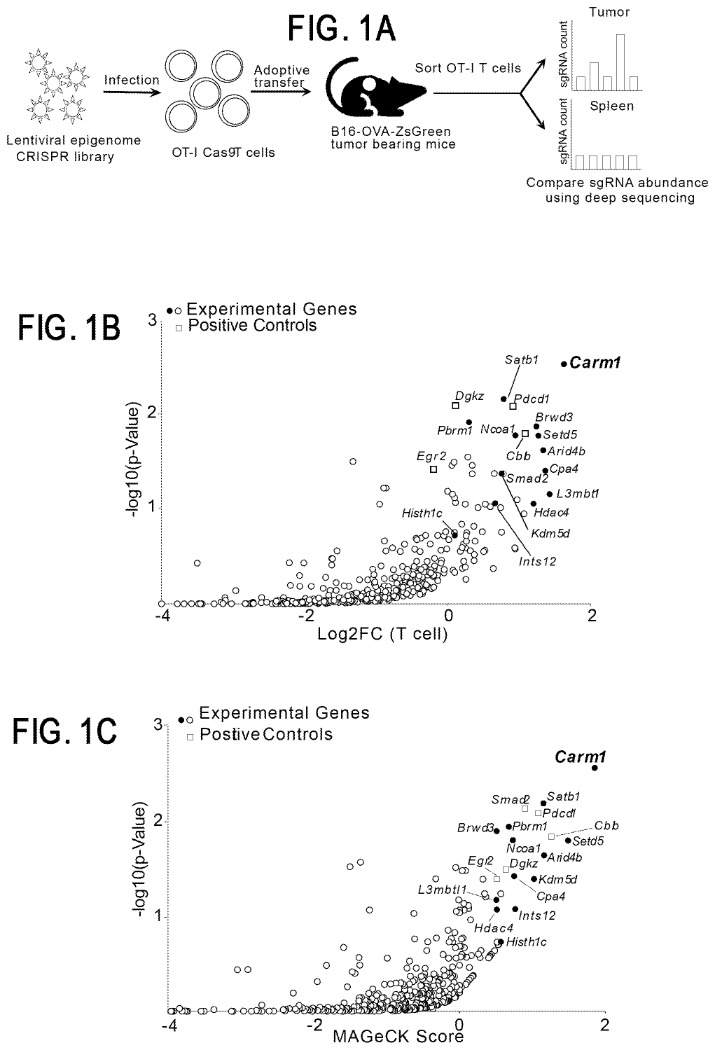

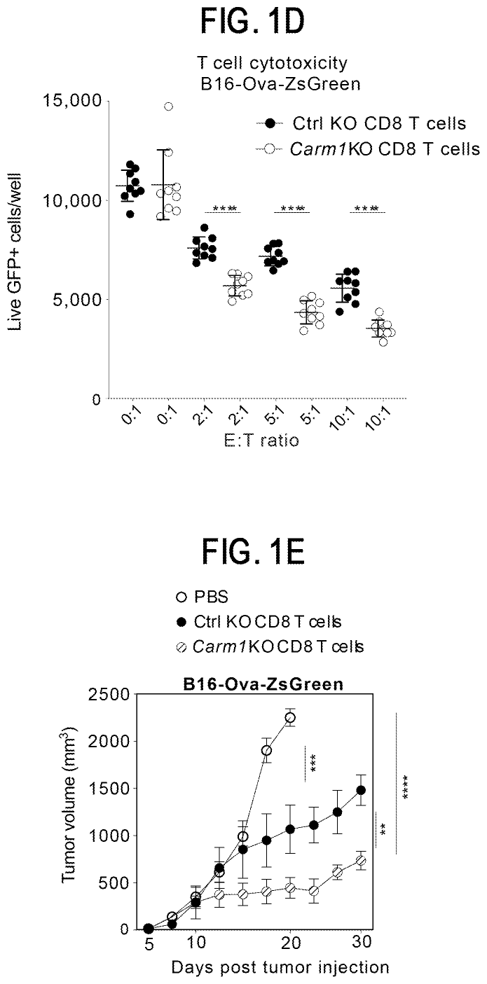

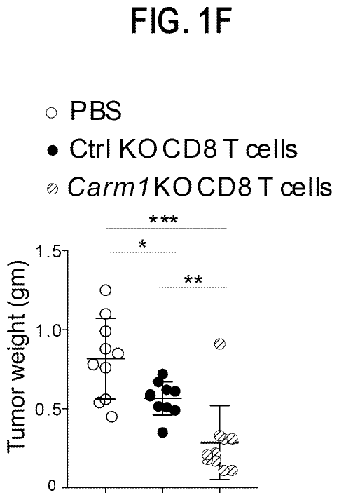

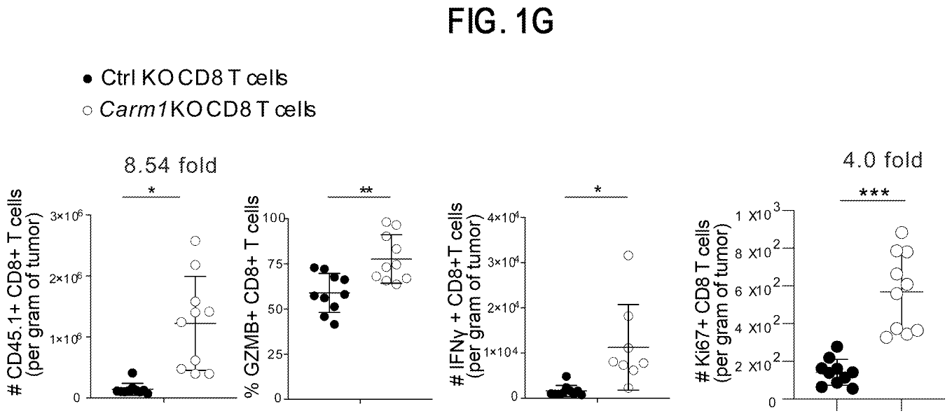

An in vivo CRISPR/Cas9 screen in tumor-specific T cells was performed to discover negative regulators of anti-tumor immunity. A gRNA library targeting epigenetic regulators was delivered into CD8 T cells using a lentiviral vector. These T cells originated from mice transgenic for Cas9 and the OT-I T cell receptor (TCR), thus yielding a gene-edited library of T cells with defined antigen specificity (FIG. 1A). A total of 426 genes representing annotated epigenetic regulators were evaluated using three gRNA pools (5 gRNAs/gene plus 100 negative control gRNAs). Edited T cells were transferred to immunocompetent mice bearing subcutaneous B16F10 melanomas which expressed the Ova antigen recognized by the OT-I TCR. Targeting of key negative regulators enhanced T cell proliferation/survival within tumors and thus resulted in enrichment of the corresponding gRNAs. Representation of gRNAs was quantified after 10 days by deep sequencing of the gRNA cassette in T cells isolated from tumors or a control organ (spleen). gRNAs targeting the positive control genes Pdcd1 and Cblb (encoding PD-1 and Cbl-b proteins, respectively) were among the top enriched gRNAs in each of the three pools, demonstrating that key negative regulators could be reproducibly identified (FIGS. 1B-1C). The top hit from the primary screen was the Carm1 gene, which encodes an arginine methyltransferase that introduces asymmetric dimethylation of histone H3 (H3R17 and H3R26 residues) and other nuclear proteins. This effect was specific for Carm1 and not seen for genes encoding other arginine methyltransferases: while gRNAs targeting Carm1 were enriched tumor-infiltrating T cells relative to spleen, gRNAs targeting Prmt1, Prmt2, Prmt5 and Prmt6 were depleted; also, no enrichment was observed for gRNAs targeting Prmt3, Prmt7 and Prmt8. These results were validated using a targeted gRNA library representing 31 top candidate genes, two positive control genes (Pdcd1 and Cblb) and a set of control gRNAs. Carm1 was again identified as the top hit in this validation screen (FIG. 8A).

Carm1-KO T cells were generated for functional experiments by electroporation of OT-I T cells with ribonucleoprotein complexes (RNPs) composed of Cas9 protein and bound gRNAs. This transient editing procedure was effective, as shown by sequencing of genomic DNA and loss of Carm1 protein following editing with two different gRNAs (FIGS. 8B-8C). A cytotoxicity assay demonstrated that Carm1-KO T cells were more effective in killing B16F10-Ova melanoma cells compared to control edited T cells (FIG. 1D and FIGS. 8D-8F). Following co-culture with B16F10-Ova tumor cells, Carm1-KO compared to control-KO T cells expressed higher levels of the CD69 activation marker, the granzyme B cytotoxicity protein and the cytokines IL-2, IFNγ and TNFα (FIGS. 8G-8H) and showed enhanced antigen-induced proliferation (FIG. 8I). These data demonstrated that Carm1 was a negative regulator of tumor-specific T cells.

### Example 2. Carm1 Inhibition in CD8 T Cells Enhances Their Anti-Tumor Function

Carm1-KO T cells were found to confer more effective anti-tumor immunity than control-KO CD8 T cells against B16F10-OVA tumors (FIGS. 1E-1F and FIG. 9A). Flow cytometry analysis revealed greatly enhanced tumor infiltration by Carm1-KO compared to control-KO CD45.1+CD8+ T cells, including increased accumulation of T cells that expressed the effector molecules granzyme B and IFNγ as well as the proliferation marker Ki67 (FIG. 1G and FIG. 9B). Enhanced anti-tumor immunity by Carm1-KO T cells was confirmed using a second gRNA (FIGS. 9C-9D).

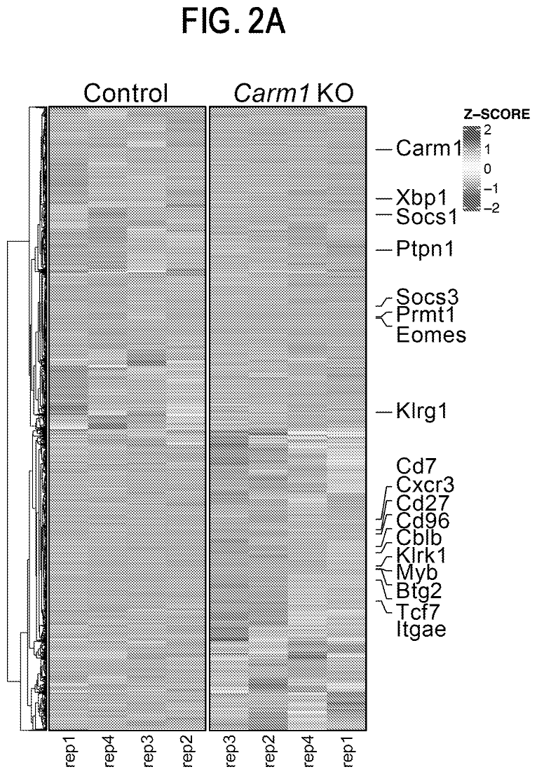

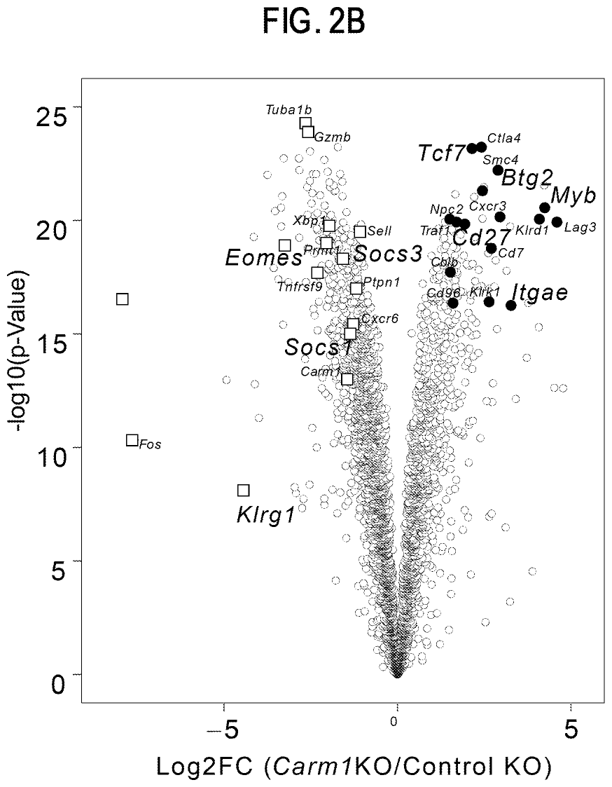

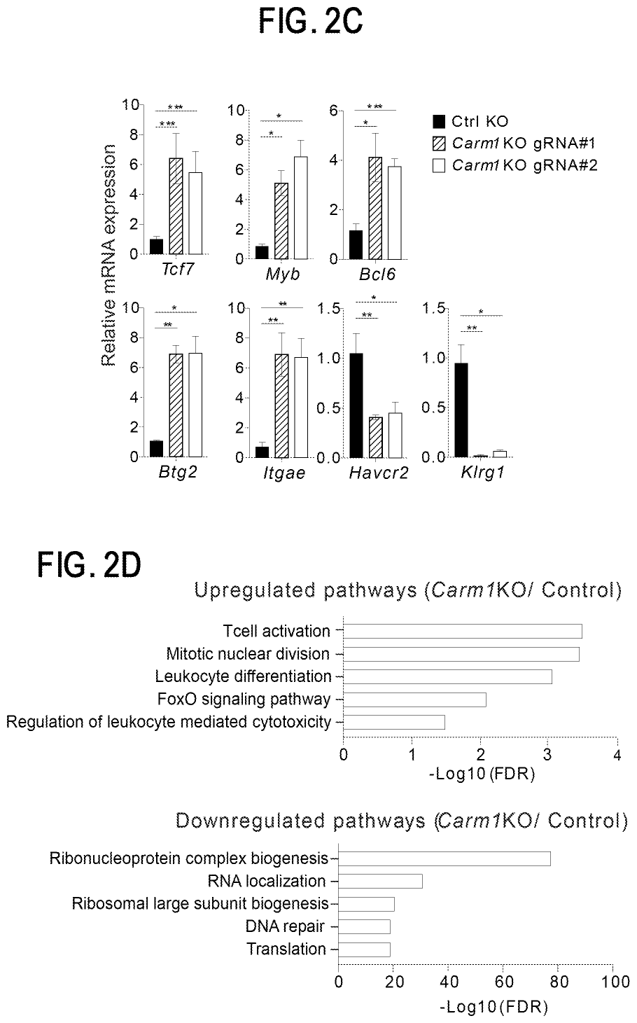

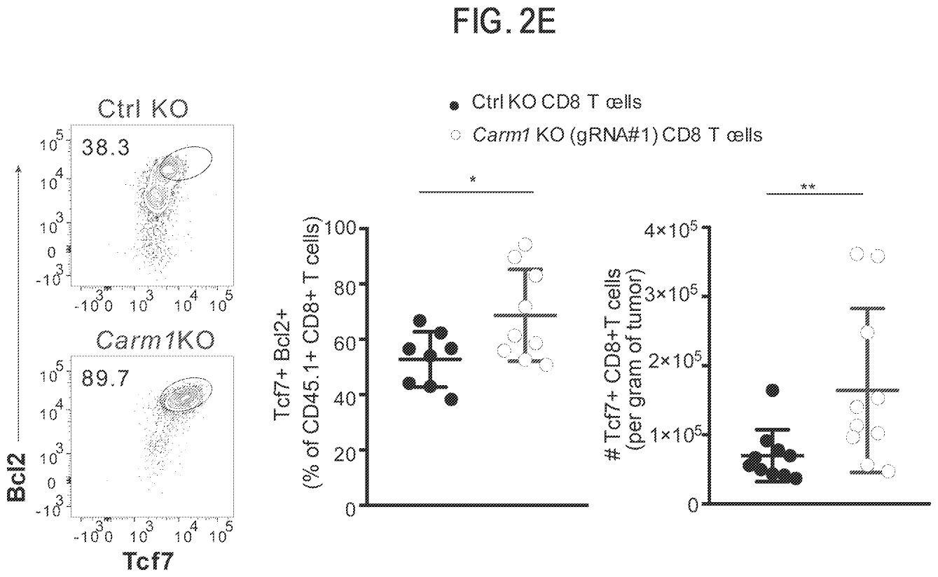

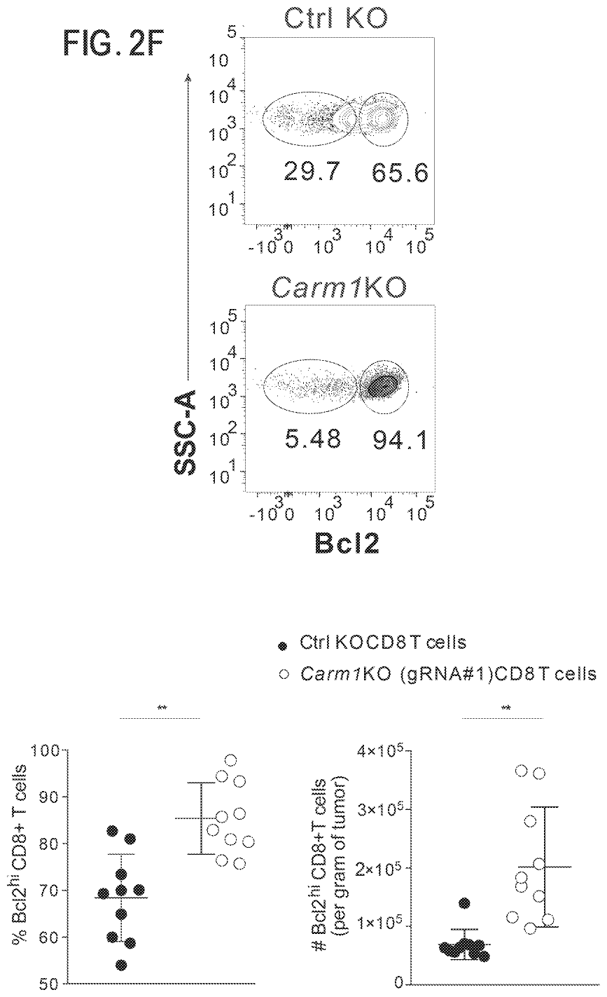

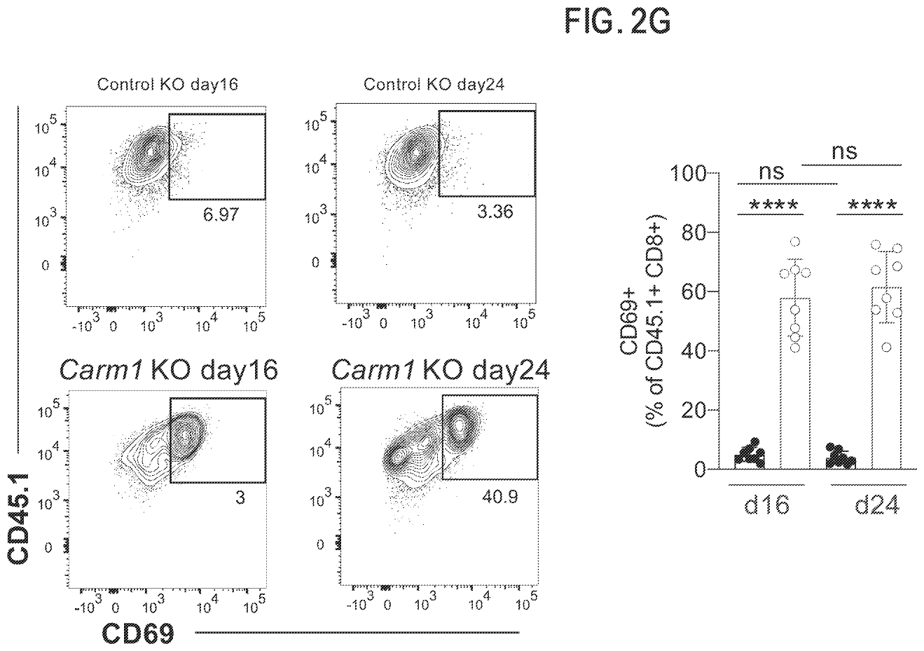

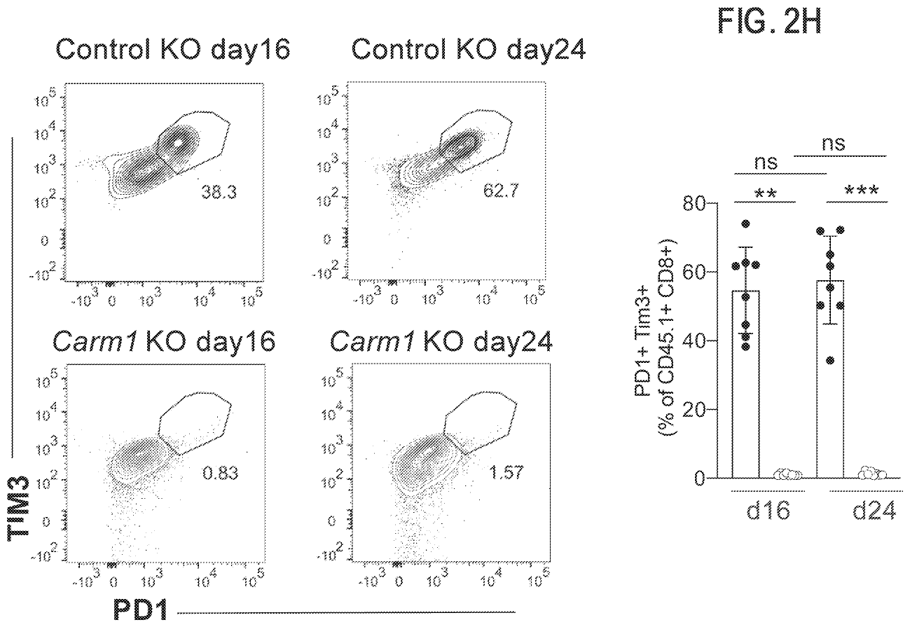

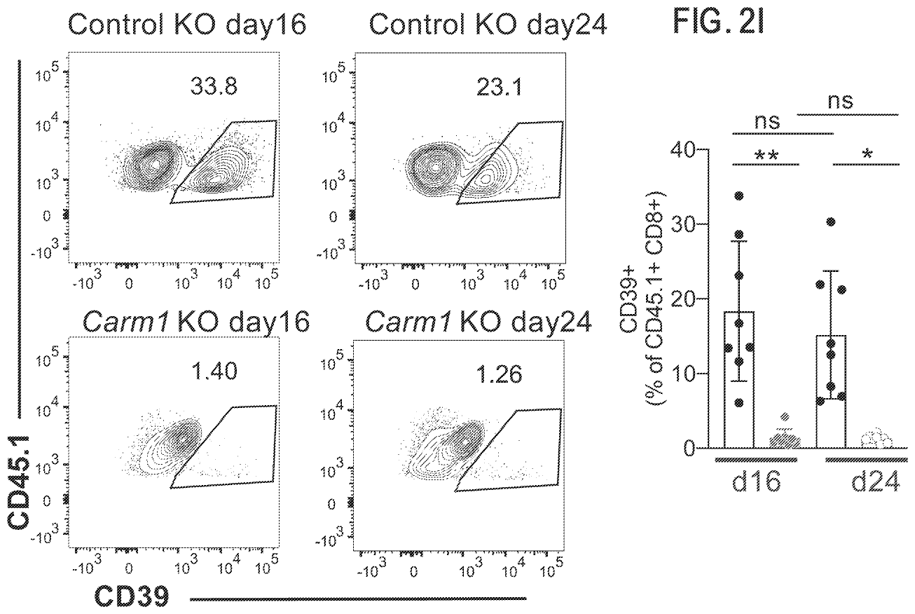

Seven days following editing, OT-I CD8 T cells were co-cultured for 24 hours with B16F10-Ova tumor cells. RNA-seq analysis demonstrated striking changes in gene expression, including upregulation of 1,143 genes and downregulation of 1,199 genes in Carm1-KO compared to control T cells (FIGS. 2A-2B). Upregulated genes encoded chemokine receptors that mediate T cell recruitment into tumors (Cxcr3) and key genes required for maintenance of memory T cell populations (transcription factors Tcf7, Myb, Bcl6; surface receptors Itgae and Cd27). Downregulated genes included those associated with terminal differentiation (Klrg1), inhibition of cytokine signaling (Socs1, Socs3) and T cell dysfunction within tumors (Egr2). To validate the RNA-seq results, qPCR analysis was performed using two different gRNAs and found that Tcf7, Myb, Bcl6 and Itgae (associated with T cell memory) were upregulated in Carm1-KO compared to control-KO CD8 T cells while Klrg1 (associated with terminal differentiation) and Havcr2 (associated with dysfunction) were downregulated in Carm1-KO T cells (FIG. 2C).

Gene set enrichment analysis (GSEA) highlighted ‘T cell activation’, ‘mitotic nuclear division’, ‘Foxo signaling’ pathway and ‘regulation of leukocyte mediated cytotoxicity’ among the top pathways for genes overexpressed in Carm1-KO compared to control T cells. Downregulated pathways related to RNA biology, protein translation and DNA repair (FIG. 2D). Genes in the Foxo signaling pathway identified by GSEA included Bcl6 and Il7r, both of which play important roles in T cell memory. A large fraction of tumor-infiltrating Carm1-KO CD8 T cells were positive for Tcf7 protein and also had high levels of Bc1-2, consistent with an increased pool of memory-like cells for Carm1-KO compared to control-KO T cells (FIG. 2E). Also, the number of tumor-infiltrating Bc1-2^{hi} cells was substantially higher for Carm1-KO compared to control-KO CD8 T cells (FIG. 2F). T cell persistence were investigated by studying tumor-infiltrating Carm1-KO and control-KO CD8 T cells at early (d16) and late (d24) time points following T cell transfer. A significantly larger population of Carm1-KO CD8 T cells expressed the CD69 activation marker at both time points (FIG. 2G). Conversely, only a small percentage of Carm1-KO T cells co-expressed the PD-1 and Tim-3 inhibitory receptors or CD39, which are markers of exhausted T cells (FIGS. 2H-2I). These data indicated that targeting of Carm1 in CD8 T cells enhanced their anti-tumor function and maintained a substantial population of tumor-infiltrating T cells that expressed memory markers.

### Example 3. Inactivation of Carm1 Gene in Tumor Cells Elicits Tumor Immunity

Analysis of RNA-seq data from 1,208 human cancer cell lines (Cancer Cell Line Encyclopedia, CCLE) revealed high-level CARM1 expression across a diverse range of human cancer cell lines (FIG. 3A). We therefore interrogated the role of Carm1 in tumor cells by inactivating the Carm1 gene in murine cancer cell lines resistant to checkpoint blockade, including B16F10 melanoma and 4T1 breast cancer models (FIGS. 3B-3C). In vitro proliferation and survival of B16F10 and 4T1 cells was not affected by inactivation of the Carm1 gene, but in vivo growth of Carm1-KO tumor cells was greatly diminished compared to control-KO tumor cells (FIG. 9E, FIG. 3D and FIG. 10A). Importantly, depletion of CD8 T cells restored the in vivo growth of Carm1-KO B16F10 cells, indicating that Carm1 inactivation in tumor cells elicited potent T cell-mediated tumor immunity (FIG. 3D and FIG. 10A). This conclusion was validated by comparing B16F10 tumor growth in immunocompetent wild-type and T cell-deficient T cell receptor a (Tcra) KO mice (FIG. 3E). Inactivation of the Carm1 gene in the 4T1 model of triple-negative breast cancer (TNBC) and the MC38 colon cancer model also substantially slowed tumor growth and conferred a survival benefit (FIGS. 3F-3H). Importantly, the number of spontaneous pulmonary metastasis was substantially reduced following orthotopic implantation of Carm1-KO compared to control-KO 4T1 tumor cells (FIG. 3G).

Next, an imaging-based T cell cytotoxicity assay was used to examine whether inactivation of the Carm1 gene sensitized tumor cells to killing by CD8 T cells. Indeed, Carm1-KO B16F10-Ova tumor cells were significantly more sensitive to T cell-mediated cytotoxicity than control-KO tumor cells, as shown by quantification of surviving live ZsGreen+ tumor cells or dying tumor cells labelled with a caspase 3/7 cell death reporter (FIGS. 3I-3J). A high-affinity small molecule inhibitor of CARM1 was reported (EZM2302) (Drew A E, Moradei O, Jacques S L, Rioux N, Boriack-Sjodin A P, Allain C, et al. Identification of a CARM1 Inhibitor with Potent In Vitro and In Vivo Activity in Preclinical Models of Multiple Myeloma. Sci Rep 2017; 7(1): 17993). Pre-treatment of B16F10-Ova tumor cells with EZM2302 also sensitized them to CD8 T cells (FIG. 3K). This CARM1 inhibitor also sensitized a human TNBC cell line to cytotoxic T cells. BT549 TNBC cells were co-cultured with human CD8 T cells that expressed a TCR specific for a NY-ESO-1 peptide presented by HLA-A2:01. Pre-treatment of these tumor cells with the CARM1 inhibitor (EZM2302) enhanced CD8 T cell-mediated cytotoxicity (FIG. 3L). These data demonstrated that inactivation of the Carm1 gene in tumor cells induced potent T cell-mediated immunity and that Carm1 deficient tumor cells were more sensitive to T cell-mediated cytotoxicity.

### Example 4. Innate Immune Activation in Carm1 Deficient Tumor Cells

RNA-seq analysis demonstrated striking changes in the transcriptome of Carm1-KO versus control-KO B16F10 tumor cells. In particular, we observed increased expression of many interferon response genes in Carm1-KO compared to control-KO tumor cells, even though these tumor cells had not been exposed to exogenous type 1 interferon or IFNγ (FIG. 4A, FIG. 10B). Gene set enrichment analysis (GSEA) also highlighted transcriptional activation of the IFN α/γ and p53 pathways in Carm1-KO tumor cells (FIG. 4B, FIG. 10C). In human melanoma, a higher type 1 interferon gene expression signature was found to correlate with increased CD8 T cell infiltration (Gajewski T F, Fuertes M B, Woo S R. Innate immune sensing of cancer: clues from an identified role for type I IFNs. Cancer Immunol Immunother 2012; 61(8): 1343-7). Importantly, there was little overlap in the genes that were differentially expressed as a consequence of Carm1 gene inactivation in tumor cells compared to T cells (FIG. 4C). In particular, the p53 pathway and interferon α/γ response pathways were only transcriptionally activated in Carm1-KO tumor cells but not Carm1-KO T cells. Validation by RT-qPCR using two Carm1 targeting gRNAs demonstrated that multiple interferon stimulated genes (ISGs) including Irf7, CCl5, Cxcl10, Ifit1, Oasl1, and Tap 1 were expressed at two to seven-fold higher levels in Carm1-KO compared to control-KO tumor cells (FIG. 4D). Importantly, pre-treatment of B16F10 cells with the CARM1 inhibitor EZM2302 also significantly increased the mRNA levels of these ISGs (FIG. 4E, FIG. 10D). EZM2302 also induced the expression of a similar set of ISGs in human breast and melanoma cancer cell lines (FIGS. 10E-10F).

The gene sets induced by type 1 interferons and IFNγ overlap substantially, and IFNγ secreted by activated T cells is an essential cytokine for protective tumor immunity (Dunn G P, Ikeda H, Bruce A T, Koebel C, Uppaluri R, Bui J, et al. Interferon-gamma and cancer immunoediting. Immunol Res 2005; 32(1-3): 231-45). Therefore, whether inactivation of the Carm1 gene in tumor cells enhanced their transcriptional response to IFNγ was investigated. Indeed, Carm1-KO B16F10 and 4T1 cells showed a heightened transcriptional response to IFNγ for all tested ISGs compared to control-KO tumor cells (FIGS. 10G-10H). IFNγ stimulation also induced higher levels of STAT1 phosphorylation in Carm1-KO compared to control-KO B16F10 cells, even though total STAT1 and STAT2 protein levels were similar between the cell lines (FIG. 11A). IFNγ inhibited the proliferation of Carm1-KO tumor cells more significantly than control-KO cells (FIG. 11B). Carm1-KO tumor cells expressed moderately higher levels of MHC class I protein (H2-K^{b}) both in the absence and following stimulation with IFNγ; PD-L1 levels were slightly higher for Carm1-KO compared to control-KO tumor cells following IFNγ stimulation (FIGS. 11C-11D). The enhanced transcriptional response of Carm1-KO tumor cells to IFNγ was attenuated when the Ifnar 1 gene was inactivated in Carm1-KO tumor cells implicating enhanced type 1 interferon signaling in this process (FIG. 11E). These data indicated that Carm1-KO tumor cells showed an increased responsiveness to IFNγ, an important cytokine secreted by activated T cells.

Next it was investigated which innate immune sensor was required for the interferon gene expression signature identified in Carm1-KO tumor cells. Inactivation of the Mavs gene, which encodes an essential adaptor protein for the double-stranded RNA sensors Rig-I and Mda-5, had no impact on expression of ISGs (FIGS. 12A-12B). In striking contrast, inactivation of the Cgas gene greatly diminished mRNA levels of ISGs, indicating that the cGAS enzyme represented a key element in the innate immune pathway activated in Carm1-KO tumor cells (FIG. 4F and FIGS. 12C-12D). Whether activation of cGAS could explain the enhanced sensitivity of Carm1-KO tumor cells to cytotoxic T cells was also tested. Indeed, Carm1-KO tumor cells were highly sensitive to CD8 T cells, while inactivation of Cgas in Carm1-KO tumor cells rendered them substantially more resistant to cytotoxic T cells (FIG. 4G). Inactivation of Cgas (without inactivation of Carm1) also rendered B16F10-Ova tumor cells more resistant to T cells compared to wild-type B16F10-Ova cells. These data indicated that the enhanced sensitivity of Carm1-KO compared to control-KO tumor cells to cytotoxic T cells required a functional cGAS-STING pathway, and that a lower level of activation of the cGAS-STING pathway in control-KO tumor cells was relevant for T cell-mediated killing.

These results strongly suggested that inactivation of Carm1 induced a DNA damage response in tumor cells. DNA damage induces rapid phosphorylation of histone H2AX (γH2AX) which provides a sensitive readout for double-stranded DNA (dsDNA) breaks (Kinner A, Wu W, Staudt C, Iliakis G. Gamma-H2AX in recognition and signaling of DNA double-strand breaks in the context of chromatin. Nucleic Acids Res 2008; 36(17): 5678-94). Immunofluorescence analysis showed that a substantial fraction of Carm1-KO B16F10 cells had multiple nuclear foci labeled with a γH2AX antibody while such foci were detected in only a small percentage of control-KO tumor cells (FIG. 4H). This conclusion was confirmed by labeling with an antibody specific for RAD51, another marker for dsDNA breaks (FIG. 4I). dsDNA breaks can induce chromosome mis-segregation during mitosis and formation of cytosolic micronuclei (Fenech M, Kirsch-Volders M, Nataraj an A T, Surralles J, Crott J W, Parry J, et al. Molecular mechanisms of micronucleus, nucleoplasmic bridge and nuclear bud formation in mammalian and human cells. Mutagenesis 2011; 26(1): 125-32). Such micronuclei tend to have fragile nuclear envelopes, resulting in exposure of their dsDNA to cGAS (Mackenzie K J, Carroll P, Martin C A, Murina O, Fluteau A, Simpson D J, et al. cGAS surveillance of micronuclei links genome instability to innate immunity. Nature 2017; 548(7668): 461-5). DAPI+ micronuclei were detected in a significantly larger percentage of Carm1-KO compared to control-KO tumor cells (FIG. 4J). A subset of these cytosolic DAPI+ micronuclei were positive for cGAS when an epitope-tagged version of cGAS was expressed in tumor cells (FIG. 12E). CARM1 inhibitor treatment also induced γH2AX foci accumulation in B16F10 melanoma cells (FIG. 12F). Using a gRNA targeting an intergenic region in the mouse genome, we confirmed that CRISPR/Cas9 based gene editing (>1 week prior to analysis) did not result in substantial dsDNA damage or ISG expression (FIGS. 12G-1211). These results demonstrated that inactivation of the Carm1 gene induced innate immune activation within tumor cells due to activation of the cGAS-STING pathway. CARM1 and p300 were previously shown to cooperate with BRCA1 and p53 to induce expression of the cell cycle inhibitor p21^{CIP1} (CDKN1A) (Lee Y H, Bedford M T, Stallcup M R. Regulated recruitment of tumor suppressor BRCA1 to the p21 gene by coactivator methylation. Genes Dev 2011; 25(2): 176-88). Failure of cell cycle inhibition following DNA damage can result in chromosome segregation during mitosis, formation of micronuclei and cGAS activation (Bakhoum S F, Cantley L C. The Multifaceted Role of Chromosomal Instability in Cancer and Its Microenvironment. Cell 2018; 174(6): 1347-60).

Interestingly, unlike tumor cells, Carm1 ablation did not induce dsDNA damage in CD8 T cells (FIG. 4K). Also, Carm1 inactivation resulted in distinct gene expression changes in T cells versus tumor cells (FIG. 4C, FIGS. 2A-2C), suggesting that Carm1 induced cell type specific consequences in T cells (enhanced function, preserved pool of memory-like cells) compared to tumor cells (DNA damage and induction of cGAS-STING signaling).

### Example 5. Carm1 Inhibition Overcomes Resistance to Checkpoint Blockade

Many human tumor types resistant to checkpoint blockade with CTLA-4 or PD-1 mAbs are poorly infiltrated by CD8 T cells (‘cold tumors’). Poor infiltration by CD8 T cells is associated with an absence of a type 1 interferon gene expression signature (Gao J, Shi L Z, Zhao H, Chen J, Xiong L, He Q, et al. Loss of IFN-gamma Pathway Genes in Tumor Cells as a Mechanism of Resistance to Anti-CTLA-4 Therapy. Cell 2016; 167(2): 397-404; Thorsson V, Gibbs D L, Brown S D, Wolf D, Bortone D S, Ou Yang T H, et al. The Immune Landscape of Cancer. Immunity 2019; 51(2): 411-2). It was hypothesized that treatment with a CARM1 inhibitor could be effective in checkpoint blockade-resistant tumors by enhancing the function of tumor-specific T cells and also increasing the sensitivity of tumor cells to cytotoxic T cells. B16F10 melanomas are resistant to monotherapy with CTLA-4 or PD-1 mAbs and even combination therapy with both checkpoint antibodies (Curran M A, Montalvo W, Yagita H, Allison J P. PD-1 and CTLA-4 combination blockade expands infiltrating T cells and reduces regulatory T and myeloid cells within B16 melanoma tumors. Proc Natl Acad Sci USA 2010; 107(9): 4275-80). Treatment of Carm1-KO tumors with CTLA-4 or PD1 blockade substantially reduced tumor growth and conferred a significant survival benefit (FIG. 5A and FIG. 13A). Importantly, small molecule-mediated Carm1 inhibition (EZM2302) also sensitized B16F10 melanomas to checkpoint blockade with a CTLA-4 mAb and resulted in a significant survival benefit (FIG. 5B). The inhibitor was administered at a dose of 150 mg/kg but optimization of drug dose for this application was not feasible due to limited availability of this compound. This inhibitor was previously only evaluated in an immunodeficient mouse model and shown to moderately slow the in vivo growth of a human multiple myeloma cell line (RPMI-8226). The highly metastatic 4T1 model of TNBC is also resistant to CTLA-4 blockade. Inactivation of the Carm1 gene sensitized 4T1 tumors to CTLA-4 blockade and conferred a marked survival benefit when compared to the three other experimental groups (FIG. 5C). CTLA-4 mAb treated mice with Carm1-KO 4T1 tumors also showed a substantial reduction in the number of spontaneous lung metastases, again in comparison to all other treatment groups (FIG. 5D).

Analysis of Carm1-KO B16F10 tumors showed a striking increase in the number of infiltrating CD8 T cells and a substantial reduction in the percentage of CD8 T cells that expressed the inhibitory PD-1 and Tim-3 inhibitory receptors (FIGS. 5E-5G). In contrast, the number of tumor-infiltrating CD4 T cells (calculated per gram of tumor) was similar between all treatment groups, although the percentage of CD4 T cells in the total T cell pool was lower in Carm1-KO compared to control-KO tumors due to the striking increase in CD8 T cell accumulation (FIG. 13B, FIG. 5E). CTLA-4 mAb treatment further enhanced CD8 T cell infiltration into Carm1-KO B16F10 tumors compared to the three other experimental groups and reduced the percentage of CD8 T cells double positive for the PD-1 and Tim-3 inhibitory receptors (FIGS. 5E-5G). Also, a substantially larger percentage of CD8 T cells from Carm1-KO compared to control-KO CTLA-4 treated tumors were positive for key functional markers including granzyme B and IFNγ (FIG. 5H and FIG. 13C). Carm1-KO tumors (treated with control or CTLA-4 mAbs) were also infiltrated by a larger number of dendritic cells, including cross-presenting cDC1 cells, as well as NK cells, when compared to control-KO tumors treated with either mAb (FIG. 5I, FIG. 13D). Similar changes were observed in Carm1-KO 4T1 tumors in particular following treatment with a CTLA-4 mAb (FIGS. 13E-13I). These data demonstrated that tumor cell-intrinsic inactivation of the Carm1 gene induced multiple significant changes in the tumor microenvironment, including enhanced infiltration by CD8 T cells, NK cells and dendritic cells. Also, CD8 T cells expressed substantially lower levels of the PD-1 and Tim-3 inhibitory receptors consistent with a reduced level of T cell exhaustion.

The impact of CARM1 inhibitor treatment on the tumor immune microenvironment was also investigated, both as monotherapy as well as in combination with PD-1 or CTLA-4 mAbs (FIGS. 14, 15 and 16). In toxicity studies with this CARM1 inhibitor, no histological changes was observed in a comprehensive analysis of major organs or weight loss between the CARM1 inhibitor versus solvent control treatment groups (FIGS. 14A-14B). CD8 T cell infiltration was substantially increased following monotherapy with the CARM1 inhibitor and was even higher when the inhibitor was combined with PD-1 or CTLA-4 mAbs (FIG. 15A). In contrast, CD8 T cell infiltration was not increased following PD-1 or CTLA-4 monotherapy compared to the isotype control antibody group. We also observed a striking increase in the number of CD8 T cells (per gram of tumor) that expressed granzyme B, IL-2, IFNγ, and perforin in all three CARM1 inhibitor treatment groups (FIGS. 15B-15E). In contrast, PD-1 expression by CD8 T cells was reduced in CARM1 inhibitor treatment groups compared the vehicle control group (FIG. 15F). CARM1 inhibitor treatment did not change the number of tumor-infiltrating CD4 T cells or FoxP3+ Tregs. However, the CD8/FoxP3 Treg ratio was substantially increased in all three Carm1 treatment groups due to the increase in CD8 T cell infiltration (FIG. 16A-16D). Interestingly, tumor-infiltrating NK cells were also increased in all three CARM1 inhibitor treatment groups and were highest when the CARM1 inhibitor was combined with PD-1 or CTLA-4 mAbs. cDC1 cells were increased when the CARM1 inhibitor was combined with PD-1 or CTLA-4 mAbs, but no changes in macrophage numbers were detected across any of the treatment groups (FIG. 16E). These data demonstrated that small molecule-mediated inhibition of Carm1 induced major favorable changes in the tumor immune microenvironment, in particular for CD8 T cells, NK cells and cDC1. These favorable changes were further enhanced when the CARM1 inhibitor was combined with PD-1 or CTLA-4 mAbs.

To examine whether re-expression of Carm1 in tumor cells reversed the phenotype induced by Carm1 inactivation, we introduced a Carm1 cDNA under the control of a doxycycline (Dox)-inducible promoter into Carm1-KO B16F10 tumor cells (FIGS. 17A-17B). Dox treatment suppressed mRNA levels of IFNγ inducible genes in Carm1-KO cells, consistent with our finding that control-KO tumor showed lower responsiveness to IFNγ than Carm1-KO tumor cells (FIGS. 17C and 10G-H). Control-KO B16F10 tumor cells transduced with the empty vector showed rapid growth, regardless of whether mice were treated with vehicle or Dox (FIG. 17D). As expected, Carm1-KO tumor cells transduced with the Carm1 vector but treated with vehicle grew slowly, with similar kinetics as Carm1-KO tumor cells transduced with the empty vector. In contrast, Dox treatment substantially accelerated growth of Carm1-KO tumor cells transduced with the Carm1 vector. Importantly, key aspects of the tumor microenvironment were also reversed by Dox-induced re-expression of Carm1 in Carm1-KO tumor cells, including the striking degree of CD8 T cell infiltration, lower levels PD-1 expression by CD8 T cells and the increase in cDC1 infiltration (FIGS. 18A-18D).

### Example 6. Tdrd3 And Med12 Are Effector Molecules of The Carm1 Pathway

Carm1 deposits H3R17me2a and H3R26me2a methylarginine marks on histone tails that are recognized by the Tudor domain-containing protein Tdrd3 (Yang Y, Lu Y, Espejo A, Wu J, Xu W, Liang S, et al. TDRD3 is an effector molecule for arginine-methylated histone marks. Molecular cell 2010; 40(6): 1016-23). Carm1 also methylates a number of other nuclear proteins, including Med12, a component of the regulatory arm of the Mediator complex (Cheng D, Vemulapalli V, Lu Y, Shen J, Aoyagi S, Fry C J, et al. CARM1 methylates MED12 to regulate its RNA-binding ability. Life Sci Alliance 2018; 1(5): e201800117). We found that inactivation of either Tdrd3 or Med12 genes in B16F10 cells increased mRNA levels of multiple ISGs, similar to inactivation of Carm1 (FIGS. 6A-6B, FIG. 19A-19D). Furthermore, this ISG gene expression signature was lost when the Cgas gene was also inactivated in Tdrd3-KO or Med12-KO B16F10 cells (FIGS. 6A-6B). Similar to Carm1 ablation, Tdrd3 or Med12 inactivation resulted in higher sensitivity to IFNγ and led to enhanced IFNγ-induced expression of ISGs (FIGS. 19E-19F). Inactivation of genes encoding other Tdrd3 or Med12 associated proteins (Top3b, Top1 and Med13) did not result in a substantial increase in ISG mRNA levels except for some upregulation of ISGs in Med13-KO tumor cells (FIGS. 19G-19I). Inactivation of Tdrd3 or Med12 genes in B16F10 tumor cells also resulted in accumulation of γH2AX positive nuclear foci and cytosolic micronuclei, as described above for Carm1-KO B16F10 cells (FIGS. 6C-6D). Finally, it was found that Tdrd3-KO tumors showed substantially attenuated growth, and that this phenotype was again CD8 T cell dependent (FIGS. 6E-6F). CTLA-4 mAb treatment further inhibited the growth of Tdrd3-KO tumor cells and resulted in survival of a large fraction of the mice (FIG. 6G).

Biochemical studies showed that Med12 was indeed a direct target of Carm1. When Med12 was immunoprecipitated from control-KO B16F10 cells, asymmetric methylation of arginine residues of Med12 could be readily detected by Western blot analysis (FIG. 6H), consistent with a previous report that identified Med12 as a target of Carm1 (Cheng D, Vemulapalli V, Lu Y, Shen J, Aoyagi S, Fry C J, et al. CARM1 methylates MED12 to regulate its RNA-binding ability. Life Sci Alliance 2018; 1(5): e201800117). This asymmetric methylation of arginine residues of Med12 was absent in Carm1-KO B16F10 tumor cells (but not in Tdrd3-KO tumor cells, as expected). Furthermore, immunoprecipitation of Med12 from nuclear lysates showed that less histone H3 was bound to Med12 in Carm1-KO compared to control-KO cells, indicating that Carm1 facilitated recruitment of Med12 to histone H3 (FIG. 6I). These results demonstrated that inactivation of the Tdrd3 and Med12 genes resulted in a similar immune-mediated phenotype as inactivation of the Carm1 gene.

Whether RNA Pol II mediated transcription was altered in Carm1-KO compared to control-KO tumor cells was further investigated. Western blot analysis of the C-terminal domain (CTD) of RNA Pol II showed increased phosphorylation of Ser2 (p-Ser2, a mark of transcriptional elongation) (Buratowski S. Progression through the RNA polymerase II CTD cycle. Mol Cell 2009; 36(4): 541-6) and Ser5 in nuclear lysates from Carm1-KO compared with control-KO cells (FIG. 20A). Pol II phosphorylation was systematically investigated using mammalian native elongating transcript sequencing (mNET-Seq) (Nojima T, Gomes T, Carmo-Fonseca M, Proudfoot N J. Mammalian NET-seq analysis defines nascent RNA profiles and associated RNA processing genome-wide. Nat Protoc 2016; 11(3): 413-28). Inactivation of the Carm1 gene increased the normalized read density of p-Ser2 CTD Pol II tags relative total Pol II tags around immediate promoter regions of genes (FIGS. 20B-20C). Also, ˜25% of the genes upregulated in Carm1-KO compared to control-KO cells were associated with higher normalized RNA Pol II pSer2 status (FIG. 20D). GSEA analysis of these overlap genes showed an enrichment in the p53 pathway, consistent with the data presented in FIG. 4 (FIG. 20E). These data provided evidence for altered transcriptional regulation in Carm1 deficient tumor cells, consistent with the identification of Med12 as a Carm1 target. Furthermore, increased abundance of alternatively spliced genes (exon gains in 638 genes and exon losses in 708 genes) were found in Carm1-KO tumor cells. Pathways analysis using these alternatively spliced genes identified DNA repair, regulation of transcription and chromatin organization to be among top enriched pathways in Carm1-KO tumor cells (FIG. 20F). Co-transcriptional R-loop structures have been linked to genome instability and are thought to be resolved through TDRD3 and TOP3B (Yang Y, McBride K M, Hensley S, Lu Y, Chedin F, Bedford M T. Arginine methylation facilitates the recruitment of TOP3B to chromatin to prevent R loop accumulation. Molecular cell 2014; 53(3): 484-97). S9.6-based DNA:RNA Immunoprecipitation approaches (DRIP-seq) (Sanz L A, Chedin F. High-resolution, strand-specific R-loop mapping via S9.6-based DNA-RNA immunoprecipitation and high-throughput sequencing. Nat Protoc 2019; 14(6): 1734-55) were used to determine whether R-loops were increased as a result of Carm1-KO. No significant trend towards R-loop gains was observed (FIG. 20G), arguing that co-transcriptional R-loops over genic regions were not a source of the genomic instability observed in Carm1-KO cells.

### Example 7. Relevance of CARM1 In Human Cancers

The relevance of CARM1 in human cancers, including a potential role of CARM1 in tumor cells, was next investigated. It was found that gene signatures for a number of important pathways were downregulated in human cancer cell lines with high versus low CARM1 mRNA levels, including the p53, MHC class I antigen presentation and interferon-related pathways (FIG. 7A). These results suggested that CARM1 also served an important role in human tumor cells by inhibiting important immune pathways.

Analysis of The Cancer Genome Atlas (TCGA) RNA-seq datasets indicated that CARM1 may regulate immune responses in human cancers. In a large number of human cancer types, CARM1 mRNA levels were negatively correlated with gene signatures of tumor infiltration by T cells and APCs as well as response to IFNγ and IFNα (FIG. 7B, FIG. 21A). Furthermore, CARM1 mRNA levels positively correlated with a gene signature for tumor infiltration by immunosuppressive myeloid derived suppressor cells (MDSC). To better understand the effect of CARM1 expression in human tumors, an analysis of differentially regulated pathways was performed for two of the cancer types investigated above [TCGA skin cutaneous melanoma (SKCM) and lung squamous cell carcinoma (LUSC)]. Tumors with high versus low CARM1 mRNA levels showed downregulation of interferon gamma and alpha response pathways, while pathways related to Myc targets V1/V2 and G2M checkpoints were upregulated (FIG. 7C).

RNA-seq data from TCGA enable analysis of a large number of tumors but lack single cell resolution. The malignant cell populations in independent human tumor scRNA-seq datasets from multiple cancer types was therefore also investigated. Again, we found that high CARM1 mRNA levels negatively correlated with p53 and DNA repair pathways as well as key immune pathways (APC infiltration and response to IFNα/IFNγ); high CARM1 mRNA levels were also associated with reduced survival in a number of human cancer types (FIGS. 7D, 21B-21C, and 22A-22B).

CARM1 gene expression in clinical immune checkpoint blockade (ICB) datasets was also examined, analyzing 16 clinical trial datasets. Low expression of CARM1 per se was not associated with ICB response. Given that MED12 was a downstream target of CARM1, the role of MED12 and CARM1 mRNA levels on response to PD-1 pathway inhibition was investigated: the cohorts were first separated based on MED12 mRNA levels, and then the correlation of CARM1 mRNA levels in MED12 low (<median) and MED12 high (>median) cohorts examined. In four clinical cohorts, it was found that ICB responders had significantly lower expression levels of both MED12 and CARM1 compared to non-responders (two trials of PD-1 blockade in melanoma, one trial of PD-L1 blockade in kidney cancer, one trial of PD-1 blockade in glioblastoma) (FIG. 7E). In addition, higher levels of a Carm1-KO gene expression signature were associated with response to ICB in four clinical cohorts; this signature was obtained from RNA-seq analysis of Carm1-KO versus control-KO B16F10 tumor cells (FIG. 23A). In TCGA RNA-seq datasets, this Carm1-KO signature positively correlated with gene signatures of CTL infiltration, antigen processing and presentation as well as interferon γ response (FIG. 23B). These data provided evidence that CARM1 expression was associated with major immune pathways in human cancers.

### Methods

### Cell Lines

B16F10, 4T1, MC38 parental cell lines were purchased from American Type Culture Collection (ATCC). B16-OVA-ZsGreen cells were generated by lentiviral transduction of the parental line with a pHAGE expression vector driving expression of an N-terminally truncated variant of chicken ovalbumin (subcloned from pcDNA3-deltaOVA, Addgene plasmid #6459525). zsGreen+ cells were sorted to purity to establish the cell line. B16-OVA-zsGreen cells were validated for expression of zsGreen and cell surface presentation of the OVA peptide SIINFEKL in complex with H2-K^{b}. Furthermore, the cells were tested for their ability to activate OT-I CD8 T cells. Murine Carm1 cDNA was synthesized and cloned into pINDUCER21-ORF-EG (Addgene Plasmid #46948) using gBlocks™ from IDT to generate the Dox-Carm1 construct. Empty vector or Dox-Carm1 transduced B16F10 control-KO or Carm1-KO cells were sorted for purity based on GFP expression. Dox-inducible Carm1 protein expression was confirmed by Western blotting. B16F10 and 4T1 cells were grown in DMEM and RPMI media, respectively, supplemented with 10% FBS and 1% Penicillin/Streptomycin. MC38 cells were grown in DMEM media supplemented with 10% fetal bovine serum (FBS), 100 IU/ml Penicillin/Streptomycin, 5 mM non-essential amino acids, 5 mM sodium pyruvate and 5 mM HEPES at 37° C. with 5% CO₂. Cell lines were validated for Mycoplasma contamination using the ATCC Universal Mycoplasma Detection Kit.

### Mice

6-8-week-old male mice were used for all experiments. WT C57BL/6 mice (JAX stock #000664), Balb/c (JAX stock #000651), and Tcra-KO mice (JAX stock #002116) were purchased from the Jackson Laboratory. OT-I (JAX stock #003831) were crossed with the CD45.1 congenic strain (JAX #002014). OT-I Cas9 double transgenic mice were generated by crossing OT-I mice (JAX stock #003831) with mice carrying a Rosa26-targeted knock-in of Streptococcus pyogenes Cas9 (JAX stock #024858) with constitutive Cas9 expression. The presence of the Cas9 transgene was verified according to genotyping protocols published by Jackson Labs. All purchased mice were acclimated for one week to housing conditions at the Dana-Farber Cancer Institute Animal Resource Facility prior to all experiments. Colonies for each strain of mice were maintained in the same animal facility. Mice were housed in pathogen-free conditions and in accordance with the animal care guidelines from the Dana-Farber Cancer Institute standing committee on Animals and the National Institutes of Health. Animal protocols were approved by the DFCI IACUC.

### In Vivo CRISPR/Cas9 Screen in Tumor Infiltrating CD8 T Cells

### A. Cloning of Epigenetic Grna Library and Virus Production

For the primary screen, we constructed three lentiviral plasmid libraries of gRNA sequences targeting a total of 426 genes that encoded epigenetic regulators. Each library contained five unique gRNA sequences that targeted 142 candidate genes. In addition, gRNAs targeting six genes previously shown to inhibit CD8 T cell accumulation in tumors were included as positive controls (Pdcd1, Ctla4, Cblb, Egr2, Smad2 and Ppp2r2d). We also included 100 gRNA sequences as negative controls. These gRNA libraries were cloned into the lentiviral plasmid vector pLKO-gRNA-Thy1.1 that drove expression of the Thy1.1 surface marker. For this purpose, the pLKO.3G vector (Addgene plasmid #14748) was modified by replacing eGFP with the Thy1.1 coding sequence. gRNA libraries were then cloned into the resulting lentiviral expression vector.

For the validation screen, a new gRNA library was constructed by targeting 31 genes selected from the top hits of the primary screens as well as Pdcd1 and Cblb as positive control genes (six gRNA/gene) (Genetic Perturbation Platform, Broad Institute). As negative controls, 186 gRNAs were added (93 non-targeting plus 93 intergenic gRNAs).

To generate lentivirus for transduction with pooled gRNA libraries, the following transfection mix was generated for each 162 cm² tissue culture flask of HEK293T cells: 7 μg of pLKO-gRNA-Thy1.1 plasmid prep containing lentiviral gRNA sequence libraries, 7 μg of pCMV-DR.9.1 and 0.7 μg of pCMV-VSV-G in 700 μl of OPTI-MEM serum-free media (Gibco) plus 42 μl of TransiT-293 transfection reagent (Minis). This transfection mix was added to low-passage HEK293T cells (80-90% confluence) in 162 cm² tissue culture flasks followed by overnight incubation. The next day, the media was removed and replaced with 20 ml of RPMI supplemented with 20% FCS. Viral supernatants were collected at 48- and 72-hours post transduction (2×20 ml supernatant total per 162 cm2 flask), passed through a 0.45 μM filtration unit (ThermoFisher) and concentrated by ultracentrifugation at 112,000×g. Viral titers were determined by transducing HEK293T cells with serial dilutions of a small aliquot of the concentrated prep and measuring the percentage of Thy1.1 positive HEK cells by flow cytometry.

### B. Transduction of OT-I Cas9 CD8 T Cells

Spleens and peripheral lymph nodes (inguinal, axillary and cervical nodes) from OT-I Cas9 mice were mechanically dissociated using 70 μm cell strainers in complete RPMI medium [containing 10% FBS+1× GlutaMax™ (Gibco), 100 U/ml penicillin-streptomycin, 1 mM sodium pyruvate, 20 mM HEPES, and 50 μM 2-mercaptoethanol]. OT-I Cas9 CD8 T cells (>97% purity) were isolated from single-cell suspensions using an EasySep™ Mouse CD8+ T Cell Isolation Kit (Stemcell Technologies) according to the manufacturer's instructions. T cells were cultured in complete RPMI media supplemented with 100 ng/ml IL-15 (Biolegend) and 5 ng/ml IL-7 (Biolegend) for 48 hours. T cells were then transduced with lentiviral gRNA libraries by spin-infection with concentrated lentivirus preps (MOI=15) using retronectin-coated (Takara Bio) 24-well non-tissue culture treated plates (2×10⁶ cells/well). Spin-infection was done at 2,000 rpm for 1.5-2 hours at 32° C. in a total volume of 1 ml of virus prep plus complete RPMI media supplemented with 5 μg/ml protamine sulphate (Sigma-Aldrich). Cells were cultured for 72 hours post-transduction in complete RPMI media supplemented with 50 ng/ml IL-15 and 2.5 ng/ml IL-7 (Biolegend) before magnetic enrichment of Thy1.1⁺ cells using an EasySep™ Mouse CD90.1 Positive Selection Kit (StemCell Technologies); this approach resulted in purity of Thy1.1+ cells of >93%.

C. In Vivo Screen with Tumor Infiltrating Cd8 T Cells

gRNA transduced Thy1.1⁺ OT-I Cas9 CD8 T cells (5×10⁶) were injected intravenously into each of 10-12 C57BL/6 mice with B16-OVA-ZsGreen tumors (>25 mm² tumor area). On day 10 following T cell transfer, tumors and spleens were isolated for recovery of transferred Thy1.1⁺CD8 T cells. Tumors were dissociated using GentleMACS C tubes (Miltenyi Biotec) on a GentleMACS dissociator (Miltenyi Biotec) with the ‘37C_m_TDK_1’ program and an enzyme mix containing 1 mg/ml collagenase D (Sigma-Aldrich), 20 U/ml DNase I (Sigma Aldrich) and 100 μg/ml hyaluronidase type V (Sigma Aldrich) in RPMI media (without additional supplements). Total tumor cell suspensions were then centrifuged at 50×g for 5 minutes, and supernatants were collected. Spleens were mechanically dissociated using 70 μm cell strainers, and total CD8 T cells were then isolated using EasySep™ Mouse CD8+ T Cell Isolation Kits (Stemcell Technologies) according to the manufacturer's instructions. Live singlet Thy1.1⁺ TCRβ⁺ CD8⁺CD4⁻ cells were sorted from tumor and spleen suspensions using a FACSAria IIIu cell sorter (BD) equipped with a 70 μm nozzle and the ‘Yield’ purity mask to ensure complete collection of events. Cell pellets were washed once with cold PBS, and genomic DNA was extracted with a Zymogen Quick-gDNA Microprep Kit following the manufacturer's protocol for suspension cells.

D. Sequencing of gRNA Libraries and Quantification of gRNA Representation

Genomic DNA isolated from tumor-infiltrating OT-I Thy1.1+CD8 T cells was subjected to PCR amplification of the gRNA cassette for Illumina sequencing of gRNA representation by the Genetic Perturbation Platform of the Broad Institute of MIT and Harvard (Cambridge, Mass.). Protocols for PCR amplification and Illumina sequencing are described in detailed at https://portals.broadinstitute.org/gpp/public/resources/protocols.

Data analysis was performed using MaGeCK (Model-based Analysis of Genome-wide CRISPR-Cas9 Knockout) (Li W, Xu H, Xiao T, Cong L, Love M I, Zhang F, et al. MAGeCK enables robust identification of essential genes from genome-scale CRISPR/Cas9 knockout screens. Genome Biol 2014; 15(12): 554). For candidate gene discovery, the normalized gRNA count table was loaded into MaGeCK by comparing tumor (experimental) and spleen (control) conditions described above. Top genes were determined based on mean log 2 fold change (LFC) for all gRNAs and false discovery rate (FDR).

Assays with Edited T Cells and Tumor Cells

### Editing of Genes in Tumor Cells

Cells were edited by electroporation with ribonucleoprotein complexes (RNP) composed of Cas9 protein with bound gRNAs. 20 μM of gRNA (designed using Genomic Perturbation Platform of Broad Institute) was mixed at equimolar ratio with Cas9 protein (obtained from UC Berkeley). Editing of genes in tumor cells was performed by nucleofection using the SF cell line 96-well nucleofector kit on 10⁵ tumor cells per nucleofection reaction. Gene editing efficiency was determined by DNA sequencing and subsequent TIDE analysis as well as Western blot analysis.

Adoptive Transfer of Edited T Cells into Tumor-Bearing Mice

Editing of OT-I CD45.1 CD8 T cells was performed by electroporation with RNP composed of Cas9 protein (20 μM) and bound gRNA (20 μM) using the P3 primary cell 96-well nucleofector kit (Lonza) (2×10⁶ cells per electroporation condition). Freshly isolated naive OT-I CD45.1+CD8 T cells were edited and then cultured with CD3+CD28+ Dynabeads (Invitrogen) for 24 h in complete RPMI media [RPMI 1640 medium (Life Technologies 11875119) containing 1% penicillin/streptomycin (Life Technologies 15140122), 50 μM β-mercaptoethanol (Life Technologies 21985023), and 1% L-glutamine (Life Technologies 25030081) supplemented with 10% FBS (Life Technologies 10437028) and 2 ng/ml IL-2+2.5 ng/mL IL-7+50 ng/mL IL-15]. Dynabeads were then removed and cells were cultured for five additional days in fresh media containing 2.5 ng/mL IL-7+50 ng/mL IL-15. Editing efficiency with Carm1 gRNA was confirmed by Western blot analysis. Carm1 or control edited T cells (1×10⁶ cells in 100 μL of PBS) were then adoptively transferred to C57BL/6 mice bearing B16-OVA-ZsGreen tumors. An additional group of mice was injected with 100 μL of PBS (no T cell control). Tumor size was measured every third day using a digital caliper.

### Generation of Primary Human CD8 T Cells Expressing NY-ESO-1 Specific TCR

Peripheral blood mononuclear cells (PBMCs) were isolated by Ficoll density gradient centrifugation from leukapheresis collars of healthy donors (Brigham and Women's Hospital Blood Bank). CD8 T cells were purified from PBMCs using CD8 Dynabeads (StemCell #19053) following the manufacturer's instructions. Isolated CD8 cells were activated for 48 hours with αCD3/αCD28 beads (Life Technologies #11132D, 1:2 ratio of beads to T cells) and grown in the presence of 30 U/mL of human IL-2 for one week. Expanded CD8 T cells were transduced with the lentivirus by spin infection to introduce the NY-ESO-1 TCR. A non-tissue culture treated 24 well plate was coated with 0.8 ml of 15 μg/ml Retronectin (Takara; Kyoto, Japan) overnight at 4° C. Wells were blocked with sterile 2% BSA for 15 minutes at room temperature and gently washed once with PBS. Next, lentivirus was added to wells of the retronectin-coated plate at a multiplicity of infection (MOI) of 15, and plates were spun for 2.5 hours at 2,000×g, 32° C. The supernatant in the wells was then carefully decanted, and wells were gently washed with 0.5 ml of PBS. 0.5×10⁶ T cells were transferred to wells containing 10 μg/ml protamine sulfate (Sigma-Aldrich) in RPMI-1640 media containing 30 U/ml IL-2 and cultured for three days. NY-ESO-1 TCR+ T cells were isolated to >90% purity by FACS and expanded with Dynabeads and IL-2 (30 U/ml).

### T Cell Cytotoxicity Assays

A Celigo image cytometer (Nexcelom) was used to study the killing of fluorescent tumor cells by CD8 T cells. Carm1-KO or control-KO B16-OVA-ZsGreen cells were washed with PBS, and 5,000 tumor cells were added per well in flat-bottom 96-well plates (8-10 replicates/group). OT-I CD8 T cells were added at different effector to target ratios. Following 24 or 48 hours of co-culture, the media was removed, and wells were washed with PBS to remove dead tumor cells and CD8 T cells. Live, adherent tumor cells were then counted using the Celigo image cytometer. As an alternative approach, apoptotic tumor cells were counted based on Caspase 3/7 activation. A Caspase-3/7 reagent (Essen Bioscience) was added directly to the culture media (0.5 μM final concentration) after 12, 24 or 48 hours of co-culture of tumor cells with T cells, and positive tumor cells were counted using the image cytometer.

T cell cytotoxicity assay using human tumor cells was performed using NY-ESO-1 transduced human CD8 T cells and BT549 human triple negative breast cancer cells which were HLA-A02*01 positive and endogenously expressed the NY-ESO-1 antigen. BT549 cells were co-cultured with human CD8 T cells that expressed a NY-ESO-1 TCR at increasing effector to target (E:T) ratios for 12-72 hours. Cytotoxicity was quantified by flow cytometry. All in vitro cytotoxicity assays were performed in human or murine T cell media (without addition of IL-2).

### RNA Extraction and RT-qPCR

Total RNA was extracted using the RNeasy Mini Kit (Qiagen) according to the manufacturer's protocol. Extracted RNA (1 μg) was transcribed into cDNA using SuperScript IV VILO master mix with ezDNase enzyme according to the manufacturer's protocol (ThermoFisher). The cDNA samples were diluted and used for real-time quantitative PCR (RT-qPCR). Taqman master mix (ThermoFisher) and gene-specific primers were used for PCR amplification and detection using a QuantStudio 6 Flex Real-Time PCR System (ThermoFisher). The RT-qPCR data were normalized to GAPDH and HPRT (housekeeping genes) and presented as fold change of gene expression in the test sample compared to the control sample.

### Protein Extraction and Immunoblotting

Whole cell extracts were prepared by lysis and sonication of cells in RIPA buffer [20 mM Tris-HCl (pH 7.5), 150 mM NaCl, 1 mM EDTA, 1 mM EGTA, 1% NP-40, 1% sodium deoxycholate, 2.5 mM sodium pyrophosphate, 1 mM β-glycerophosphate, 1 mM Na3VO4, 1 μg/ml leupeptin] supplemented with Halt Protease and Phosphatase Inhibitor Cocktail (ThermoFisher). Protein concentrations were determined using a Bradford Protein Assay (Bio-Rad). Protein samples (20 □g) were resolved by SDS-PAGE using 4-12% NuPAGE Novex Bis-Tris mini gels (ThermoFisher) and transferred onto a PVDF membrane (Bio-Rad). Blots were blocked in 5% Blocker powder (Bio-Rad), 0.2% Tween in PBS and then incubated overnight with primary antibodies. Following incubation with secondary detection reagents and subsequent washing, blots were incubated in Western Lightening Plus-ECL substrate (PerkinElmer). Luminescence was captured using a ChemiDoc MP system (Bio-Rad Laboratories).

### Tumor Cell Colony Formation Assay

B16F10, 4T1 and MC38 cells were trypsinized and transferred into fresh media, counted and diluted appropriately for seeding into 6-well plates at a density of 500 cells/well. Cells were allowed to grow for 5-6 days, with fresh media added at day 3. Cells were washed with PBS and stained with crystal violet solution (0.5% w/v crystal violet powder, 80% v/v H₂O and 20% v/v methanol). Number of colonies and colony areas were quantified using ImageJ software based on the user's manual.

### Immunofluorescence Imaging

Cells were grown on glass coverslips, washed with PBS and fixed in 4% paraformaldehyde in PBS for 20 min at room temperature. Cells were permeabilized in 0.2% Triton X-100 for 15 min before blocking for 30 min with 10% serum-containing blocker (ThermoFisher). Coverslips were then incubated with primary antibody overnight at 4° C. in a humidified chamber, followed by incubation with a secondary antibody for 1 hour. Primary and secondary antibodies were diluted in blocking buffer, and all incubations were performed at room temperature. Coverslips were mounted using Prolong Gold Antifade Mounting Medium with DAPI (ThermoFisher). Imaging was performed using a Hamamatsu ORCA-Flash4.0 V3 Digital CMOS camera and Nikon Ti-E Motorized Microscope 2000U microscope with Plan Apo Lamda 60×/1.40 Oil Ph3 DM objectives. Images were captured with Nikon Elements Acquisition Software. All scoring was performed under blinded conditions. Three independent experiments with three biological replicates per group were performed.

### Quantification of Micronuclei in Tumor Cells

Tumor cells were stained with DAPI, and the percentage of cells that were positive for cytoplasmic micronuclei was determined using Nikon Ti inverted microscope using Plan Apo λ, 100×/1.45 Oil DIC objective lens. Micronuclei were defined as discrete DNA aggregates separate from the primary nucleus for cells in which the morphology of the primary nucleus was normal. Cells with apoptotic appearance were excluded from the analysis. All scoring was performed under blinded conditions. Three independent experiments with three biological replicates per group (>100 cells counted per replicate) were performed.

### Mouse Tumor Models

Female BALB/c (Jackson Laboratory #000651) or C57BL/6J (Jackson Laboratory #000664) mice of 4-6 weeks of age were purchased from The Jackson Laboratory. B16F10 or MC38 tumor cells (2×10⁵) were injected subcutaneously in 50 μL of PBS into syngeneic C57BL/6J mice. 4T1 TNBC cells (1×10⁵) were injected in 100 μl of PBS supplemented with Matrigel orthotopically into the mammary fat pads of syngeneic BALB/c mice. Mice with similar tumor burden were randomized into treatment groups. Depletion of CD8 T cells in BALB/c and C57BL/6J mice was achieved by IP injection of 100 μg of CD8β mAb (Bio X Cell, Clone 53-5.8 #BE0223) in 100 μL of PBS on days −1, day 0, and then every 3rd day post tumor inoculation. Mice receiving an isotype control mAb (Bio X Cell, clone HRPN #BE0088) at the same dose in PBS were used as controls. CD8 T cell depletion was confirmed by labeling of CD8 T cells from spleens with a CD8 mAb (Biolegend #100741) followed by flow cytometric analysis (BD Fortessa, BD Biosciences). CD8 T cells were significantly depleted within 24 hours following administration of CD8β antibody and at the experimental endpoint.

For checkpoint blockade experiments, tumor-bearing mice were administered with anti-CTLA4 mAb (clone 9H10, #BP0131, 100 μg/injection) or corresponding isotype control Ab (polyclonal Syrian hamster IgG, 100 μg/mouse). Alternatively, mice received anti-PD1 (clone 29F.1A12, #BE0273, 200 m/injection) or rat IgG2a isotype control Ab, anti-trinitrophenol (Clone: 2A3, 200 μg/injection) starting on day 7 post tumor inoculation and then every 3rd day. The specific endpoint for each experiment is indicated in the figure legends.

For CARM1 inhibitor experiments, mice received CARM1 inhibitor EZM2302 (dose of 150 mg/kg in 100 μL) or vehicle (5% Dextrose) twice daily via oral gavage. Inhibitor treatment was performed for 14 days because limited quantities of the compound were available.

### 4T1 Metastasis Assay

Carm1-KO or control-KO 4T1 cells (105 cells) were injected into the mammary fat pad of 6-week old BALB/c mice. Three weeks later, lung tissue was washed three times with PBS and fixed in Bouin's solution (10 mL per lung) for 4-5 days. Visible metastatic nodules were counted under a stereomicroscope (Leica).

### Doxycycline—Inducible Expression of Carm1 In Vivo

C57Bl/6 mice bearing Carm1-KO B16F10 tumor cells (transduced with Dox-Carm1 or empty vector constructs) were fed a doxycycline containing diet (625 ppm, Envigo Teclad) until the experimental endpoint (18 days). Mice receiving the regular feed were used as controls. Intra-tumoral expression of Carm1 was confirmed by Western blotting at the experimental endpoint.

### Flow Cytometry Analysis of Tumor—Infiltrating Immune Cells

Tumors were excised on day 18 following tumor cell inoculation and cut into small pieces using sterile scalpels in serum-free RPMI 1640 media (ThermoFisher #11875093). Tissue was dissociated in 1 mg/ml Collagenase Type IV (Sigma-Aldrich #C5138), 20 units/ml DNAse Type IV (Sigma-Aldrich #D5205), 0.1 mg/ml Hyaluronidase Type V (Sigma-Aldrich #H6254) using GentleMACS C or M tubes on the GentleMACS™ Dissociator (Miltenyi Biotec #130-093-235) followed by incubation at 37° C. for 20 min. The resulting cell suspension was passed through a 70 μm filter and pelleted by centrifugation at 300×g for 5 min. To remove red blood cells, ACK lysis buffer (3× by volume) was added for 45-60 seconds followed by 2 volumes of RPMI to stop red cell lysis. Pelleted cells from pooled supernatants (>300×g or 1500 rpm, 5 min) were resuspend in the appropriate buffer for flow cytometric analysis of tumors.

Single cell suspensions were stained with 5 μg/mL of Fc receptor blocking anti-mouse CD16/CD32 antibody (clone 2.4G2, BD PharMingen) at 4° C. for 5 min before staining of surface proteins with an antibody cocktail at 4° C. for 30 min in a volume of 100 μL. Cells were then washed twice with PBS, stained with LIVE/DEAD Fixable Dead Cell Stain Kit (Molecular Probes) at 4° C. for 15 min and washed twice with staining buffer (PBS supplemented with 1% BSA and 2 mM EDTA). Finally, cells were fixed by incubation in BD Cytofix Fixation Buffer (BD Biosciences) at 4° C. for 30 min. Samples were analyzed using a BD LSR Fortessa X-20 cell analyzer and BD FACSDiva Software version 8.0. For intracellular staining, cells were stained with surface markers, fixed in Fix/Perm buffer (eBioscience) for 15 min, washed in permeabilization buffer (eBioscience) twice and stained with primary antibodies targeting intracellular proteins in permeabilization buffer for 30 min at 4° C. Data analysis was performed on FlowJo 10.

### Bulk RNA-Seq Analysis

B16F10 tumor cells or OT-I CD8 T cells were edited with Carm1 or control gRNAs, and loss of Carm1 protein expression was confirmed by Western blot analysis. Three biological replicates were used to extract total RNA using the RNeasy Plus Mini Kit (Qiagen #74134) according to the manufacturer's protocol. RNA quality was checked using an Agilent BioAnalyzer 2000 instrument. RNA with an integrity number of greater than 9.5 was used for subsequent analyses. RNA-seq analysis was performed by GeneWiz. The standard mRNA library preparation TruSeq RNA Sample Prep Kit v2 (Illumina) was used for library preparation. DNA concentration of libraries was quantified by Qubit (Invitrogen), and equal quantities of DNA were mixed for sequencing. Single-end 75 bp sequencing was performed for edited tumor cells, whereas paired-end 150 bp sequencing was performed for edited CD8 T cells on an Illumina NextSeq 500 instrument. Gene count quantification was performed with RSEM (Li B, Dewey C N. RSEM: accurate transcript quantification from RNA-Seq data with or without a reference genome. BMC Bioinformatics 2011; 12: 323). In the Carm1 KO tumors, we derived a list of genes from the tumor Carm1 KO RNA-seq study, where differential expression study (DES) was performed between the Carm1 KO group and the control group. Using the cutoff of “abs(og2FC)>0.05 & FDR<0.05 & TMP>1”, we obtained 146 upregulated genes and 18 downregulated genes. Together, these 164 genes represent a gene signature of ‘Carm1 knockout’ or ‘Carm1 inhibition’, such that the derived gene signature is enriched in Carm1 deficient cells. Statistics for differentially expressed genes were calculated by DESeq2 (Love M I, Huber W, Anders S. Moderated estimation of fold change and dispersion for RNA-seq data with DESeq2. Genome Biol 2014; 15(12): 550). Criteria of log 2FC>1, FDR<0.05, and average TPM>1 were utilized to call significantly differentially expressed genes for the CD8 T cell KO analysis and criteria of FDR<0.01 was used for tumor cell KO experiment.

Mammalian Native Elongating Transcript Sequencing (mNET-Seq)

The samples were processed as recommended previously (Nojima T, Gomes T, Carmo-Fonseca M, Proudfoot N J. Mammalian NET-seq analysis defines nascent RNA profiles and associated RNA processing genome-wide. Nat Protoc 2016; 11(3): 413-28). Briefly, three sets of 8×10⁶ cells per experimental condition were processed separately and pooled after MNase treatment. Protein G Dynabeads (ThermoFisher, 10004D) were coupled with the following antibodies: RNAPII Ser2-P (Abcam, Rabbit Poly Ab #5095, 5 μg/IP), RNAPII CTD (clone 8WG16, Biolegend #664912, 5 μg/IP). NEBNext Small RNA-seq Library Prep kit (#E7300S) was used for library synthesis. Prior to library preparation, sample quality assessed using the Agilent RNA 6000 Pico Kit (#5067-1513). Samples were processed per manufacturer's protocol for Next-Generation Sequencing (NGS) with the exception of the size selection step, which was performed as described previously (29). We selected barcoded fragments of 150-250 bp, which correspond to RNA fragments of 23 bp-123 bp. Samples were sequenced on the Illumina Nextseq500 platform using a PE75 flow cell. Analysis was performed by the Molecular Biology Core Facility at Dana Farber Cancer Institute as described previously (Nojima T, Gomes T, Carmo-Fonseca M, Proudfoot N J. Mammalian NET-seq analysis defines nascent RNA profiles and associated RNA processing genome-wide. Nat Protoc 2016; 11(3): 413-28).

### Computational Analyses

mNET-Seq Data Analysis

mNET-Seq reads were processed as described in (Nojima T, Gomes T, Grosso A R F, Kimura H, Dye M J, Dhir S, et al. Mammalian NET-Seq Reveals Genome-wide Nascent Transcription Coupled to RNA Processing. Cell 2015; 161(3): 526-40). Briefly, adapters were removed using software tool Cutadapt (version 1.18), and the trimmed reads were aligned to the mouse genome (mm10) using TopHat (version 2.1.1). Location and strandness of the 5′-end of the second read in each concordant read pair were identified and used for further analysis. R coding environment (r-project.org) was used to compute read frequencies and perform statistical analyses.

### Cancer Cell Line Encyclopedia (CCLE) Analysis

To evaluate CARM1 expression and its association with different molecular phenotypes in human cancer cell lines, we collected and curated RNA-seq and mutational profiles of 1,208 cell lines from Depmap (Ghandi M, Huang F W, Jane-Valbuena J, Kryukov G V, Lo C C, McDonald E R, 3rd, et al. Next-generation characterization of the Cancer Cell Line Encyclopedia. Nature 2019; 569(7757): 503-8). To investigate associations between CARM1 expression and biomarkers and pathways, we fit linear regression models with biomarker and pathway score as output variable and CARM1 mRNA level as input variable, and with cancer type adjusted. Adjusted p-values were retrieved and reported for each biomarker and pathway.

### TCGA Data Analysis

Cancer data sets were collected with both patient survival duration and tumor gene expression profiles from the TCGA database (Weinstein J N, Collisson E A, Mills G B, Shaw K R, Ozenberger B A, Ellrott K, et al. The Cancer Genome Atlas Pan-Cancer analysis project. Nat Genet 2013; 45(10): 1113-20). When clinical information was available, we separated the breast cancer data sets into the PAM50 (Prediction Analysis of Microarray 50) subtypes (Parker J S, Mullins M, Cheang M C, Leung S, Voduc D, Vickery T, et al. Supervised risk predictor of breast cancer based on intrinsic subtypes. J Clin Oncol 2009; 27(8): 1160-7) of luminal A, luminal B, Her2 positive, and basal. Each PAM50 subtype is known to have a distinct genomics profile (Hoadley K A, Yau C, Wolf D M, Cherniack A D, Tamborero D, Ng S, et al. Multiplatform analysis of 12 cancer types reveals molecular classification within and across tissues of origin. Cell 2014; 158(4): 929-44) and degree of cytotoxic T cell infiltration (Miyan M, Schmidt-Mende J, Kiessling R, Poschke I, de Boniface J. Differential tumor infiltration by T-cells characterizes intrinsic molecular subtypes in breast cancer. J Transl Med 2016; 14(1): 227). Among all TCGA cancers, melanoma is annotated in terms of primary and metastatic sites; Head and neck cancer (HNSC) is annotated corresponding to its HPV status. The CARM1 expression level was compared between cancer tissues and their matched normal tissue when both datasets were available. For each sample, the transcriptomic profile was log 2(1+TPM) transformed. We standardized the log scale transcriptome data across patients by quantile-normalization, and further normalized the expression of each gene by subtracting the average of all samples, where a zero value indicated the average expression. The correlations between CARM1 expression and pathway scores were assessed by Spearman correlations. To further assess the clinical relevance of CARM1 expression in cancer, we examined whether CARM1 expression and MED12 were linked to survival benefits in multiple cancer types using Cox regression model.

### Human Single-Cell RNA-Seq Data Analysis

CARM1 expression in human malignant cells was evaluated at single cell resolution. Human malignant cell scRNA-seq data from basal cell carcinoma (GSE123813) (3,551 cells) (Tirosh I, Izar B, Prakadan S M, Wadsworth M R, 2nd, Treacy D, Trombetta J J, et al. Dissecting the multicellular ecosystem of metastatic melanoma by single-cell RNA-seq. Science 2016; 352(6282): 189-96), head and neck squamous cell carcinoma (GSE103322) (2,488 cells) (Zhang Z, Liu R, Jin R, Fan Y, Li T, Shuai Y, et al. Integrating Clinical and Genetic Analysis of Perineural Invasion in Head and Neck Squamous Cell Carcinoma. Front Oncol 2019; 9:434), acute myeloid leukemia (GSE116256) (12,489 cells) (van Galen P, Hovestadt V, Wadsworth Ii M H, Hughes T K, Griffin G K, Battaglia S, et al. Single-Cell RNA-Seq Reveals AML Hierarchies Relevant to Disease Progression and Immunity. Cell 2019; 176(6): 1265-81.e24), acute lymphoblastic leukemia (GSE132509) (21,370 cells), multiple myeloma (GSE141299) (16,840 cells), non-small cell lung cancer (GSE143423) (9,237 cells) were retrieved and processed. For each collected dataset, quality control, clustering, and cell-type annotation was uniformly performed. The annotated malignant cells were confirmed by inferCNV (Patel A P, Tirosh I, Trombetta J J, Shalek A K, Gillespie S M, Wakimoto H, et al. Single-cell RNA-seq highlights intratumoral heterogeneity in primary glioblastoma. Science 2014; 344(6190): 1396-401) based on their copy number variations (CNVs). The pathway scores for the single cell data were derived using AddModuleScore module from Seurat package (Butler A, Hoffman P, Smibert P, Papalexi E, Satija R. Integrating single-cell transcriptomic data across different conditions, technologies, and species. Nature biotechnology 2018; 36(5): 411-20). Statistical comparisons were made with two-sided unpaired Mann-Whitney tests and Spearman correlations.

### Gene Ontology and Pathway Enrichment Analysis

Gene ontology and pathway enrichment were performed with Metascape (Zhou Y, Zhou B, Pache L, Chang M, Khodabakhshi A H, Tanaseichuk O, et al. Metascape provides a biologist-oriented resource for the analysis of systems-level datasets. Nat Commun 2019; 10(1): 1523) or GSEA/mSigDB (Liberzon A, Birger C, Thorvaldsdottir H, Ghandi M, Mesirov J P, Tamayo P. The Molecular Signatures Database (MSigDB) hallmark gene set collection. Cell Syst 2015; 1(6): 417-25). For the bulk tumor Carm1 knockout in our studies, differentially expressed genes were selected for pathway enrichment studies. Pathway signature genes were obtained from the GSEA/mSigDB hallmark gene set collection. In bulk RNA data, pathway signature scores were calculated with mean normalized mRNA expression in each data set. Spearman correlations were calculated for the expression patterns of the pathway signatures in the CCLE, TCGA, and scRNA-seq data. The correlations were adjusted with estimated purity score where both data were available in the TCGA data.

### Statistical Analyses

Statistical analyses were performed using R3.6 and GraphPad Prism 6 software. Each experiment was performed 2-3 times as indicated. Unpaired Student's t-test, 2-way ANOVA or unpaired two-sided Mann-Whitney test were used as indicated for comparisons between two groups, *p<0.05, **p<0.01, ***p<0.001, ****p<0.0001, ns (non-significant). For in vivo studies, sample size was determined as a function of effect size ((difference in means)/(SD)=2.0) for a two-sample t-test comparison assuming a significance level of 5%, a power of 90%, and a two-sided t-test. Normal distribution was confirmed using normal probability plot (GraphPad Prism 6.0, GraphPad Software, San Diego, Calif.), variance was assessed within and between groups. The means of groups were compared using Student's t-test. The growth of primary tumors over time was analyzed using two-way ANOVA with multiple comparisons. For comparing mouse survival curves, a Log-rank (Mantel-Cox) test was used. All p-values are two-sided, and statistical significance was evaluated at the 0.05 level.

### Data and Materials Availability

All transcriptomics data generated during this study (RNA-seq, mNET-seq and DRIP-seq) have been deposited at NCBI GEO (Gene Expression Omnibus) with accession numbers GSE144917, GSE149139 and GSE148905.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1. A method of treating a subject having a cancer, comprising:
a) reducing expression of a Carm1 gene and/or a Carm1 effector gene in a cell of the subject; and/or
b) reducing activity of a Carm1 protein and/or a Carm1 effector protein in a cell of the subject,
wherein the cancer is resistant to immunotherapy and/or checkpoint blockade treatment.

2. The method of claim 1, wherein the Carm1 effector gene is a Tdrd3 gene and the Carm1 effector protein is a Tdrd3 protein.

3. The method of claim 1, wherein the Carm1 effector gene is a Med12 gene and the Carm1 effector protein is a Med12 protein.

4. The method of claim 1, wherein the reducing step comprises administering to the subject an inhibiting agent, wherein the inhibiting agent inhibits the expression of the Carm1 gene or the Carm1 effector gene and/or the activity of the Carm1 protein or the Carm1 effector protein in the subject.

5. The method of claim 4, wherein the inhibiting agent is selected from the group consisting of a polynucleotide, a polypeptide, an antibody, a small molecule, a protein degrader, and a combination thereof.

6. The method of claim 4, wherein the inhibiting agent comprises EZM2302 or TP-064.

7. The method of claim 5, wherein the protein degrader comprises a Carm1 protein degrader, a Tdrd3 protein degrader, and/or a Med12 protein degrader.

8. The method of claim 7, wherein the protein degrader comprises a Carm1 protein degrader.

9. The method of claim 1, wherein the reducing step comprises silencing the Carm1 gene or the Carm1 effector gene in the subject by shRNA mediated knockdown of mRNA or inactivation of genes.

10. The method of claim 1, wherein the reducing step comprises modifying the Carm1 gene or the Carm1 effector gene to decrease the expression of the Carm1 gene or the Carm1 effector gene.

11. The method of claim 10, wherein the modifying step comprises modifying the Carm1 gene or the Carm1 effector gene by a CRISPR/Cas system.

12. The method of claim 1, wherein the cell is an immune cell.

13. The method of claim 12, wherein the immune cell is an immune effector cell, wherein the reduced expression of the Carm1 gene or the Carm1 effector gene and/or reduced activity of the Carm1 protein or the Carm1 effector protein enhances the cytotoxic function of the immune effector cell and/or reduces exhaustion of the immune effector cell.

14. The method of claim 12, wherein the immune effector cell is selected from the group consisting of a cytotoxic T cell, a tumor-infiltrating lymphocyte (TIL), a Natural Killer T cell (NKT), a cytotoxic T lymphocyte (CTL), a dendritic cell, a CD8 T cell and a CD4 T cell.

15. The method of claim 1, wherein the cell is a cancer cell.

16-18. (canceled)

19. The method of claim 1, further comprising:
administering to the subject an immune cell having tumor specificity to the cancer and having reduced expression of the Carm1 gene or the Carm1 effector gene and/or reduced activity of the Carm1 protein or the Carm1 effector protein.

20-22. (canceled)

23. The method of claim 1, wherein the cancer is a melanoma, carcinoma, sarcomas, adenocarcinoma, lymphoma, leukemia, kidney, breast, lung, bladder, colon, ovarian, prostate, pancreas, stomach, brain, head and neck, skin, uterine, testicular, glioma, esophagus, or liver cancer.

24. The method of claim 1, wherein the cancer is resistant to checkpoint blockade treatment with a CTLA-4, PD-L1, TIM-3, LAG3, TIGIT, or PD-1 antibody blockade therapy.

25. The method of claim 24, wherein the checkpoint blockade is selected from a group consisting of Nivolumab, Pembrolizumab, Ipilimumab, Atezolizumab, Avelumab, Durvalumab, Cemiplimab, and a combination thereof.

26. The method of claim 1, further comprising:
administering to the subject a pharmaceutically effective amount of a second therapeutic agent for treating cancer in the subject.

27. The method of claim 26, wherein the second therapeutic agent is selected from the group consisting of a chemotherapy agent, an immunotherapy agent, a checkpoint blockade agent, a toxin, a radiolabel, a siRNA, a cancer vaccine, a small molecule, a peptide, an antibody, a genetically engineered cell, a CAR T cell, a cytokine and a combination thereof.

28. (canceled)

29. A method of treating cancer in a subject, comprising:
reducing expression of a Carm1 gene or a Carm1 effector gene and/or activity of a Carm1 protein or a Carm1 effector protein in a cell of the subject.

30. The method of claim 29, further comprising:
administering to the subject a pharmaceutically effective amount of a second therapeutic agent for treating cancer in the subject.

31-37. (canceled)

38. A method of sensitizing a cancer cell to an immune effector cell, comprising:
inhibiting expression and/or activity of a Carm1 gene or protein, or a Carm1 effector gene or protein in a cancer cell by one or more inhibiting agents, wherein the inhibiting sensitizes the cancer cell to an immune effector cell.

39-45. (canceled)

46. A method of increasing the anti-tumor function of an immune effector cell, comprising:
reducing expression and/or activity of a Carm1 gene or protein, or a Carm1 effector gene or protein in the immune effector cell, thereby increasing the anti-tumor function of the immune effector cell.

47-56. (canceled)

57. The method of claim 46, wherein the reducing step comprises silencing the CARM1 gene or the Carm1 effector gene in the immune effector cell.

58. The method of claim 46, wherein the reducing step comprises degrading the Carm1 protein or the Carm1 effector protein in the immune effector cell.

59-60. (canceled)

61. An immune effector cell, the immune cell having an inhibiting agent of a Carm1 gene/protein or a Carm1 effector gene/protein, wherein the inhibiting agent inhibits expression of the Carm1 gene or Carm1 effector gene, and/or activity of the Carm1 protein or the Carm1 effector protein.

62-69. (canceled)

70. The immune effector cell of claim 61, wherein the immune effector cell is tumor specific.

71. The immune effector cell of claim 61, wherein the immune effector cell expresses a tumor-specific T-cell receptor or a chimeric antigen receptor (CAR).

72-77. (canceled)

78. A composition comprising the immune effector cell of claim 61 and a pharmaceutically acceptable carrier.

79. The composition of claim 78, further comprising a second therapeutic agent.

80-82. (canceled)

83. A method of treating cancer in a subject, comprising:
administering to a subject having cancer the immune effector cell of claim 61.

84. The method of claim 83, wherein the immune effector cell is autologous.

85. The method of claim 83, wherein the immune effector cell is specific to a cancer cell of the subject.

86. The method of claim 83, further comprising administering to the subject having cancer a second therapeutic agent, or a composition comprising a second therapeutic agent and a pharmaceutically acceptable carrier.

87-88. (canceled)
